SEQUENCE LISTING (377 sequences)

SEQ ID NO: 1 - DNA, 291 bp, Homo sapiens
  misc_feature (1)...(291): n = A,T,C or G
cgcggggacaaactacttcatcaaggtgcacgtcggcgacnaggacttcgtacacctgcgagtgttccaatctctccctcatgaaaacaagcccttgaccttatctaactaccagaccaacaaagccaagcatgatgagctgacctatttctgatcctgactttggacaaggcccttcagccagaanactgacaaagtcatcctccgtctaccanancgtgcacttgtgatcctaaaataagcttcatctncgggctgtgccccttggggtggaaggggcaggattctgca

SEQ ID NO: 2 - DNA, 549 bp, Homo sapiens
  misc_feature (1)...(549): n = A,T,C or G
gcttagttttacattcacaaagtatttgtatttgataagtcttaaaaatgatttaagttgaagcctaacagaagtacttgttcattcctcagttacctgcaaataatggaaataatggtgcagtcagttttatttgattgtgtaatgctttatactaggggtctcaaacccctgggtggcattagattcttacaggagcgtgaaccctattgtgaactgcacatgcaagagatctaggttgtgcgctcattatgagaatctaatacccgatgatctgaggtggaacagtctcatnctgaaaaccatctaccccaacacctgtgtctgtggaaattgtctccacgaaaccggtccctgttgaaaaaaggttaggggaccactgctttatacagtgtttgaagtaggcacacttacagttactatttctggttctttatgactgttgaggggggcagaattctatgtttgattcacagtaaaacttttcatgaatcattgaggggttaactgttatcacttgccaatggaaantggatattgaaacatttt

SEQ ID NO: 3 - DNA, 298 bp, Homo sapiens
  misc_feature (1)...(298): n = A,T,C or G
gtggtgcgcgcccgcnagcagatcgtagctggctgctggcacanaaaggcatctatttttcaatggtcagtgtccaggctggaacaaagcgccagtgaactctgactgtataagatgttaaatactttttaatatttgtttagatatgacatttattcaaagttaaaagcaaacactnncagaattatgaagaggtatctgtttaacatttnctcagtcaagttcagagtcttcagagacttcgtaattaaaggaacagagtgagagacatcatcaagtggagagaaatcataggttt

SEQ ID NO: 4 - DNA, 218 bp, Homo sapiens
ccgggcaggtaaaaaacctttgaaaaatatacagcttgatattatttacataaaatatgaatccaggttccaatatcaaacaaacattgctatgtcagaaacacagtggaaggcaggaacgtaactcactgccttttagatgcaaagactaatagacacgttctcctatctcgactatctttgttacctgttatcctcaaacataaattattaggcac

SEQ ID NO: 5 - DNA, 218 bp, Homo sapiens
cctgacattcctgccttcttataataagaaaaataaaacaaaatagtgttgaagtgttggggcggcgaaaatttttggggggtggaatggagagagaatgggcgatgtttctcagggctgcttcaagcgggattaggggcggcgtgggaacctagagtgggagagattaagctgaagggaggtcttgtggtaaggggtgatattgtggggatgttaaa

SEQ ID NO: 6 - DNA, 495 bp, Homo sapiens
  misc_feature (1)...(495): n = A,T,C or G
aaaaagaaaaaaaaagccaaatacattttctgacattgtaagattgccttactgtctgtcattccttattgctggcccctttctcaggccggaggccaagtggtggagaaggaaaggaaatgatcgaacgggcatgttgtcaagtgggcatgccactgggaaataccaccagtttaccctgaaacattgtcctcagaggagtaggaaagtggattttgaatctctattttgctcaaaagttcagttcctgagatactgatgactgagagtgctgctgggaaattttcaggattgtgtggtcttttggggttttttgttttttttttttaagacaaagttgaccgctgttcactgtccacgtgatcagttgtaagattacaatgctgcatgctagttggttacataagatacaattccagtgatggaaggcggttataatggatggtggtgtgtacaanatggcactgccatctttgagcagagcccagctctgca

SEQ ID NO: 7 - DNA, 363 bp, Homo sapiens
  misc_feature (1)...(363): n = A,T,C or G
ccttcggacagtagataagtatccaaagcagtgtgggcaaaaagagtaagcaacatcccctgggatttttcccatgtgggtatcactgagcaaagctgctgtaggaagatatgtagattaatgggaaatgcagggttattatggaggaaataactcacaggatgatttcatagcctggctaaattactacttgattaactcctgataaaactcagagcgcacaaagcgaggcagagaatccttttccatcagggcatggattcttttctgggccatgtcaaagctgctcanggaaaggttccaccaggtttcttcattgtgatgtccttagtgaagtggtcaatattcacctctttaggagcc

SEQ ID NO: 8 - DNA, 403 bp, Homo sapiens
ctgtccaatggcaacatgaccctcactctactcagcgtcaaaaggaacgatgcaggatcctatgaatgtgaaatacagaacccagcgagtgccaaccgcagtgacccagtcaccctgaatgtcctctatggcccagatggccccaccatttccccctcaaaggccaattaccgtccaggggaaaatctgaacctctcctgccacgcagcctctaacccacctgcacagtactcttggtttatcaatgggacgttccagcaatccacacaagagctctttatccccaacatcactgtgaataatagcggatcctatatgtgccaagcccataactcagccactggcctcaataggaccacagtcacgatgaccacagtctctggaagtgctcctgtcctctcag

SEQ ID NO: 9 - DNA, 331 bp, Homo sapiens
tttttaactcctctcgctctgatgggacatttgttacccttttttcatagtgaaattgtgtttcaggcttagtctgacctttctggtttcttcattttcttccattacttaggaaagagtggaaactccactaaaatttctctgtgttgttacagtcttagaggttgcagtactatattgtaagctttggtgtttgtttaattagcaatagggatggtaggattcaaatgtgtgtcatttagaagtggaagctattagcaccaatgacataaatacatacaagacacacaactaaaatgtcatgttattaacagttattaggttgtcattt

SEQ ID NO: 10 - DNA, 253 bp, Homo sapiens
ccaggccccaggtctcctatttgggagaaccactgccctctgcctgcctctccaactactgctgggctgcggcccaggcgccttcaacgaccattttagggttctgatgaaagcaccttcggcttctaaggtgcaggctgggaaacaaggtgggggcccacatagcctggtgtctcagcatggagcttagtgccaagtcctgtgccagagacacctgatgtgtaaagagggaagagggcacacttgggagtgg

SEQ ID NO: 11 - DNA, 298 bp, Homo sapiens
  misc_feature (1)...(298): n = A,T,C or G
cctacagacttanctcttcttggacacacccacggngcggccacggcngccagtggtcttggtgtgctggcctcngacacnaaggccccagaagtgacgcagccctctatgggcccgaatcttcttcagtcgctccaggtcttcacggagcttgttgtccagaccattggctaggacctggctgtattttccatcctttacatccttctgtctgttcaagaaccagtctgggatcttgtactggcgtggattctgcataatggtgatcacacgttccacctnatnctcagtgagttct

SEQ ID NO: 12 - DNA, 344 bp, Homo sapiens
ctgtagtcccagttactcgggaggctgaggcaggagaatcgcttgaacccgggaggtggagattgcagtgagcccagatcgcaccactgcactccagtctggcaacagagcaagactccatctcaaaaagaaaagaaaagaagactctgacctgtactcttgaatacaagtttctgataccactgcactgtctgagaatttccaaaactttaatgaactaactgacagcttcatgaaactgtccaccaagatcaagcagagaaaataattaatttcatgggactaaaggaactaatgaggataatattttcataattttttatttgaaattttgctgattcttt

SEQ ID NO: 13 - DNA, 230 bp, Homo sapiens
ccttatttctcttgtcctttcgtacagggaggaatttgaagtagatagaaaccgacctggattactccggtctgaactcagatcacgtaggactttaatcgttgaacaaacgaacctttaatagcggctgcaccatcgggatgtcctgatccaacatcgaggtcgtaaaccctattgttgatatggactctagaataggattgcgctgttatccctagggtaacttgttc

SEQ ID NO: 14 - DNA, 216 bp, Homo sapiens
cctgacattcctgccttcttataataagaaaaataaaacaaaatagtgttgaagtgttggggcggcgaaaatttttggggggtggaatggagagagaatgggcgatgtttctcagggctgcttcaagcgggattaggggcggcgtgggaacctagagtgggagagattaagctgaagggaggtcttgtggtaaggggtgatattgtggggatgtta

SEQ ID NO: 15 - DNA, 159 bp, Homo sapiens
ctggtggtgattgcacacgacgtggatcccatcgagctggttgtcttcttgcctgccctgtgtcgtaaaatgggggtcccttactgcattatcaagggaaaggcaagactgggacgtctagtccacaggaagacctgcaccactgtcgccttcacacag

SEQ ID NO: 16 - DNA, 462 bp, Homo sapiens
  misc_feature (1)...(462): n = A,T,C or G
ccatcacaagccacgagggctggatgataccttaacatgagacagccaaatgcttaggcagataaaatggggtccctggagaatctccaagcgtcccaagaatgtttacattagatgcttttgggtcggtgagggaacctgcccagggcttgtctgggcatcccacagtgaactggagcctgacgtacgcactgggggaagtgggtggggccacggggaattcttccacggggaagagaagcctgctctcttccgctcctgtagtgactgtaccagccagaccaggaagaccctggggtccangggggaccacgttctccactgagaccgttagctcctggtttcccactttcacccttgacaccctgagggccagggcttcccctaggacctggcatgcctggtggtcctgcaagaccccgtgctccagtgatcccagcaatcccaagtgggcctggagct

SEQ ID NO: 17 - DNA, 103 bp, Homo sapiens
  misc_feature (1)...(103): n = A,T,C or G
cgccccaacccctncttggtctaatgaaatgcanttnttantgcanagatgttntaaggtgcaatatatntnttcctttcccgtggttttagagccaanctca

SEQ ID NO: 18 - DNA, 365 bp, Homo sapiens
aaatgtggtcaggggttttatagtattttttgtttaatctttttggttattgaaaaaaatagaacagtccactgtccagcagaggctgcttcaactctattgctcgcagggctcattctgcatggatctgtgtttcaggatgctgcaaggacaactctgcgggcaggaaggccccttgacccaacgctgtagcataggtcctgctctgtggatggggaaagccagggggcacatacgtccccatgccgccccctccaaagactcctcgctggtgctgaggcagggagtggtaatcttccaggttatcatactgggacacaacagtcacactgctctggcgcttgccgtgtggttggtactggtac

SEQ ID NO: 19 - DNA, 289 bp, Homo sapiens
ctgtaccctccttcccctctggcagggagagaaggggcctcccagggacttcccctcccccttagaacaggggggtgcgagctggtatggatgccctcctgggcttcctgggggctctgcccactccagactccagtttgtccaccccctgcagggtcctacatgcctaagagagcccttgtggtagaggccagcttgctaggcagctaggcaggagacccctacaaggtccaggtaaaggccagggctgccagagccagcagactggtgaggtgggacccagcccagg

SEQ ID NO: 20 - DNA, 479 bp, Homo sapiens
ctgtcctctgttactcagatacagttccaaaactaagcgattatataagcacatccatattttagggctactctaagttaaaaaccttttctcttgtttcagagttatttacatcaaattaagacatttacaaattgttcatagtatacaatagcccaaatatgattttcacctatgctgtgtaaagaagttaagcattcgtaagtttgtctaataaattcagtgcacttttttccataacacgagctattctaaatgttttacatttctttcagtgcatatttccaaattcattaaacagaatgaaatcaatgttattaaatggctatatcataatattcaagcatattatggaatctataccacagtgggattcacgtcaatactataattcactctagaaaaacatcacaggcacacacaaaataaagaacaaaatttgatttttttttataaatgtaaagtatactatctacttt

SEQ ID NO: 21 - DNA, 343 bp, Homo sapiens
aaattttttaggttaattttcttgctgtgatatatatgaggaatttactactttatgtcctgctctctaaactacatcctgaactcgacgtcctgaggtataatacaacagagcactttttgaggcaattgaaaaaccaacctacactcttcggtgcttagagagatctgctgtctcccaaataagcttttgtatctgccagtgaatttactgtactccaaatgattgctttcttttctggtgatatctgtgcttctcataattactgaaagctgcaatattttagtaataccttcgggatcactgtcccccatcttccgtgttagagcaaagtgaagagttt

SEQ ID NO: 22 - DNA, 599 bp, Homo sapiens
  misc_feature (1)...(599): n = A,T,C or G
ccattgctcaacttgaatggctgcctgggtcgggcagaaggccaggtcctcatggcttcccatccctaatgaccggaatacatgggctgccaggtcagatgtgggccacatgggaagtcccagctctattctagaaaatgcatgtaccatcagcttactgatagacatttactgaacttgggtatgccagatccacagggggccccagagatgagggggataagaaggtttctgaaggcatggtacagaaggtgccagcagaggtatgggctaggggaggcagggagagcacagagcaggcatcctaaaggaggcagcatttgtgttggagcttgaagaagtggattgtttgcaccgcctgggcaaagggaaggtgtgtgttcagggcatcgagagtactgcacaaaggctgaagcccanggcagtagagaagagaatccactaagaaggagccaatgaagaaaaaaaagagaaaagaatcgaaggtggatagggaaagacatgctgtcccgcagaggttaaaggggttctcacctcaagccagcagttctcaaaccttgtcagcagtggagccctttgttctgatgacagcctactca

SEQ ID NO: 23 - DNA, 153 bp, Homo sapiens
aaaaaggtttatgtgtgtcgaggcagttgtaaaggatttactgcagaatcaagcccacttttaggcttaggaccaggttctaactatctaaaaatattgactgataacaaaaagtgttctaaatgtggctattctgatccatagttgtttttt

SEQ ID NO: 24 - DNA, 555 bp, Homo sapiens
  misc_feature (1)...(555): n = A,T,C or G
aaaggaanccancaccatntcagagtacataagtggctatcagagaagccagccgatatggattggcctgcacgacccacagaagangcagcannggcagtggattgatggggccatgtatctgtacagatcctggtntggcaagtccatgggtgggaacaagcactgtgctgagatgagctccaataacaaccttttaacttggagcatgcaacgaatgcaacaagcgccaacacttcctgtgcaagtaccgaccatagagcaagaatcaagattctgctaactcctgcacagncccgtcctnttcctttctgctagcctggctaaatctgctcattatttcagaggggaaacctancaaactaagagtgataagggccctactacactggcttttttaggcttagagacagaaactttagcattggcccagtagtggcttntagctctaaatgtttgccccgccatccctttccacagtatccttcttccctcctcccctgtctctggctgtctcgagcagtctagaagagtgcatctncagcctatgaaaca

SEQ ID NO: 25 - DNA, 271 bp, Homo sapiens
cctaggagagggcgggggctgctgtgatccgagagctccctgacgccccaaccttccccgaacgcagctaacgagctcgtgacatccgctgacatcgccaccggtctgctttggagggatctagcgagagtcacctaccccacccctactgccaggggaggggtcgttgcccccacgagggagagaaaaacaaggactataatgcacttcgcaaaatgtaaggggccggcttcacgccagcggggccttctgggactttgaattcaaccag

SEQ ID NO: 26 - DNA, 210 bp, Homo sapiens
aaaatgggcttttgcttttctaggtcattaacgttttttatttagtttctttagccaatagtggctgagtttcgcacttgattttcaatattttatagtaagaaatgacaaactgctttggttcatttcataaacaaactctgcatttagataactattaaaggttgttaagatgaagatttactgtttctttgttactcgttggtacag

SEQ ID NO: 27 - DNA, 282 bp, Homo sapiens
ctgcgtgaagatccacaaccagctcatctcgtccgtctccaacatcacctgccccaactttgatgccagcatttgcatcccgggctccatcacattcatgcccaatggatgctgcaaggcctgcacccctcgcaatgagaccagggtgccctgctccaccgtccccgtcaccacggaggtttcgtacgccggctgcaccaagaccgtcctcatgaatcattgctccgggtcctgcgggacatttgtcatgtactcggccagggcccaggccctggaccacag

SEQ ID NO: 28 - DNA, 333 bp, Homo sapiens
  misc_feature (1)...(333): n = A,T,C or G
gtgtcggcagggttgacctccgtggcgaggtaggtgccgtcttccacgcagtgggtaacgggcttctggctgcacctcttgggttggcagatgatgccacttccaccctccaggcagacacagttcttgcagtcgaactcgaagtgctccccaaactctctgggcacattgtcaggtcccacacagccgcaggtcttcacgcagacatcaaagccaggagcgtagttcatggtgccctcaggacagaagcagccttccaccaggactgtgttgttctgctgggaggagctgggatttgcacgtgggctcttntgcangggccacaggacctcg

SEQ ID NO: 29 - DNA, 220 bp, Homo sapiens
aaatgtctgcatgcagccagccatcaaatagtgaatggtctctctttggctggaattacaaaactcagagaaatgtgtcatcaggagaacatcataacccatgaaggataaaagccccaaatggtggtaactgataatagcactaatgctttaagatttggtcacactctcacctaggtgagcgcattgagccagtggtgctaaatgctacatactccaa

SEQ ID NO: 30 - DNA, 435 bp, Homo sapiens
ccagaggagatctgcagaggggctgcaagttctggtctcagggtgggtaaagggtcaaagaggtggctctagggcagagctgtgtgggaccaagggctttgctgacaacagcctcaactccagacctctctgtggtctgtttctcctgccaggtccctgttgtgcccagtgccatgccttagatggaattgagtgtgccagtcctaggacctttctacgagaaaataaaccttgtataaagtataccggacactacttcataaccactttactctactccttcttcctgggatgttttggtgtggatcgattctgtttgggacacactggcactgcagtagggaagctgttgacgcttggaggacttgggatttggtggtttgttgaccttattttgctaattactggagggctgatgccaagtgatggcagcaa

SEQ ID NO: 31 - DNA, 400 bp, Homo sapiens
ccagccacgcttacgttcccatcacactgatgactccgggtttggcgagcacaggagcgcaaaccttttcacattctttctgtgatccaaatttgttttcgtttccaccacaacctccataccagaatcttgcacagcttttggtgtttggatcatagtaccattttaatatgaaatccctgcaagttccttcgtctttcggcaacttgcatatatctgtttcagtgagagccaatggttctgtgctcaccattagattgatggttgaactagaagctgaccttgctggctgtggaggtgggggctgagatttctttgtactgaaacttccgtggtaggtggctctgacctgagacctcaggtagcagaccacagccacatggtatgtctgcccagcgag

SEQ ID NO: 32 - DNA, 325 bp, Homo sapiens
ctgcagtttttgactcgtcctgggaaactggcactgagactcaggggtgtaacatttcacctccctgaaatcaaatccagaaatctcaggcgagcagcgtatataaaaagccacaggggaaaaaaggaatacggactcagcaactcttaggtgctctgagcctcttcccaagccttctggttctgtgagcatttcattgaaagaaaatggaataacagagttttagaagaaaaactgtatttggtcttgcaagagaaaagtatattcatataaaactcagttctcaactatttgccaaggttacttcttttgtttccaacattta

SEQ ID NO: 33 - DNA, 292 bp, Homo sapiens
gcttctaggaggtggcacggtgcacgccaagatggctgtgtccacagaggagctggaggccacggttcaggaagtcctggggagactgaagagccaccagtttttccagtccacatgggacactgttgccttcattgttttcctcaccttcatgggcaccgtgctgctcctgctgctgctggtcgtcgcccactgctgctgctgcagctcccccgggccccgcagggaaagccccaggaaggaaagacccaagggagtggataacttggccctggaaccctgaccctgtgtc

SEQ ID NO: 34 - DNA, 112 bp, Homo sapiens
ccaatgacactgaccactactttctgcgctatgctgtgctgccgcgggaggtggtctgcaccgaaaacctcaccccctggaagaagctcttgccctgtagttccaaggcagg

SEQ ID NO: 35 - DNA, 556 bp, Homo sapiens
aaaatccctattgcaagcctaacactgacctcgctagtaactcttaaggcaatcaagacggaacatgtgtttggcccccagatgcacgaatcctgccctcccctcaacctttgttcatcctaacagaccaacctggctcctgcattaatatggagtggggagaacagcaaaacaattcactgtatgtacaaaagacaattcagtgcaaacctagaaacttctcttagtcaatagtttccaattttctgagacgaggtcttgctccatcacccaggccggagtgcagtggcacgatcttagctcactgcaaactccacctcccaggctcacgggatcctcccacctcagcctcccgagtagctgggactacaggcatgcaccaccacacccagctaatttttgtatttttagtagagacagggttttgccatgttgcccaggctggtctcgaactcctgggctcaagcaattcgcccacctgagcctcccaaagtgctgggattacaggcgtgaaccaccacacccggcctcaaattttcttattgcccttgagcaa

SEQ ID NO: 36 - DNA, 404 bp, Homo sapiens
ttggttacttcttttaatgtattcaaaaatgttgaacacatacagaactgaattaagaagcaacaactgccctatggaagagctgtattagtacagaatgcttttaagaaccaaggacaaattttcagtattgaagaagacaacatacataaaaagcactccaaattcatttctaattccttcaataccatgctaaagttcttttttagagggtatgtctcttaacaactttacataattcacaatgagaatgtgacaacatgtcaatttggcaatcaacacttcttcattgcaccttacttactttatgcatgcggcccacacattacttcagctcaagaggctggggtaattctgtccctaaggcaatcaaggagctcagcacaaaccttgaaatcattttt

SEQ ID NO: 37 - DNA, 344 bp, Homo sapiens
tgccaggttcacacatcccaggaaaaaagaagcataaaaagcattagcagtcagtgactgatgataatgctgcaataatgggaatggttttgtttctaaaccaaattatttctaaatcaaatcatttattgctttgtttctaaagcaattgagtcactaagtttgtgaactgtaggagaacacatcaagattgaatcctgtgttaagcagaaggtaaaaccagagccaggcgcagtggctcgtgcctgtaattccaaaaccttggcaggaagatcgattgaggccaggagctcaagacgagcctgggcaacatagaaagaccctatctttacaaaaaaaacttt

SEQ ID NO: 38 - DNA, 343 bp, Homo sapiens
cagaaagctgtggatgagatgaacggaaaggagctcaatggaaaacaaatttatgttggtcgagctcagaaaaaggtggaacggcagacggaacttaagcgcaaatttgaacagatgaaacaagataggatcaccagataccagggtgttaatctttatgtgaaaaatcttgatgatggtattgatgatgaacgtctccggaaagagttttctccatttggtacaatcactagtgcaaaggttatgatggagggtggtcgcagcaaagggtttggttttgtatgtttctcctccccagaagaagccactaaagcagttacagaaatgaacggtagaattgtgg

SEQ ID NO: 39 - DNA, 272 bp, Homo sapiens
cattacataaaaggacatacctctacctagcaatgaccatactgcatgaagagggactaagatgaaggaaagaaacaaaaaggcaagtcaaagaaagacaggagtgtaaggtcctaaggaaggggcaaataatcaaaggtctcatctgatcaggagcaatgccaatccaatccagttctaggtcccaagtaaagaaacatagtctgagaaaagaggccagggatacagcttgggatgttcagagagtggaaatgacagaggtggattatttt

SEQ ID NO: 40 - DNA, 414 bp, Homo sapiens
ctggtccagcagctcgagcagtgggagttccgagtctgtggagtttttcttgttgattctcagttcatggtggagtcattcaagtttatttctggcatcttggcagccctgagtgccatgatctctctagaaattccgcaagtcaacatcatgacaaaaatggatctgctgagtaaaaaagcaaaaaaggaaattgagaaatttttagatccagacatgtattctttattagaagattctacaagtgacttaagaagcaaaaaattcaagaaactgactaaagctatatgtggactgattgatgactacagcatggttcgatttttaccttacgatcagtcagatgaagaaagcatgaacattgtattgcagcatattgattttgccattcaatatggagaagacctagaattt

SEQ ID NO: 41 - DNA, 174 bp, Homo sapiens
cctacgagaaaatccttttcactgaggccacccggatcctcttcttcaacacacccaaaaagatgacagactacgccaagaagcgagggtgggtcctgggccccaacaactactacagttttgccagccagcagcagaagccggaagacaccaccattccctccacagaactgg

SEQ ID NO: 42 - DNA, 260 bp, Homo sapiens
ctggtggtgagctagctcatctccacaacacccaccaaggctccaaagacacctctcagcttagctttcactggtggattggatgctgttctcatttgatctatagatcctagcattttggagtttgatgaaagaagggcaggagagtattaagaatcaaaggtcttagctgggcttggtggctcatgcctgtaattccagcactttgggaggccgaggagggtggatcacttgaggccaggagtttgagaccaccctgg

SEQ ID NO: 43 - DNA, 566 bp, Homo sapiens
cctctgtgcaagcagcacataggatctggatgtaggttgaggatagatcctcacccaccagtggggtaactttcccagcaattctgaaactaaaataaggaaggcacattcccagagccctgctgagtaggggcttcaggctattttcactctacacaaaatgggggagaggagttccctctccactaatttttcacccataaacctccacatcactaggaacctaaaggggaactccaaaggccaacacatccttggtggttatatgtgttgtcctgacaacctcctgctccagaaatgccaggagcattggatatgtcattgggagcatcaggcagtccaacatcggagggagaaaggcccagagatgaggatctgagtcaggctggcaaggctggagtcagaaagtgaccattaggcaactggtcactacaattggtggctacaaagaagtggtcacagtcaccaaaataaagaggtttacaacaacggtttccccttaggtcattttgaccaggacagtaccctaaaggaaataaggcagcatcgcataaagcaagagcccc

SEQ ID NO: 44 - DNA, 344 bp, Homo sapiens
ccactggctgagttattggcctggcaggtatagagtccgctgttcttctcagtgatgttggagataaagagctcttgtgtgtgttgctggatgttcccatcaatcagccaagaatactgtgcaggtgggttagaggctgcatggcaggagaggctgaggttcacccctggacggtaataggtgtatgagggggaaatggtggggtcgtctgggccatagaggacattcaggatgactgggtcgctgtggtcaacacttaattcgttctggattccacactcatagggtcctacatcattccttgtgacactgagtagagtgagggtcctgttgtcattggacag

SEQ ID NO: 45 - DNA, 404 bp, Homo sapiens; identical to SEQ ID NO: 36

SEQ ID NO: 46 - DNA, 215 bp, Homo sapiens
  misc_feature (1)...(215): n = A,T,C or G
gtgggtgacagtgatgccaggctcgcccactactgcactggacacagcctcaccaatgccaccttcataataatggtcctccacggtgaggatcctgcccttggtggcacgagcgctgtcgagaatgagttttctgtccaggggcttgatggtgaannggtccagcacgcggatgttgatcttttctttcttcagcagttcggcagcggccaagg

SEQ ID NO: 47 - DNA, 425 bp, Homo sapiens
aaattataagtattgtgaattcacactctcaggctattgtctgacttgatctacgtctcataaagcctgtacctgagtggagtggaaggtggagtcttaggttaatcagttactgactctaccctcaccctctttcaattgaggtaaactttgctgtttttctttttcataaagcattctcaaattgttgagtttattgctgaaaaaaatctccatgactttacagatagaattacaaactaaatgatgtcttgtatttagaagcagagtacagacctaacgaactgttagattctccaccatcacttagggtttgcccagaagcaacaccagagaattacagacaacgcgcttttgctgaacaagcatttgtagcttgtacaatggcagaatgggccaaaagcttagtgttgtgacctgttttt

SEQ ID NO: 48 - DNA, 423 bp, Homo sapiens
ctgctgcaacattaccgtctgcaagtgcaacaccagcctgtgcaaagagaagccctccgtgtgcccgctgggattcgaagtgaagagcaagatggtgcctggaaggtgctgtcctttctactggtgtgagtccaagggggtgtgtgttcacgggaatgctgagtaccagcccggttctccagtttattcctccaagtgccaggactgcgtgtgcacggacaaggtggacaacaacaccctgctcaacgtcatcgcctgcacccacgtgccctgcaacacctcctgcagccctggcttcgaactcatggaggcccccggggagtgctgtaagaagtgtgaacagacgcactgtatcatcaaacggcccgacaaccagcacgtcatcctgaagcccggggacttcaagagcgacccgaagaacaa

SEQ ID NO: 49 - DNA, 121 bp, Homo sapiens
ccagggcggtacgaatcgtctcctggcactgtgcaggcccacagctgagaactggcctctacaaatcccagagaccgtgcgtaacacacatcaagacagaacctgttgccattttcagcca

SEQ ID NO: 50 - DNA, 253 bp, Homo sapiens
  misc_feature (1)...(253): n = A,T,C or G
ctggggcggcctatgccgagtggcgcccatggcgaagagggctcagctcgcatgtggaagactctcaccttcttcgtcgcgctccccggggtggcagtcannatgctgaatgtgtacctgaagtcncaccacggagagcacgagagacccgagttcatcgcctacccccatctncgcatcaggaccaanccgtttccctggggagatggtaaccatactctattccataaccctcatgtgaatccacttccaa

SEQ ID NO: 51 - DNA, 228 bp, Homo sapiens
ctgaaagtaaacagaatggattgccagttacatgtatgcctgcccagttccctttttatttgcagaagctgtgagttttgttcacaattaggttcctaggagcaaaacctcaaggattgatttattgttttcaactccaaggcacactgttaataaacgagcagggtgttttctctcttcctttctaatatatggagtttcgaagaataaaatatgagagcaatattt

SEQ ID NO: 52 - DNA, 217 bp, Homo sapiens
  misc_feature (1)...(217): n = A,T,C or G
ctgactagttcccctaataatcggtgcccccgatatggcgtntccccgcataaacaacataagcttctgactcttacctncctctctcctactcctgctcgcatctgctatagtggaggccggagcaggaacaggttgaacagtctaccctcccttagcagggaactactcccaccctggagcctccgtagacctaaccatcttctccttacaccta

SEQ ID NO: 53 - DNA, 186 bp, Homo sapiens
aaattttcattgagttgtccatctccagcatatagggcttcaggagcagagcagaccttgtttttagtggttccatgggataaaatgggattggaggagctagaagaattcagggtctggtccaatctgccagtcttcctgaaatatcgaaaatacaccagggctgctatatcagagccaccctgg

SEQ ID NO: 54 - DNA, 164 bp, Homo sapiens
caggcgcagcccagcctcgaaatgcagaacgacgccggcgagttcgtggacctgtacgtgccgcggaaatgctccgctagcaatcgcatcatcggtgccaaggaccacgcatccatccagatgaacgtggccgaggttgacaaggtcacaggcaggtttaatgg

SEQ ID NO: 55 - DNA, 330 bp, Homo sapiens
ctgtgatgaacagtacttgtgtcagttctgtgaacatgaaactaatgatccagaagacttgcatagccatgtggtaaatgagcatgcatgtaaattaatagagttaagtgataagtataacaatggtgaacatggacagtatagcctcttaagcaaaattacctttgacaaatgtaaaaacttctttgtatgtcaagtatgtggttttcggagtagacttcacacaaatgttaacaggcatgttgctattgaacatacaaaaatttttccccatgtttgtgatgactgtgggaaaggcttttcaagtatgctagaatattgcaagcattt

SEQ ID NO: 56 - DNA, 408 bp, Homo sapiens
cctagtatgaggagcgttatggagtggaagtgaaatcacatggctaggccggaggtcattaggagggctgagagggcccctgttaggggtcatgggctgggttttactatatgataggcatgtgattggtgggtcattatgtgttgtcgtgcaggtagaggcttactagaagtgtgaaaacgtaggcttggattaaggcgacagcgatttctaggatagtcagtagaattagaattgtgaaaatgataagtgtagagggaaggttaatggttgatattgctagggtggcgcttccaattaggtgcatgagtaggtggcctgcagtaatgttagcggttaggcgtacggccagggctattggttgaatgagtaggctgatggtttcgataataactagtatggggataa

SEQ ID NO: 57 - DNA, 218 bp, Homo sapiens
ccttatgacatgtgctgtggctcccagcaccagtttaggtacttggagtgcagcagggaagaaaataacttggctgctctgcacgctgggggcttcactcagcggcatctagacagacacataattggccgggcgtggcggctcacgcctgtaatcccaaaacctgggaggccgaggcaggccgatcacttgaggtcaggagttcgagaccagcctgg

SEQ ID NO: 58 - DNA, 390 bp, Homo sapiens
ccaagaacgtgcaataaattggaagtttgccccggggcagcaagaatttatgctgccattgaaaagcaggtaccagtgccccttttcagacagtttttgattcgctctagacttttttttttttaatagggagggaaaaaatttgataattttcttttttctacatgcacttaagactaaaacacaggtttggattaattttatttgcttcctttttccgcttttcttcccgcagagcctgatgggagaatgtccagggcagggaaaccacattttttgtaggtgataactcaatgaaaattggtgcttattttttacacttctctcttgtggctctcttgtggtgctatctgttttaaggtctccttgaaggcgcactggggtccctgg

SEQ ID NO: 59 - DNA, 516 bp, Homo sapiens
ttgttttgcttcttccttaaagcatttgcaacagctacagtctaaaattgcttctttaccaaggatatttacagaaaagactctgaccagagatcgagaccatcctagccaacatcgtgaaaccccatctctactaaaaatacaaaaatgagctgggcttggtggcgcacacctgtagtcccagttactcgggaggctgaggcaggagaatcgcttgaacccgggaggtggagattgcagtgagcccagatcgcaccactgcactccagtctggcaacagagcaagactccatctcaaaaagaaaagaaaagaagactctgacctgtactcttgaatacaagtttctgataccactgcactgtctgagaatttccaaaaccttaatgaactaactgacagcttcatgaaactgtccaccaagatcaagcagagaaaataattaatttcatgggactaaatgaactaatgaggataatattttcataattttttatttgaaattttgctgattcttt

SEQ ID NO: 60 - DNA, 222 bp, Homo sapiens
cctctttttaccagctccgaggtgattttcatattgaattgcaaattcgaagaagcagcttcaaatctgccggggcttctcccgccttttttcccggcggcgggagaagtagattgaagccagttgattagggtgcttagctgttaactaagtgtttgtgggtttaagtcccattggtctagtaagggcttagcttaattaaagtggctgatttgcgttcag

SEQ ID NO: 61 - DNA, 350 bp, Homo sapiens
aaaaaactcaaaaagctgggaattaagtggtttcagtaataatgctataccgaggtgcttgcattgtatttcataattttgttacaaaccaaaattatttttaatgagaacagtcttgggttcagaggtgtgatgccagaatgtattttcgtactgttaggcccttggaacagataccggtgctttctgaaagatgaaagaaatgcaatgggtgctcttcatgcaaggttgcaaacctaccaagaatgcataatagtctcacttttccccaataaagagatgcgtgtgactagttttggacttttaaccttaatgggggttgcatgtctcctattgttaatcattgtcag

SEQ ID NO: 62 - DNA, 391 bp, Homo sapiens
aaaaaccagatcgctacccatgagaagaaagctcatgaaaactggctcaaagctcgtgctgcagaaagagctatagctgaagagaaaagggaagctgccaatttgagacacaaattattagaattaacacaaaagatggcaatgctgcaagaagaacctgtgattgtaaaaccaatgccaggaaaaccaaatacacaaaaccctccacggagaggtcctctgagccagaatggctcttttggcccatcccctgtgagtggtggagaatgctcccctccattgacagtggagccacccgtgagacctctctctgctactctcaatcgaagagatatgcctagaagtgaatttggatcagtggacgggcctctacctcatcctcgatggtcag

SEQ ID NO: 63 - DNA, 439 bp, Homo sapiens
  misc_feature (1)...(439): n = A,T,C or G
aaaataggccctgagtataagagcatgaagagctgcctttatgtcggcatggcgagcgacaacgtcgatgctgctgagctcgcggagaccattgcggccacagcccgggagatagaggagaactcgaggcttctggaaaacatgacagaagtggttcggaaaggcattcaggaagctcaagtggagctgcagaaggcaagtgaagaacggcttctggaagagggggtgttgcggcagatccctgtagtgggctccgtgctgaattggttttctccggtccaggctttacagaagggaagaacttttnaacttgacagcaggctctctggagtccacagaacccatatatgtctacaaagcacaaggtgcaggagtcacgctgcctccaacgccctcgggcagtcgcaccaagcagaggcttccaggccagaagcctttt

SEQ ID NO: 64 - DNA, 249 bp, Homo sapiens
aaaacattttttagtctgtaatacactccacttgaagcacttaagtcttccttaaatgacttttcttaagtaatgatactgtgtgttttcccaaagcacacagtatcattacttaagaaaatttttataaattactatctgttgaaaaggtgtccttttcctttcttctagtatttttttcttaccaaaattcactaatcttgaatgtttgtgatattaaatttcaaatgcagaatacttgactcattt

SEQ ID NO: 65 - DNA, 229 bp, Homo sapiens
  misc_feature (1)...(229): n = A,T,C or G
ggagctcangcggtgatgttcgctcacctgctgctcacctactgctgcgtggcccagttnctaacagaccacagacggatctgctggggactcctgcatataaagtctgccatcatggatgtnntcgcagaagcaaatggcacctttgccttaaaccttttgaaaacgctgggtaaagacaactcgaagaatgtgnntttctcacccatgagcatgtnctgtgccctgg

SEQ ID NO: 66 - DNA, 195 bp, Homo sapiens
ccacagacccccaggtcattgtgttcactgtactctgtgggcaaggatgggtccagaagaccccacttcaggcactaagaggggctggacctggcggcaggaagccaaagagactgggcctaggccaggagttcccaaatgtgaggggcgagaaacaagacaagctcctcccttgagaattccctgtggattttt

SEQ ID NO: 67 - DNA, 425 bp, Homo sapiens
ctgtcaaccttgacaaattgtggactttggtcagtgaacagacacgggtgaatgctgctaaaaacaagactggggctgctcccatcattgatgtggtgcgatcgggctactataaagttctgggaaagggaaagctcccaaagcagcctgtcatcgtgaaggccaaattcttcagcagaagagctgaggagaagattaagagtgttgggggggcctgtgtcctggtggcttgaagccacatggagggagtttcattaaatgctaactactttttccttgtggtgtgagtgtaggttcttcagtggcacctctacatcctgtgtgcattgggagcccaggttctagtacttagggtatgaagacatggggtcctctcctgacttccctcaaatatatggtaaacgtaagaccaacacagacgttgg

SEQ ID NO: 68 - DNA, 471 bp, Homo sapiens
ctgtgtgactgcctgtccctacaactacctttctacggacgtgggatcctgcaccctcgtctgccccctgcacaaccaagaggtgacagcagaggatggaacacagcggtgtgagaagtgcagcaagccctgtgcccgagtgtgctatggtctgggcatggagcacttgcgagaggtgagggcagttaccagtgccaatatccaggagtttgctggctgcaagaagatctttgggagcctggcatttctgccggagagctttgatggggacccagcctccaacactgccccgctccagccagagcagctccaagtgtttgagactctggaagagatcacaggttacctatacatctcagcatggccggacagcctgcctgacctcagcgtcttccagaacctgcaagtaatccggggacgaattctgcacaatggcgcctactcgctgaccctgcaagggctgggcatcag

SEQ ID NO: 69 - DNA, 352 bp, Homo sapiens
gtgtcctttatcttactttatctgtacagtaatcctgtgagaaagacaggacagaaaccactgtgcctattttacagatacgaaaactgagacacaggtaaggggcttgtctgtagtcccatagctagcagatggctggagccaagactgaggctcgttcttcaatgctgagccagggctccttccgctgcaccacaagaacgctagaccactcgccaccagccttctcattccctcttcctccattctaatcatttctagctggctggcctccacagagcataggaaaacagccagggccgggcacggtggctcatgcctgtaatctcaacactctgggaggccaaggcag

SEQ ID NO: 70 - DNA, 519 bp, Homo sapiens
aaaaaaagctatgtcttcactccaaaatgacagagacagactactgaaggaattgaagaatctgcagcagcaacacttacagattaatcaagagatcactgagttacatccactgaaggctcaacttcaggagtatcaagataagacaaaagcatttcagattatgcaagaagagctcaggcaggaaaacctctcctggcagcatgagctgcatcagctcaggatggagaagagttcctgggaaatacatgagaggagaatgaaggaacagtaccttatggctatctcagataaagatcagcagctcagtcatctgcagaatcttataagggaattgaggtcttcttcctcccagactcagcctctcaaagtgcaataccaaagacaggcatccccagagacatcagcttccccagatgggtcacaaaatctggtttatgagacagaacttctcaggacccagctcaatgacagcttaaaggaaattcaccaaaaggagttaagaattcagcaaacctc

SEQ ID NO: 71 - DNA, 434 bp, Homo sapiens
ctgtatgtgataatgaaagggtttttctttcttatgttaaatacaagcgaagtgattaactggaagatagcgtctgattgcgaggaaatcagtgattcagatggtgtgggaatggcacctggggatgggggaggcaggacggagatggaggaagctggtgcagcctagcctgccttgtgccaaggacacccaagggcagagggactgagctctgggggaggacagatttgacataactggtccagcctcacagtttacaggtcctggagggtgaggaacagacgtgggagcaccagagggacagagctgatggcctgacgctctcttcaggagggcacccccaaggggcctctgcttcctcagtgccccctgagctttatcagcagaggggtgttttccagccacaaggagctgtatctaacactaatgccttt

SEQ ID NO: 72 - DNA, 295 bp, Homo sapiens
  misc_feature (1)...(295): n = A,T,C or G
ccattctagtgatccaaaggccgtaatgttccccacctacaaatatgttgacatcaacacatttcncctctctgctgatgacatanntggcattcagtccctgtatggagacccaaaagagaaccaacgcttgccaaatcctgacaattcagaaccagctctctgtgaccccaatttgagttttgatgctgtcactaccgtgggaaataagatctttttcttcaaagacaggttcttctggctgaaggtttctgagagaccaaagaccagtgttaatttaatttcttccttatgg

SEQ ID NO: 73 - DNA, 118 bp, Homo sapiens
  misc_feature (1)...(118): n = A,T,C or G
ctgctgtctgacnatgaaaccaaagacgacatgnncatgtcctcctactcggtggtcagcacgggctncctgcaatgtgaagaccttgcanacnacacggtgctggtgggcggggagg

SEQ ID NO: 74 - DNA, 633 bp, Homo sapiens
  misc_feature (1)...(633): n = A,T,C or G
ccttggtcgcccaacagcccattggcaaccttctcatgtctggtcacagagaactgatttgtcctcagtacctctccgtccaccttcatgtacacagtgggcaccaccttcacaaagtactggaacatcatggaggcttggggcgcagtgacattggtgtggtccagggggttcacaatgcctggatagtcctccccaaatgacaggtgctggatgtagtgggtcatgttgatgttgtcaaggccaaagctctgcaagtcatggacgtgcacatgggactgctggaagctcttcccaggggcaaagtggaagtttccggccaccttattgacttccaagaagccatacacctggcagccttcattcttctgctcctgcatcttctggctgaagccctctcgccggcactgctcaatagtatctgggttcttgaaggcccagcctctacggcgatatgcctcccgcacatcttcacaggtgttacagcacttgatatcttctgcctcagcaccatagcagctctcacagcgatcagggtccagggagtcagggtcaaacaccgtcacctcgactttncccaagctcatgccgntcagcctctgagctcacggggatgccatctttatctagtcg

SEQ ID NO: 75 - DNA, 305 bp, Homo sapiens
ttgccaaagcctccgattatgatgggtattactatgaagaagattattacaaatgcatgggctgtgacgataacgttgtagatgtggtcgttacctagaaggttgcctggctggcccagctcggctcgaataaggaggcttagagctgtgcctaggactccagctcatgcgccgaataataggtatagtgttccaatgtctttgtggtttgtagagaatagtcaacggtcggcgaacatcagtgggggtgaggtaaaatggctgagtgaagcattggactgtaaatctaaaagacaggggttagg

SEQ ID NO: 76 - DNA, 611 bp, Homo sapiens
  misc_feature (1)...(611): n = A,T,C or G
ctgtttcataggctggagatgcactcttctagactgctcgagacagccagagacaggggaggagggaagaaggatactgtggaaagggatggcggggcaaacatttagagctagaagccactactgggccaatgctaaagtttctgtctctaagcctaaaaaagccagtgtagtagggcccttatcactcttagtttgctaggtttcccctctgaaataatgagcagatttagccaggctagcagaaaggaagaggacggggctgtgcaggagttagcagaatcttgattcttgctctatggtcggtacttgcacaggaagtgttggcgcttgttgcattcgttgctgctccaagttaaaaagttgttattggagctcatctcagcacagtgcttgttcccacccatggacttgccagaccaggatctgtacagatacatggccccatcaatccactgccactgctgcctcttctgtgggtcgtgcaggccaatccatatcggctggcttctctgatagccacttatgtactctgctatggtgctggcttcctttaaactcangatagatgccaggtgggctccgtttccgtaagactgacactcgagctc

SEQ ID NO: 77 - DNA, 267 bp, Homo sapiens
ctggtatcagagaagtcagtagaggtcactgagaccggcagtctttcttgctttttgcattagtgccctcaggacacacagcaaacagtgatcatgagaagagtgagctcaatagttttccatcaagtgtgcttaaaattccatgcagtcgccataagggtacaacttctgaggtatggtcaacctatggtacattagtaaatgataaggggaggaagaaatgaaaacctaaacgtctactgcaatgaaaaccaatagcaatgtcag

SEQ ID NO: 78 - DNA, 295 bp, Homo sapiens
  misc_feature (1)...(295): n = A,T,C or G
aaatatttatcagtctaaacttgtgcagtgtagtaaacatgcaagttgttacgattgagctgtattaccataagtagaattttaagtaaactggtgaatttgggcaataaatgtttttgctttttgtttgatttttttttacaagctaactgttagaggtatacatttatttatctgttgtacagatttgattatgattttaatgtttgaaagattgcacttgtttgcttttactatatgtggggtaaaatatattttntgntcacagtatatgaaaatatggagtaatttacct

SEQ ID NO: 79 - DNA, 320 bp, Homo sapiens
  misc_feature (1)...(320): n = A,T,C or G
tttttttttttttttttttttttgggganaacagggtgtcgctatattacccagtcaggtctcgaactctggacctcaagtgacccacctgcctcggcctcccaaggtgctgggatagcaggcgtgagccactgtgcccagcctcacctaatggtttcttagcaaacttcagtanaatgtttanaacgcggccctgataaacttgagtgctggtaggaggtgctacctcgctcaatctgtgagcaaccagccctgtgccctggatgcttggcgggtggagagaaanacagtgttatgtgggcaagcctccaaactcacca

SEQ ID NO: 80 - DNA, 133 bp, Homo sapiens
  misc_feature (1)...(133): n = A,T,C or G
tgagggtcttactcttttagtataaatagtaccgntaacttccaattaactagttttgacaacattcaaaaaagagtaataaacttcgccttaattttaataatcaacaccctcctagccttactactaataa

SEQ ID NO: 81 - DNA, 406 bp, Homo sapiens
  misc_feature (1)...(406): n = A,T,C or G
ctgtgggggcctcctttcctagtttctgaatgatcttcctgtggctctgtgagcaggcccagcatggggaatgggctaaaaggcttatacatctcttttggccctcagatgcacttacccttttctttggtgccctctttccccaagagaatattcaggccaattttgcttttttccttgtttctgcattagtaagacattataaactagcaacttgtaatacctctaactctcactgtcttatgttagtataaagtacctcaaggtaataagaatgtggaacttaaatgccacttacagaaagtcaaacaaagcccatgtcacactttgatgaatncaaagtattaaatcttancaactgatgaagtaaaaagctatttttgctaangtttaactattggacttt

SEQ ID NO: 82 - DNA, 340 bp, Homo sapiens
aaaaattatgagccttttctagcccccaccttcccaaccctcagagaaggacagtaaagaaatcagtggatccaggtatttacctgttgttgaattgtgaggttgtgaggtagacgtgtaacaaggacaaggaagtttggggatctgcttggagaatgaaggtttattcaaaacaagtggacaggtcaggggtaacgggtgatgagggcacctggcttttgtaatcatgtggggactgtcccctgggaggtgcagcaaaaatcagaacggagacagaagctcacagtctggttttagctgccaactcctatggaagtccatagctgactcctatggaagg

SEQ ID NO: 83 - DNA, 380 bp, Homo sapiens
  misc_feature (1)...(380): n = A,T,C or G
gtgcgcaccaccacacccggccaattttggtntcttttgtaatgacanaatnncgccatgttgcccangctgatctcaaactcncgggctcaagcaatccacccacttcggcctaccaaagtgctaggatcacaggcatgagccatagcacctggcccacattttcttttgttaaatgaagttaatctatgtnctagtaaatanacaattatgtttccaacacaacagaaatctatttcaacactaaacatcactgaacgattttgctaaggttttcatgctagatgtgtcttactaacaaaggtaacacaattccacagttctgtttttgaataaaaggnatatatgttatatatctgaaaacttacacaagatgttca

SEQ ID NO: 84 - DNA, 529 bp, Homo sapiens
aaaataatttagttttgcttgcttccattgatcagtcttttacttgaggcattaaatatctaattaaatcgtgaaatggcagtatagtccatgatatctaaggagttggcaagcttaacaaaacccattttttataaatgtccatcctcctgcatttgttgataccactaacaaaatgctttgtaacagacttgcggttaattatgcaaatgatagtttgtgataattggtccagttttacgaacaacagatttctaaattagagaggttaacaagacagatgattactatgcctcatgtgctgtgtgctctttgaaaggaatgacagcagactacaaagcaaataagatatactgagcctcaacagattgcctgctcctcagagtctctcctatttttgtattacccagctttctttttaatacaaatgttatttatagtttacaatgaatgcactgcataaaaactttgtagcttcattattgtaaaacatattcaagatcctacagtaagagtgaaacattcacaa

SEQ ID NO: 85 - DNA, 525 bp, Homo sapiens
aaatccagaagaaaacaaaaaattctggacttggatttttgaggttagaaagaatttttgaagcagaaatcccaataaatatgctgatcttcctaagaatgaataaatcacagaattttaagccaaaaggaaagggcacataaagattatccagtccaacctcattttacagacagggagggtgacctgcccaaagtcacatgactaaaaggagaaggggtggccttggaatacagatgttctgacttctagggtcttatcttttaatgttgcctttttgtcctcaaagctgcctgcttattgggttggaagaactcacatcttatgaagggttagaccctgccttgaaaatcagtatggtaggctgggcgtgggcccttacgcctgtaatcctatcgctttgggaggcccagccaggtggattgcttgagcccagcagcttcagaccagcctgggcaacacggcaaaaccccatctctacaaaaaaattcaaaaattagctgggcatggtggtgcacacacctc

SEQ ID NO: 86 - DNA, 430 bp, Homo sapiens
aaaaaatatttagctttgcagttcctgaccccttaatgcctgacccttccaagcaaccaaagaaccagcttaatcctattggttcattacaggaattggctattcatcatggctggagacttcctgaatataccctttcccaggagggaggacctgctcataagagagaatatactacaatttgcaggctagagtcatttatggaaactggaaagggggcatcaaaaaagcaagccaaaaggaatgctgctgagaaatttcttgccaaatttagtaatatttctccagagaaccacatttctttaacaaatgtagtaggacattctttaggatgtacttggcattccttgaggaattctcctggtgaaaagatcaacttactgaaaagaagcctccttagtattccaaatacagattacatccagacctc

SEQ ID NO: 87 - DNA, 408 bp, Homo sapiens
  misc_feature (1)...(408): n = A,T,C or G
ccatgtacatatgggtcctcgaagacaagccatgaaagagatgtccatcgatcaagccaaatatcagcgatggcttattaagaacaaaatgaaggcattttatgctccagtacatgcagatgacttgagagaaggtgcacagtatttgatgcaggctgctggtcttggtcgtatgaagccaaacacacttgtccttggatttaagaaagattggttgcaagcagatatgagggatgtggatatgtatataaacttatttcatgatgcttttgacatacaatatggagtagtggttattcgcctaaaagaaggtctggatatatctcatcttcaaggacaagaagaattattgtcatcacaagagaaatctcctggcaccaaangatgtggtagtnagtgtggaatata

SEQ ID NO: 88 - DNA, 502 bp, Homo sapiens
aaaaaagtttccacttgacactttgatccctgatggaaaacgcataatctgggacagtagaaagggcttcatcatatcaaatgcaacgtacaaagaaatagggcttctgacctgtgaagcaacagtcaatgggcatttgtataagacaaactatctcacacatcgacaaaccaatacaatcatagatgtccaaataagcacaccacgcccagtcaaattacttagaggccatactcttgtcctcaattgtactgctaccactcccttgaacacgagagttcaaatgacctggagttaccctgatgaaaaaaataagagagcttccgtaaggcgacgaattgaccaaagcaattcccatgccaacatattctacagtgttcttactattgacaaaatgcagaacaaagacaaaggactttatacttgtcgtgtaaggagtggaccatcattcaaatctgttaacacctcagtgcatatatatgataaagcattcatcactgtg

SEQ ID NO: 89 - DNA, 329 bp, Homo sapiens
  misc_feature (1)...(329): n = A,T,C or G
ttgtgatcgtggtgtgcgtcagcttcctggtgttcatgattatcctgggggtatttcggatccgggccgcacatcggcggaccatgcgggatcaggacaccgggaaggagaacgagatggactgggacgactctgccctgaccatcaccgtcaaccccatggagacctatgaggaccagcacagcagtgaggaggaggaggaagaggaagaggaagaggaaagcgaggacggcgaagaagaggatgacatcaccagcgccgagtcggagagcagcgaggaggaggagggggagcanggcgacccccagaacgcaacccggcagcagcag

SEQ ID NO: 90 - DNA, 166 bp, Homo sapiens
  misc_feature (1)...(166): n = A,T,C or G
tgcttcttccttaaagcatttgnnacagctacagtctaaaattgcttctttaccaacgatatttacagaaaagactctgaccagagatcgagaccatnntagccaacatcgtggaaccccatctctactaaaaatacaaaaatgagctgggcttggtggcgcgcac

SEQ ID NO: 91 - DNA, 333 bp, Homo sapiens
ctggctgcccaccaggccgtgtatgtgaggtcaaggctgaagcccggaactgctgggccacccgtggtctctgtgtcctgtctgtgggtgccaacctcaccacctttgatggggcccgtggtgccaccacctctcctggtgtctatgagctctcttcccgctgcccaggactacagaataccatcccctggtaccgtgtagttgccgaagtccagatctgccatggcaaaacggaggctgtgggccaggtccacatcttcttccaggatgggatggtgacgttgactccaaacaagggtgtgtgggtgaatggtctccgagtggatctcccag

SEQ ID NO: 92 - DNA, 357 bp, Homo sapiens
aaaagggaggtgggggtagaagtaaaaggatgatcatgggagggagctgaggggttaatatatatacatacatacacatatatatatttttgtaaataaacaggaactgattttctgcctccatcccacccatgagggctgcaggcactacaaaagagctgactactgagaattctggaaaacaaggttttttttatttgtagctatagctacaacttggcggcatgggggagggtgggaatgtcctggagggtctcccagccctccgcaagcagagtacaaaggctgctcggggggccggccgagggcgcgggtgcagcagtgaaagcagcagcactaaacctggtgcccccctca

SEQ ID NO: 93 - DNA, 246 bp, Homo sapiens
gctccagcctctggggcgcattccaaccttccagcctgcgacctgcggagaaaaaaaattacttattttcttgccccatacataccttgaggcgagcaaaaaaattaaattttaaccatgagggaaatcgtgcacatccaggctggtcagtgtggcaaccagatcggtgccaagttctgggaggtgatcagtgatgaacatggcatcgaccccaccggcacctaccacggggacagcgacctgcag

SEQ ID NO: 94 - DNA, 454 bp, Homo sapiens
  misc_feature (1)...(454): n = A,T,C or G
ctgaagcaagtagatgctttttcaaaaggaaaccaaagcaattgtttatatgcttggaagatgtcttattcattggaggctgaatgctgagtctgtttttgaaaactgcattttcttgaggcaggtcgcacgttctaggagtccacactgatgcaagcacagaaaagaaggaagccaaggagaagtgatcctgggggttttctcaagcccatagttccagaaggtgcaataccagcattggtttatgatcagtctttcaatcaacaatttgatgattagggatctctacattcgtatttcaggtcagagaagaacaccatttcttgagagaagacaaacaaccctagtctaccaccagcataggttttatcatagatgggtcctgagtcagccatgattttcttnccttcatacatcaccactctaatgaaacccgtctttggcctgtggctga

SEQ ID NO: 95 - DNA, 50 bp, Homo sapiens
  misc_feature (1)...(50): n = A,T,C or G
tctacctttgcaggaacgcgctcatgttcaacaacganctcatggccgac

SEQ ID NO: 96 - DNA, 324 bp, Homo sapiens
ctgtttcccaaaggggtcacactcaagccccgcagaccacacaagaatcacaaaccacgaggtccgtctcccccatgactgacaccaagacagtcaccaccccaggttcttccttcacagccagtgggcactcgccctcagaaattgttcctcgggacgcacccaccataagtgcagcaacaacctttgccccagctcccaccggggatggtcacacaacccaggccccgaccacagcactgcaggcagcacccagcagccatgatgccaccctggggccctcaggaggcacgtcactttccaaaacaggtgcccttactctgg

SEQ ID NO: 97 - DNA, 298 bp, Homo sapiens
aaactagtgtcagtgacactaggaatataataaaggtaacacagcaagaagcacagaactactccctcttcatctccatattttcataatttcttgtgtttcaaatagggaaacatcttcctcaaagtctgcctagtgagatatggcctactggttgcctcatagctttgtacagattatgaggactgaaaataattgggcatttacccatcttggtatctgttgtatcctttatctgtgtgtgctgatttgatcttttttcagtttcacataccttatctaaggtttcccaggattt

SEQ ID NO: 98 - DNA, 366 bp, Homo sapiens
  misc_feature (1)...(366): n = A,T,C or G
ctggcaggaggcccactcactgcccaagtcatggcaacaggccggagcagcccangagatgggcctaaaatgttctggatcccttgggtcctantgttatgttccagtctgcccacctgtgctcaggatgcanncctgggatccagcacccatggaagcttctgntgggatggngtcacctatgggttttgaaccantgtggtatggtccttgggagctctgntctgagcttgccacactgntgagagcacccactgtcctgaccagagtctcantggtcctgacccccaatgngggcaggggctgggcaggagggtggggtctgctgtgggttcagangactccacctnctggctggtttacctg

SEQ ID NO: 99 - DNA, 292 bp, Homo sapiens
cctcggaggggcagccttcggggcggggagcgtgagcgcgccaaggccatccctgagatctacctgacccgcctgccagcagtcctcctgacatggactatgaccctgaggcacgaattctctgtgcgctgtatgttgttgtctctatcctgctggagctggctgaggggcctacctctgtctcttcctaactacaaaagccctttctccccacaagcctctgggttttccctttaccagtctgtcctcactgccatcgccactaccatcctgtcaccagtgggacctcttt

SEQ ID NO: 100 - DNA, 343 bp, Homo sapiens
tgtagtcccagttactcgggaggctgaggcaggagaatcgcttgaacccgggaggtggagattgcagtgagcccagatcgcaccactgcactccagtctggcaacagagcaagactccatctcaaaaagaaaagaaaagaagactctgacctgtactcttgaatacaagtttctgataccactgcactgtctgagaatttccaaaactttaatgaactaactgacagcttcatgaaactgtccaccaagatcaagcagagaaaataattaatttcatgggactaaatgaactaatgaggataatattttcataattttttatttgaaattttgctgattcttt

SEQ ID NO: 101 - DNA, 172 bp, Homo sapiens
aaacaatccttgaattttccatgttatcagaagttgttaacagcatcgagacggaagtatatgaaatataaggactgaaataaaagtgaatttgaaagatggctaatctactagattaggtaaagggggaacgggtaagtggtggggaggagtagggaacgatggggtggtt

SEQ ID NO: 102 - DNA, 194 bp, Homo sapiens
ggtcagtgatgggggagtaccactggtctgtgtgctagaggagggtgttgctgacctgaatctgaatttctaacaggctcacagatgaggccagcaccactggtctgagggccatgcccaggcacacgatgttctcataactcggctgcaatgattatatatggtggaggcaagctggggccaaggtagttcat

SEQ ID NO: 103 - DNA, 342 bp, Homo sapiens
gtgctcggggtaatgacggtgctcgaggcagtgatggtcaaccaggccctcctggtcctcctggaactgccggattccctggatcccctggtgctaagggtgaagttggacctgcagggtctcctggttcaaatggtgcccctggacaaagaggagaacctggacctcagggacacgctggtgctcaaggtcctcctggccctcctgggattaatggtagtcctggtggtaaaggcgaaatgggtcccgctggcattcctggagctcctggactgatgggagcccggggtcctccaggaccagccggtgctaatggtgctcctggactgcgaggtggtgcag

SEQ ID NO: 104 - DNA, 282 bp, Homo sapiens
ctgcgtgaagatccacaaccagctcatctcgtccgtctccaacatcacctgccccaactttgatgccagcatttgcatcccgggctccatcacattcatgcccaatggatgctgcaagacctgcacccctcgcaatgagaccagggtgccctgctccaccgtccccgtcaccacggaggtttcgtacgccggctgcaccaagaccgtcctcatgaatcattgctccgggtcctgcgggacatttgtcatgtactcggccaaggcccaggccctggaccacag

SEQ ID NO: 105 - DNA, 297 bp, Homo sapiens
ctggctgagaaacgagagcacgagaaagaagtgcttcagaaggcaatagaagagaacaacaacttcagtaaaatggcagaagagaaactgacccacaaaatggaagctaataaagagaaccgagaggcacaaatggctgccaaactggaacgtttgcgagagaaggataagcacattgaagaagtgcggaagaacaaagaatccaaagaccctgctgacgagactgaagctgactaatttgttctgagaactgactttctccccatccccttcctaaatatccaaagactgtactgg

SEQ ID NO: 106 - DNA, 210 bp, Homo sapiens
ctgacagccagcagtaccttcccaaccattagagtgagtcaccctagaagcaaattctccagctccagtgcatcctttagataactgccactctggtcactatcttatctacaacctcatgagaaacctcagccagaaccacccagctaagttgcctctgaattcccgagccacagaaactgggagataatgtttactgtttaagacttt

SEQ ID NO: 107 - DNA, 338 bp, Homo sapiens
agatggcggacattcagactgagcgtgcctaccaaaagcagccgaccatctttcaaaacaagaagagggtcctgctgggagaaactggcaaggagaagctcccgcggtactacaagaacatcggtctgggcttcaagacacccaaggaggctattgagggcacctgcattgacaagaaatgccccttcactggtaatgtgtccattcgagggcggatcctctctggcgtggtgaccaagatgaagatgcagaggaccattgtcatccgccgagactatctgcactacatccgcaagtacaaccgcttcgagaagcgccacaagaacatgtctgtacac

SEQ ID NO: 108 - DNA, 426 bp, Homo sapiens
  misc_feature (1)...(426): n = A,T,C or G
ctgatgatgtagaagtatatgattgaacgaccagagccagaattccaagacctaaacgaaaaggcacgagcacttaaacaaattctcagtaagatcccagatgagatcaatgacagantgaggnttctgcagacaatcaaggatatagctngtgcaataaaagaacttnttgatacagtgaataatgtcttcaagaaatatcaatnccagaaccgnagggcacttgaacaccaaaagaaagaatttgnnaagnactccaaaagtttcagtgatactctgaaaacgtattttaaagatggcaaggcaataaatgtgttcgtaagtgccaaccgactaattcatnaaaccaacttaatacttcanaccttcaaaactgtggnctgaaagttgtatatgttaaagagatgtacntctcagtggcagtat

SEQ ID NO: 109 - DNA, 79 bp, Homo sapiens
  misc_feature (1)...(79): n = A,T,C or G
aatcancaaaatttcaaataaaaaattatgaaaatattatcctcattagttnatttantcccatgaaattaattatttt

SEQ ID NO: 110 - DNA, 421 bp, Homo sapiens
  misc_feature (1)...(421): n = A,T,C or G
cgctggggcctcatagttgagcacgtagtagtcgtggacatacatgaggacggctattggctgtccgatgatgagcgacagccacacacccaaattggaccgcttaagagttgcactttccaaagtcaacttctaagtctacaaggacagcaacaatgtttcagtggattctgaagttacatgtatcaacaatttccccggaaagctaaccctccaccgggaactccaggtgaatgaatgagtgagggaattcgccagattgagttacaaagcctttccaacgattatcaagagcaggtgctcggttacaacacagaggtatcctccttcacagcctttggaccttgctgcgtggagattttcacagataagaggggggaaatagagagacaggccttnctccccggccatccacacctta

SEQ ID NO: 111 - DNA, 274 bp, Homo sapiens
ctgtcaaccttgacaaattgtggactttggtcagtgaacagacacgggtgaatgctgctaaaaacaagactggggctgctcccatcattgatgtggtgcgatcgggctactataaagttctgggaaagggaaagctcccaaagcagcctgtcatcgtgaaggccaaattcttcagcagaagagctgaggagaagattaagagtgttgggggggcctgtgtcctggtggcttgaagccacatggagggagtttcattaaatgctaactacttttt

SEQ ID NO: 112 - DNA, 76 bp, Homo sapiens
  misc_feature (1)...(76): n = A,T,C or G
ccagagagaagagggccaggangctgcaacaggctggcaganaggctggncangtagtanccaccctctccagtaa

SEQ ID NO: 113 - DNA, 228 bp, Homo sapiens
cccactgaagccgtggggacgcgcccagcggagctaatcagattacctggctggtgtttgcttgttctggagtgatcttctgactggaaaagaactatgtcatggatcaaggaaggagagctgtcactttgggagcggttctgtgccaacatcataaaggcaggcccaatgccgaaacacattgcattcataatggacgggaaccgtcgctatgccaagaagtgccag

SEQ ID NO: 114 - DNA, 489 bp, Homo sapiens
gtggaacagactgtcctccatgtcagttccttctggcttcaggccctcaattctttccctttgagcttttttagaccccagatctcctaggcccaggctctctcttgaccccagagaagccactgtcaggaaaggaagtgaaccctactgaagccagagaattcaccccggccaaagcaggccctctgggtccagcccctcattccacaccacaccagtattgcatccatctactgcagctacacatcctgagggcagcaccacccactctggcctgctggcccatcgcaggactagcccaggcacctgccgggcattgcaggatatccagtggggcctgtgactgctccctgatgcgtcagaagagaagtgttgcactttagtggaggagctgaggagcacctgcccccttgtagcttgagttccttttggtaacagtagcagcctccatggtggtgtctgggacgcacgtgcacccgctgccttcag

SEQ ID NO: 115 - DNA, 501 bp, Homo sapiens
  misc_feature (1)...(501): n = A,T,C or G
ctgcaccatgccatctatagagataggaacggtgggtggtgggaccaacctactacctcagcaagcctgtttgcagatgctaggtgttcaaggagcatgcaaagataatcctggagaaaatgcccggcagcttgcccgaattgtgtgtgggaccgtaatggctggggaattgtcacttatggcagcattggcagcaggacatcttgtcaaaagtcacatgattcacaacaggtcgaagatcaatttacaagacctccaaggagcttgcaccaagaagacagcctgaatagcccgacagttctgaactggaacatgggcattgggttctaaaggactaacataaaatctgtaaattaaaaaagctcaatgcattgtcttgtggaggatgaatagatgtgatcactgagacagccacttggtttttggctctttcagagaggtctcangttctttccatgcagactcctcagatctgaacacagtttagtgctttacatgctg

SEQ ID NO: 116 - DNA, 452 bp, Homo sapiens
  misc_feature (1)...(452): n = A,T,C or G
ccatattctcatcatatcctctctgtgtggagtctgcctgttgtcacaaaaaccttgaccctacatcaagttacaccttaacaaagggaagatacaggcatcagataaaaggtacttgtttgaaaggcagccataagggagaactgaacttaaaaaaaaaaaaanaaaaaaaattccaagctggtttcaacagtactttgtttccagaacaaagaaatgtttctaaccacatcttgtaccccttcctcatcaactccagactaccacagacctttttccaaaactgtgtgtcacacatccaggtcttgtgctttanagctgcctctcaggcaattttagccagccatttctccaagtcctggatgtcagcagagcccacgtcccctcttncacccttggcactgcactccangaactccactttgaggggcaactgtganaattcaaactct

SEQ ID NO: 117 - DNA, 385 bp, Homo sapiens
aaaacattgttttctaaacactaacaaaaaaaattaagggcaaactgaaaatacaaatgagatttacaggcactgtgtgtagaatgtgcaaaaattcacttagcttttcttttgtttttttggtgttgctttaagaaactttatcaaatatatttcttacaaatataaagctttctctcccaattgaaggcaattaaaaaaattcaaagtttatcaatactcagtacacaggtgaaccagtcaaattcattttctttctggaaaagaataacaaaccaatatttaggatgttcagagactcaacaaaaaccattctagaaatcacccagaacaattgttttctgttgccaaagccttttgttcttcaaaagtcaccatccaccag

SEQ ID NO: 118 - DNA, 286 bp, Homo sapiens
ttggtttgcctttttccttcctaactttcccatatgtagaagaagccattaagattgcttactgtgaaaagaaatgtggaaactgctctctcacgactctcaaagatgaagacttttgtaaacgtgtatctttggctactgtggataaaacagttgaaactccatcgcctcattaccatcatgagcatcatcacaatcatggacatcagcaccttggcagcagtgagctttcagagaatcagcaaccaggagcaccaaatgctcctactcatcctgctcctccagg

SEQ ID NO: 119 - DNA, 275 bp, Homo sapiens
gtggtgaggtttctgaagaattatccctgaaactgccaccaaatgtggtagaagaatctgcccgagcttctgtctcagttttgggagacatattaggctctgccatgcaaaacacacaaaatcttctccagatgccctatggctgtggagagcagaatatggtcctctttgctcctaacatctatgtactggattatctaaatgaaacacagcagcttactccagaggtcaagtccaaggccattggctatctcaacactggttaccagagacag

SEQ ID NO: 120 - DNA, 70 bp, Homo sapiens
  misc_feature (1)...(70): n = A,T,C or G
cttgagacttgaaaccacaanaagtgtganaagactggctagtgtggaagcatantgaacacactgatta

SEQ ID NO: 121 - DNA, 168 bp, Homo sapiens
aaaagcgacctttttgtccattacagaagtaacgtatttattgtagaaatgtaatagataaaaatgaaataattattcatattctcactattccacaaatgtctgtgattaacagattcattgtcaactttagttctcattctgcacatatgtaagttatgtttgtat

SEQ ID NO: 122 - DNA, 342 bp, Homo sapiens; identical to SEQ ID NO: 103

SEQ ID NO: 123 - DNA, 443 bp, Homo sapiens
aaacttacttcatttattatttgttactctttatttctccctagtatgttttggacatttgaatgtcctcttctgtgaatttttcatgtttgttgcctatatctctattttggttttagaagttaaattattacttaaaagaactttttaataagtttgaatgttaaattttgacctctcatgtgcattgcaaatttttttcctcaagtatctttttcttttttttagatagtgtttttgaaagtcttcatggtgatatgcactatattcagtatatgtatgttttcctacttctcttgtaaaactgttgcatgatccaacttcagcaatgaattgtgcctagtggagaacctctatagatcttaaaaaatgaattattctttagcagtgtattactcacatgggtgcaatctttagccccagggaggtcaataatgtctttt

SEQ ID NO: 124 - DNA, 145 bp, Homo sapiens
ctgaacctgagaaggaggaggcggccaaggaagaagccaccaaggaggaagaagccatcaaagaggaggtggtcaaggagcccaaggatgaggcacagaatgagggcccggctacagagtcagaggccccgctgaaggaggatgg

SEQ ID NO: 125 - DNA, 391 bp, Homo sapiens
ctgatttgtttactgaacactgtcacattaaatgatggtgcctaggtaaaaacgctgcacacactcccctccacccccaccccttacccatgttgagacgtggctgcctgtcatgagatgagatctgcttgagtaaagccatatacattacagcaagcattccagattcttaaaatgaccaaacactttggtattaatacaatgtattccctgttttctcaaatatacaaaatatacatttccagttttagttgtggttttcttgtttttttttgtttttgttgtttttacacaggaatagttaggtctgtcatttgagggagcccaggggacctggaacgggtcacacgggcagtgctcagttctggtgcctcttcatatgcagggccag

SEQ ID NO: 126 - DNA, 306 bp, Homo sapiens
aaaaatcactacatcaaatgggatagagagtaagaagacaggagagagaggagaaaccatgttttttgttttgagtcaggagggtctcactctgtcactcaagctgaagcacagtggcacaatcacagctcactgcagcctcaacctcccaggctcaagcgatcctactacttaagcctcccaagttgctgagactacaggcacaagccaccatgcccagcccaatttgattgtgtttcatacagatagccagttttcccagcaccaacccggacttgttaaatagcctgttcttttctcactttt

SEQ ID NO: 127 - DNA, 153 bp, Homo sapiens
aaaaaatcccacttttcgaaaatatctgacaatcaagggcacagagactagcgtaatgctgattctcactggcgcaaacagcttgtggatcgcataggccaccacgaaggtacttgtgcctgctgccatttttgactgtaccagggactcttt

SEQ ID NO: 128 - DNA, 134 bp, Homo sapiens
  misc_feature (1)...(134): n = A,T,C or G
gctttcattcctgttcanaantcaatgcccttgacggggctgatgtgtnnagctgntaacanncacccatcccagtgtcaggangatttgannnaggagtttggangagagtgggaaggaatgactgcttanga

SEQ ID NO: 129 - DNA, 246 bp, Homo sapiens
aaaggctttcattcctgttcagaagtcaatgcccttgacggggctgatgtgttgagctgctaacagtcacccatcccagtgtcaggaagatttgatagaggagtttggaggagagtgggaaggaatgactgcttaggaggggagagagcctggcaatgaaatgtggcccagggcaccagcctgacagccccgagggacccctgggtgtgtttgaggctttcatagttcagatttctgcatgcccgg

SEQ ID NO: 130 - DNA, 460 bp, Homo sapiens
  misc_feature (1)...(460): n = A,T,C or G
cacaattctaccgttcatttntgtaactgctttagtggcttcttctggggaggagaaacatacaaaaccaaaccctttgctgcgaccaccctccatcataacctttgcactagtgattgnaccaaatggagaaaactctttccggagacgttcatcatcaataccatcatcaagatttttcacataaagattaacaccctggtatctggtgatcctatcttgtttcatctgttcaaatttgcgcttannttccgtctgccgttccacctttttctgagctcgaccaacataaatttgttttccattgagctcctttccgttcatctcatncacagctttntgtgcatcttcatgcctttcaaagcttacaaatccaaatcctttggattttncactttcatcagtcattactttcacacttaaggcaggcccaaacttgccaaagagatccttaaggcgc

SEQ ID NO: 131 - DNA, 464 bp, Homo sapiens
  misc_feature (1)...(464): n = A,T,C or G
tgacctgnatctctctgctattaatgacaaaagcatcgncaaaaagacnccacagttagcaaaaacaatatcaaagaaacctgagtcaacatcattttctgcccctcggaaaaagagcccggatttatctgaagcaatggaaatgatggagtctcanacactactgctgacgctactatccgtaaagatggagaacaatcttgctgagtttgaaagaagggcagaaaagaatttattaataatgtgtaaggagaaggagaagctacagaaaaaggcccacgagctgaagcgcaggcttctcctctctcagaggaagcgggagctggcagatgtcctggatgcccagatcgagatgctcagccccttcgagtgcgaggcgaggtggctcctgacgctaanaggtgtgcatcaccttcgaccaggccaacctgaccgtcaagctgccagatggatacnaattcaag

SEQ ID NO: 132 - DNA, 303 bp, Homo sapiens
ctgcggtggtcaggtcccggtattcccggtacatgttgtgggtgccgctccgggagtcatagcgcagccagatcccgaagttcttcacccgcaggggggacttctcaaacacctgcccacagtagacaatctcccctgaagacttcttcatcttctttaactgagatacaaagtaccagaagcgggacttggcgacgacatgattaggcgcaaagattcgcatgcggtagaggggcggcgtgtggcatttgggggtgggcaggcagcgacccactaccttgtactctcgtagcgtgcccgagg

SEQ ID NO: 133 - DNA, 273 bp, Homo sapiens
gtggatgatgtctgtggcgatggcattcaagaagttgcggtcgtgggagacgactaggatggtggagggccacgtctgcaggtaattctccagccacaggatggccctgacatccagcatgtttgtaggttcatctaacagcagaagatctggcctagcaaagagggcccgggccagggccagcctcatcctccagccacaaacagcaccattgtattgttgaatgtttatgtaactgatggcttttctataatgtaatttttgaatgttcag

SEQ ID NO: 134 - DNA, 507 bp, Homo sapiens
ctggtccttcaggcaaaatttggaggtcacaatgaactccaagcctgacacaaagatattctacagtttcacagctatcatttgtacatattaagttgattcactctttttgagcaaatctacctagaaaacggcaaattaatatattcctttacatacaactttgtgtctcaaaattcttgaaaaacaagagcagatgactttgtattcaaagactaccaaagtatgtatttgattttcacatgcaaacaacttaaaaccttataaatctcatgtcaactctgcatgatgccttgaaggaaatgacatacaaagtttgctaactgtgcaaaatattaaattgctaaaacattttacataatgaaataatacatgtaaatgttgaagttgacacatgaaattaacatggcataagaacttatcacatttcagatattttctttagtaacaagtttttgtttttatagttcctggtacacagcaaagtttatcacgaaagataaaaat

SEQ ID NO: 135 - DNA, 148 bp, Homo sapiens
ctcggcggccacagacatcacgtcctccctatcagacgaccaggtacccgaggctttcctggtcatgctgctgatccagttcagtaccatggtggttgaccgcgccctctacctgcgcaagaccgtgctgggcaagctggccttccag

SEQ ID NO: 136 - DNA, 150 bp, Homo sapiens
ctgctaagaagcagacattgtctctacaagctcagagagaagagaaagcaaaagcctccgagctctccaaaaagaaagcatctgccctgttgttcagcagtgatgaggaggaccagtggaatattcctgcttcacagacccacttagcat

SEQ ID NO: 137 - DNA, 179 bp, Homo sapiens
aaatgcactgttctggttcctaacttgaagcagttgtccttgtgagaaccggtctttgcctttagctcatgtcgtgtttcacagcaaagagggtacagaaccatcactggtccaggttaatgtacaaaattttctggcaatgcctgattaaaaaaataaaattggcttgttgagaacag

SEQ ID NO: 138 - DNA, 249 bp, Homo sapiens
ctgcactggaagcttccagggatgttgatgcagcggtaggagcagatgtggcccccggtgggcagggcgcactcgtcgatgtcttcacaggtgactccatccacatcgctgagctggtagcctcgccggcagtaacactggtaggagccgtagacgttggcacactcctggctacaggggctgctgctgcactcattgatgtcttcacatgacctgccatccacagagagccggaagcccacggaacag

SEQ ID NO: 139 - DNA, 237 bp, Homo sapiens
  misc_feature (1)...(237): n = A,T,C or G
aaaaccatcataacaaaaagggtccattgtcttatgatccactggaaagaggaccgactcatcatttatggctatgacttggcagtgactccaatgtgatatcctgtaattttatcttcagttatgctatagcatgtacatttccattctcttgtcgaagtttctttcgttcctcancttctccttcatatttcctgacgtattgtcttctaagctggactgtaataacagcaacag

SEQ ID NO: 140 - DNA, 342 bp, Homo sapiens
cttccatcatgaaacgggatgacagcaacaataagactttggctgagcaaaacactaagaatcctaaaagcactactggtagaagttccaaatctaaagaggagccattatttccatttaatttggatgaatttgttactgtggatgaggttatagaagaagtgaatccttctcaggccaagcagaatccactaaagggaaaaaggaaagaaactctcaaaaatgttcctttctctgaacttaacttaaagaagaaaaaggggaaaacttccactcctcgtggtgttgagggagaactatcttttgtgacattggatgagattggggaagaggaagatgcag

SEQ ID NO: 141 - DNA, 226 bp, Homo sapiens
gtcctctagagaatcccctgagagctccgttcctcaccatggactggacctggaggatcctcttcttggtgtcagcagccacaggagcccactccctggtgcaagtggtgcagtctggggctgaagtgaagatgcctggggcctcagtgcaggtctcctgcaggacttccggatacaccttcaccgactactacatacattgggtgcgacaggcccctggacaagg

SEQ ID NO: 142 - DNA, 235 bp, Homo sapiens
  misc_feature (1)...(235): n = A,T,C or G
ccagcgacctcccggttcaattcttcagtccggctggtgaaccaggcttcagcatccttccggttctgctcggccatgacctcatattggcttcgcatgtcactcaggatcttggcgagatcggtgcccggagcggaatccacctccacactgacctggcctcccacttggcccctcagcgtactgatttcctcctcatggttcttcttcaggtaggccagctcttncttcagga

SEQ ID NO: 143 - DNA, 508 bp, Homo sapiens
ctgacaaaaagtgtcagagccagaggccaacctctgctagatgaagcagcagcacatgactccatttctatctgataaggagacagagaagaggcatctcgaacagatgaaaaaccaaaggctggtgtcctaaaaaaacagattggcttcaaagaaaacactaaggaagacccacagagctgtattaattttagtaaaaataatcatatgccaacaggggaattgaaccactttctaaatcatagtatgaactcatctcttcagatacttggtaagtggtcaaagcttgtttttataattactttcactgtcttgggcaaaaagtctttcttatctttggtccttaggtgtggtatcagtttcttccatttttttatgtgttacaaaacaatcttttttttacttgacatcaacaaccaaggtgcagtataaacacgaagttgctgatattgttgcttttatacacataaaataccaacatctcccatacattttataggctatacga

SEQ ID NO: 144 - DNA, 382 bp, Homo sapiens
cctgccgtcgatgccagggaggccgacaggaccttcttttccagcggggccgatatttccaggggaaccaggaagacctctgggtcccatgagaccaggctccccagggcgaccagcatctccattaggtcctcggactccagcagggccacttgcaccacgactaccaggagggcccatgacgccagctctgccatcagctccaggaagaccacgagaaccaggactacctctcagcccaggaggtcctggagggccggcagatccagcttccccattagggcctctctttccttcttcaccactgggaccaggaggaccttggggcccagcagagccgggctcacccttgttaccgctctctcctttggagccagacctg

SEQ ID NO: 145 - DNA, 109 bp, Homo sapiens
gctaacatgccttggttcaagggatggaaagtcacccgtaaggatggcaatgccagtggaaccacgctgcttgaggctctggactgcatcctaccaccaactcgtccaa

SEQ ID NO: 146 - DNA, 87 bp, Homo sapiens
gtgaagtaccacggagaaatcatattggaaagttactacttagccatctgacttgacttccttggttatcaaataattacatattct

SEQ ID NO: 147 - DNA, 396 bp, Homo sapiens
aaagataaaaccaacatgtccagtgctatccttatgcatggtaatcgtccgttcaaagggcctgtcacgaatggtcatggtaatcttctctccaaaagcctgtttgagcaccttgtgcgctttatcagagctccatcctgcacagttttcaccattgatctgaagtacttggtccccaaatctcagaccaaccaatgaggctggagaattagcctggactagctgaacaaatataccattatctattgatttaagcctgagtccaatttctccatcttgatccttacacaaaatgacttcacgaatcccttgcttaatttctgctctacgaattccaacatcattaccagttacaggagccaccatatagtttatactggaaggtcttgctaccaa

SEQ ID NO: 148 - DNA, 503 bp, Homo sapiens
aaatcccaatttcccatcttcatcttcagaaaccatttcaaacgtatcaaactgtaatttcttcataacagccacatatttttcttcaagtgactttaatactgacaaaggtttgggtttcatagccgccttcttggagtattcacccagttttttttcctgatttgcttgccgcaaactggtggtggctgcataaactatctcagcagtcttttggatgtctggtaccaaaagagtaagtccttctggttcttgatcagacgcatctggttttactcctgttttaacattttcccttttagatcttaaacggttggtataggtatcaacacaggtcttcattttggctaacaatgtaccaacagaagtttgacattctgactgttcttcttcctcttcaccgttctctgtagaaaggggcaacaataggggcaccatggcagcacaagaagcaatggcccgaagcaattccagcagtgcccgatagagtggcacatgtcttg

SEQ ID NO: 149 - DNA, 196 bp, Homo sapiens
ccattaaaagttatttacaacagtgggagaaaaaaagacaagaagttgtttcacactacagacctccccccaccccaaagcctaatacttgcttaccaagtcaaaaaagagacacagttgattcacaggctggaggtttgaacttgagtaagacatttataaaaacctagacggggcagtgtcctccccagcccag

SEQ ID NO: 150 - DNA, 147 bp, Homo sapiens
  misc_feature (1)...(147): n = A,T,C or G
ccttctctgaaaaaagagaaggaattacttattaaaactaagcacacttagcaacttctttccnatcctatctttattcgtttgcctngtgccaaatttttctngccctttttaatttgcaaaccttnaaaaaaaaaaccaaaaaac

SEQ ID NO: 151 - DNA, 419 bp, Homo sapiens
ctgcgctatggcgaagacggcctggcaggcgagagcgttgagttccagaacctggctacgcttaagccttccaacaaggcttttgagaagaagttccgctttgattataccaatgagagggccctgcggcgcactctgcaggaggacctggtgaaggacgtgctgagcaacgcacacatccagaacgagttggagcgggaatttgagcggatgcgggaggaccggcaagtgcgtgtcctgctcttcagaagtggagtgaagggcgtgttctgtgcaggtgcagacctgaaggagcgggaacagatgagtgaagcagaggtgggggtgtttgtccagcgactccggggcctgatgaatgacatcgcagccttccctgcacccaccattgcggctatggatgggtttgccttgggcggagg

SEQ ID NO: 152 - DNA, 241 bp, Homo sapiens
gtgccagtcaagatgcctggctcaggccatcaggagctggttagccccattccacccccagccctgcatgcagggtccagccattgtctttgggggaaacaggcagaataagtggaggatggagctggggcttgggctcctctaggtaccttctgagagctttgacaagccagaaagaagctaccaggttgagggtgctggtcttctggactcaggagagacatgttcgccgaggatatca

SEQ ID NO: 153 - DNA, 271 bp, Homo sapiens
ctgtctcaccagctccctaactcatgtgtacctgcaccttcctcttgaaatctgaacattataataccacaagccactttcagcctccagtgggaaggctccagccacacgccgatatttcgtcctgcttcccgtcatctcatatctaaaagtcatggcttaagttaggcaataaaacctgtggctttaggcatctttagtaaaaaagctgaacaaatcccaaatttattcccattttcttgagaaataaacttcataaaacaacagacag

SEQ ID NO: 154 - DNA, 120 bp, Homo sapiens
ccatggcgctcgggtgcgcgcagtgcacgcgggttatcaccggagtgggactggtgacttcattagaagaggaaggaagacctgagctggcctgtgaatatgctccgccccctgcatcag

SEQ ID NO: 155 - DNA, 92 bp, Homo sapiens
ccatggcccaggtcacccaccccctggtccacatcactgaggaagtagaagagaacaggacacaagatggcaagcctgagagaattgcccag

SEQ ID NO: 156 - DNA, 501 bp, Homo sapiens
  misc_feature (1)...(501): n = A,T,C or G
gtgtgagccactgcaccaggcaaactgcgatcttttagnggtgcctnttctctcttttgacttaaggatgttgtcccttaaggaaacctggaggctactactgtgatacactacttgagagatggattgttgcgctttcttctacagtctttacaaggagtagattataaagacagaagatgttaaccattgcattaatgtttggaagctgacagtcttctagatttctgctagcaaactgatatgaggtagagtcctgaaagatctttcagcaatttcattttcttgggataagtgagtcactttcagaacagtatgtgttgtagaattttttggttgtggctgctctactcagattgcatagaggtttttttgntttctgntttctgnttgnttgntttggtcagattttttgaaacatcctcaaagtgactattcagttttcaggatgatacactatgaagatgtttcaaaaaatcttcatagtgtatcatccacctc

SEQ ID NO: 157 - DNA, 527 bp, Homo sapiens
aaaggagccagcaccatagcagagtacataagtggctatcagagaagccagccgatatggattggcctgcacgacccacagaagaggcagcagtggcagtggattgatggggccatgtatctgtacagatcctggtctggcaagtccatgggtgggaacaagcactgtgctgagatgagctccaataacaactttttaacttggagcagcaacgaatgcaacaagcgccaacacttcctgtgcaagtaccgaccatagagcaagaatcaagattctgctaactcctgcacagccccgtcctcttcctttctgctagcctggctaaatctgctcattatttcagaggggaaacctagcaaactaagagtgataagggccctactacactggcttttttaggcttagagacagaaactttagcattggcccagtagtggcttctagctctaaatgtttgccccgccatccctttccacagtatccttcttccctcctcccctgtctctggctgtctcgagcagtctaga

SEQ ID NO: 158 - DNA, 323 bp, Homo sapiens
ccacttacacttgtgaccagtgtggggcagagacctaccagccgatccagtctcccactttcatgcctctgatcatgtgcccaagccaggagcgccaaaccaaccgctcaggagggcggctgtatctgcagacacggggctccagattcatcaaattccaggagatgaagatgcaagaacatagtgatcaggtgcctgtgggaaatatccctcgtagtatcacggtgctggtagaaggagagaacataaggattgcccagcctggagaccacgtcagcgtcactggtattttcttgccaatcctgcgcactgggttccgacag

SEQ ID NO: 159 - DNA, 541 bp, Homo sapiens
  misc_feature (1)...(541): n = A,T,C or G
ctgctatgtggtggccgctgtggctgacactgagtgaaggtgtttgaaatgcaggagaggatatcccagcaaattgggatcacatgcttttgtctccacagcaaccagccactgcaggcagcatgtctttcctcccctgctctctgcttgctgttgttttgacgctattctgcttgcatgtcttctggttgggatgtggagttgttgctggactctcaggcgaagctgaagtcattgaagtgtgtgaagctctgtgcttgcatgagggcaagcaaggaatggctgtgcctgaggctgctctgggaaactccttgccccttgacctcttttgagagcattcacgtggtcttcttgctcatccccttataaatgtgctttgcctgcctcagcctcatggtcagagcagtggagactggagccctgtttgcacgttctagttgttcggagaaagcctaggttctgggctcangtccagatgcagcggggattctgttctctgactgtggcgaccttgctttggttcttgttgaagtgaaccaag

SEQ ID NO: 160 - DNA, 378 bp, Homo sapiens
cctgggagatcccagggtcctccaccctccccctgaccacatacaaaggcactctagttcaagggtgaaaagtctcacccaggaggaacagccctccttgaagcaatggcagggccagcagggaggtgggcatggcagggaatggagtgagccagacagacttcacctccttactggacacagggtcaagggcgagtttcaattgctgctccctttactttctctacctgtgactactccctggaccaatcctgaggagggcacattttccagaagccacgtgataggggctggtttctgtggagccggaggcagagacactgaacttgagctcacctcctaacaccggcagtaaacttcctggaactttgccctcag

SEQ ID NO: 161 - DNA, 388 bp, Homo sapiens
  misc_feature (1)...(388): n = A,T,C or G
ctgaagaagaagctgccgacctcccaacaaagcctacaaagatctccaagtttggatttgccataggtagtcagacgacaaagaaagcatcagccatatccatcaaacttggatcaagtaagcctaaagaaactgttccaaatattgaacaacagggtggacagatgatcatgaagcaactatatatgttagatgacaagaaagaacctcataattcacagtcattttccaataaatgtttatgatgagttttgatttctcatgatttcctttataaattccccaggataaactaagttgctctangatgagcttgggaagctaggttaaaacaggaacgaggcatcacaggatagaaacaatcctggtgggattcacctatcaccag

SEQ ID NO: 162 - DNA, 300 bp, Homo sapiens
ctgccaaaatctgctggaatcctttgatggtctccttcaggggtaccagcttccccatatgacctgtgaagacctcagcaacctggaatggctgagacaagaaacgctgtattttccgtgcacgggacacggtcaacttgtcttcctcagaaagttcatccatacccaggatggcaatgatatcctggagggatttgtagtcctgcaggatcttttgcaccccacgggcaacatcgtaatgctcactgccaacaatgttgggatccatgatacgagaggtggagtctagaggatccacag

SEQ ID NO: 163 - DNA, 197 bp, Homo sapiens
aaaactacaaacacaatattgactaaaaaaggaaaaaaaagggaataacatgtatctaataaaatattcagtataaaaagaggactaatggaattaagtggcccctttccccatttttacattctaaacaatgattccatcaagacaaatcattaaaaagtgttattacactgattttttttttttaataagaagga

SEQ ID NO: 164 - DNA, 548 bp, Homo sapiens
cttcttttggtggtaaataggtatttatttgaaatgaaaaaaaaattacttaagtacctggactattgcatttaatcatgtattgtaattgtgttactctacctttttgcatcagagacaaatatacaatgaacattcagatatcacagactgcacactagatagtaattcttcaggtcttttacataaccaccaagaaacagatattggtttctgcaatatagtataaaagtccacaatcaatccagtcttagccagtatcttcaatttacttctgttgctgtacaaataattggagaagggctttccttgcagaggaaatacatggactgtagaagatactcctcagggtgtcaggaggtgaaaatgaagcttctgaggtttgcaagaaaatgtttacaaataagagtctggcatttagtatcctcgcatgcatctccagcatgggaaactataacacggctggccccaggctcgtcctgtctggctgcctctttgtaagaggggagaagattgcacagtgtgatggagctcattttcagcagagt

SEQ ID NO: 165 - DNA, 485 bp, Homo sapiens
aaacagaacgagacaccagctaggattataactttagcattctatagcagtctgctcacacagccctcctccatgctggctcttgggccacactgttcccacatggagcttgagtctcctccaacacattccatgagcttcaagtgcagagacatggtgtacacttcgggctgttctacagagcactccagaccatacgtggctgaatacgtgagtgagtgtttttctgtccacttataaaccatgttgatattaagcataaatataatccaaatcagctttccttttcttggcctaaaggaatatgatgggattaaaacagaagtgaattaagcaaagatccactattctgaacaaataacatagaagtgattgaacaatttggacccactaaattttgtgtctagctgtaaaatggacattgtgataaaaacaggatttgaaggaaaatgaatagctaatttgtcaattaaataattaaaact

SEQ ID NO: 166 - DNA, 198 bp, Homo sapiens
  misc_feature (1)...(198): n = A,T,C or G
agcgtggtcgcgggtcgaggtntgccacccggctcttcttaacctgttttgttttctgctcagcacggttaaaagaccaacgtgtgtggatcaaatataaaggccacacctttcagaccgaacctactcaaagatcctttactttgcaatagtttgaactggagaaccaaagacgggagacgaatgaaagcaaagatg

SEQ ID NO: 167 - DNA, 539 bp, Homo sapiens
ctgtttcataggctggagatgcactcttctagactgctcgagacagccagagacaggggaggagggaagaaggatactgtggaaagggatggcggggcaaacatttagagctagaagccactactgggccaatgctaaagtttctgtctctaagcctaaaaaagccagtgtagtagggcccttatcactcttagtttgctaggtttcccctctgaaataatgagcagatttagccaggctagcagaaaggaagaggacggggctgtgcaggagttagcagaatcttgattcttgctctatggtcggtacttgcacaggaagtgttggcgcttgttgcattcgttgctgctccaagttaaaaagttgttattggagctcatctcagcacagtgcttgttcccacccatggacttgccagaccaggatctgtacagatacatggccccatcaatccactgccactgctgcctcttctgtgggtcgtgcaggccaatccatatcggctggcttctctgatagccacttatgtactctgctat

SEQ ID NO: 168 - DNA, 555 bp, Homo sapiens
ccatctgatctataaatgcggtggcatcgacaaaagaaccattgaaaaatttgagaaggaggctgctgagatgggaaagggctccttcaagtatgcctgggtcttggataaactgaaagctgagcgtgaacgtggtatcaccattgatatctccttgtggaaatttgagaccagcaagtactatgtgactatcattgatgccccaggacacagagactttatcaaaaacatgattacagggacatctcaggctgactgtgctgtcctgattgttgctgctggtgttggtgaatttgaagctggtatctccaagaatgggcagacccgagagcatgcccttctggcttacacactgggtgtgaaacaactaattgtcggtgttaacaaaatggattccactgagccaccctacagccagaagagatatgaggaaattgttaaggaagtcagcacttacattaagaaaattggctacaaccccgacacagtagcatttgtgccaatttctggttggaatggtgacaacatgctggagccaagtgctaacatgccttg

SEQ ID NO: 169 - DNA, 193 bp, Homo sapiens
ctgcggcccatgatgtcagagctggaagagagggcacgtcagcagaggggccacctccatttgctggagacaagcatagatgggattctggctgatgtgaagaacttggagaacattagggacaacctgcccccaggctgctacaatacccaggctcttgagcaacagtgaagctgccataaatatttctcaa

SEQ ID NO: 170 - DNA, 207 bp, Homo sapiens
  misc_feature (1)...(207): n = A,T,C or G
aaaggcagacactgagtcagtattaatagattaactaaactgcactgtaatttagataaaattactgtgtctcactgtntattacatgcaaaatccacataaattgtcatttaaccaacagtactgnacgagcgaacatctcgatatatgaaaactgcatcatcaattcaacgttttggtacttgaaactgcatcataaatgcaaca

SEQ ID NO: 171 - DNA, 265 bp, Homo sapiens
cctggcttccctgccagtccctgtccttcacactatgagggagagtcctgacttgaaatcagaagacctgagcatctattcttggctctgccacttattattgtgtgaccaataatctctctaggtttcagttacctccttcataagtgctctgtgcagtaaggaaggagaggggaagcaatggtctgtggtgctaagggagagccagatggtgctggtgtctgaaggaggagggagaatattctgagcaggggcaatgatgtgg

SEQ ID NO: 172 - DNA, 449 bp, Homo sapiens
  misc_feature (1)...(449): n = A,T,C or G
ccatgattctgtcttttcaatgactgtggcttctactcnaacaanatccttnncnaggagtggcttgccaagcagnntgaagttgtctgccccaaccagcaggaccttnnccagtcgaattnnctctccacacgcaaggnctanttcatttccaattaanatcaggtcttcagaggtcaccttccactggcggctggcaaagtgcaccacggcaaatagcctgccatactgccccgtgacgatcatctcattcaccttcttcacgacctctgcatggtgtctggtctcctcaactgggtctggcagaacaacttctggacanngtggtgaactcagggatgttttaggaacatatcctggtanatatgaagtgctctgtgaattgaaccttcgagaancagaccaaagggaggctgctccgggccccgaaggtctcangatgctgtggc

SEQ ID NO: 173 - DNA, 367 bp, Homo sapiens
cgagcggccgcccggcaggtccattggcgtaaaccttgaagcgatccaagccacagcgaatggacagatcaaagaactgtccgggaccaaatgggttgtgggtgatcttcttctcctcggatccccacgagccattcagaaggctgttccggaccacggtaccgttgcccatgcggggattaatgtgcagagctatgtcccctgaggagcccaccttgaagttgatagcaaagctcttgcctgtgggaggcacatagcccttgatgatgatggttcttcgagctgtgagccctccttgcagcctcccgaaatatggcacaggcgggttgaaggttgggggtccttccatggtgggcaggctgttcag

SEQ ID NO: 174 - DNA, 458 bp, Homo sapiens
ggcagccatctccttctcggcatcatggccgccctcagaccccttgtgaagcccaagatcgtcaaaaagagaaccaagaagttcatccggcaccagtcagatcgatatgtcaaaattaagcgtaactggcggaaacccagaggcattgacaacagggttcgtagaagattcaagggccagatcttgatgcccaacattggttatggaagcaacaaaaaaacaaagcacatgctgcccagtggcttccggaagttcctggtccacaacgtcaaggagctggaagtgctgctgatgtgcaacaaatcttactgtgccgagatcgctcacaatgtttcctccaagaaccgcaaagccatcgtggaaagagctgcccaactggccatcagagtcaccaaccccaatgccaggctgcgcagtgaagaaaatgagtaggcagctcatgtgcacgttttctgttt

SEQ ID NO: 175 - DNA, 325 bp, Homo sapiens
ccttctcatttgaggggattcctcaagactcaaccccacaggcccccactgtaggaaacaagccagagaaagcagcattcagagaatgggggacagagaaggggaaagatatgatcccaaatgcagtacaaagttggcgtctggttctgacacaaaccagatactgaagcactcacggtcaggtcagcaacctcctttgatggacccccaaaagctgactgaccaggcaaactgctttcaaggaatgaaagagtggagggtagggcttgtagcaaacaagccagtttcagtcactctgttcccccaggagaacaacctttagcac

SEQ ID NO: 176 - DNA, 195 bp, Homo sapiens
gtggtctgagctcggcctatgggggcctcacaagccccggcctcagctacagcctgggctccagctttggctctggcgcgggctccagctccttcagccgcaccagctcctccagggccgtggttgtgaagaagatcgagacacgtgatgggaagctggtgtctgagtcctctgacgtcctgcccaagtgaacag

SEQ ID NO: 177 - DNA, 214 bp, Homo sapiens
  misc_feature (1)...(214): n = A,T,C or G
ctgccacccggctcttcttaacctgttttgttttctgctcancacggttaaaagaccaacgtgtgtggatcaaatataaaggccacacctttcagaccgaacctactcaaagatcctttactttgcaatanttngaactggagaaccaaagacgggagacgannnaaagcaaagnngctcaaagaaccaaaggaaagacctgaaggaatccaca

SEQ ID NO: 178 - DNA, 310 bp, Homo sapiens
cctgtgggcttttcccaacaagcaggctcagtgccagcctctgtgtcagcctccagggcacgccaaccttctcatggtgccccaagccccaccccaatgcacacataggaagtctccaggctgcttgggcagaggcacaatcattttagattaaaaaaaattgaacaaagagaccctcttgcgagaggtgagatgaggccctgccatgcaaaggagtcccagcagaggaggaagaattccatcctggagttcaagtttctgtgcagagacaggacctggggacagagaacggtcctccacccaatttcag

SEQ ID NO: 179 - DNA, 386 bp, Homo sapiens
cccgccttccccggtcccagcccctcccagttcccccagggacggccacttcctggtccccgacgcaaccatggctgaagaacaaccgcaggtcgaattgttcgtgaaggctggcagtgatggggccaagattgggaactgcccattctcccagagactgttcatggtactgtggctcaagggagtcaccttcaatgttaccaccgttgacaccaaaaggcggaccgagacagtgcagaagctgtgcccaggggggcagctcccattcctgctgtatggcactgaagtgcacacagacaccaacaagattgaggaatttctggaggcagtgctgtgccctcccaggtaccccaagctggcagctctgaaccctgagtccaacacag

SEQ ID NO: 180 - DNA, 304 bp, Homo sapiens
gtggagttactggcctactccttccccatgagccctccctgtctgcactgcccaggccagagggtagagcacaggggtttccccatactacctccccactgggtccagtcttgacaaaggcaggaagccagctagggtgggggcgatagggtcagcgggtatgtcccactgttggaggtcactggtattctgtttgtttttgttttgtttcgttttgttttttgagacagggtctcgttctgtcgcttagctggagtgcggtggcgtgatcatggcactgctattcttgaagcactccacccac

SEQ ID NO: 181 - DNA, 341 bp, Homo sapiens
ctgcctcccttgaaactctcttcccaatcaagggctcccaaggagctgcaggccaagtcctctgctcctatttagcaagaggcaggcggcaattcgggctgatctccccatcacccttcatttaaccgcaaaaaagtcaccaaccaacttctcagaccccctgggcaatccagggtttcttgtttcctaagctcctatggaacaagcaatcagttctttcttggacttttggttcaattccttctcattcagaggaaatatggttgccgtgtaggcagatgtctcctaggagcgtgtgtgtgtaagagcctgtgtgaaattcagccaggttagcaccaagg

SEQ ID NO: 182 - DNA, 533 bp, Homo sapiens
  misc_feature (1)...(533): n = A,T,C or G
ctgaacaacaatggctatgaaggcattgtcgttgcaatcgaccncaatgtgccagaagatgaaacactcattcaacaaataaaggacatggtgacccaggcatctctgtatctgtttgaagctacaggaaagcgactttatttcaaaaatgttgccattttgattcctgaaacatggaagacaaaggctgactatgtgagaccaaaacttgagacctacaaaaatgctgatgttctggttgctgagtctactcctccaggtaatgatgaaccctacactgagcanatgggcaactgtggagagaagggtgaaaggatccacctcactcctgatttcattgcaggaaaaaagttagctgaatatggaccacaaggtagggcatttgtccatgagtgggctcatctacgatggggagtatttgacgagtacaataatgatgagaaattctacttatccaatggaagaatacaagcagtaagatgttcagcaggtattactggtacaaatgtagtaaagaagtgtcanggaggcag

SEQ ID NO: 183 - DNA, 200 bp, Homo sapiens
ctgctccttgtcatctccggagctccagacggtgcgcagggcacgctcctggttcctccgtgccacccggatcaggtagaccatggaggctcccaggaagaggatcaacaccatcacgaacagccccgccagaaccaccacctttgagccaaggggcaggggaggattctcctgctggcggaagcagcggagcatgcagg

SEQ ID NO: 184 - DNA, 72 bp, Homo sapiens
  misc_feature (1)...(72): n = A,T,C or G
ctgagcancacaggccaggaggccacagtgtaagcaataacagatctgccacatgcagaagcaaatatcagg

SEQ ID NO: 185 - DNA, 217 bp, Homo sapiens
aaaaactctggcttggatgttacacagaccaacaacccaaacagcagcaacaacaacacaaactccccccacccccttcttcatcagccccaagattgtgaaaatgacaggaagtccaggttggctctggcatttatagcactgacatacattcagcccagagaagctctggtgacaggctctctaaacaagtccctgttcgggccccctggtcagg

SEQ ID NO: 186 - DNA, 328 bp, Homo sapiens
aaaatctcaaactaaaaatgggaatcatacttaaacattagcattcccaggagagttggagcaagacctctgtgcccactatcactcaacatttcattatttaagtcttagcaagtgcaaaacagaaaagatgcataaatattagagaaggaaagttattttttgcaaatggcgtattttttatctggaaataccagagaatcaagtgagaaactttgaagaataaaataattcagtaatgatgcttcctaccctaggttaattaatataaagaagagttaaattcctatggcatatttctggtaacaaaaacccaggatcttgtttt

SEQ ID NO: 187 - DNA, 575 bp, Homo sapiens
  misc_feature (1)...(575): n = A,T,C or G
ctgagcagccctggatctttgccgtactgtgactgggctctttgccctatttttccctctgtctgtgcccctggatggcaggctgaagtcagaggggctgtttcattctcagccccctcagcagcactgggggaagaaagcattgtcacaacaggttctttctggccctcacccaacagcctgggcacttggccctcctcctccttgacagccctcccccttcctgcaaaggacaggggcgacaggggttggtgttgggattggctcccgctgcctgacaaccacaagtttatttggaaggctagcgggaagcccagcggctggcgtttcccttgactaaggaacagggtgcccatcagagtggggcgggcagctttgggaaggacacaagaagcagtaagagtgtaaagaggatgctggcctgggctcacaccaatgccacagtcagcttcctttctgcccactgtgcctctcaccttgcgtggnttggtgacagtctcaccagtctctctcagaggctacagatccagctccccgattccgtgaatcagctactccgctatctgagagagctg

SEQ ID NO: 188 - DNA, 325 bp, Homo sapiens
cctgtggccctgcagaagagcccacgtgcaaatccagctcctcccagcagaacaacacagtcctggtggaaggctgcttctgtcctgagggcaccatgaactacgctcctggctttgatgtctgcgtgaagacctgcggctgtgtgggacctgacaatgtgcccagagagtttggggagcacttcgagttcgactgcaagaactgtgtctgcctggagggtggaagtggcatcatctgccaacccaagaggtgcagccagaaacccgttacccactgcgtggaagacggcacctacctcgccacggaggtcaaccctgccgacac

SEQ ID NO: 189 - DNA, 222 bp, Homo sapiens
ctgtatcactgaaaatttcctgatattgtatgaaaaggaaaaagaaaaagatttcaaagtgatccaggctacagtctcaatgctgtaaaactacgtcggcgcccagccaggtgctgcaaaggagctcagaaaaatgaaaagagccgaaccaggctagtggaattccagatctccctgctttagacacttcactttcatgttattgtaagatttttttttttt

SEQ ID NO: 190 - DNA, 178 bp, Homo sapiens
ctggaacaactcagtgcaatgggatttttgaaccgtgaagcaaacttgcaagctctaatagcaacaggaggtgatatcaatgcagctattgaaaggttactgggctcccagccatcatagcagcatttctgtatcttgaaaaaatgtaatttatttttgataacggctcttaaacttt

SEQ ID NO: 191 - DNA, 291 bp, Homo sapiens
cctgagccaccgtgccttgccacaaactatttttaatcattaaaaaagaaaaaagaaaataaggcaggccggcttctcacattacatgcttttagaaaaaggtctcatccttgaagcagctttgttatatgcagagcacagtactggcttcaaaaaatatataaaggttctgtgcactggcactgtttacatgtgaagaattgccatcaacttctgtgaaaattagcaagctggcacagtggctcacgcctgtaatctcagcacctcaggaggccgaggtgggcggatcac

SEQ ID NO: 192 - DNA, 363 bp, Homo sapiens
ctgccaaatgggaagaatagaagaatttgcccctaaacccctcctgtgtgctgaccctgtgctagacagtgctggagacatagttgggggtggagaactgcccttatggagcttgcagtccagtgaggtggacagacctgtccccagacagtgatggcccaaaatggtcaggactttaatggaggaggtgaggtgttgaaagcacaggcagagtggtcagggctgaagtcggagaagcacagggactaggcccaatccagcctggaaagtcagggaggacttcctagaggaagggacatcgaactaagacctgaactatgagaaataggcaggaagaagttgtacctgactcatttttctcag

SEQ ID NO: 193 - DNA, 201 bp, Homo sapiens
caggtactcatagtagctgtcggccctggcgcccagcgtgaatacgcccaggtgggtgaagaggccactgtgggtattgatgaacatgggcaccagcccatccttcttcccagacaggccgtggatgtgctgtgtcaccttctccactgcctcctgaaacttcttatcccctgtgagacgggagagctcccggaactccag

SEQ ID NO: 194 - DNA, 367 bp, Homo sapiens
  misc_feature (1)...(367): n = A,T,C or G
aaagttgaactaanattctatcttggacaaccagctatcaccaggctcggtaggtttgtcgcctctacctataaatcttcccactattttgctacatagacgggtgtgctcttttagctgttcttaggtagctcgtctggtttcgggggtcttagctttggctctccttgcaaagttatttctagttaattcattatgcagaaggtataggggttagtccttgctatattatgcttggttataatttttcatctttcccttgcggtactatatctattgcgccaggtttcaatttctatcgcctatactttatttgggtaaatggtttggctaaggttgtctggtagtaaggtggagtgggtttggg

SEQ ID NO: 195 - DNA, 315 bp, Homo sapiens
aaaaatatttacgtcttacaggagctggataatccaggtgcaaaacgaattctagagcttgaccagtttaaggggcagcagggacaaaaacgtttccaagacatgatgggccacggatctgactactcactcagtgaagtgctgtgggtctgtgccaacctctttagtgatgtccaattcaagatgagtcataagaggatcatgctgttcaccaatgaagacaacccccatggcaatgacagtgccaaagccagccgggccaggaccaaagccggtgatctccgagatacaggcatcttccttgacttgatgcac

SEQ ID NO: 196 - DNA, 179 bp, Homo sapiens
ctgtgcaaatgggcatgggggtgcatggaagggaggaagagcaccgggcccctgaacctgccccttttaaggagggggaggagccgtcaggccaggaaggggaaatagtgcaaggcagagcccaggctgcaaaaggggtccagcacccagcgaggaagggggggtgtcccccaccccca

SEQ ID NO: 197 - DNA, 423 bp, Homo sapiens
ttgttcttcgggtcgctcttgaagtccccgggcttcaggatgacgtgctggttgtcgggccgtttgatgatacagtgcgtctgttcacacttcttacagcactccccgggggcctccatgagttcgaagccagggctgcaggaggtgttgcagggcacgtgggtgcaggcgatgacgttgagcagggtgttgttgtccaccttgtccgtgcacacgcagtcctggcacttggaggaataaactggagaaccgggctggtactcagcattcccgtgaacacacacccccttggactcacaccagtagaaaggacagcaccttccaggcaccatcttgctcttcacttcgaatcccagcgggcacacggagggcttctctttgcacaggctggtgttgcacttgcagacggtaatgttgcagcag

SEQ ID NO: 198 - DNA, 372 bp, Homo sapiens
aaatgttctcatcagtttcttgccatgttgttaactatacaacctggctaaagatgaatatttttctactggtattttaatttttgacctaaatgtttaagcattcggaatgagaaaactatacagatttgagaaatgatgctaaatttatagttttcagtaacttaaaaagctaacatgagagcatgccaaaatttgctaagtcttacaaagatcaagggctgtccgcaacagggaagaacagttttgaaaatttatgaactatcttatttttaggtaggttttgaaagctttttgtctaagtgaattcttatgccttggtcagagtaataactgaaggagttgcttatcttggctttcgagtctgagttt

SEQ ID NO: 199 - DNA, 502 bp, Homo sapiens
  misc_feature (1)...(502): n = A,T,C or G
ctgcagcctgggactgaccgggaggctctgattatttacccaccacaggtaggttgtgttctgaatctcaggttcacaggttaaggctacagcatcctcatcctccacggggttggagttgttgctggtgatgaagggtttgggtggctctgcatagactgtgatcgtcgtgactgtggtcctattgaggccagtgtctgagttatgggcttggcacgtataggatccactattattcacagtgatgttggggataaagagctcttgggtggattgctggaaagtcccattgacaaaccaagagtactgtgcaggtgggttagaggctgcgtggcaggagaggttcagattttcccctgatctgtaagatgtgtttagaggggaaatggtgggggcatccgggccatagaggacattcaggatgactgaatcactgcgcctggcactcactgggttctgggtttcacatttgtagcttgctgtgtcatttcttgngacattg

SEQ ID NO: 200 - DNA, 609 bp, Homo sapiens
  misc_feature (1)...(609): n = A,T,C or G
ctgaaaagacagaagaatctaaggccgctgctccagctcctgtgtcggaggctgtgtgtcggacctccatgtgtagtatacagtcagcaccccctgagccggctaccttgaagggcacagtgccagatgatgctgtagaagccttggctgatagcctggggaaaaaggaagcagatccagaagatggaaaacctgtgatggataaagtcaaggagaaggccaaagaagaagaccgtgaaaagcttggtgaaaaagaagaaacaattcctcctgattatagattagaagaggtcaaggataaagatggaaagccactcctgccaaaagagtctaaggaacagcttccacccatgagtgaagacttccttctggatgctttgtctgaggacttctctggtccacaaaatgcttcatctcttaaatttgaagatgctaaacttgctgctgccatctctgaagtggtttcccaaaccccagcttcaacgacccaagctggagccccaccccgtgatacctcgcagagtgacaaagacctcgatgatgccttggataaactctctgacagtctangacaaangcagcctgacccagatgagaacaaaccaatgg

SEQ ID NO: 201 - DNA, 173 bp, Homo sapiens
ctgcctcagaatgtcagaagggtgccattttcagttcttctcctgtctgactgtagggtatagaacaggcagccctaagtgctgctttctgcgcaaatgttttttgattacaaatctctaactaggtttgaaatgttttatagaataagacaatattcttttcaacaaacttt

SEQ ID NO: 202 - DNA, 182 bp, Homo sapiens
gtgccacactggcccttggtgttgttgccaaaccggtggtagggcagcctgacggagaaggacaggccattgtaggagacgaggacacccagctcggggatgtccaccacgtagttgatgccagactggtacacctccagcccgtacttcttgtagggcagtgccaccgcctgcctgttcac

SEQ ID NO: 203 - DNA, 106 bp, Homo sapiens
gtgcctttgaggcagcagggttccacctgaatgagcatctctataacatgatcatccgacgctactcagatgaaagtgggaacatggattttgacaacttcatcag

SEQ ID NO: 204 - DNA, 178 bp, Homo sapiens
tctctccaccctctgcagctcatcgacaggaccgagtccctaaaccgctccatagagaagagtaacagtgtgaagaaatcccagccagacttgcccatctccaagattgatcagtggctggaacaatacacccaggccatcgagaccgctggccggacccccaagctagcccgccagg

SEQ ID NO: 205 - DNA, 518 bp, Homo sapiens
ctgcacaaacaatggaatggtatttagtcctctgataacaattcggttgtgaacatcccgagctaggatgtgaagggctccggtacaaccttcaactatttcttccatgcggaccccctccacaaattgctgctgtgtcccacccatggacgtacggcgctgggtatcctgatgtgcacgaacaagcaactgaactagtcgtggaatggcaccctgctcacgcaaaggtgcatgatttgcgggacaaagggcaagatttcgaatcaatccaacagtagcctttatcagaggccagtgggatggtgggtgtaagagcttaaccacaactggtagtccatagtgaaggcgaactgcattctgggccatctctgcttcttggtgtcggctggtcagatgacgaagagcacagatggcaggctcagtgatgtcttccctgtcaccagcccgaaggacagtacgcacaagagcctctataccacccacttggcagaccatcatcttgttcttataattattgc

SEQ ID NO: 206 - DNA, 367 bp, Homo sapiens
  misc_feature (1)...(367): n = A,T,C or G
aaagttgaactaagattctatcttggacaaccagctatcaccaggctcggtaggtttgtcgcctctacctataaatcttcccactattttgctacatagacgggtgtgctcttttagctgttcttaggtagctcgtctggtttcgggggtcttagctttggctctccttgcaaagttatttctagttaattcattatgcagaaggtataggggttagtccttgctatattatgcttggntataatttttcatctttcccttgcggtactatatctattgcgccaggtttcaatttctatcgcctatactttatttgggtaaatggtttggctaaggttgtctggtantaaggtggagtgggtttggg

SEQ ID NO: 207 - DNA, 145 bp, Homo sapiens
aaaaaaattagattttagctggagcttttgactaatgtaaagtaaatgccaaactaccgacttgatagggatgtttttgtaagttaattttctaagactttttcacatccaaagtgatgctttgctttgggttttaactgtttgg

SEQ ID NO: 208 - DNA, 193 bp, Homo sapiens
  misc_feature (1)...(193): n = A,T,C or G
ggccgagcccccgatcccccactacgctgccatgtggcagcagcaggagctactagaatattctcagcacaggaatgaggcttccttggtttccatgtctgtaagggttactgatcacttaccttcttctctttcagacttgaatctgnngacatttctttattgatatggcaaattgcttgcagatattttt

SEQ ID NO: 209 - DNA, 255 bp, Homo sapiens
ccaggatgtagggccatccttaggggtggcggcctctgcccagaggggagaggttaataggtcaaggatgggggccctgagggtatcaaaaggacagggacagttcccagttcccactatccagcatgctctccactggcccaggactccatcgacacatcccaaagattccaatcaaagtttgaggttggctctcccaaactttcctctgcagagccattcctgcaggctccctcatgctggcaagcaccctcc

SEQ ID NO: 210 - DNA, 351 bp, Homo sapiens
ctgaaagtgcttctcagctcgccccatgtaagttctcattccatgtaaatgacattttccagttacaactggtactgagattttgcctctctctttccttactcatcctcccaaatgtctttgtgggagccatatcagtggataccaagctctgtatccatttgtcccctgccctccacaatgtgtgacatagaacagggactttggccctgggaaagcaaaagctcccagtaaggaatcctgtgcccaatgatgtaaaacaattccaaacatccaggaatttttgtatcatagagcgaattacttcctatcttttcattagaggctatgaggacttctaattagtctcag

SEQ ID NO: 211 - DNA, 236 bp, Homo sapiens
aaaaacccagaagatggggcagctcagagactggtttcctaatacacaagacctagcaggaaatgatcaagaaaatattaggcatgcagataggaacaactctgatgataatcatttggcttcagaagatactagtgccaagcaaagtggtgagccagacgcctgtcataggcttcgtcctgagggtccagcatgaggaagatcaaatcaaaccttgataataaagtatgaggcag

SEQ ID NO: 212 - DNA, 135 bp, Homo sapiens
ggacagggtttcactgtgttagccagaatggtcttgatctcctggccttgtgatccgctcacctcagcctcccaaagtgctgggattacaggcgtgagccaccacacccagacttttttttttcttctttttttt

SEQ ID NO: 213 - DNA, 567 bp, Homo sapiens
gtgcgcagccccgtcaccaacattgctcgcaccagcttcttccacgttaagcggtccaacatttggctggcagcagtcaccaagcagaatgtcaacgctgccatggtcttcgaattcctctataagatgtgtgacgtgatggctgcctactttggcaagatcagcgaggaaaacatcaagaacaattttgtgctcatatatgagctgctggatgagattctagactttggctacccacagaattccgagacaggcgcgctgaaaaccttcatcacgcagcagggcatcaagagtcagcatcagacaaaagaagagcagtcacagatcaccagccaggtaactgggcagattggctggcggcgagagggtatcaagtatcgtcggaatgagctcttcctggatgtgctggagagtgtgaacctgctcatgtccccacaagggcaggtgctgagtgcccatgtgtcgggccgggtggtgatgaagagctacctgagtggcatgcctgaatgcaagtttgggatgaatgacaaggttgttattgaaaagcagggcaaaggcacagacctc

SEQ ID NO: 214 - DNA, 470 bp, Homo sapiens
aaaaaaccatggaggtaaagaaaaagattaaactgcacatcaggtagtcaataaaggcagaatttactgtgcatgaacccacatgagcaaagggtaagaacaggcaaatcagagaaaaatccaaacagaaacaaggacagcaacaacaacaaacctctttgaactcagacaaaaggcaattaaactaacaagcaatacaatgcaatttttagcctttcatattttcaagcattaaagagtgctggagaggacgctggaacgggcgctttcattttggatagtaatcttgtaatatttctgaaacatatgcctacatagtatttctgggaatccaacctatataaataaaagcaccagtatgtattacagcagtgttattttgaaaaaaaataaaaaaaggaaataaaagacgatcaataacgaaatggttgaatgcctttttggtacatcaacaagtactgtgtattcag

SEQ ID NO: 215 - DNA, 504 bp, Homo sapiens
  misc_feature (1)...(504): n = A,T,C or G
ttggtgcacaaaatactgtcatttgctcaaagctggctgccaaatgtttggtgatgaaggcannaatgaatggctcaaaacttgggagaagagcaaaacctgaaggggccctccagaacaatgatgggctttatgatcctgactgcgatgagagcgggctctttaaggccaagcagtgcaacggcacctccacgtgctggtgtgtgaacactgctggggtcagaagaacagacaaggacactgaaataacctgctctgagcgagtgagaacctactggatcatcattgaactaaaacacaaagcaagagaaaaaccttatgatagtaaaagtttgcggactgcacttcagaaggagatcacaacgcgttatcaactggatccaaaatttatcacgagtattttgtatgagaataatgttatcactattgatctggntcaaaattcttctcaaaaaactcagaatgatgtggacatagctgatgtggcttattattttgaaaaag

SEQ ID NO: 216 - DNA, 208 bp, Homo sapiens
gtgttccccaccttgggcatcatgcaccacaacaaacaggccactgagaatgcagaggaggaagtgaggcgaattctggggctgctggatgcttacttgaagacgaggacttttctggtgggcgaacgagtgacattggctgacatcacagttgtctgcaccctgttgtggctctataagcaggttctagagccttctttccgccagg

SEQ ID NO: 217 - DNA, 316 bp, Homo sapiens
ccagctctgtctcatacttgactctaaagtcatcagcagcaagacgggcattgtcaatctgcagaacgatgcgggcattgtccacagtatttgcgaagatctgagccctcaggtcctcgatgatcttgaagtaatggctccagtctctgacctggggtcccttcttctccaagtgctcccggattttgctctccagcctccggttctcggtctccaggctcctcactctgtccaggtaagaggccaggcggtcgttcaggctttgcatggtctccttctcgttctggatgcctcccattcctgccagacccccggc

SEQ ID NO: 218 - DNA, 327 bp, Homo sapiens
ctgaagaaaactatcttcccagtcatgagaactcggggtgtcgattgagcagcctctcctcaacaatggagatttttcagaaaagtgagtctggtttaagggacacaaatcccacagagtttcttggcacaatgtttcagtaagcatgagtgagacccaaaaacgtccaatgagagaccaacaggcccctgacctaagtcagagctagactgcagaactctcagcccagctctgcagcagacatctgcgtcatcctcccagaaattcaactcaggccacacttctggcaaaagaagtttcaccctgactgtccttgttttcagtcag

SEQ ID NO: 219 - DNA, 215 bp, Homo sapiens
aaaatgtgaatgccaggaatggtgctgttgatatgaatattaggacaaggagcagacgtttcatcaggactaggtgtctctggcggagtctgtggaggaataaacaaagatactcgtgcaatgttggatatttctgatttcagatcgaccttatcaacagcctgaatagcaatgaaaagatctgtgccattttcaaaagtaatgttttctggttt

SEQ ID NO: 220 - DNA, 344 bp, Homo sapiens
gtggttgtagagcgactgcacataggtgaagacacacttggggtcaggcttcttgcccatgatcatcatgtcgtccacctccaccaggggcacacagtccaccagcatccgtggggccccgagcaggggttaggactttttggtttttaccagccccttctggaccagacagcggtagaattcctggatgtacgtgtacacgcacttccagtcaggctctcgaagccgcaccatgtcctctgtatccaggagctgcgggcagtccgcatgggtctccgcagatgagaaggccacctcgaagttctggcgtcggttctgagggctaagctgcccatagtcgaagg

SEQ ID NO: 221 - DNA, 262 bp, Homo sapiens
gtgaatggggcgggctgggccacaggaagcctcaccccaggacgggagaacgtgggtggaggccacctgccttggggagtccagaggagcagcagccgtgagagctgtgggggccggattctggcagggcaggggcgtttgtcctgcagggatgggggtcgctgtgtgtccgcctctaggggctgtcaggcggtagctactgcaccagccggtaggtgatgtacctgccagccgtctcactgggccggatgatcttcaccac

SEQ ID NO: 222 - DNA, 309 bp, Homo sapiens
aaatggggtcattttacatacattattttgcattctgatttcttttacttgacatgaatgtattgtcaataagtatcgatctaattcaaaaatagctgcataacatatggatgtatctaatttatccgaacattctttattaaaagtctatgagtggtctggcgcagtggctcacgcctgtaatcccagcaccccggggggccgaggtgggctgatcaccctgaggtcaggagagaccggcctagccaacatgctgaaaccccgtctctactaataatacaaaaattagccaagtgtggtggcgcgcac

SEQ ID NO: 223 - DNA, 279 bp, Homo sapiens
ctgccccccacccttcccttcgatgacaacgtttgcaggcttcagggggaccagggaacaaagctggggcctggcagccccactacgctgccagccggggagaacaagtcacaattacaaattatcacaacaattagcgcctgtacttgggggatctgcaaattgaggaggccccagctcctcattgtacacgggtctatttggcagtgaccttgctctggagacgatgatattccttcagcctgagggaattgatgttgatgaacccggtggcatcag

SEQ ID NO: 224 - DNA, 607 bp, Homo sapiens
aaaatactgtcatttgctcaaagctggctgccaaatgtttggtgatgaaggcagaaatgaatggctcaaaacttgggagaagagcaaaacctgaaggggccctccagaacaatgatgggctttatgatcctgactgcgatgagagcgggctctttaaggccaagcagtgcaacggcacctccatgtgctggtgtgtgaacactgctggggtcagaagaacagacaaggacactgaaataacctgctctgagcgagtgagaacctactggatcatcattgaactaaaacacaaagcaagagaaaaaccttatgatagtaaaagtttgcggactgcacttcagaaggagatcacaacgcgttatcaactggatccaaaatttatcacgagtattttgtatgagaataatgttatcactattgatctggttcaaaattcttctcaaaaaactcagaatgatgtggacatagctgatgtggcttattattttgaaaaagatgttaaaggtgaatccttgtttcattctaagaaaatggacctgacagtaaatggggaacaactggatctggatcctggtcaaactttaatttattatgttgatgaaaaagc

SEQ ID NO: 225 - DNA, 100 bp, Homo sapiens
ctgtgtctttagagctattgccacattagcctttgcactgtatagcgtctggctttatggaacttaagtttaccaaatataaaaagaaacttctgctttt

SEQ ID NO: 226 - DNA, 260 bp, Homo sapiens
ccactgataactcagtagccatctgaatagtcatgcggtttaagaatacatccttgtataatctgacatacaaatttgtcatttcctgcacatgcacaccattgttaaaaaaaaaagccagtaatagtgtctggatcggtcaggagcacggcctctgagtcccctgtaatttagttaagctaaattaatacctcataccaaatggctccaggaaaactgtcctgcaggtcagaagggagcccaagaagaaaagcacttgg

SEQ ID NO: 227 - DNA, 168 bp, Homo sapiens
ctgtcaccatcaagcccttgctcttgggttcattcttcgggagccccaggggcctatctatggggaagggagtgggtgtgacttgggagccaatggaggggtgggatgggtgagagaaaagggcagaattcagatctgttttgtcttggattcttctggaactagagg

SEQ ID NO: 228 - DNA, 200 bp, Homo sapiens
aaaaataactggaatctggaggccagtcaaacctttttggacagatatctcctcgaaatacttctttattaaagtatttgaaggagatgacttcttagacctttggcttgacattctgggattcccagactatattgttctttcataagcaaataatctgttatcttttagattcttcagaataaatacatctttacttt

SEQ ID NO: 229 - DNA, 149 bp, Homo sapiens
ccagaacaccgtgggctgttaacttgccttgagttggaagcggtttgcatttacgcctgtaaatgtattcattcttaatttatgtaaggttttttttgtacgcaattctcgattctttacctgcccgggcggccgctcgagccctatag

SEQ ID NO: 230 - DNA, 287 bp, Homo sapiens
aaataaccaagggcctccagaggcccctgttcttccttttgccctgtctaaaatccaatgaaatatattaagtgttaaactggtatgaagaggtggaactaaattctttgaaacacaaggtggagtatcactttttacttaaactttgagtcctttacatttataactgctattcaaaaaaaattagacaaagacatctagatttagattaacgtgatcaaagggattattgtggatcattaaaggaaacttaacattaagccttcatgtaccaaatactaatattt

SEQ ID NO: 231 - DNA, 287 bp, Homo sapiens; identical to SEQ ID NO: 230

SEQ ID NO: 232 - DNA, 222 bp, Homo sapiens
ctggggccactgtcggcatcatgattggagtgctggttggggtggctctgatatagcagccctggtgtattttcgatatttcaggaagactggcagattggaccagaccctgaattcttctagctcctccaatcccattttatcccatggaaccactaaaaacaaggtctgctctgctcctgaagccctatatgctggagatggacaactcaatgaaaattt

SEQ ID NO: 233 - DNA, 536 bp, Homo sapiens
ccaacatggtaaaaccccatctctactaaaaatacaaaaattagctgagcgtggtggcgggcacctgtaatcccagctactcaggagactgaggcaggagaatcatttgaacccgggaggcagatgttgccagtgagctgagatcacgccattgcactccagcctgggcgacaagagcaaaactcaaaaaaaaaaaaaaaaaaggaaaaacaaaacccagaatgcagaaactgcattggtattttgagctagttacaggcagtaacacttctactaggaaggaaaatatataatttatccaaaaatagttctaagatatagaaaaacatctcaatcctctagggccaagacctgccccttatttacattcacaaagccattaagtgaacccagaactgaccagcagacagtgaggcagggcctgctctgtggcacatgccagtcacctactgcgtaagtggacaaagagaaagcagaaggtaatggagagagttttcatttgcttatttagtgaaaaacagaggaaaatcactcga

SEQ ID NO: 234 - DNA, 562 bp, Homo sapiens
ttggtgcacaaaatactgtcatttgctcaaagctggctgccaaatgtttggtgatgaaggcagaaatgaatggctcaaaacttgggagaagagcaaaacctgaaggggccctccagaacaatgatgggctttatgatcctgactgcgatgagagcgggctctttaaggccaagcagtgcaacggcacctccacgtgctggtgtgtgaacactgctggggtcagaagaacagacaaggacactgaaataacctgctctgagcgagtgagaacctactggatcatcattgaactaaaacacaaagcaagagaaaaaccttatgatagtaaaagtttgcggactgcacttcagaaggagatcacaacgcgttatcaactggatccaaaatttatcacgagtattttgtatgagaataatgttatcactattgatctggttcaaaattcttctcaaaaaactcagaatgatgtggacatagctgatgtggcttattattttgaaaaagatgttaaaggtgaatccttgtttcatttctaagaaaatggacctgacagtaaatgggg

SEQ ID NO: 235 - DNA, 313 bp, Homo sapiens
ctggtgtgatgggaacttcagtggctacatctgctagcaaaattattcctcaaggggccgatagcacaatgcttgccacgaaaaccgtgaaacatggtgcacctagtgacctgagatccgaactgggctcatcaagggcagcggcactgcagaggtggagctgaagaagggagccactctcaaaatcacgctggataacgcctacatggaaaagtgtgacgagaacatcctgtggctggactacaagaacatctgcaaggtggtggaagtgggcagcaagatctacgtggatgatgggcttatttctctccag

SEQ ID NO: 236 - DNA, 172 bp, Homo sapiens
gtgcgcgccaccaagcccagctcatttttgtatttttagtagagatggggtttcacgatgttggctaggatggtctcgatctctggtcagagtcttttctgtaaatatccttggtaaagaagcaattttagactgtagctgttgcaaatgctttaaggaagaagcaaaacaa

SEQ ID NO: 237 - DNA, 454 bp, Homo sapiens
  misc_feature (1)...(454): n = A,T,C or G
ctggggcatgtttatttcctcctgggcctggcaggctgggagcagaatatanacaaaggcactggggcacctgggtctggcacggtctggancttggccgnctgggtagcaaccgtnaagggtgtgccagggcgngcanggactggagtnatnctnccagaactgagagagggccctcggggcatgggggcatcacaagtgctaggcttggcacaggtacaggggagaggttacggagtgggtgtgtgcagggcctggtgggaatggggagacccgtggacagagcttgttagagtgtcctagagccagggggaactccangcagggcaaattgggccctggatgttgagaagctgggtaacaagtactgagagaacaaaagcttgtgggtcagcangccccacaaagatgtgactgcagacaggatcggccctgggagagaccgaggctccag

SEQ ID NO: 238 - DNA, 331 bp, Homo sapiens
aaaatactaagtcatcttacgtttccattttattaacgggatgttgcaatcgtttgtaaactaataaacttataaagtgattggcacaaagactccttgagcaaaagctgtgcagttaagtacaaaaagatacttaatttggagactcttacagtaatttttgccatgtcaaaacaatggcttttacattgaaagattaatagaaactctacatatgttaatttttttatagaacctgactcaaatcaaggtactctccattttattgccttacctgaatcagtcctttttggttggtaatagatttttttatacacccacgtttgattta

SEQ ID NO: 239 - DNA, 353 bp, Homo sapiens
gtggtgcagtgggtgagctttgctgattccgatatagtgcccccagccagtacctgggtgttccccaccttgggcatcatgcaccacaacaaacaggccactgagaatgcaaaggaggaagtgaggcgaattctggggctgctggatgcttacttgaagacgaggacttttctggtgggcgaacgagtgacattggctgacatcacagttgtctgcaccctgttgtggctctataagcaggttctagagccttctttccgccaggccatgtggcaagcgacaggcacaaaacaattttccaagtcaataggaaaaacctcagagctgaaatctttatatgctgtactacacag

SEQ ID NO: 240 - DNA, 356 bp, Homo sapiens
aaagaactacaagccctcagactctacctagcggcggcagtgcccgcggtctgctatacgatgtactccattcggtttaagctctgggcactttccaagtctctgttgtcctgtttgtttgtccagtttgggtgttttgtcggcgtgccgttggggggcttctcttctctgtctaccagcgtgtacgccggctgcttggcaaaccgggctttctgctggtgtttgtccatgtcgtcctcttctacttcagaattgtgtgtccttattttagacattttggagttcttgttctcgtaatccttgatggggaccgtgttggccccatgtttctcaatggggtttttgatctggttcag

SEQ ID NO: 241 - DNA, 425 bp, Homo sapiens
  misc_feature (1)...(425): n = A,T,C or G
gtggagtccacaatcttgggaaggcgtctatttcttcttgtgtgtactcatctagacgtttaggtatttttcgtggttgaggaagctnctctactaaattcttaagaatatcttctggaatatactcatctggaaaaagatgcaacctttccatcattgttcttctgtgaaggttttttggcagcatgccataaatagctagttttacaattgccactggatccctcaggtgaagctgancagctgttacttgtctaaatccacctgggtagtcagtgcatggtacacaggtttatgtaatccctgaagtcttatagatgccatagcngcaagtttgccaggtggctgcattttcccatctaagagataccatattctagcaaaagtggcccattgntggggcgccctagagaaactcgacacct

SEQ ID NO: 242 - DNA, 101 bp, Homo sapiens
  misc_feature (1)...(101): n = A,T,C or G
ctgtctgggaccacacggncaccggcctnctgtgagcggatccactcactgtctcgccagtcctgtgagttgttgctgccacagcagtggaactnctgctg

SEQ ID NO: 243 - DNA, 284 bp, Homo sapiens
  misc_feature (1)...(284): n = A,T,C or G
gtgatctgccagccttggcttcccaaagctctaggattacaggcgtgagccactgcacccagccaagaggtagtttcttaaaggttanttgcagcanaatctgaaaccataaaaaggaaattttcatgctctgttacattaaaattggntggcccatcttgaactttgaatggactgcttacccatgcatgattctgtaacatcatggntggttatttggaaaatattggttcactgaattgtgaanatcttccaaatattgaaacatttcantatgtattttt

SEQ ID NO: 244 - DNA, 266 bp, Homo sapiens
ctgagctcaccatagtctaatagaaaacaaccgaaaccaaataattcaagcactgcttattacaattttactgggtctctattttaccctcctacaagcctcagagtacttcgagtctcccttcaccatttccgacggcatctacggctcaacattttttgtagccacaggcttccacggacttcacgtcattattggctcaactttcctcactatctgcttcatccgccaactaatatttcactttacagccaaagtgatgtttg

SEQ ID NO: 245 - DNA, 432 bp, Homo sapiens
ctgctcaggatgctgaggcaggagaatcacttgaacctaggaggcataggttgcagtgaactgagatcgcgccactgcactccagcctgggtgacagagtgagactctgtgatacagtttccatctggctctcagtcttaggacctgtggaactagccaccctgttgtgaggcagccagaccacatgaagaggccatgtgtaggtgttctggctgatagccccagccatcagtcagtattaaccactagacaagcaagtggacaagccttcagatgatgccagctccagcctttgagctgccccagctaataccacatggagcagacactatccctgcagagtcactgttttaagctactaagtgttaagtgttttttgaggtagcaatagataactgggaaattctcaaacctattttgcatcctcttttt

SEQ ID NO: 246 - DNA, 367 bp, Homo sapiens
ctgctgtcctaaacccaacttctctgtgatgcccggatttcttgattttgatccagtagctgctcattttcctgccttttacatttaggagattcaagctctgtcatttcctctagctgcccctgaagtccgtccttcctgcagggcccaactccacgtagagtgagtgcagccacacagcagtaaccagatagagcagcctcccctgcagacatgagcaaagaagggatccagagagccaaggctgtatcatagattcttgtggggtcaaaggggcagtcagtatgtcccggcccctcatccagtggtaccagaggatccagcagtcctgggtggcagtcagcaataaggcggcggccaccgttgg

SEQ ID NO: 247 - DNA, 105 bp, Homo sapiens
aaatgacagaagcgtatatgaattcaagaaaatttaagctgcaaaaatgtatttgctataaaatgagaagtctcactgatagaggttctttattgctcatttttt

SEQ ID NO: 248 - DNA, 538 bp, Homo sapiens
  misc_feature (1)...(538): n = A,T,C or G
ccattgaagagttgatttgaacgatgacgtcccatggtgggtgaaaatctatcctggataactcctggaccagtaccaattccgctacctggcatttgtccaaacatatcagcaagtcctccaagtgggtccctatccattttcatcctgggtggcatgaacggtccctccagaaagaagtcacttctcatcccttgagccataggagcaggaataaacacccctagatcttttactgcatcttgacgaatttgattgatcgtctttggtccattgtcaagaaaagccttgcgaggaacccaatggtgttctcgcaactctacggtatcctgcagcaggaaacgaatccttgctggcaattccttacttaacatcaaggagcacattcgggcaaagtactgatccattaaggacttggctcgttcatggtctaatctangtcccactgtcctcattatctgacagaggcactccaaatcctctcccatatctttgagttggactctcttcttcttttccaaaagtgttttgatgcact

SEQ ID NO: 249 - DNA, 557 bp, Homo sapiens
  misc_feature (1)...(557): n = A,T,C or G
ctgacacagaggtggtggaagactccttgaggcagcgtaaaagtcagcatgctgacaagggactgtagatttaatgatgcgttttcaagaatacacaccaaaacaatatgtcagcttccctttggcctgcagtttgtaccaaatccttaatttttcttgaatgagcaagcttctcttaaaagatgctctctagtcatttggtctcatggcagtaagcctcatgtatactaaggagagtcttccaggtgtgacaatcaggatatagaaaaacaaacgtagtgttgggatctgtttggagactgggatgggaacaagttcatttacttaggggtcagagagtctcgaccagaggaggccattcccagtcctaatcagcaccttccagagacaaggctgcaggccctgtgaaatgaaagccaagcaggagccttggctcctgagcatccccaaagtgtaacgtagaagccttgcatccttttcttgngtaaagtatttatttttgtcaaattgcaggaaacatcaggcaccacagtgcatgaaaaatctttcacagctag

SEQ ID NO: 250 - DNA, 465 bp, Homo sapiens
  misc_feature (1)...(465): n = A,T,C or G
ctgtgtcaaaagaaaaacagagtctacaaatcttactaaatacaaccattggcaaacagtcatatttaatggttcagaaaaggagagacagtaaaggaatttttcttttcaactttgctccggaggaggctgtctggagcccattcctttggcctcgacttttcagtgttataactgtccttgaagtaagctggctaagcagaggaaaacttgttcttgttttctttttaacccttaccccctgccacataatcacatctttacacttctttttttttttttttaanatgggaagtcggagtctcgctctgtcgcccaggctggagtgcagtggtgccatctcggctcattgtaacctctgcctcccaggttcaagtgattcaagccattcttgtgcctctgcctcctgagtagctgggattataggcncacaccaccacacctggcgaattttttttttttttt

SEQ ID NO: 251 - DNA, 429 bp, Homo sapiens
  misc_feature (1)...(429): n = A,T,C or G
ctgtagataacaatagcaaagtgaagacagcatctaagctgtgttcagccctgactctttctggtcttgtggaagtgaaagagctgcagcgggagcccctaacccctgaggaagtacagtctgttcgagaacaccttggtcatgaaagtgacaacctgctgtttgttcagatcacaggcaaaaaaccaaactttgaagtgggttcttctaggcagcttaagctttccatcaccaanaagtcttctccttcagtgaaacctgctgnggaccctgctgntgccaagctgtggaccctctcagccaacgatatggaggacgacagcatggatctcattgactcagatgagctgctggatccagaagatttgaagaagccagatccagcttccctgcgggctgcttcttgtggggaagggaaaaagaggaagg

SEQ ID NO: 252 - DNA, 559 bp, Homo sapiens
  misc_feature (1)...(559): n = A,T,C or G
aactactgaattccaagctgcctcggnggcaggagacctgtgttgatgccatcaaagtgccagagaaaatcatgaatatgatcgaagaaataaagaccccagcctctacccccgtgtctggaactcctcanncttcacccatgatcgagagaagcaatgtggttaggaaagattacgacaccctttctaaatgctcaccaaagatgccccccgctccttcaggcagagcatataccagtcccttgatcgatatgtttaataacccagccacggctgccccgaattcacaaagggtaaataattcaacaggtacttccgaagatcccagtttacagcgatcagtttcggttgcaacgggactgaacatgatgaagaagcagaaagtgaagaccatcttcccgcacactgcgggctccaacaagaccttactcagctttgcacagggagatgtcatcacgctgctcatccccgaggagaaggatggctggctctatggagaacacgacgtgtccaaggcgaggggttggttcccgtcgtcgtacacgaagttgctggaaga

SEQ ID NO: 253 - DNA, 181 bp, Homo sapiens
ccaggatggcctcgcgctccctatcggcgtccggcagggtggacttgaactggtcatgggctgagatcaggccctcaatctcctcgatggtatggacgatgaacatgtcctggaggtcctccatggcgctctccatccagttgttgaagggggccgcgcgcttggcgtattccaggtgcag

SEQ ID NO: 254 - DNA, 137 bp, Homo sapiens
ccaccacctcggctccaaaggtaaaagagacgccacggtcgttctcgccccagccctgcacgtccttgtcagggtcagaccacagcaggtcacacagcaggccctggtcaggcacatctgtgggccgcatgatccgc

SEQ ID NO: 255 - DNA, 193 bp, Homo sapiens; identical to SEQ ID NO: 169

SEQ ID NO: 256 - DNA, 532 bp, Homo sapiens
  misc_feature (1)...(532): n = A,T,C or G
ctgcgtgagctcactgtcagacaagatggaagaagaagggctggagtgtccaaactcttcctctgaaaaacgctattttcctgaatccctggattccagcgatggggatgaggaagaggttttggcctgtgaggatttggaacttaacccctttgatggattgccatattcatcacgttattataaacttctgaaagaaagagaagatcttcctatatggaaagaaaaatactcctttatggagaacctgcttcaaaatcaaatcgtgattgtttcaggagatgctaaatgtggtaagagcgctcaggttcctcagtggtgtgctgaatattgtctttccatccactaccagcacgggggcgtgatatgcacacaggtccacaagcagactgtggtccagcccgccctgcgggtggcggatgaaaaatggatgttaacattggtcatganggtttggctacnntgatccctttcggagaactgctgtaccaangaaacaatcctgangnattgnactgatgatatgctncaa

SEQ ID NO: 257 - DNA, 300 bp, Homo sapiens
  misc_feature (1)...(300): n = A,T,C or G
ctaataattaggctgtgggtggttgtgttgattcaaattatgtgttttttggaaagtcatgtcagtggtagtaatataattgttgggacgattagttttancattggagtaggtttaggttatgtacgtantctaggccatatgtgttgganattganactantanggctaggcccaccnntgcttcgcanncggcaaagactagtatggcaataggcacaatattggctaanagggagtgggtgttganggttatnanantagctntantgaacancgatagcattattccttctaggc

SEQ ID NO: 258 - DNA, 308 bp, Homo sapiens
ggccagcccaccctcctggggctgacatgagccattccctgtgatgttcactctcctcccaaagcaaaccacagccaagcctgtctgagctgggagtccccttccccagcagagctcccagtccctgcatacccagcggggtggcgactcgggaagagctgagctggagacggctctagaccaagtccggccaaccaggcggttctgtaatcctctcccagggcccatggaagttaggcttccatcaggcgcacttttcccaccaggggctctgggaggacgtgtcttctaaagtgttccgtgctcac

SEQ ID NO: 259 - DNA, 344 bp, Homo sapiens
cctaacctccatgtgcaggcccagtttggcactctttctgactattttgatgccctgtacaagaggacaggggtggagccaggggcccggcctccagggtttcctgtgctgagcggggatttcttctcctatgcggaccgggaggatcattactggacaggctattacacttcccggcccttctacaagagcttagaccgagtccctaaaccgctccatagagaagagtaacagtgtgaagaaatcccagccagacttgcccatctccaagattgatcagtggctggaacaatacacccaggccatcgagaccgctggccggacccccaagctagcccgccagg

SEQ ID NO: 260 - DNA, 416 bp, Homo sapiens
cctacagacttatttcttcttggacacacccacggcgcggccacggcggccagtggtcttggtgtgctggcctcggacacgaaggccccagaagtgacgcagccctctatgggcccgaatcttcttcagtcgctccaggtcttcacggagcttgttgtccagaccattggctaggacctggctgtattttccatcctttacatccttctgtctgttcaagaaccagtctgggatcttgtactggcgtggattctgcataatggtgatcacacgttccacctcatcctcagtgagttctcccgccctcttggtgaggtcaatgtctgctttcctcaacaccacatgagcatatcttcggcccacacccttaatggcagtgatggcaaaggctattttccgccgcccatcgatgttgg

SEQ ID NO: 261 - DNA, 189 bp, Homo sapiens
aaaacaagtgtgatgccatatcaagtccatgttattctctcacagtgtactctataagaggtgtgggtgtctgtttggtcaggatgttagaaagtgctgataagtagcatgatcagtgtatgcgaaaaggtttttaggaagtatggcaaaaatgttgtattggctatgatggtgacatgatatagtcag

SEQ ID NO: 262 - DNA, 219 bp, Homo sapiens
ctgtatcaacacatcccagcatcgtcaaggagacactgcctctgctgctgcagcatctctggcaagtgaacagagggaatatggttgcacaatccagtgacgttattgctgtctgtcagagcctcagacagatggcagaaaaatgtcagcaggaccctgagagttgctggtatttccaccagacagctataccttgcctgcttgccttggctgtgcagg

SEQ ID NO: 263 - DNA, 193 bp, Homo sapiens
aaagtttgtgctataaaattgtgcaaatatgttaaggattgagacccaccaatgcactactgtaatatttcgcttcctaaatttcttccacctacagataatagacaacaagtctgagaaactaaggctaaccaaacttagatataaatcctaccaataaaatttttcagttttaagttttacagtttgattt

SEQ ID NO: 264 - DNA, 605 bp, Homo sapiens
  misc_feature (1)...(605): n = A,T,C or G
tcaggaggcagcgctctcgggacgtctccaccatggcctgggctctgctattcctcaccctcctcactcagggcacagggtcctgggcccagtctgccctgactcagcctgcctccgtgtctgggtctcctggacagtcgatcaccatctcctgcactggaaccagcagtgacgttggtggttataaccatgtctcctggtaccaacaacacccaggcaaagcccccaaactcatgatttatgatgtcactagtcggccctcaggggtttctaatcgcttctctggctccaagtctggcaacacggcctccctgaccatctctgggctccaggctgaggacgaggctgattattactgcagctcatatactagcatcatcactgtggtattcggcggagggaccaaggtgaccgtcctaggtcagcccaaggctgccccctcggtcactctgttcccgccctcctctgaggagcttcaagccaacaaggccacactggtgtgtctcataagtgacttctacccgggagccgtgacagtggcctggaggnagatagcagccccgtcaaggcgggagtggagaccaccacaccctncaaacaaagca

SEQ ID NO: 265 - DNA, 593 bp, Homo sapiens
ctgttactgaagaggaaccctgtcatgttcttccaacacttcattgaatgtatttttcactttaataactatgagaagcatgagaagtacaacaagttcccccagtcagagagagaagcggctgttttcattgaagggaaagtcaaacaaagagagacgaatgaaaatctacaaatttcttctagagcacttcacagatgaacagcgattcaacatcacttccaaaatctgccttagtattttggcgtgctttgctgatggcatcctacccctggacctggacgccagtgagttactctcagacacgtttgaggtcctcagctcaaaggagatcaagcttttggcaatgagatctaaaccagacaaagacctccttatggaagaagatgacatggccttggcaaatgtagtcatgcaggaagctcagaagaagctcatctcacaagttcagaagaggaatttcatagaaaatattattccaattatcatctccctgaagactgtgctggagaaaaataagatcccagctttgcgggaactcatgcactatctcagggaggtgatgcaggattaccgagatgagctcaaggact

SEQ ID NO: 266 - DNA, 461 bp, Homo sapiens
ctgatccagcgcttccaggatgagcacgaggactggtccattatcatcttcaccaacacgtgcaagacctgccagattctgtgcatgatgctgcgcaaattcagcttccccaccgtggctctgcactccatgatgaagcagaaagaacgctttgccgccctagccaagttcaagtccagcatctaccggatcctgatcgcaacagacgtggcctcccggggcctggacatccctacggtacaggtggtcatcaaccacaacacccccgggctccccaagacctccaaggatccttgcaggagttgaaggagagggctctgagccgatacaacctcgtgcggggccagggtccagagaggctggtgtctggctccgacgacttcaccttattcctgtggtccccagcagaggacaaaaagcctctcactcggatgacaggacaccaagctctcatcaaccag

SEQ ID NO: 267 - DNA, 489 bp, Homo sapiens
ccttcgagaagatccctagtgagactttgaaccgtatcctgggcgacccagaagccctgagagacctgctgaacaaccacatcttgaagtcagctatgtgtggtgaagccatcgttgcggggctgtctgtagagaccctggagggcacgacactggaggtgggctgcagcggggacatgctcactatcaacgggaaggcgatcatctccaataaagacatcctagccaccaacggggtgatccactacattgatgagctactcatcccagactcagccaagacactatttgaattggctgcagagtctgatgtgtccacagccattgaccttttcagacaagccggcctcggcaatcatctctctggaagtgagcggttgaccctcctggctcccctgaattctgtattcaaagatggaacccctccaattgatgcccatacaaggaatttgcttcggaaccacataattaaagaccagacctgcccgg

SEQ ID NO: 268 - DNA, 242 bp, Homo sapiens
  misc_feature (1)...(242): n = A,T,C or G
aaataaaaaagctatatnnnaaagtaacctaggagggccaggcacagtggctcatgcctattacctcancactttgggaggcagaggccagaggactgctcgagcccaggagtttgagaccagcctgggcaacatggggagaccccatctcttcagaaaacaaaaaggtcagccaggcatagtggcacacttggtggtcccagctattcangangctgaggtggtggatcacacctcggccg

SEQ ID NO: 269 - DNA, 320 bp, Homo sapiens
aaagaatttattaagcctgttataccacacagtatgttttatacactgacatacaactccctaataagataaagcaaagacaaaaaagtttatcttattagaaacaagatacaccaccacttattgtcttcagacattattgcactttaactttcttaatttgacaaagcattcaagaaacatctgcagactagttttaacagacaaataacacctgtaagcagacatgactgtcctaaattgtttattaagtatgaattttacaaactttacttatattagcggtaacggtggagctggagagtattgcgccttctcca

SEQ ID NO: 270 - DNA, 400 bp, Homo sapiens
aaatccgcgccctgcacacgcaattcatttagaccttttcgtgaatcttctccactttcacaaacaacctatccagatcattcctcaggtcatctagtaaacccttggctgattccagattgttctcgttggtttctattttgaccgagtatgcaaccaaactgtccacagcagtcctgagcattttcaagtccgcctccacttggctgactgaggctttcaggttgtctagagaagaaagtctgtccaggaagtcctgaggaggcagacgggcggcctgggcttggtcctgactgagcagcgtgtgcacctgctcctgcaccttctggagtgattccacggtgctggggagctcgcccacactcctcttcagctcctccacgtcaccgtagagcaccag

SEQ ID NO: 271 - DNA, 536 bp, Homo sapiens
aaaaaagcaacttccagggttgtcattgtacaggttttgcccagtctcctatagcatggtatagtgataactgattttttataacaatgactcagaggcattgaagatccataactatcttctgaattatcacagaaagaagaaagttagaagagtttaatgttaagtgtattaaaaatcatattctaattcttttaatttggttatctgagtatgataatataggagagctcagataacaagaaaaggcaattggttagaacactccattcccacaggatgtgcattaacagactttttactgcatatgtctttatatagtttgcaaactaattcaaccattttacacagcattaatttttttttaactgggttgacattgggctgaaacatttgcttatcatcttataattattttttcctgttctttaatggattttacccccatctgacatagtgtttggactttagtgtatgtgacacttcaagatcatctctgcccattctgatgatagttacaatgaggttacccatgg

SEQ ID NO: 272 - DNA, 424 bp, Homo sapiens
aaaatgagcagtgttcgtcttcgtaaagaaattaagagaagaggcaaggaccccacagaacacatacctgaaataattctgaataattttacaacacggctgggtcattcaattggacgtatgtttgcatctctctttcctcataatcctcaatttatcggaaggcaggttgccacattccacaatcaacgggattacatattcttcagatttcacagatacatattcaggagtgaaaagaaagtgggaattcaggaacttggaccacgttttaccttaaaattaaggtctcttcagaaaggaacctttgattctaaatatggagagtatgaatgggtccataagccccgggaaatggatacaagtagaagaaaattccatttataaagtactgagagaatgatattggattttgctgaacagg

SEQ ID NO: 273 - DNA, 232 bp, Homo sapiens
aaataaaaaagctatatgtaaaagtaacctaggagggccaggcacagtggctcatgcctattacctcagcactttgggaggcagaggccagaggactgctcgagcccaggagtttgagaccagcctgggcaacatggggagaccccatctcttcagaaaacaaaaaggtcagccaggcatagtggcacacttggtggtcccagctattcaggaggctgaggtggtggatcac

SEQ ID NO: 274 - DNA, 112 bp, Homo sapiens
gtgatccacccgcctcggcctcccaaagtgctgggattacaggcttgagcccccgcgcccagccatcaaaatgctttttatttctgcatatgttgaatactttttacaattt

SEQ ID NO: 275 - DNA, 468 bp, Homo sapiens
aaaaagcagcctgggcaagagaagtgggtgggtttaggagaatccctttcgaaaaattcagagcattattattaatccttcttaaattaaatgcagggccaagcatgctgcacgtggaatctggacaattttttgataaactttaaggctgctaaataatttacagaaactgtgaatgcattttcattttacgaggcaaaagagaaaatattcaagattgcatagcaattttattttttgaaatggttatcctaaagaatttccttaaattcagattttgcaaaattcctactctccaagtcatcaagtgaacactaaaagcaactttactcgtgaatacagtggactctttacgaggcatgcatttttcataaatctaggccaaagtgaactaattgagatttaattctaaattcatcctgtgatttctgcatataatattggtataaaaccagtaaaaatactttt

SEQ ID NO: 276 - DNA, 461 bp, Homo sapiens
  misc_feature (1)...(461): n = A,T,C or G
ccttctcctgagctgaaagttctttggcagatgagcaagaaactgaaagctgatgtacctgactggctctgtaagatcagaaaactgtatccagaataagccctatggattaacccctgagtacccagagtaaaaactaatttacagaacttccttattgatctgctggttcttccagatcatattctggctattggtatggctggcctttctgaaggtaccctgcttgtctattttcctgactcagctcttgcctgcctttttcacatgttgctgcaattagactcaccgtgaggactacagtcaatttcagtctatcttgngcccaatacaacaaggatttttaatagtnncaacccacacctcacccactaggactnaatgttcacaacangaaggaccattgctgcatactncttgaccancaacttttttgaagatatttttaagtgcngagtagg

SEQ ID NO: 277 - DNA, 549 bp, Homo sapiens
  misc_feature (1)...(549): n = A,T,C or G
gggaagatggcggacattcagactgagcgtgcctaccaaaagcagccgaccatctttcaaaacaagaagagggtcctgctgggagaaactggcaaggagaagctcccgcggtactacaagaacatcggtctgggcttcaagacacccaaggaggctattgagggcacctacattgacaagaaatgccccttcactggtaatgtgtccattcgagggcggatcctctctggcgtggtgaccaagatgaagatgcagaggaccattgtcatccgccgagactatctgcactacatccgcaagtacaaccgcttcgagaagcgccacaagaacatgtctgtacacctgtccccctgcttcagggacgtccagatcggtgacatcgtcacagtgggcgagtgccggcctctgagcaagacagtgcgcttcaacgtgctcaaggtcaccaangctgccggcaccaagaagcagttccagaagttctgangctggacatcggcccgctccccacaatgaaataaagttattttctcattcccaaaaaaaaaaaaa

SEQ ID NO: 278 - DNA, 344 bp, Homo sapiens
ctgtagtcccagttactcgggaggctgaggcaggagaatcgcttgaacccgggaggtggagattgcagtgagcccagatcgcaccactgcactccagtctggcaacagagcaagactccatctcaaaaagaaaagaaaagaagactctgacctgtactcttgaatacaagtttctgataccactgcactgtctgagaatttccaaaactttaatgaactaactgacagcttcatgaaactgtccaccaagatcaagcagagaaaataattaatttcatgggactaaatgaactaatgaggataatattttcataattttttatttgaaattttgctgattcttt

SEQ ID NO: 279 - DNA, 145 bp, Homo sapiens
  misc_feature (1)...(145): n = A,T,C or G
ccaacttggggggctgngtccacccagcccgnccgtcctgtgggctgcacagctcaccttgttccctcctgccccggttcgagagccgagtctgtgggcactctctgccttcatgcacctgtcctttctaacacgtcgccttcaa

SEQ ID NO: 280 - DNA, 410 bp, Homo sapiens
  misc_feature (1)...(410): n = A,T,C or G
ccattactgattttcaattaatttatgcataaatgagacccaaactatcactaattttcagctatatgaattgatagccacttgacatcagtgaaaggtacagtagggagtagatgaaattgtatttttaatgaaaaggctttgatgggagattcaagatttttggttttttttttttttgagacagggtcttgccctgtcacccaggctcgagtgcactggagtgatcacagctcactggccgcaagtgatcctcctgccttggccccttaagtgccagggttacaggcatgagctaccatgcctggcagaaattcaagatttggataaacttacttctttgccaagcctgttcttcaagttattcanaactgggtgtataccttgtcctcatatgtatcttgtccctg

SEQ ID NO: 281 - DNA, 377 bp, Homo sapiens
ccattttcatcctgggtggttagggcccctgtgggagcagatgggcactgtcaccaatttggctctgccaggacagggcaggccctgcggcctctggctgaatcacccactgtccactccagagggtccatcgagaatagctgtccaagcaaggctgtacctacgtacaaactaaagctaccgctcattcatctgctgtccaggaaagcttaggagacattcctgcctttctacatggaaaaaaaaatagtacaagttttggaattttctgtaattaaacaaggcatattcatgtactacatatttcagcactaaggcggttgcttcactttatatctatataaaaaaagtggtaaaagtcttttccttttgtgcag

SEQ ID NO: 282 - DNA, 529 bp, Homo sapiens
  misc_feature (1)...(529): n = A,T,C or G
agacattactggttatagaattaccacaacccctacaaacggccagcagggaaattctttggaagaagtggtccatgctgatcagagctcctgcacttttgataacctgagtcccggcctggagtacaatgtcagtgtttacactgtcaaggatgacaaggaaagtgtccctatctctgataccatcatcccagaggtgccccaactcactgacctaagctttgttgatataaccgattcaagcatcggcctgaggtggaccccgctaaactcttccaccattattgggtaccgcatcacagtagttgcggcaggagaaggtatccctatttttgaagattttgtggactcctcagtaggatactacacagtcacagggctggagccgggcattgactatgatatcagcgttatcactctcattaatggcggcgagagtgcccctactacactgacacaacaaacggntgttcctnctnccactgacctgcgattcaccaacattggtccagacaccatgcgtgtcacc

SEQ ID NO: 283 - DNA, 558 bp, Homo sapiens
  misc_feature (1)...(558): n = A,T,C or G
ccagcacctctagaagggaatttcttttcttaatatcacagttcccaaacttaagacattatgatcaagattttctatctttatttgcactagtatctaaaatactcagtgaaatcttttttgctacactatatttacttttacagactttccattaccacatacatacaccatcatgctaagaaaccaccaagtttttcttctaatcccccactaaaattaacaggtttcaacaaacttgaaattataggggaactatggggaaaaccagagaagtatatggaagaaggaagaagtgtgaataggtcctacagaattttacaatcactttgccaagacaactataaatactatgaataattacttgaaatcaggttgtgtagaatctatagttctcttaaaaacaagttttgattctcaatattgcatttttataccaaataaaaaggatttagatctaacgtattttagtngcatacttactacctgcanactaaattcatttctcangtactctaaaaaacttcaatagaacaaactttatgagatgctataact

SEQ ID NO: 284 - DNA, 356 bp, Homo sapiens
aaaaaataaatggtatcttatttaattgtcctgttccttcccactccccgcctcctaggatgttagcccaagctcagggtaggcccagggggctgggagaaatgaagccacccatggggactggggaccaggggccttcagcatggcttctaggttccctcctccccctaccccatctcctacctccacagtacagactgtccccaacttaacagtggttcaacttaaaccatgtttcaactttacaattggtctgttggggtattaaatgaatttgtgacttaggatattttcatttatgatgggtttatcaggaagtaaccccatggtaagttgaggcatatctgtatatattt

SEQ ID NO: 285 - DNA, 184 bp, Homo sapiens
ctggactagtagaaactcgctgggaaggtggtctgaagccaggtgcctttgagttatcagggtgcatgttttccaagtgtccaagcactgagttacccaggaacgctgactgaacagtgaaagaggcatctgtagcaactcgtgaggacagtggaccatctccccagccctggttagctggcac

SEQ ID NO: 286 - DNA, 537 bp, Homo sapiens
ctgttacagtgacaagagataaaaagatagacctgcagaaaaaacaaactcaaagaaatgtgttcagatgtaatgtaattggagtgaaaaactgtgggaaaagtggagttcttcaggctcttcttggaagaaacttaatgaggcagaagaaaattcgtgaagatcataaatcctactatgcgattaacactgtttatgtatatggacaagagaaatacttgttgttgcatgatatctcagaatcggaatttctaactgaagctgagatcatttgtgatgttgtatgcctggtatatgatgtcagcaatcccaaatcctttgaatactgtgccaggatttttaagcaacactttatggacagcagaataccttgcttaatcgtagctgcaaagtcagacctgcatgaagttaaacaagaatacagtatttcacctactgatttctgcaggaaacacaaaatgcctccaccacaagccttcacttgcaatactgctgatgcccccagtaaggatatctttgttaaattgacaacaatgg

SEQ ID NO: 287 - DNA, 342 bp, Homo sapiens; identical to SEQ ID NO: 103

SEQ ID NO: 288 - DNA, 562 bp, Homo sapiens
aaatgcagtccactctgctttttgaagaggctttggttcagctcccaaatctcgattgcttgacgcagtctcctatgagaatactcagaaggtgtcttcttaaacaacaaacctatttttagtggtggagccgctcttagtagctgtgtctgcgtgggactgataaccaatcactatctttggaggaagtcctaacctttccttgtataccctccctatatgtgtaacagcttctctgttttcacattcagtagtccatattgctatcttatcacctttagctctaacattaacaacagcgccacatacatcatcactgtagtcatcaaaagattctccaataaggcacagaagtgtctctagccaaaagcgatcgaggtcacttcgtctctgctgtttgttcaatgtaattagccatcgtcctccccgtttgtttttctcatcttcccacataggctcaataccatccttaaaaagtgagtagtcacagccaggcattaaattactagacaactggatatggttgtacagagcccaaaagtcttcaacagtatcaaacttg

SEQ ID NO: 289 - DNA, 422 bp, Homo sapiens
aaacaaaaagttttgggtctgtctttggagtatttgtaacttctaaattttgaaatgactgaattaggaatttggatgcttattcttttagtctgtttgcctaaaaaccaatttacaatctgactgtctcttgggagagggaggtgccttgcaaactttcacattaagaatgtgcctgaggctgctttactctggaatagtctcagatctaaaatttcctctatataaggtggcatatgttaagttttgcttcattggaccgtttagaatgctatgtaaaatgttgccattctgttagattgctaactatatacccatctctgatttggctctccttaagtgataggatttgttattctaaaggtgataaacttgaaaatatcagaatctgagttttacttgaaattttgcagaatacccag

SEQ ID NO: 290 - DNA, 564 bp, Homo sapiens
  misc_feature (1)...(564): n = A,T,C or G
ctgtccaatggcaacaggaccctcactctattcaatgtcacaagaaatgacgcaagagcctatgtatgtggaatccagaactcagtgagtgcaaaccgcagtgacccagtcaccctggatgtcctctatgggccggacacccccatcatttcccccccagactcgtcttacctttcgggagcgaacctcaacctctcctgccactcggcctctaacccatccccgcagtattcttggcgtatcaatgggataccgcagcaacacacacaagttctctttatcgccaaaatcacgccaaataataacgggacctatgcctgttttgtctctaacttggctactggccgcaataattccatagtcaagagcatcacagtctctgcatctggaacttctcctggtctctcagaagtgtaacattctgagtcaacagcagacagagagctggagtaaagaagtcagtgggttacttgggagtgatcagcctgactctgaaatgacttttgataccaacataaagcaagagtctgggtcttctacttcttcatacagtggctatgaang

SEQ ID NO: 291 - DNA, 536 bp, Homo sapiens
ttggacctcctggctctctgctgtacatccgtggatccatcatgtccattttgagacgggaagatagtcttcaggaaagacacacaaaggtaacttgtgcagagggagatggcaaatttataacttctcagaaacacagtaatgataagtaaccaaggacttccaccaaagtcagtcccacgatgacgatggtcagccagagtattgataacctgatttctggtcctccccaaccagctccctgtccctgcttctgggtgctccttccttcctgagctcccagggttcctcaaggtcacttttggcgacaaaacataaaaaacaaatgatggcaggatggcaggaagaacctcatacccaagcagagtgccaggttttacagcctccgctcagccattcatatcctaagcaacaaaacatcagcaggatgcggaaggtcccgatagtaaaccatctccatcacatccatgtagccatccgtccatcaacctcttagaatcatccagaaacaagtcactcttcatctgtccagcaaa

SEQ ID NO: 292 - DNA, 578 bp, Homo sapiens
ctgccatcacatcggacatattggaggcccttggaagagacggtcacttcacactctttgctcccaccaatgaggcttttgagaaacttccacgaggtgtcctagaaaggatcatgggagacaaagtggcttccgaagctcctatgaagtaccacatcttaaatactctccagtgttctgagtctattatgggaggagcagtctttgagacgctggaaggaaatacaattgagataggatgtgacggtgacagtataacagtaaatggaatcaaaatggtgaacaaaaaggatattgtgacaaataatggtgtgatccatttgattgatcaggtcctaattcctgattctgccaaacaagttattgagctggctggaaaacagcaaaccaccttcacggatcttgtggcccaattaggcttggcatctgctctgaggccagatggagaatacactttgctggcacctgtgaataatgcattttctgatgatactctcagcatggatcagcgcctccttaaattaattctgcagaatcacatattgaaagtaaaagttggccttaatgagctttacaac

SEQ ID NO: 293 - DNA, 281 bp, Homo sapiens
ctgagtgccaggcgtggggtgacccccattacgtcactctggatgggcaccgattcgatttccaaggcacctgcgagtacctgctgagtgcaccctgccacagaccacccttgggggctgagaacttcactgtcactgtagccaatgagcaccggggcagccaggctgtcagctacacccgcagtgtcaccctgcaaatctacaaccacagcctgacactgagtgcccgctggccccggaagctacaggtcgacggcgtgttcgtggctctgcctttccag

SEQ ID NO: 294 - DNA, 187 bp, Homo sapiens
ctggtggcaggccaggccctcgcccacacactcgtcctctggccggttggcagtgtggagcagagcttggtgcgggttccgaaagagctggtcccagggcaccgtgtgcacgaagcagaggtgggtgttatggtggatgagggccagtccactgcccagttccctcagtgagcgcagccccagccag

SEQ ID NO: 295 - DNA, 306 bp, Homo sapiens
  misc_feature (1)...(306): n = A,T,C or G
ctgcggtttgntcaatggagctgctgattggggaaataattttcaacactatcctgaattatgtgcctgtctagataagcagagaccatgccaaagctataatggaaaacaagtttacaaagagacctgtatttctttcataaaagacttcttggcaaaaaatttgattatagttattggaatagcatttggactggcagttattgagatactgggtttggtgttttctatggtcctgtattgccagatcgggaacaaatgaatctgtggatgcatcaagctatcgtcagtcaaanccctttacct

SEQ ID NO: 296 - DNA, 381 bp, Homo sapiens
  misc_feature (1)...(381): n = A,T,C or G
gcgggatggggatgatgaggctattgttttttgtgaattcttcgataatggcccatttgggcaaaaagccggttagcgggggcaggcctcctagggagaggagggtggatggaattaagggtgttagtcatgttagcttgtttcaggtgcgagatagtagtagggttgtggtgctggagtttaagttgagtagtaggaatgcggtagtagttaggataatataaatagttaaattaagaatggttatgttagggttgtacggtagaactgctattattcatcctatgtgggtaattgaggagtatgctaagattttgcgtanctgggtttggtttaatccacctcaactgcctgctatgatggataagattgagaacctcg

SEQ ID NO: 297 - DNA, 410 bp, Homo sapiens
cgcttctgagctaaaagcttccatgaaggggctgggaaccgacgaggactctctcattgagatcatctgctccagaaccaaccaggagctgcaggaaattaacagagtctacaaggaaatgtacaagactgatctggagaaggacattatttcggacacatctggtgacttccgcaagctgatggttgccctggcaaagggtagaagagcagaggatggctctgtcattgattatgaactgattgaccaagatgctcgggatctctatgacgctggagtgaagaggaaaggaactgatgttcccaagtggatcagcatcatgaccgagcggagcgtgccccacctccagaaagtatttgataggtacaagagttacagcccttatgacatgttggaaagcatcaggaaag

SEQ ID NO: 298 - DNA, 260 bp, Homo sapiens
  misc_feature (1)...(260): n = A,T,C or G
gctttttttttttttttttttttttttttttgcacaatggtttattaaaggaatgtatggcccacatcaacctancaaggattctactggtaaaccttcccatggccaaaggaaaaacaagcaggagttgagtggctggggtggggtgcaggcaatggaaanagggcaaaagggtgtaaaanctgaagggggctanaagcttactcctgagtttnttccttntgtcttnaaatctttacttnttatggccaaanacccag

SEQ ID NO: 299 - DNA, 281 bp, Homo sapiens
ccaaaaagatgctggggcagattgtggacaagtagaagcacctccttcccctctgcgacattgaatggcgtggattcaatagtgagcttggcagtggtgggcgggttccagaaggttagaagtgaggctgtgagcaggagcctctgccaggggatgcaccatctgtggggaggggccgagggagactccatggtctctgctgtctgctctgtcctcctctgtggagaagagcttgagttccaggaacgttttgtcaaggctgctgtgactgtctggtctgc

SEQ ID NO: 300 - DNA, 600 bp, Homo sapiens
cctaccacaataataaaaaaccgtcaattacatcatcacattaaaataagccagatgtacaaaagtctgagacagtgaagacaaaaggacaacacaagatatttgttgaaaaatgtttgtgctctttgggcacttaattaaacattgcaaaatcaacatcatcttcttcttcatcagactctgcaaaatattttacttctttcctagcccgaccggttcgtggcagagaaggtggctcagtagggaagtctgaggggaagatgtccacatctgaatcctgatcaaaagatgtcttcttcggtttcttgcttgttgttttggatgttttcctgccagggttataatcgccttcattttcagagccagatgctttccttttctttgcccctcggcctttaccttttggtgttgtagtcttctttggaatgccaaattctgaatccgagtcagagtttacagcctctactactttcttctgttttggggctctcttgggcttagggactgtatctgaagacggttttccctttttagcagctaccgtttttacttggaactttatctgtctgtttcagaccaaatgatggtgaaaaaacagaa

SEQ ID NO: 301 - DNA, 305 bp, Homo sapiens
ccttctctgaaaaaagagaaggaattacttattaaaactaagcacacttagcaacttctttcccaatcctatctttattcgtttgcctggtgccaaatttttctggccctttttaatttgcaaaccttaaaaaaaaaaacaaagaaacaaaaacaccaaacacacacatatctcacacatagcactaagctagaagcagatataaatgggaccactgtgaatcaaaggggaaaaattccaggaaaaaaaaattccaatagcttcacagtttaactgaggttttggaaaaacttaagtgaattcag

SEQ ID NO: 302 - DNA, 222 bp, Homo sapiens
ccaaacttgcatttgcattttgcactcatgacgatgatgatgcccatggcgcacagaaccccagcgcagatgagcccgccaacctggaggctgtgccagtcatagtagaaaggactgtttttatcttctaggtcattggcgtccaggacaggaaagcctgccaggaacacaagcaggcccagggtcaccttctgcatgtcagagcgctggcctgtgtggttc

SEQ ID NO: 303 - DNA, 195 bp, Homo sapiens
ctgattttatttccttctcaaaaaaagttatttacagaaggtatatatcaacaatctgacaggcagtgaacttgacatgattagctggcatgattttttcttttttttcccccaaacattgtttttgtggccttgaattttaagacaaatattctacacggcatattgcacaggatggatggcaaaaaaaagttt

SEQ ID NO: 304 - DNA, 172 bp, Homo sapiens
ttgttttgtctcttccttaaagcatttgcaacagctacagtctaaaattgcttctttaccaaggatatttacagaaaagactctgaccagagatcgagaccatcctagccaacatcgtgaaaccccatctctactaaaaatacaaaaatgagctgggcttggtggcgcgcac

SEQ ID NO: 305 - DNA, 146 bp, Homo sapiens
ctgaaaagggtggcagagaaggaggcagtcaataagatgtccctgcacaacctcgccacggtctttggccccacgctgctccggccctccgagaaggagagcaagctccctgccaaccccagccagcctatcaccatgactgacag

SEQ ID NO: 306 - DNA, 377 bp, Homo sapiens
ctgtttacagaaatatagttgcgagtatacaaatgttccaatagaagcaaaatatctttttaatatttaacaagttatcacagatagctaaaaacatagatgcaaatgaaattcccccagagaacaaactgaaaatatctggtatcagtgctctgaaatcccaactatgaaagccatatacacaaaaatgtaacccttatatcattgcaggacaatggaagaaggcagttcagtggttgatcagtgtgctcaagcaaataaaattaaataaaaattaaaaatggcagaatggtagctaaaccacttgagaacaggttaatgaaattattggtactatacttaaaacattaagtaaaagaagtgaatgaaactcattt

SEQ ID NO: 307 - DNA, 246 bp, Homo sapiens
aaaacagtgtcaaacttctcatgcatggagcatgaattcttactaatagagactgtagttttttttcttgtctttggtaaatatataaaagaccttaatttttcttttttaatgaatggagaaaacatgagaaaaccagatggacctgttagtactacatttttaaggcattttatatttgatggtgccgtacttttaataataataaaactgaagttttttagtggcaatactgatttatttttt

SEQ ID NO: 308 - DNA, 191 bp, Homo sapiens
aaacgcaaagtagttggctgggcagggcttgatgaggccacacttgtactttttaacctggatctccttggtgggcgaggctgccagccagcgcggcagacggatggtcttcatgctgaagctcatgtagcttcgaataaacatccatgtcgtgactatggcaaagatgagggccaggaggcgaagcacac

SEQ ID NO: 309 - DNA, 342 bp, Homo sapiens
ctgtgtgcccctcctacatcaggggtaaggcccagctccccatcagcttccttgaactgtaaatcagattagatttggggatctgggctcagtctcaggagcagataaaactgggacactcagccttggggaagacaaagaaaagccacataggaaagagatagacagaccatgggcaagggaagattgcacagggaatgtgacatcagggaacagatgagggaggaggaggcgcggcggcctcggggagaggacgggaagcctgtcaggaaggggcccgggaagcaggaggaggtgtgtgtcatcgatgccctgctggctgacatcaggaagggcttccag

SEQ ID NO: 310 - DNA, 381 bp, Homo sapiens
ccagtttgccctctcaggctcctgggatggaaccctgcgcctctgggatctcacaacattgtctctggatctcgagataaaaccatcaagctatggaataccctgggtgtgtgcaaatacactgtccaggctggagtgcagtagtgcgatctcggctcactgcaagctctgcttcccgggttcacgccattctcctgcctcagcctcccgagtcgctgggactacaggcgcctgccatcaggatgagagccactcagagtgggtgtcttgtgtccgcttctcgcccaacagcagcaaccctatcatcgtctcctgtggctgggacaagctggtcaaggtatggaacctggctaactgcaagctgaagaccaaccacattgg

SEQ ID NO: 311 - DNA, 240 bp, Homo sapiens
  misc_feature (1)...(240): n = A,T,C or G
caaccgtggcatcncgcgaatncggggcaccagctaccagagccctcacggcatccccatannacctgctggaccggctgcttatcgnctccaccaccccctacagcgagaaagacacgaagcagatcctccgcatccggtgcgaggaagaagatgtggagatgagtgaggacncctacacggtgctgacccgcatcgggctggagacgtcactgcgctacgccatccagctcatcacag

SEQ ID NO: 312 - DNA, 263 bp, Homo sapiens
ctggagaaggagtgaatcctccccatgtacttgctacctgagaagcagttgacataggatgtacagatgggtgaaaagttggctgctgcaaatcaagcagatcattgggcagctttgatgtgctgttagaagaactaggggtagaaaatatgtcaatggctggtgcagtcattatccctcctgctgaggtggatacaggagaggctgcagttgttaaagaggtatgaggtttctttgcaagttcttttaggcgctgttccttt

SEQ ID NO: 313 - DNA, 300 bp, Homo sapiens
aaacaagatttgctgcatttccggcaatgccctgtgcatgccatggtccctagacacctcagttcattgtggtccttgtggcttctctctctagcagcacctcctgtcccttgaccttaactctgatggttcttcacctcctgccagcaaccccaaacccaagtgccttcagaggataaatatcaatggaacgcagagatgaacatctaacccactagaggaaaccagtttggtgatatatgagactttatgtggagtgaaaattgggcatgccattacattgctttttcttgtttgttt

SEQ ID NO: 314 - DNA, 123 bp, Homo sapiens
ctgcagcccccgtctcggcccccaccagtggctatcaggagtttgtacatgcggtggagcagggtggcacccaggtcagtgcggtggtgggcttgggtcccccaggagaggctggttacaagg

SEQ ID NO: 315 - DNA, 371 bp, Homo sapiens
ggaagggatggtgacaggaagaggcgtggtgtcacctgtggatactgaggaaaggctggtgacaggaagaggggtggcctgacctgtggatgcagaggaagtgtcggtgacaggaagaggcgtggtgtcacctgtggatactgaggaaaggctggtgagaggaagaggggtggcgtgaccggtggatgctgaggaagcatcggtgacaggaagagtgctggtgtcacctctggatgctgaggaagggctggtgacatgaagaggggtggcgtgacctgtggataatgaggaagcattggtgacaggaagaggggtggtgtcacctgtggatgctgaggaagtgctggtgacaggaacaggggtggtgtcac

SEQ ID NO: 316 - DNA, 270 bp, Homo sapiens
  misc_feature (1)...(270): n = A,T,C or G
ctggcctctgccttcagggtaccaccgtctccaggacacaaatgggcagcagaaaaatgtcaccttgttgatactcagcagctcatctattgggacaaaacttccatctcggccaagggaaatactctgttgagtgaccagcggggcccagcccccagccctatttatctcatcaatatggttcanggaagataaaaaagagtgttctatgggatagaaaggtgggaataagaaaaaactaagtggctgggcacggtgagtcacgcctgt

SEQ ID NO: 317 - DNA, 344 bp, Homo sapiens; identical to SEQ ID NO: 278

SEQ ID NO: 318 - DNA, 601 bp, Homo sapiens
  misc_feature (1)...(601): n = A,T,C or G
ctggaattggcttacagcacatgctttgtttcatgttatgggtgaggacctacatacactcttactttagcagtcacttaaccttctccagcaaggcagttgtggggttcactaggatttagtgcctgatcttttttttgggaaggggcgggaatgaatgtgttggggctgggagggaagcagaagaaaatgggagtgtgagtgagtgtgcatgtgtctgaagttcaccattgcccccacctgcacctagcaaggaacaggtgtttgatgtattttgctcatgactgcagtatgcatgtatttttttccttctctgtgttttctaaacttacactaaaggattcatcaaatcatcttgttcagatggctcaggattgtatttattttgcttaccccgtgctcttgggttctatagtatttctataattatgtaacgagaatagtgttgcactgtaatctatcatatagagctatatgtatggaaaattttgancaattttttaagaaatgtatnctgtttgcaaaggcacagtaaagtttgcatcttatagantataggcaaataaagctaanaattaaaccttatttaacacaaaccaca

SEQ ID NO: 319 - DNA, 465 bp, Homo sapiens
aaatgacttcagctaacttaccccaagagaaaaatctggcattgatctcttggttagcattattaccaatatcaactagcactattaaaatcaaagttgaaatggtacattcatttgccaaaaaaaaaaaaagaaaaaaaaggcttaaaggcaaagaaggtgaatcaacgtgcaaattagcatctggcccaattgcaaaattcatttcctggatgtgagggattgaaccatgcacacttgcaagcaagatgaagggcaaacagatgacatcaaatcaaaattaaccccacaaagaatccagaagacctcagatggtaaaggacagaggtctacgtctactgctgttagtgctcaggtatccatcctgttttccctaatctcctgattctgatccaagagtctttgagacctatgtcctaggccatcctttcatctagaaatggaaaccatggctgtggcaccagg

SEQ ID NO: 320 - DNA, 204 bp, Homo sapiens
ccttgtgctgggacaacctctctcttgccttacctcagagagggactatgccctgacccctcctttctgaaaatcagtgccctccctgttgctctaggaggctcctgctggcttggtagaagacagaattcgatctgcctgtccctttttcccctggggtttgacacacaggctcctctcagcatgaggtggagcagtgaccag

SEQ ID NO: 321 - DNA, 420 bp, Homo sapiens
ctcgcgtcgcatttggccgcctccctaccgctccaagcccagccctcagccatggcatgccccctggatcaggccattggcctcctcgtggccatcttccacaagtactccggcagggagggtgacaagcacaccctgagcaagaaggagctgaaggagctgatccagaaggagctcaccattggctcgaagctgcaggatgctgaaattgcaaggctgatggaagacttggaccggaacaaggaccaggaggtgaacttccaggagtatgtcaccttcctgggggccttggctttgatctacaatgaagccctcaagggctgaaaataaatagggaagatggagacaccctctgggggtcctctctgagtcaaatccagtggtgggtaattgtacaataaattttttttggtcaaattt

SEQ ID NO: 322 - DNA, 314 bp, Homo sapiens
ctccgcccagccagatgtcccgagtgcgccaaggactgtcctctcacccactcctggattctgccctgacctccatcctggacactgccttaataacatagacccttccactgacaccctcgctctcacaccccctccagggcaggggcccttagagtcttggttgccaaacagatttgcagatcaaggagaacccaggagtttcaaagaagcgctagtaaggtctctgagatccttgcactagctacatcctcagggtaggaggaagatggcttccagaagcatgcggctgctcctattgctgagctgcctgg

SEQ ID NO: 323 - DNA, 423 bp, Homo sapiens
ccaccagctcatcgcaatcattgacgataggcagcttccctttcttgctacgctgcaggatctcatttgcctctttcaacgtcacacctgctggagccaccaccagttcaatccttggcgtcatcacctcactgaggagggtggtgtggtccttctcagcaagaaagtcgatgtctcgggaggtgacgatgcccaccagcttgctgcccatggtgcccgtctcagtgatggggatgccagagaagccatgccgcatcttggcctccagcacatcgcccacagtgtgcgaggggctcagcaccacagggtccgtgatgaagccctgttcaaacttcttgaccttccgcacctcgttggcctggaactctggggtgcagttgtggtgaatgaaaccaatacctcccatcagagccatggcaatgg

SEQ ID NO: 324 - DNA, 427 bp, Homo sapiens
ctgcatcgcggcccacgacaagagggggaggtacgggaccctgttcacgatggaccgggtgctgaccccccaatggggactgtcatggatgtcctgaagggagacaatcgctttagcatgctggtagctgccatccagtctgcaggactgacggagaccctcaaccgggaaggagtctacacagtctttgctcccacaaatgaagccttccgagccctgccaccaagagaacggagcagactcttgggagatgccaaggaacttgccaacatcctgaaataccacattggtgatgaaatcctggttagcggaggcatcggggccctggtgcggctaaagtctctccaaggtgacaagctggaagtcagcttgaaaaacaatgtggtgagtgtcaacaaggagcctgttgccgagcctgacatcatgg

SEQ ID NO: 325 - DNA, 401 bp, Homo sapiens
ctggtaacccttccacaccccaatcttcatggaccagagatcttggatgttccttccacagttcaaaagacccctttcgtcacccaccctgggtatgacactggaaatggtattcagcttcctggcacttctggtcagcaacccagtgttgggcaacaaatgatctttgaggaacatggttttaggcggaccacaccgcccacaacggccacccccataaggcataggccaagaccatacccgccgaatgtaggacaagaagctctctctcagacaaccatctcatgggccccattccaggacacttctgagtacatcattccatgtcatcctgttggcactgatgaagaacccttacagttcagggttcctggaacttctaccagtgccactctgacagg

SEQ ID NO: 326 - DNA, 263 bp, Homo sapiens; identical to SEQ ID NO: 312

SEQ ID NO: 327 - DNA, 344 bp, Homo sapiens
ctgtccaatgacaacaggaccctcactctactcagtgtcacaaggaatgatgtaggaccctatgagtgtggaatccagaacaaattaagtgttgaccacagcgacccagtcatcctgaatgtcctctatggcccagacgaccccaccatttccccctcatacacctattaccgtccaggggtgaacctcagcctctcctgccatgcagcctctaacccacctgcacagtattcttggctgattgatgggaacatccagcaacacacacaagagctctttatctccaacatcactgagaagaacagcggactctatacctgccaggccaataactcagccagtgg

SEQ ID NO: 328 - DNA, 512 bp, Homo sapiens
  misc_feature (1)...(512): n = A,T,C or G
gaatgcggctgttaagacctgcaataatccagaatggctactctgatctatgttgataaggaaaatggagaaccaggcacccgtgtggttgctaaggatgggctgaagctggggtctggaccttcaatcaaagccttagatgggagatctcaagtttcaacaccacgttttggcaaaacgttcgatgccccaccagccttacctaaagctactagaaaggctttgggaactgtcaacagagctacagaaaagtctgtaaagaccaagggacccctcaaacaaaaacagccaagcttttctgccaaaaagatgactgagaagactgttaaagcaaaaagctctgttcctgcctcagatgatgcctatccagaaatagaaaaattctttcccttcaatcctctagactttgagagttttgacctgcctgaagagcaccagattgcgcacctccccttgagtggagtgcctctcatgatccttgacnangagagagancttgaaaagctgtttca

SEQ ID NO: 329 - DNA, 364 bp, Homo sapiens
ctgtgttcctaaagttcgtctttcgcttggctcaggacaaagcggtgtaacgagtcaaggtctctgcctccactgtgctcactgactttcttccctcctcggaaaagcaataacgtggggtagcctcgtaccgaatacttgctgcagatattccgttcagcagtgcagtctacttcggcgatcttgacccccgccagaccagggaattcctttttagagagttcctcccaagtaggagccagagtcttacaatgaccacaccatggagcataaaacttgatgaaggttattccttctgcaatggtgtcatcgaagttattttcagtgagtgccaacacagtgcccttgtcagcctcgggctcag

SEQ ID NO: 330 - DNA, 221 bp, Homo sapiens
cacccggccgccttctaactgtgactccccgcactccccaaaaagaatccgaaaaaccacaaagaaacaccaggcgtacctggtgcgcgagagcgtatccccaactgggacttccgaggcaacttgaactcagaacactacagcggagacgccacccggtgcttgaggcgggaccgaggcgcacagagaccgaggcgcatagagaccgaggcacagcccag

SEQ ID NO: 331 - DNA, 520 bp, Homo sapiens
gttggtgcacaaaatactgccatttgctcaaagctggctgccaaatgtttggtgatgaaggcagaaatgaatggctcaaaacttgggagaagagcaaaacctgaaggggccctccagaacaatgatgggctttatgatcctgactgcgatgagagcgggctctttaaggccaagcagtgcaacggcacctccacgtgctggtgtgtgaacactgctggggtcagaagaacagacaaggacactgaaataacctgctctgagcgagtgagaacctactggatcatcattgaactaaaacacaaagcaagagaaaaaccttatgatagtaaaagtttgcggactgcacttcagaaggagatcacaacgcgttatcaactggatccaaaatttatcacgagtattttgtatgagaataatgttatcactattgatctggttcaaaattcttctcaaaaaactcagaatgatgtggacatagctgatgtggcttattattttgaaaaagatgttaaaggtgaat

SEQ ID NO: 332 - DNA, 305 bp, Homo sapiens
ccaccccggagatgacacgaggctcacatgactctagacacttggtggaaagtgaggcgagaaaaacaatgacttgggccaattacacgactgcaaagctagagctgccaacagggctccagggagcttggcttctgtagaagttctaaggaagcggtacgaactccacggcggtggggcgctaactagcagggacccctgcaagtgttggtcgggggcctcgagctgcctgagctgacacgagggggaggggtctgtgtagccaacaggtgaccgaagggcttgcctgcccacagcttacttgg

SEQ ID NO: 333 - DNA, 377 bp, Homo sapiens
  misc_feature (1)...(377): n = A,T,C or G
ccttaaatatcagactttgtaacaggtagaaatattacagaataatttaagacactacaatgggggcaaatgaaatnngaaaatttttantgagttacccgtactcattacattttcagtgcttttacaaggaaaaaaggtgatatgtttaattttaacattttaattggctagctcttgcccttatatgactttaatgtctgtgagtcattcccagcttaaattaacaattgttagtattagtctcacacataagtgccatacattttatcctcatggatgtgatgcactgaaaagttagttgctctccttttttcttttttttgtcgtgcatattttatttctgtagtttctggntagctaccctaaagtgattt

SEQ ID NO: 334 - DNA, 251 bp, Homo sapiens
  misc_feature (1)...(251): n=A,T,C or G
cagtcaaaaacttcacncaatnggaaaataaangnttnttcaatgaataatcaaacaaaaattatccaggaccttatagggttttcagtatgtnccaggcttgatgcncatnttaaaanacaggacattatnttgctgggatcattagggtatgactgaaagngaaaaacagtaatttgtaaaacatttacctaataatagctttcccaaacagtacttcccctggaattaaaacaggaaatacaatttat

SEQ ID NO: 335 - DNA, 513 bp, Homo sapiens
aaaaagaaaaaaaaagccaaatacattttctgacattgtaagattgccttactgtctgtcattccttattgctggcccctttctcaggccggaggccaagtggtggagaaggaaaggaaatgatcgaacgggcatgttgtcaagtgggcatgccactgggaaataccaccagtttaccctgaaacattgtcctcagaggagtaggaaagtggattttgaatctctattttgctcaaaagttcagttcctgagatactgatgactgagagtgctgctgggaaattttcaggattgtgtggtcttttggggttttttgttttttttttttaagacaaagttgaccgctgttcactgtccacgtgatcagttgtaagattacaatgctgcatgctagttggttacataagatacaattccagtgatggaaggcggttataatggatggtggtgtgtacaagatggcactgccatctttgagcagagcccagctctgcagcgccacttcatcttttt

SEQ ID NO: 336 - DNA, 343 bp, Homo sapiens; identical to SEQ ID NO: 21

SEQ ID NO: 337 - DNA, 647 bp, Homo sapiens
cctctgtgcaagcarcacataggatctggatgtaggttgaggataratcctcacccaccagkggggtaaytttcccagcaattctgaaactaaaataaggaaggcacwttcccarasccctgytgagtaggggcttcaggctattttcactctacacaaaatgggggagaggagtyccctctccactaatttttcacccataaacctccacatcactaggaacctaaaggggaactccaaaggccaacacatccttggtggttatatgtgttgtcctgacaacctcctgctccagaaatgccaggagcattggatatgtcattgggagcatcaggcagtccaacatcggagggagaaaggcccagagatgaggatctgagtcaggctggcaaggctggagtcagaaagtgaccattaggcaactggtcactacaattggtggctacaaagaagtggtcacagtcaccaaaataaagaggtttacaacaacggtttccccttaggtcattttgaccaggacagtaccctaaaggaaataaggcagcatcgcataaagcaagagcccccctcagtaatccacggagatggaggggtcctcatcctggttgcaaaacaggagatggaaaggcccaggggtggcgactcag

SEQ ID NO: 338 - DNA, 515 bp, Homo sapiens
aaaaaaargywwkktctycactccaaaatgacagagacagactactgaaggaattgaagaatctgcagcagcaacmcytacagattawycarrrgwycactgagttacatccactgaaggctcaacttcaggagtatcaagataagacraaagcatttcagattatgcaagaagagctcaggcaggaaaacctctcctggcagcatgagctgcatcagctcaggatggagaagagttcctgggaaatacatgagaggagaatgaaggaacagtaccttatggctatctcagataaagatcagcagctcagtcatctgcagaatcttataagggaattgaggtcttcttcctcccagactcagcctctcaaagtgcaataccaaagacaggcatccccagagacatcagcttccccagatgggtcacaaaatctggtttatgagacagaacttctcaggacccagctcaatgacagcttaaaggaaattcaccaaaaggagttaagaattcagcaa

SEQ ID NO: 339 - DNA, 438 bp, Homo sapiens
aaaataggccctgagtataagagcatgaagagctgcctttatgtcggcatggcgagcgacaacgtcgatgctgctgagctcgcggagaccattgcggccacagcccgggagatagaggagaactcgaggcttctggaaaacatgacagaagtggttcggaaaggcattcaggaagctcaagtggagctgcagaaggcaagtgaagaacggcttctggaagagggggtgttgcggcagatccctgtagtgggctccgtgctgaattggttttctccggtccaggctttacagaagggaagaacttttaacttgacagcaggctctctggagtccacagaacccatatatgtctacaaagcacaaggtgcaggagtcacgctgcctccaacgccctcgggcagtcgcaccaagcagaggcttccaggccagaagcctttt

SEQ ID NO: 340 - DNA, 451 bp, Homo sapiens
ctgatgatgtagaagtatatgattgaacgaccagagccagaattccaagacctaaacgaaaaggcacgagcacttaaacaaattctcagtaagatcccagatgagatcaatgacagagtgaggtttctgcagacaatcaaggatatagctagtgcaataaaagaacttcttgatacagtgaataatgtcttcaagaaatatcaataccagaaccgcagggcacttgaacaccaaaagaaagaatttgtaaagtactccaaaagtttcagtgatactctgaaaacgtattttaaagatggcaaggcaataaatgtgttcgtaagtgccaaccgactaattcatcaaaccaacttaatacttcagaccttcaaaactgtggcctgaaagttgtatatgttaaagagatgtacttctcagtggcagtattgaactgcctttatctgtaaatttt

SEQ ID NO: 341 - DNA, 237 bp, Homo sapiens
aaaaccatcataacaaaaagggtccattgtcttatgatccactggaaagaggaccgactcatcatttatggctatgacttggcagtgactccaatgtgatatcctgtaattttatcttcagttatgctatagcatgtacatttccattctcttgtcgaagtttctttcgttcctcagcttctccttcatatttcctgacgtattgtcttctaagctggactgtaataacagcaacag

SEQ ID NO: 342 - DNA, 512 bp, Homo sapiens
  misc_feature (1)...(512): n = A,T,C or G
tgtaaaacgacggccagtgaattgtaatacgactcactatagggcgaattgggccctctagatgcatgctngagcggccgcccagtgtgatggatatctgcagaattcgcccttgagcggccgcccgggcgggtcctgggagatcccagggtcctccaccctccccctgaccacatacaaaggcactctagttcaagggtgaaaagtctcacccaggaggaacagccctccttgaagcaatggcagggccagcagggaggtgggcatggcagggaatggagtgagccagacagacttcacctccttactggacacagggtcaagggcgagtttcaattgctgctccctttactttctctacctgtgactactccctggaccaatcctgaggagggcacattttccagaagccacgtgataggggctggtttctgtggagccggaggcagagacactgaacttgagctcacctcctaacaccggcagtaaacttcctggaactttgccctcag

SEQ ID NO: 343 - DNA, 372 bp, Homo sapiens; identical to SEQ ID NO: 198

SEQ ID NO: 344 - DNA, 308 bp, Homo sapiens; identical to SEQ ID NO: 258

SEQ ID NO: 345 - DNA, 513 bp, Homo sapiens
gaatgcggctgttaagacctgcaataatccagaatggctactctgatctatgttgataaggaaaatggagaaccaggcacccgtgtggttgctaaggatgggctgaagctggggtctggaccttcaatcaaagccttagatgggagatctcaagtttcaacaccacgttttggcaaaacgttcgatgccccaccagccttacctaaagctactagaaaggctttgggaactgtcaacagagctacagaaaagtctgtaaagaccaagggacccctcaaacaaaaacagccaagcttttctgccaaaaagatgactgagaagactgttaaagcaaaaagctctgttcctgcctcagatgatgcctatccagaaatagaaaaattctttcccttcaatcctctagactttgagagttttgacctgcctgaagagcaccagattgcgcacctccccttgagtggagtgcctctcatgatccttgacgaggagagagagcttgaaaagctgtttcag

SEQ ID NO: 346 - DNA, 744 bp, Homo sapiens
aaaaaatacaggagtcgatagcagcagttggtgacgagatggcactcagaaacggcgttgacgtaatttaggacgtggaatcataagcgaaacagcacactgtttgaataaagagcagagtcggtatttatatttgkttytcttttgtcatgattatttgatttttaagktgctccagctaaggcatttttttgtattagtatttctattagggaacctttcttattaggtggtttgtattgtctggtttctaacatgcaggtagctgtttggcagttaaacacgtttagagtaatttgagttacaacgtgtgaaactgagcaaaaaagcagtgataagtttgggttaccataccaaatatttgttttcccactggaaaaaagtaagttttagaaaatagttaacctttgcagcatttgtttacagtttacagttccagaagtgcgtcgaaatggattacataactgctcttttattcctggtgttcacatctgtcccaggctgacacctgctcttggctggcccactttggtatgggctttaatttcactaccccaaacacgatactgtcatctgctttataataatgctcaagatgcctgataaaaatctcattttgcagccagacaagccttgaatccttttggcactaactgcaaaggaagatttttttctctagatatgcattagcagctagtgctccagttagaagcacgaactataaccttgataagtaaacagcag

SEQ ID NO: 347 - DNA, 392 bp, Homo sapiens
ctggtgctaggttacgagggctggctggccggctaccagatgaattttgagactgcaaaatcccgagtgacccagagcaactttgcagttggctacaagactgatgaattccagcttcacactaatgtgaatgacgggacagagtttggcggctccatttaccagaaagtgaacaagaagttggagaccgctgtcaatcttgcctggacagcaggaaacagtaacacgcgcttcggaatagcagccaagtatcagattgaccctgacgcctgcttctcggctaaagtgaacaactccagcctgataggtttaggatacactcagactctaaagccaggtattaaactgacactgtcagctcttctggatggcaagaacgtcaatgctggtgg

SEQ ID NO: 348 - DNA, 476 bp, Homo sapiens
  misc_feature (1)...(476): n = A,T,C or G
gattacgccaagcttgggtacccgagctcggatccactagtaacggccgcccccgggctgcargaattcmkaggtgccagaagccgagttcctasaccccakctggatttttttttttccaatttsgtggcggcgggcggctggggaagcagctctgtggtctcccggcgggtgaggccgagacagagaccaacctctaggcggcgccctggcgttgagtccgcgctgaacaagattctctcgcagctctgcgtcccgcgccgggtgaaggggcctgttcctgcaggcgctcggggctcggcctggagctggcggccggcacgtcgcctttggcgccctctctcggaaccacgccgtcccccgcgcgtcttcgtagactgcaggagtccagggcgtctggggactgtgacggcscccaaggcgggggatgtggcggctntgggtcgccgccttctgcccccgcctctgtgaggcct

SEQ ID NO: 349 - DNA, 732 bp, Homo sapiens
cctgggtggtggagcgaatgggccgattccaccggatcctggagcctggtttgaacatcctcatccctgtgttagaccggatccgatatgtgcagagtctcaaggaaattgtcatcaacgtgcctgagcagtcggctgtgactctcgacaatgtaactctgcaaatcgatggagtcctttacctgcgcatcatggacccttacaaggcaagctacggtgtggaggaccctgagtatgccgtcacccagctagctcaaacaaccatgagatcagagctcggcaaactctctctggacaaagtcttccgggaacgggagtccctgaatgccagcattgtggatgccatcaaccaagctgctgactgctggggtatccgctgcctccgttatgagatcaaggatatccatgtgccaccccgggtgaaagagtctatgcagatgcaggtggaggcagagcggcggaaacgggccacagttctagagtctgaggggacccgagagtcggccatcaatgtggcagaagggaagaaacaggcccagatcctggcctccgaagcagaaaaggctgaacagataaatcaggcagcaggagaggccagtgcagttctggcgaaggccaaggctaaagctgaagctattcgaatcctggctgcagctctgacacaacataatggagatgcagcagattcactgactgtggccgagcagtatgtcagcgcgttctccaaactgg

SEQ ID NO: 350 - DNA, 938 bp, Homo sapiens
ttttcaagctgacttccagcttgtcaccttggagagactttagccgcaccagggccccgatgcctccgctaaccaggatttcatcaccaatgtggtatttcaggatgttggcaagttccttggcatctcccaagagtctgctccgttctcttggtggcagggctcggaaggcttcatttgtgggagcaaagactgtgtagactccttcccggttgagggtctccgtcagtcctgcagactggatggcagctaccagcatgctaaagcgattgtctcccttcaggacatccatgacagtccccattgggggggtcagcacccggtccatcgtgaacagggtcccgtacctccccctcttgtcgtgggccgcgatgcagctgttctcaatgcagaggctattacgataaacaaaaactctcagttttttgccgcccagagtttccagggtctgtccatggtacagatacttagaggccagctggtctttaattatgtggttccgaagcaaattccttgtatgggcatcaattggaggggttccatctttgaatacagaattcaggggagccaggagggtcaaccgctcacttccagagagatgattgccgaggccggcttgtctgaaaaggtcaatggctgtggacacatcagactctgcagccaattcaaatagtgtcttggctgagtctgggatgagtagctcatcaatgtagtggatcaccccgttggtggctaggatgtctttattggagatgatcgccttcccgttgatagtgagcatgtccccgctgcagcccacctccagtgtcgtgccctccagggtctctacagacagccccgcaacgatggcttcagcacacatagctaacttcaagatgtggttgttcagcaggtctctcagggcttctgggtcgcccaggatacggttcaaagtctca

SEQ ID NO: 351 - DNA, 793 bp, Homo sapiens
aaaaatctacctgttcctgacttaaaacaaaaggaaagaaactacctttttataatgcacaactgttgatggtaggctgtatagtttttagtctgtgtagttaatttaatttgcagtttgtgcggcagattgctctgccaagatacttgaacactgtgttttattgtggtaattatgttttgtgattcaaacttctgtgtactgggtgatgcacccattgtgattgtggaagatagaattcaatttgaactcaggttgtttatgaggggaaaaaaacagttgcatagagtatagctctgtagtggaatatgtcttctgtataactaggctgttaacctatgattgtaaagtagctgtaagaatttcccagtgaaataaaaaaaaaattttaagtgttctcggggatgcatagattcatcattttctccaccttaaaaatgcgggcatttaagtctgtccattatctatatagtcctgtcttgtctattgtatatataatctatatgattaaagaaaatatgcataatcagacaagcttgaatattgtttttgcaccagacgaacagtgaggaaattcggagctatacatatgtgcagaaggttactacctagggtttatgcttaattttaatcggaggaaatgaatgctgattgtaacggagttaattttattgataataaattatacactatgaaaccgccattgggctactgtagatttgtatccttgatgaatctggggtttccatcagactgaacttacactgtatattttgcaatagttacctcaagg

SEQ ID NO: 352 - DNA, 671 bp, Homo sapiens
ccagttagattatgggtccaaagggattccagacacttctgagccagtcagctaccacaactctggagtaaaatatgctgcatccgggcaagaatctttaagactgaaccacaaagaggtaaggctctccaaagagatggagcgaccctgggttaggcagccttctgccccagagaaacactccagagactgctacaaggaggaagaacacctcactcagtcaatcgtcccaccccctaaaccagagaggagtcatagcctcaaactccatcatacccagaacgtggagagggaccccagtgtgctgtaccagtaccaaccacacggcaagcgccagagcagtgtgactgttgtgtcccagtatgataacctggaagattaccactccctgcctcagcaccagcgaggagtctttggagggggcggcatggggacgtatgtgccccctggctttccccatccacagagcaggacctatgctacagcgttgggtcaaggggccttcctgcccgcagagttgtccttgcagcatcctgaaacacagatccatgcagaatgagccctgcgagcaatagagttgaagcagcctctgctggacagtggactgttctatttttttcaataaccaaaaagattaaacaaaaaatactataaaacccctgaccacattt

SEQ ID NO: 353 - DNA, 571 bp, Homo sapiens
ccaacatggtgaaaccctctctccactaaaaatacaaaaattagccaggaatggtggcgggcgcctgtagtcccagctacttgggaggctgaggcaggagaatcgcttgaacccgggaggtggaggctgcagtgagccaggatcgcgccactgcactccagcctgggcaacaagagcgaaactccatctcaaaaaaaaaaaaaaaagtgctgttaatacaatcaggatggacaggaagtaaagaggtaaaaattctacgcaccttgagatataagccagatttttaagacgaaaaccaagattttgtttaagaaaaaaagaaggaaggattacgctgcagacgggccattctagggggcagcttccctcccctccttccctcttatcagccagagacagaaactaaaaaccagggtttagggcagatgaaagcctaaacagaaagaaggatgggggtcgggagaaaggaaaaaacagcaactgcctagatacaagcagagaagacaaaggcctcattccaggtcagctgggcaattctctcaggcttgccatcttgtgtcctgttttcttc

SEQ ID NO: 354 - DNA, 368 bp, Homo sapiens
cctccaccatggccttcaagcagatggagcagatctctcagttcctgcaagcagctgagcgctatggcattaacaccactgacatcttccaaactgtggacctctgggaaggaaagaacatggcctgtgtgcagcggacgctgatgaatctgggtgggctggcagtagcccgagatgatgggctcttctctggggatcccaactggttccctaagaaatccaaggagaatcctcggaacttctcggataaccagctgcaagagggcaagaacgtgatcgggttacagatgggcaccaaccgcggggcgtctcaggcaggcatgactggctacgggatgccacgccagatcctctgatcccaccccagg

SEQ ID NO: 355 - DNA, 509 bp, Homo sapiens
aaaattaggctgaatgtacttcatgtgatttgtcaaccatagtttatcagagattatggacttaattgattggtatattagtgacatcaacttgacacaagattagacaaaaaattccttacaaaaatactgtgtaactatttctcaaacttgtgggatttttcaaaagctcagtatatgaatcatcatactgtttgaaattgctaatgacagagtaagtaacactaatattggtcattgatcttcgttcatgaattagtctacagaaaaaaaatgttctgtaaaattagtctgttgaaaatgttttccaaacaatgttactttgaaaattgagtttatgtttgacctaaatgggctaaaattacattagataaactaaaattctgtccgtgtaactataaattttgtgaatgcattttcctggtgtttgaaaaagaagggggggagaattccaggtgccttaatataaagtttgaagcttcatccaccaaagttaaatagagctattt

SEQ ID NO: 356 - DNA, 241 bp, Homo sapiens
cctcggaattccctttcagctccagctttacccacatcagctgctagacgggtacgggcaaaatcaagagggtacacaaaacacagggatgtggcccctgcggcaccacccgatgccagattccctgcaaagtagcgccaaaactgggttctcttgtccacaccacccaggaagatctgcttgtatttatctttgaaggcgaagttaagagcctgggtggggaagtatctgatgacattgg

SEQ ID NO: 357 - DNA, 234 bp, Homo sapiens
ccaccagcaggaatgcagcggattcctctgtcccaagtgctcccagaaggcaggattctgaagaccactccagcgatatgttcaactatgaagaatactgcaccgccaacgcagtcactgggccttgccgtgcatccttcccacgctggtactttgacgtggagaggaactcctgcaataacttcatctatggaggctgccggggcaataagaacagctaccgctctgaggagg

SEQ ID NO: 358 - DNA, 615 bp, Homo sapiens
tcccccccccccccaaaaaaaagccatccccccgctctgccccgtcgcacattcggcccccgcgactcggccagagcggcgctggcagaggagtgtccggcaggagggccaacgcccgctgttcggtttgcgacacgcagcagggaggtgggcggcagcgtcgccggcttccagacaccaatgggaatcccaatggggaagtcgatgctggtgcttctcaccttcttggccttcgcctcgtgctgcattgctgcttaccgccccagtgagaccctgtgcggcggggagctggtggacaccctccagttcgtctgtggggaccgcggcttctacttcagcaggcccgcaagccgtgtgagccgtcgcagccgtggcatcgttgaggagtgctgtttccgcagctgtgacctggccctcctggagacgtactgtgctacccccgccaagtccgagagggacgtgtcgacccctccgaccgtgcttccggacaacttccccagataccccgtgggcaagttcttccaatatgacacctggaagcagtccacccagcgcctgcgcaggggccttgcctgccctcctgcgtgcccgcggggtcacgtgctcgccaaggag

SEQ ID NO: 359 - DNA, 201 bp, Homo sapiens
ccaaactgggagaagatggcgtacagggacttttttagctctgcagtgggggaaaaaaagacctttgagctcccctttttagaagaagcgcagcccaaatagaagaccatcagccaattgatggggtacccagaatgcctcgggtccataatctctcaagacctaacaatcacagaagcagccaggaaccactcaagatgg

SEQ ID NO: 360 - DNA, 419 bp, Homo sapiens
ctggtagggagcaattctattatttggcattgcatggctgggttgaattaaaacagggagtgagaacaggtgagtctagaagtccaactctgaaaaggaccactgtacatttgaacacacggctgtgttaaagatgctgctaatgtcagtcactgggtgcactaaaggatctcttattttatgtaaaacgttgggattgacaagatagatctgatactctgttaagttaccctctgaagctacttcttgtgaaatactaatgacagcatcatcctgccaagcgaaagaggcaggcataagcaaggacaaattaaaagggggtaagagccttatcatgatgaggagtcttgttttgacatcttgggaaaagctgtccatagtgtgaagtcgtcaatttctcaccatggtttgcagtttgc

SEQ ID NO: 361 - DNA, 792 bp, Homo sapiens
gcgtccctctgcctgcccactcagtggcaacacccgggagctgttttgtcctttgtggagcctcagcagttccctctttcagaactcactgccaagagccctgaacaggagccaccatgcagtgcttcagcttcattaagaccatgatgatcctcttcaatttgctcatctttctgtgtggtgcagccctgttggcagtgggcatctgggtgtcaatcgatggggcatcctttctgaagatcttcgggccactgtcgtccagtgccatgcagtttgtcaacgtgggctacttcctcatcgcagccggcgttgtggtctttgctcttggtttcctgggctgctatggtgctaagactgagagcaagtgtgccctcgtgacgttcttcttcatcctcctcctcatcttcattgctgaggttgcagctgctgtggtcgccttggtgtacaccacaatggctgagcacttcctgacgttgctggtagtgcctgccatcaagaaagattatggttcccaggaagacttcactcaagtgtggaacaccaccatgaaagggctcaagtgctgtggcttcaccaactatacggattttgaggactcaccctacttcaaagagaacagtgcctttcccccattctgttgcaatgacaacgtcaccaacacagccaatgaaacctgcaccaagcaaaaggctcacgaccaaaaagtagagggttgcttcaatcagcttttgtatgacatccgaactaatgcagtcaccgtgggtggtgtggcagctggaattgggggcctcg

SEQ ID NO: 362 - DNA, 141 bp, Homo sapiens
aaaggagttggaggagagggagggggaggacatggcaccattccagaaaccagcattgttacaacaccatagccagtatatttagtttggcttttcctaacatagaaatcttcaaagctggggaagtggaaataaagtttt

SEQ ID NO: 363 - DNA, 219 bp, Homo sapiens
  misc_feature (1)...(219): n = A,T,C or G
gctggcagaggagtgtccggcaggagggccaacgcccgctgttcggtttgcgacacgcancagggaggtgggcggcagcgtcgccggcttccagacaccaatgggaatcccaatggggaagtcgatgctggngcttctcaccttcttggccttcgcctcgtgctgcattgctgcttaccgccccaatgagaccctgtgcggnggggagctggnggacac

SEQ ID NO: 364 - DNA, 268 bp, Homo sapiens
  misc_feature (1)...(268): n = A,T,C or G
naccactcgctccaccttctccaccaactaccggnccctgggctctgnccaggcncccagctacngcgcccggncggtcancagcncggccagcgtctatgcaggcgctgggggctctggntcccggatctccgtgtcccgntccaccancttnangggcggtatggggnccgggggccnggccaccgggatagccnggggnntggcaggantgggaggcntccagaacgagaagganaccatgcagagactgaacgaccgcctggcc

SEQ ID NO: 365 - DNA, 151 bp, Homo sapiens
  misc_feature (1)...(151): n = A,T,C or G
aaggggcttttagaaatatttaaggacaacataaggtattaatattggaaaaaaactgtacatattttcaagcacaacnctgaaatattgcaacagngtttaactgaattgttttacctgcccgggcggccgctcgaaagggcgaattcca

SEQ ID NO: 366 - DNA, 304 bp, Homo sapiens
  misc_feature (1)...(304): n = A,T,C or G
gccagtgtgatggatatctgcagaattcgcccttagcgtggtcgcggccgaggtaaactacanagggttttccagctattatttcctttagtttctaaaagtaacgacttatattaatgttttataaaagatagtgatgaaaaaaaggtaatgctgaaataaaggcgcttttagaaatatttaaggacaacataaggtattaatattggaaaaaaactgtacatattttcaagcacaacactgaaatattgcagcagtgtttaactgaattgttttacctgcccgggcggccgctcgaaagggc

SEQ ID NO: 367 - DNA, 501 bp, Homo sapiens
caggtccttcgagaagatccctagtgagactttgaaccgtatcctgggcgacccagaagccctgagagacctgctgaacaaccacatcttgaagttagctatgtgtgctgaagccatcgttgcggggctgtctgtagagaccctggagggcacgacactggaggtgggctgcagcggggacatgctcactatcaacgggaaggcgatcatctccaataaagacatcctagccaccaacggggtgatccactacattgatgagctactcatcccagactcagccaagacactatttgaattggctgcagagtctgatgtgtccacagccattgaccttttcagacaagccggcctcggcaatcatctctctggaagtgagcggttgaccctcctggctcccctgaattctgtattcaaagatggaacccctccaattgatgcccatacaaggaatttgcttcggaaccacataattaaagaccagctggcctctaagtatct

SEQ ID NO: 368 - DNA, 581 bp, Homo sapiens
  misc_feature (1)...(581): n = A,T,C or G
cgtcagtgcgatggatatctgcncaattcgccctttgagcggccgcccgggcaggtccttcgataagatccctagtgagactttgaaccgtatcctgggcgacccagaagccctgagagacctgctgaacaaccacatcttgaagttagctatgtgtgctgaagccatcgttgcggggctgtctgtagagaccctggagggcacgacactggaggtgggctgcagcggggacatgctcactatcaacgggaaggcgatcatctccaataaagacatcctagccaccaacggggtgatccactacattgatgagctactcatcccagactcagccaagacactatttgaattggctgcagagtctgatgtgtccacagccattgaccttttcagacaagccggctcggcaatcatctctctggaagtgagcggttgaccctcctggctcccctgaattctgtattcaaagatggaacccctccaattgatgcccatacaaggaatttgcttcggaaccacataattaaagaccagctggcctctaagtatctgtccatggacagaccctgggaaactctggg

SEQ ID NO: 369 - DNA, 381 bp, Homo sapiens
aattgtgttttaattgtaaaaatggcagggggtggaattattactctatacattcaacagagactgaatagatatgaaagctgattttttttaattaccatgcttcacaatgttaagttatatggggagcaacagcaaacaggtgctaatttgttttggatatagtataagcagtgtctgtgttttgaaagaatagaacacagtttgtagtgccactgttgttttgggggggcttttttcttttcggaaatcttaaaccttaagatactaaggacgttgttttggttgtactttggaattcttagtcacaaaatatattttgtttacaaaaatttctgtaaaacaggttataacagtgtttacctcggccgcgaccacgct

SEQ ID NO: 370 - DNA, 501 bp, Homo sapiens
gtaaagtacattatgagaacaacagccctttcctgaccatcaccagcatgacccgagtcattgaagtctctcactggggtaatattgctgtggaagaaaatgtggacttaaagcacacaggagctgtgcttaaggggcctttctcacgctatgattaccagagacagccagatagtggaatatcctccatccgttcttttaagaccatccttcctgctgctgcccaggatgtttattaccgggatgagattggcaatgtttctaccagccacctccttattttggatgactctgtagagatggaaatccggcctcgcttccctctctttggcgggtggaagacccattacatcgttggctacaacctcccaagctatgagtacctctataatttgggtgaccagtatgcactgaagatgaggtttgtggaccatgtgtttgatgaacaagtgatagattctctgactgtgaagatcatcctgcctgaaggagccaagaaca

SEQ ID NO: 371 - DNA, 439 bp, Homo sapiens
  misc_feature (1)...(439): n = A,T,C or G
gtctgtcctctgttactcanatacagnnccaaaactaagcgattatataagcacatccatattttagggctactctaagttaaaaaccttttctcttgtttcagagttatttacatcaaattaagacatttacaaattgttcatagtatacaatagcccaaatatgattttcacctatgctgtgtaaagaagttaagcattcgtaagtttgtctaataaattcagtgcacttttttccataacacgagctattctaaatgttttacatttctttcagtgcatatttccaaattcattaaacagaatgaaatcaatgttattaaatggctatatcataatattcaagcatattatggaatctataccacagtgggattcacgtcaatactataattcactctagaaaaacatcacaggcacacacaaaataaagaacaaa

SEQ ID NO: 372 - DNA, 162 bp, Homo sapiens
  misc_feature (1)...(162): n = A,T,C or G
taatnaaattcattgcacttttttccataacacgagctatcctaagtgttgtacatttctttcagtgcatatttncaaattcattaaacacaatgaaatcaatgntattaaatggcnatatcataanattcaancatattatggaatgtataccacaccggg

SEQ ID NO: 373 - DNA, 301 bp, Homo sapiens
ggtccgttgactattctctacaaaccacaaagacattggaacactatacctattattcggcgcatgagctggagtcctaggcacagctctaagcctccttattcgagccgagctgggccagccaggcaaccttctaggtaacgaccacatctacaacgttatcgtcacagcccatgcatttgtaataatcttcttcatagtaatacccatcataatcggaggctttggcaaacctgcccgggcggccaagggcgaattccagcacactggcggccgttactagtggatccgagctcggtac

SEQ ID NO: 374 - DNA, 471 bp, Homo sapiens
catgtccctgatctcagtgaggtcctccttggtgaacacaaagcccacattcccccggatatgaggcagcagtttctccagagctgggttgttttccaggtgccctcggatggccttgcgcatcatggtgttcttgcccatcagcaccacagccttcccgcgaagggacatgcggatctgctgcatctgcttggagcccacattgtctgctcccacaatgaaacatttcggataatcatccaatagttggatgatcttaaggaagtagttggacttccaggtcgccctgtcttccctgggcatcacggcggtgcgtcagggattgccacgcagggtttacctcggccgcgaccacgctaagggcgaattccagcacactggcggccgttactagtggatccgagctcggtaccaagcttggcgtaatcatggtcatagctgtttcctgtgtgaaattgttatccgctcaca

SEQ ID NO: 375 - DNA, 287 bp, Homo sapiens
tatgtagcatttagcaccactggctcaatgcgctcacctaggtgagagtgtgaccaaatcttaaagcattagtgctattatcagttaccaccatttggggcttttatccttcatgggttatgatgttctcctgatgacacatttctctgagttttgtaattccagccaaagagagaccattcactatttgatggctggctgcatgcagacatttacctcggccgcgaccacgctaagggcgaattccagcacactggcggccgttactagtggatccgagctcggta

SEQ ID NO: 376 - DNA, 309 bp, Homo sapiens
tccaaccaagagtgtgctccagatgtgtttgggccctacctggcacagagtcctgctcctgggaaaggaaaggaccacagcaaacaccattctttttgccgtacttcctagaagcactggaagaggactggtgatggtggagggtgagagggtgccgtttcctgctccagctccagaccttgtctgcagaaaacatctgcagtgcagcaaatccatgtccagccaggcaaccagacctgcccgggcggccgcccgaaagggcgaattccagcacactggcggccgttactagtggatccgagctcggta

SEQ ID NO: 377 - DNA, 490 bp, Homo sapiens
  misc_feature (1)...(490): n = A,T,C or G
gtaccacatcgaggtgaaccgtgttcctgctggcaactgggttctgattgaaggtgttgatcaaccaattgtgaanacagcaaccataaccgaaccccgaggcaatgaggaggctcanattttccgacccttgaagttcaataccacatctgttatcaagattgctgtggagccagtcaacccctcagagctgcccaagatgcttgatggcctgcgcaaggtcaacaagagctatccatccctcaccaccaaggtggaggagtctggcaagcatgtgatcctgggcactggggagctctacctggactgtgtgatgcatgatttgcggaagatgtactcanagatagacatcaaggtggctgacccagttgtcacgttttgtgagacggtggtggaaacatcctccctcaagtgctttgctgaaacgcctaataagaagaacaagatcaccatgattgctgagcctcttgagaagggcctggcagaggac

(19) United States
(12) Patent Application Publication
(10) Pub. No.: US 2002/0086303 A1
(43) Pub. Date: Jul. 4, 2002

(54) Compositions and methods for the therapy and diagnosis of colon cancer

(76) Inventors: Madeleine Joy Meagher, Seattle, WA (US); Gordon E. King, Shoreline, WA (US); Jiangchun Xu, Bellevue, WA (US); Heather Secrist, Seattle, WA (US)

Correspondence Address: SEED INTELLECTUAL PROPERTY LAW GROUP PLLC, 701 FIFTH AVE, SUITE 6300, SEATTLE, WA, 98104-7092, US

(21) Appl. No.: 09/878,134

(22) Filed: Jun. 7, 2001

Related U.S. Application Data
(60) Provisional application No. 60/210,667, filed on Jun. 9, 2000.
(60) Provisional application No. 60/252,614, filed on Nov. 22, 2000.

(51) Int. Cl. C12Q 1/68; G01N 33/574; C07H 21/04; A61K 39/395; C12N 9/00; A61K 48/00

(52) U.S. Cl. 435/6; 435/7.23; 435/69.1; 435/325; 435/320.1; 424/155.1; 536/23.2; 435/183; 530/388.8; 514/44

(57) ABSTRACT

Compositions and methods for the therapy and diagnosis of cancer, such as colon cancer, are disclosed. Compositions may comprise one or more colon tumor proteins, immunogenic portions thereof, or polynucleotides that encode such portions. Alternatively, a therapeutic composition may comprise an antigen presenting cell that expresses a colon tumor protein, or a T cell that is specific for cells expressing such a protein. Such compositions may be used, for example, for the prevention and treatment of diseases such as colon cancer. Diagnostic methods based on detecting a colon tumor protein, or mRNA encoding such a protein, in a sample are also provided.

### CROSS REFERENCE TO RELATED APPLICATIONS

This application claims priority to U.S. Provisional Application No. 60/210,667 filed Jun. 9, 2000, and No. 60/252,614 filed Nov. 22, 2000, incorporated by reference in their entirety herein.

### TECHNICAL FIELD OF THE INVENTION

The present invention relates generally to therapy and diagnosis of cancer, such as colon cancer. The invention is more specifically related to polypeptides comprising at least a portion of a colon tumor protein, and to polynucleotides encoding such polypeptides. Such polypeptides and polynucleotides may be used in vaccines and pharmaceutical compositions for prevention and treatment of colon cancer and for the diagnosis and monitoring of such cancers.

### BACKGROUND OF THE INVENTION

Cancer is a significant health problem throughout the world. Although advances have been made in detection and therapy of cancer, no vaccine or other universally successful method for prevention or treatment is currently available. Current therapies, which are generally based on a combination of chemotherapy or surgery and radiation, continue to prove inadequate in many patients.

Colon cancer is the second most frequently diagnosed malignancy in the United States as well as the second most common cause of cancer death. The five-year survival rate for patients with colorectal cancer detected in an early localized stage is 92%; unfortunately, only 37% of colorectal cancer is diagnosed at this stage. The survival rate drops to 64% if the cancer is allowed to spread to adjacent organs or lymph nodes, and to 7% in patients with distant metastases.

The prognosis of colon cancer is directly related to the degree of penetration of the tumor through the bowel wall and the presence or absence of nodal involvement, consequently, early detection and treatment are especially important. Currently, diagnosis is aided by the use of screening assays for fecal occult blood, sigmoidoscopy, colonoscopy and double contrast barium enemas. Treatment regimens are determined by the type and stage of the cancer, and include surgery, radiation therapy and/or chemotherapy. Recurrence following surgery (the most common form of therapy) is a major problem and is often the ultimate cause of death. In spite of considerable research into therapies for the disease, colon cancer remains difficult to diagnose and treat. In spite of considerable research into therapies for these and other cancers, colon cancer remains difficult to diagnose and treat effectively. Accordingly, there is a need in the art for improved methods for detecting and treating such cancers. The present invention fulfills these needs and further provides other related advantages.

### SUMMARY OF THE INVENTION

In one aspect, the present invention provides polynucleotide compositions comprising a sequence selected from the group consisting of:

(a) sequences provided in SEQ ID NO:1-377;

(b) complements of the sequences provided in SEQ ID NO:1-377;

(c) sequences consisting of at least 20, 25, 30, 35, 40, 45, 50, 75 and 100 contiguous residues of a sequence provided in SEQ ID NO:1-377;

(d) sequences that hybridize to a sequence provided in SEQ ID NO:1-377, under moderate or highly stringent conditions;

(e) sequences having at least 75%, 80%, 85%, 90%, 95%, 96%, 97%, 98% or 99% identity to a sequence of SEQ ID NO:1-377;

(f) degenerate variants of a sequence provided in SEQ ID NO:1-377.

In one preferred embodiment, the polynucleotide compositions of the invention are expressed in at least about 20%, more preferably in at least about 30%, and most preferably in at least about 50% of colon tumors samples tested, at a level that is at least about 2-fold, preferably at least about 5-fold, and most preferably at least about 10-fold higher than that for normal tissues.

The present invention, in another aspect, provides polypeptide compositions comprising an amino acid sequence that is encoded by a polynucleotide sequence described above.

In certain preferred embodiments, the polypeptides and/or polynucleotides of the present invention are immunogenic, i.e., they are capable of eliciting an immune response, particularly a humoral and/or cellular immune response, as further described herein.

The present invention further provides fragments, variants and/or derivatives of the disclosed polypeptide and/or polynucleotide sequences, wherein the fragments, variants and/or derivatives preferably have a level of immunogenic activity of at least about 50%, preferably at least about 70% and more preferably at least about 90% of the level of immunogenic activity of a polypeptide sequence encoded by a polynucleotide sequence set forth in SEQ ID NO:1-377.

The present invention further provides polynucleotides that encode a polypeptide described above, expression vectors comprising such polynucleotides and host cells transformed or transfected with such expression vectors.

Within other aspects, the present invention provides pharmaceutical compositions comprising a polypeptide or polynucleotide as described above and a physiologically acceptable carrier.

Within a related aspect of the present invention, the pharmaceutical compositions, e.g., vaccine compositions, are provided for prophylactic or therapeutic applications. Such compositions generally comprise an immunogenic polypeptide or polynucleotide of the invention and an immunostimulant, such as an adjuvant.

The present invention further provides pharmaceutical compositions that comprise: (a) an antibody or antigen-binding fragment thereof that specifically binds to a polypeptide of the present invention, or a fragment thereof, and (b) a physiologically acceptable carrier.

Within further aspects, the present invention provides pharmaceutical compositions comprising: (a) an antigen presenting cell that expresses a polypeptide as described above and (b) a pharmaceutically acceptable carrier or excipient. Illustrative antigen presenting cells include dendritic cells, macrophages, monocytes, fibroblasts and B cells.

Within related aspects, pharmaceutical compositions are provided that comprise: (a) an antigen presenting cell that expresses a polypeptide as described above and (b) an immunostimulant.

The present invention further provides, in other aspects, fusion proteins that comprise at least one polypeptide as described above, as well as polynucleotides encoding such fusion proteins, typically in the form of pharmaceutical compositions, e.g., vaccine compositions, comprising a physiologically acceptable carrier and/or an immunostimulant. The fusions proteins may comprise multiple immunogenic polypeptides or portions/variants thereof, as described herein, and may further comprise one or more polypeptide segments for facilitating the expression, purification and/or immunogenicity of the polypeptide(s).

Within further aspects, the present invention provides methods for stimulating an immune response in a patient, preferably a T cell response in a human patient, comprising administering a pharmaceutical composition described herein. The patient may be afflicted with colon cancer, in which case the methods provide treatment for the disease, or patient considered at risk for such a disease may be treated prophylactically.

Within further aspects, the present invention provides methods for inhibiting the development of a cancer in a patient, comprising administering to a patient a pharmaceutical composition as recited above. The patient may be afflicted with colon cancer, in which case the methods provide treatment for the disease, or patient considered at risk for such a disease may be treated prophylactically.

The present invention further provides, within other aspects, methods for removing tumor cells from a biological sample, comprising contacting a biological sample with T cells that specifically react with a polypeptide of the present invention, wherein the step of contacting is performed under conditions and for a time sufficient to permit the removal of cells expressing the protein from the sample.

Within related aspects, methods are provided for inhibiting the development of a cancer in a patient, comprising administering to a patient a biological sample treated as described above.

Methods are further provided, within other aspects, for stimulating and/or expanding T cells specific for a polypeptide of the present invention, comprising contacting T cells with one or more of: (i) a polypeptide as described above; (ii) a polynucleotide encoding such a polypeptide; and/or (iii) an antigen presenting cell that expresses such a polypeptide; under conditions and for a time sufficient to permit the stimulation and/or expansion of T cells. Isolated T cell populations comprising T cells prepared as described above are also provided.

Within further aspects, the present invention provides methods for inhibiting the development of a cancer in a patient, comprising administering to a patient an effective amount of a T cell population as described above.

The present invention further provides methods for inhibiting the development of a cancer in a patient, comprising the steps of: (a) incubating CD4⁺ and/or CD8⁺ T cells isolated from a patient with one or more of: (i) a polypeptide comprising at least an immunogenic portion of polypeptide disclosed herein; (ii) a polynucleotide encoding such a polypeptide; and (iii) an antigen-presenting cell that expressed such a polypeptide; and (b) administering to the patient an effective amount of the proliferated T cells, and thereby inhibiting the development of a cancer in the patient. Proliferated cells may, but need not, be cloned prior to administration to the patient.

Within further aspects, the present invention provides methods for determining the presence or absence of a cancer, preferably a colon cancer, in a patient comprising: (a) contacting a biological sample obtained from a patient with a binding agent that binds to a polypeptide as recited above; (b) detecting in the sample an amount of polypeptide that binds to the binding agent; and (c) comparing the amount of polypeptide with a predetermined cut-off value, and therefrom determining the presence or absence of a cancer in the patient. Within preferred embodiments, the binding agent is an antibody, more preferably a monoclonal antibody.

The present invention also provides, within other aspects, methods for monitoring the progression of a cancer in a patient. Such methods comprise the steps of: (a) contacting a biological sample obtained from a patient at a first point in time with a binding agent that binds to a polypeptide as recited above; (b) detecting in the sample an amount of polypeptide that binds to the binding agent; (c) repeating steps (a) and (b) using a biological sample obtained from the patient at a subsequent point in time; and (d) comparing the amount of polypeptide detected in step (c) with the amount detected in step (b) and therefrom monitoring the progression of the cancer in the patient.

The present invention further provides, within other aspects, methods for determining the presence or absence of a cancer in a patient, comprising the steps of: (a) contacting a biological sample, e.g., tumor sample, serum sample, etc., obtained from a patient with an oligonucleotide that hybridizes to a polynucleotide that encodes a polypeptide of the present invention; (b) detecting in the sample a level of a polynucleotide, preferably mRNA, that hybridizes to the oligonucleotide; and (c) comparing the level of polynucleotide that hybridizes to the oligonucleotide with a predetermined cut-off value, and therefrom determining the presence or absence of a cancer in the patient. Within certain embodiments, the amount of mRNA is detected via polymerase chain reaction using, for example, at least one oligonucleotide primer that hybridizes to a polynucleotide encoding a polypeptide as recited above, or a complement of such a polynucleotide. Within other embodiments, the amount of mRNA is detected using a hybridization technique, employing an oligonucleotide probe that hybridizes to a polynucleotide that encodes a polypeptide as recited above, or a complement of such a polynucleotide.

In related aspects, methods are provided for monitoring the progression of a cancer in a patient, comprising the steps of: (a) contacting a biological sample obtained from a patient with an oligonucleotide that hybridizes to a polynucleotide that encodes a polypeptide of the present invention; (b) detecting in the sample an amount of a polynucleotide that hybridizes to the oligonucleotide; (c) repeating steps (a) and (b) using a biological sample obtained from the patient at a subsequent point in time; and (d) comparing the amount of polynucleotide detected in step (c) with the amount detected in step (b) and therefrom monitoring the progression of the cancer in the patient.

Within further aspects, the present invention provides antibodies, such as monoclonal antibodies, that bind to a polypeptide as described above, as well as diagnostic kits comprising such antibodies. Diagnostic kits comprising one or more oligonucleotide probes or primers as described above are also provided.

These and other aspects of the present invention will become apparent upon reference to the following detailed description. All references disclosed herein are hereby incorporated by reference in their entirety as if each was incorporated individually.

### BRIEF DESCRIPTION OF THE SEQUENCE IDENTIFIERS

SEQ ID NO:1 is the determined cDNA sequence for clone R0089:A03

SEQ ID NO:2 is the determined cDNA sequence for clone R0089:A05

SEQ ID NO:3 is the determined cDNA sequence for clone R0089:A06

SEQ ID NO:4 is the determined cDNA sequence for clone R0089:A07

SEQ ID NO:5 is the determined cDNA sequence for clone R0089:A08

SEQ ID NO:6 is the determined cDNA sequence for clone R0089:A09

SEQ ID NO:7 is the determined cDNA sequence for clone R0089:A11

SEQ ID NO:8 is the determined cDNA sequence for clone R0089:A12

SEQ ID NO:9 is the determined cDNA sequence for clone R0089:B02

SEQ ID NO:10 is the determined cDNA sequence for clone R0089:B03

SEQ ID NO:11 is the determined cDNA sequence for clone R0089:B05

SEQ ID NO:12 is the determined cDNA sequence for clone R0089:B06

SEQ ID NO:13 is the determined cDNA sequence for clone R0089:B07

SEQ ID NO:14 is the determined cDNA sequence for clone R0089:B08

SEQ ID NO:15 is the determined cDNA sequence for clone R0089:B09

SEQ ID NO:16 is the determined cDNA sequence for clone R0089:B10

SEQ ID NO:17 is the determined cDNA sequence for clone R0089:B11

SEQ ID NO:18 is the determined cDNA sequence for clone R0089:B12

SEQ ID NO:19 is the determined cDNA sequence for clone R0089:C01

SEQ ID NO:20 is the determined cDNA sequence for clone R0089:C03

SEQ ID NO:21 is the determined cDNA sequence for clone R0089:C04

SEQ ID NO:22 is the determined cDNA sequence for clone R0089:C05

SEQ ID NO:23 is the determined cDNA sequence for clone R0089:C06

SEQ ID NO:24 is the determined cDNA sequence for clone R0089:C07

SEQ ID NO:25 is the determined cDNA sequence for clone R0089:C08

SEQ ID NO:26 is the determined cDNA sequence for clone R0089:C09

SEQ ID NO:27 is the determined cDNA sequence for clone R0089:C10

SEQ ID NO:28 is the determined cDNA sequence for clone R0089:C11

SEQ ID NO:29 is the determined cDNA sequence for clone R0089:C12

SEQ ID NO:30 is the determined cDNA sequence for clone R0089:D01

SEQ ID NO:31 is the determined cDNA sequence for clone R0089:D02

SEQ ID NO:32 is the determined cDNA sequence for clone R0089:D03

SEQ ID NO:33 is the determined cDNA sequence for clone R0089:D04

SEQ ID NO:34 is the determined cDNA sequence for clone R0089:D05

SEQ ID NO:35 is the determined cDNA sequence for clone R0089:D06

SEQ ID NO:36 is the determined cDNA sequence for clone R0089:D07

SEQ ID NO:37 is the determined cDNA sequence for clone R0089:D08

SEQ ID NO:38 is the determined cDNA sequence for clone R0089:D09

SEQ ID NO:39 is the determined cDNA sequence for clone R0089:D10

SEQ ID NO:40 is the determined cDNA sequence for clone R0089:D11

SEQ ID NO:41 is the determined cDNA sequence for clone R0089:D12

SEQ ID NO:42 is the determined cDNA sequence for clone R0089:E01

SEQ ID NO:43 is the determined cDNA sequence for clone R0089:E02

SEQ ID NO:44 is the determined cDNA sequence for clone R0089:E03

SEQ ID NO:45 is the determined cDNA sequence for clone R0089:E05

SEQ ID NO:46 is the determined cDNA sequence for clone R0089:E06

SEQ ID NO:47 is the determined cDNA sequence for clone R0089:E08

SEQ ID NO:48 is the determined cDNA sequence for clone R0089:E09

SEQ ID NO:49 is the determined cDNA sequence for clone R0089:E10

SEQ ID NO:50 is the determined cDNA sequence for clone R0089:E11

SEQ ID NO:51 is the determined cDNA sequence for clone R0089:F01

SEQ ID NO:52 is the determined cDNA sequence for clone R0089:F02

SEQ ID NO:53 is the determined cDNA sequence for clone R0089:F03

SEQ ID NO:54 is the determined cDNA sequence for clone R0089:F04

SEQ ID NO:55 is the determined cDNA sequence for clone R0089:F05

SEQ ID NO:56 is the determined cDNA sequence for clone R0089:F06

SEQ ID NO:57 is the determined cDNA sequence for clone R0089:F07

SEQ ID NO:58 is the determined cDNA sequence for clone R0089:F08

SEQ ID NO:59 is the determined cDNA sequence for clone R0089:F10

SEQ ID NO:60 is the determined cDNA sequence for clone R0089:F11

SEQ ID NO:61 is the determined cDNA sequence for clone R0089:F12

SEQ ID NO:62 is the determined cDNA sequence for clone R0089:G01

SEQ ID NO:63 is the determined cDNA sequence for clone R0089:G02

SEQ ID NO:64 is the determined cDNA sequence for clone R0089:G03

SEQ ID NO:65 is the determined cDNA sequence for clone R0089:G04

SEQ ID NO:66 is the determined cDNA sequence for clone R0089:G05

SEQ ID NO:67 is the determined cDNA sequence for clone R0089:G06

SEQ ID NO:68 is the determined cDNA sequence for clone R0089:G07

SEQ ID NO:69 is the determined cDNA sequence for clone R0089:G09

SEQ ID NO:70 is the determined cDNA sequence for clone R0089:G10

SEQ ID NO:71 is the determined cDNA sequence for clone R0089:G11

SEQ ID NO:72 is the determined cDNA sequence for clone R0089:G12

SEQ ID NO:73 is the determined cDNA sequence for clone R0089:H01

SEQ ID NO:74 is the determined cDNA sequence for clone R0089:H02

SEQ ID NO:75 is the determined cDNA sequence for clone R0089:H03

SEQ ID NO:76 is the determined cDNA sequence for clone R0089:H04

SEQ ID NO:77 is the determined cDNA sequence for clone R0089:H06

SEQ ID NO:78 is the determined cDNA sequence for clone R0089:H07

SEQ ID NO:79 is the determined cDNA sequence for clone R0089:H08

SEQ ID NO:80 is the determined cDNA sequence for clone R0089:H09

SEQ ID NO:81 is the determined cDNA sequence for clone R0089:H10

SEQ ID NO:82 is the determined cDNA sequence for clone R0089:H11

SEQ ID NO:83 is the determined cDNA sequence for clone R0090:A12

SEQ ID NO:84 is the determined cDNA sequence for clone R0090:A05

SEQ ID NO:85 is the determined cDNA sequence for clone R0090:A06

SEQ ID NO:86 is the determined cDNA sequence for clone R0090:A07

SEQ ID NO:87 is the determined cDNA sequence for clone R0090:A08

SEQ ID NO:88 is the determined cDNA sequence for clone R0090:A09

SEQ ID NO:89 is the determined cDNA sequence for clone R0090:A11

SEQ ID NO:90 is the determined cDNA sequence for clone R0090:A12

SEQ ID NO:91 is the determined cDNA sequence for clone R0090:B02

SEQ ID NO:92 is the determined cDNA sequence for clone R0090:B04

SEQ ID NO:93 is the determined cDNA sequence for clone R0090:B05

SEQ ID NO:94 is the determined cDNA sequence for clone R0090:B06

SEQ ID NO:95 is the determined cDNA sequence for clone R0090:B07

SEQ ID NO:96 is the determined cDNA sequence for clone R0090:B08

SEQ ID NO:97 is the determined cDNA sequence for clone R0090:B10

SEQ ID NO:98 is the determined cDNA sequence for clone R0090:B12

SEQ ID NO:99 is the determined cDNA sequence for clone R0090:C01

SEQ ID NO:100 is the determined cDNA sequence for clone R0090:C02

SEQ ID NO:101 is the determined cDNA sequence for clone R0090:C03

SEQ ID NO:102 is the determined cDNA sequence for clone R0090:C04

SEQ ID NO:103 is the determined cDNA sequence for clone R0090:C06

SEQ ID NO:104 is the determined cDNA sequence for clone R0090:C07

SEQ ID NO:105 is the determined cDNA sequence for clone R0090:C08

SEQ ID NO:106 is the determined cDNA sequence for clone R0090:C09

SEQ ID NO:107 is the determined cDNA sequence for clone R0090:C10

SEQ ID NO:108 is the determined cDNA sequence for clone R0090:C11

SEQ ID NO:109 is the determined cDNA sequence for clone R0090:C12

SEQ ID NO:110 is the determined cDNA sequence for clone R0090:D01

SEQ ID NO:111 is the determined cDNA sequence for clone R0090:D02

SEQ ID NO:112 is the determined cDNA sequence for clone R0090:D03

SEQ ID NO:113 is the determined cDNA sequence for clone R0090:D04

SEQ ID NO:114 is the determined cDNA sequence for clone R0090:D05

SEQ ID NO:115 is the determined cDNA sequence for clone R0090:D06

SEQ ID NO:116 is the determined cDNA sequence for clone R0090:D07

SEQ ID NO:117 is the determined cDNA sequence for clone R0090:D08

SEQ ID NO:118 is the determined cDNA sequence for clone R0090:D09

SEQ ID NO:119 is the determined cDNA sequence for clone R0090:D10

SEQ ID NO:120 is the determined cDNA sequence for clone R0090:D11

SEQ ID NO:121 is the determined cDNA sequence for clone R0090:D12

SEQ ID NO:122 is the determined cDNA sequence for clone R0090:E01

SEQ ID NO:123 is the determined cDNA sequence for clone R0090:E02

SEQ ID NO:124 is the determined cDNA sequence for clone R0090:E03

SEQ ID NO:125 is the determined cDNA sequence for clone R0090:E04

SEQ ID NO:126 is the determined cDNA sequence for clone R0090:E05

SEQ ID NO:127 is the determined cDNA sequence for clone R0090:E06

SEQ ID NO:128 is the determined cDNA sequence for clone R0090:E07

SEQ ID NO:129 is the determined cDNA sequence for clone R0090:E08

SEQ ID NO:130 is the determined cDNA sequence for clone R0090:E09

SEQ ID NO:131 is the determined cDNA sequence for clone R0090:E11

SEQ ID NO:132 is the determined cDNA sequence for clone R0090:E12

SEQ ID NO:133 is the determined cDNA sequence for clone R0090:F02

SEQ ID NO:134 is the determined cDNA sequence for clone R0090:F03

SEQ ID NO:135 is the determined cDNA sequence for clone R0090:F04

SEQ ID NO:136 is the determined cDNA sequence for clone R0090:F05

SEQ ID NO:137 is the determined cDNA sequence for clone R0090:F06

SEQ ID NO:138 is the determined cDNA sequence for clone R0090:F07

SEQ ID NO:139 is the determined cDNA sequence for clone R0090:F08

SEQ ID NO:140 is the determined cDNA sequence for clone R0090:F09

SEQ ID NO:141 is the determined cDNA sequence for clone R0090:F10

SEQ ID NO:142 is the determined cDNA sequence for clone R0090:F11

SEQ ID NO:143 is the determined cDNA sequence for clone R0090:F12

SEQ ID NO:144 is the determined cDNA sequence for clone R0090:G01

SEQ ID NO:145 is the determined cDNA sequence for clone R0090:G02

SEQ ID NO:146 is the determined cDNA sequence for clone R0090:G03

SEQ ID NO:147 is the determined cDNA sequence for clone R0090:G05

SEQ ID NO:148 is the determined cDNA sequence for clone R0090:G06

SEQ ID NO:149 is the determined cDNA sequence for clone R0090:G07

SEQ ID NO:150 is the determined cDNA sequence for clone R0090:G08

SEQ ID NO:151 is the determined cDNA sequence for clone R0090:G09

SEQ ID NO:152 is the determined cDNA sequence for clone R0090:G10

SEQ ID NO:153 is the determined cDNA sequence for clone R0090:G11

SEQ ID NO:154 is the determined cDNA sequence for clone R0090:G12

SEQ ID NO:155 is the determined cDNA sequence for clone R0090:H05

SEQ ID NO:156 is the determined cDNA sequence for clone R0090:H06

SEQ ID NO:157 is the determined cDNA sequence for clone R0090:H07

SEQ ID NO:158 is the determined cDNA sequence for clone R0090:H08

SEQ ID NO:159 is the determined cDNA sequence for clone R0090:H09

SEQ ID NO:160 is the determined cDNA sequence for clone R0090:H10

SEQ ID NO:161 is the determined cDNA sequence for clone R0090:H11

SEQ ID NO:162 is the determined cDNA sequence for clone R0091:A02

SEQ ID NO:163 is the determined cDNA sequence for clone R0091:A03

SEQ ID NO:164 is the determined cDNA sequence for clone R0091:A05

SEQ ID NO:165 is the determined cDNA sequence for clone R0091:A06

SEQ ID NO:166 is the determined cDNA sequence for clone R0091:A08

SEQ ID NO:167 is the determined cDNA sequence for clone R0091:A09

SEQ ID NO:168 is the determined cDNA sequence for clone R0091:A11

SEQ ID NO:169 is the determined cDNA sequence for clone R0091:A12

SEQ ID NO:170 is the determined cDNA sequence for clone R0091:B01

SEQ ID NO:171 is the determined cDNA sequence for clone R0091:B02

SEQ ID NO:172 is the determined cDNA sequence for clone R0091:B03

SEQ ID NO:173 is the determined cDNA sequence for clone R0091:B04

SEQ ID NO:174 is the determined cDNA sequence for clone R0091:B05

SEQ ID NO:175 is the determined cDNA sequence for clone R0091:B06

SEQ ID NO:176 is the determined cDNA sequence for clone R0091:B07

SEQ ID NO:177 is the determined cDNA sequence for clone R0091:B08

SEQ ID NO:178 is the determined cDNA sequence for clone R0091:B9

SEQ ID NO:179 is the determined cDNA sequence for clone R0091:B11

SEQ ID NO:180 is the determined cDNA sequence for clone R0091:B12

SEQ ID NO:181 is the determined cDNA sequence for clone R0091:C01

SEQ ID NO:182 is the determined cDNA sequence for clone R0091:C02

SEQ ID NO:183 is the determined cDNA sequence for clone R0091:C03

SEQ ID NO:184 is the determined cDNA sequence for clone R0091:C04

SEQ ID NO:185 is the determined cDNA sequence for clone R0091:C05

SEQ ID NO:186 is the determined cDNA sequence for clone R0091:C06

SEQ ID NO:187 is the determined cDNA sequence for clone R0091:C07

SEQ ID NO:188 is the determined cDNA sequence for clone R0091:C08

SEQ ID NO:189 is the determined cDNA sequence for clone R0091:C09

SEQ ID NO:190 is the determined cDNA sequence for clone R0091:C10

SEQ ID NO:191 is the determined cDNA sequence for clone R0091:C11

SEQ ID NO:192 is the determined cDNA sequence for clone R0091:C12

SEQ ID NO:193 is the determined cDNA sequence for clone R0091:D01

SEQ ID NO:194 is the determined cDNA sequence for clone R0091:D02

SEQ ID NO:195 is the determined cDNA sequence for clone R0091:D03

SEQ ID NO:196 is the determined cDNA sequence for clone R0091:D04

SEQ ID NO:197 is the determined cDNA sequence for clone R0091:D05

SEQ ID NO:198 is the determined cDNA sequence for clone R0091:D06

SEQ ID NO:199 is the determined cDNA sequence for clone R0091:D07

SEQ ID NO:200 is the determined cDNA sequence for clone R0091:D08

SEQ ID NO:201 is the determined cDNA sequence for clone R0091:D09

SEQ ID NO:202 is the determined cDNA sequence for clone R0091:D10

SEQ ID NO:203 is the determined cDNA sequence for clone R0091:D11

SEQ ID NO:204 is the determined cDNA sequence for clone R0091:D12

SEQ ID NO:205 is the determined cDNA sequence for clone R0091:E01

SEQ ID NO:206 is the determined cDNA sequence for clone R0091:E02

SEQ ID NO:207 is the determined cDNA sequence for clone R0091:E03

SEQ ID NO:208 is the determined cDNA sequence for clone R0091:E04

SEQ ID NO:209 is the determined cDNA sequence for clone R0091:E05

SEQ ID NO:210 is the determined cDNA sequence for clone R0091:E06

SEQ ID NO:211 is the determined cDNA sequence for clone R0091:E07

SEQ ID NO:212 is the determined cDNA sequence for clone R0091:E08

SEQ ID NO:213 is the determined cDNA sequence for clone R0091:E09

SEQ ID NO:214 is the determined cDNA sequence for clone R0091 E10

SEQ ID NO:215 is the determined cDNA sequence for clone R0091:E11

SEQ ID NO:216 is the determined cDNA sequence for clone R0091:E12

SEQ ID NO:217 is the determined cDNA sequence for clone R0091:F02

SEQ ID NO:218 is the determined cDNA sequence for clone R0091:F03

SEQ ID NO:219 is the determined cDNA sequence for clone R0091:F04

SEQ ID NO:220 is the determined cDNA sequence for clone R0091:F05

SEQ ID NO:221 is the determined cDNA sequence for clone R0091:F07

SEQ ID NO:222 is the determined cDNA sequence for clone R0091:F09

SEQ ID NO:223 is the determined cDNA sequence for clone R0091:F10

SEQ ID NO:224 is the determined cDNA sequence for clone R0091:F11

SEQ ID NO:225 is the determined cDNA sequence for clone R0091:F12

SEQ ID NO:226 is the determined cDNA sequence for clone R0091:G01

SEQ ID NO:227 is the determined cDNA sequence for clone R0091:G02

SEQ ID NO:228 is the determined cDNA sequence for clone R0091:G04

SEQ ID NO:229 is the determined cDNA sequence for clone R0091:G05

SEQ ID NO:230 is the determined cDNA sequence for clone R0091:G06

SEQ ID NO:231 is the determined cDNA sequence for clone R0091:G07

SEQ ID NO:232 is the determined cDNA sequence for clone R0091:G08

SEQ ID NO:233 is the determined cDNA sequence for clone R0091:G09

SEQ ID NO:234 is the determined cDNA sequence for clone R0091:G10

SEQ ID NO:235 is the determined cDNA sequence for clone R0091:G11

SEQ ID NO:236 is the determined cDNA sequence for clone R0091:G12

SEQ ID NO:237 is the determined cDNA sequence for clone R0091:H01

SEQ ID NO:238 is the determined cDNA sequence for clone R0091:H02

SEQ ID NO:239 is the determined cDNA sequence for clone R0091:H03

SEQ ID NO:240 is the determined cDNA sequence for clone R0091:H04

SEQ ID NO:241 is the determined cDNA sequence for clone R0091:H05

SEQ ID NO:242 is the determined cDNA sequence for clone R0091:H06

SEQ ID NO:243 is the determined cDNA sequence for clone R0091:H07

SEQ ID NO:244 is the determined cDNA sequence for clone R0091:H08

SEQ ID NO:245 is the determined cDNA sequence for clone R0091:H09

SEQ ID NO:246 is the determined cDNA sequence for clone R0091:H10

SEQ ID NO:247 is the determined cDNA sequence for clone R0091:H11

SEQ ID NO:248 is the determined cDNA sequence for clone R0092:A03

SEQ ID NO:249 is the determined cDNA sequence for clone R0092:A05

SEQ ID NO:250 is the determined cDNA sequence for clone R0092:A06

SEQ ID NO:251 is the determined cDNA sequence for clone R0092:A07

SEQ ID NO:252 is the determined cDNA sequence for clone R0092:A09

SEQ ID NO:253 is the determined cDNA sequence for clone R0092:A10

SEQ ID NO:254 is the determined cDNA sequence for clone R0092:A11

SEQ ID NO:255 is the determined cDNA sequence for clone R0092:B01

SEQ ID NO:256 is the determined cDNA sequence for clone R0092:B02

SEQ ID NO:257 is the determined cDNA sequence for clone R0092:B03

SEQ ID NO:258 is the determined cDNA sequence for clone R0092:B04

SEQ ID NO:259 is the determined cDNA sequence for clone R0092:B05

SEQ ID NO:260 is the determined cDNA sequence for clone R0092:B08

SEQ ID NO:261 is the determined cDNA sequence for clone R0092:B09

SEQ ID NO:262 is the determined cDNA sequence for clone R0092:B10

SEQ ID NO:263 is the determined cDNA sequence for clone R0092:B11

SEQ ID NO:264 is the determined cDNA sequence for clone R0092:B12

SEQ ID NO:265 is the determined cDNA sequence for clone R0092:C02

SEQ ID NO:266 is the determined cDNA sequence for clone R0092:C03

SEQ ID NO:267 is the determined cDNA sequence for clone R0092:C04

SEQ ID NO:268 is the determined cDNA sequence for clone R0092:C05

SEQ ID NO:269 is the determined cDNA sequence for clone R0092:C6

SEQ ID NO:270 is the determined cDNA sequence for clone R0092:C07

SEQ ID NO:271 is the determined cDNA sequence for clone R0092:C08

SEQ ID NO:272 is the determined cDNA sequence for clone R0092:C09

SEQ ID NO:273 is the determined cDNA sequence for clone R0092:C10

SEQ ID NO:274 is the determined cDNA sequence for clone R0092:C11

SEQ ID NO:275 is the determined cDNA sequence for clone R0092:C12

SEQ ID NO:276 is the determined cDNA sequence for clone R0092:D02

SEQ ID NO:277 is the determined cDNA sequence for clone R0092:D03

SEQ ID NO:278 is the determined cDNA sequence for clone R0092:C04

SEQ ID NO:279 is the determined cDNA sequence for clone R0092:D05

SEQ ID NO:280 is the determined cDNA sequence for clone R0092:D06

SEQ ID NO:281 is the determined cDNA sequence for clone R0092:D07

SEQ ID NO:282 is the determined cDNA sequence for clone R0092:D08

SEQ ID NO:283 is the determined cDNA sequence for clone R0092:D09

SEQ ID NO:284 is the determined cDNA sequence for clone R0092:D10

SEQ ID NO:285 is the determined cDNA sequence for clone R0092:D11

SEQ ID NO:286 is the determined cDNA sequence for clone R0092:D12

SEQ ID NO:287 is the determined cDNA sequence for clone R0092:E01

SEQ ID NO:288 is the determined cDNA sequence for clone R0092:E02

SEQ ID NO:289 is the determined cDNA sequence for clone R0092:E03

SEQ ID NO:290 is the determined cDNA sequence for clone R0092:E04

SEQ ID NO:291 is the determined cDNA sequence for clone R0092:E05

SEQ ID NO:292 is the determined cDNA sequence for clone R0092:E06

SEQ ID NO:293 is the determined cDNA sequence for clone R0092:E07

SEQ ID NO:294 is the determined cDNA sequence for clone R0092:E08

SEQ ID NO:295 is the determined cDNA sequence for clone R0092:E09

SEQ ID NO:296 is the determined cDNA sequence for clone R0092:E10

SEQ ID NO:297 is the determined cDNA sequence for clone R0092:E11

SEQ ID NO:298 is the determined cDNA sequence for clone R0092:E12

SEQ ID NO:299 is the determined cDNA sequence for clone R0092:F01

SEQ ID NO:300 is the determined cDNA sequence for clone R0092:F02

SEQ ID NO:301 is the determined cDNA sequence for clone R0092:F03

SEQ ID NO:302 is the determined cDNA sequence for clone R0092:F04

SEQ ID NO:303 is the determined cDNA sequence for clone R0092:F05

SEQ ID NO:304 is the determined cDNA sequence for clone R0092:F06

SEQ ID NO:305 is the determined cDNA sequence for clone R0092:F07

SEQ ID NO:306 is the determined cDNA sequence for clone R0092:F08

SEQ ID NO:307 is the determined cDNA sequence for clone R0092:F09

SEQ ID NO:308 is the determined cDNA sequence for clone R0092:F10

SEQ ID NO:309 is the determined cDNA sequence for clone R0092:F11

SEQ ID NO:310 is the determined cDNA sequence for clone R0092:F12

SEQ ID NO:311 is the determined cDNA sequence for clone R0092:G01

SEQ ID NO:312 is the determined cDNA sequence for clone R0092:G02

SEQ ID NO:313 is the determined cDNA sequence for clone R0092:G03

SEQ ID NO:314 is the determined cDNA sequence for clone R0092:G04

SEQ ID NO:315 is the determined cDNA sequence for clone R0092:G05

SEQ ID NO:316 is the determined cDNA sequence for clone R0092:G06

SEQ ID NO:317 is the determined cDNA sequence for clone R0092:G07

SEQ ID NO:318 is the determined cDNA sequence for clone R0092:G08

SEQ ID NO:319 is the determined cDNA sequence for clone R0092:G09

SEQ ID NO:320 is the determined cDNA sequence for clone R0092:G10

SEQ ID NO:321 is the determined cDNA sequence for clone R0092:G11

SEQ ID NO:322 is the determined cDNA sequence for clone R0092:G12

SEQ ID NO:323 is the determined cDNA sequence for clone R0092:H01

SEQ ID NO:324 is the determined cDNA sequence for clone R0092:H02

SEQ ID NO:325 is the determined cDNA sequence for clone R0092:H03

SEQ ID NO:326 is the determined cDNA sequence for clone R0092:H04

SEQ ID NO:327 is the determined cDNA sequence for clone R0092:H05

SEQ ID NO:328 is the determined cDNA sequence for clone R0092:H06

SEQ ID NO:329 is the determined cDNA sequence for clone R0092:H07

SEQ ID NO:330 is the determined cDNA sequence for clone R0092:H08

SEQ ID NO:331 is the determined cDNA sequence for clone R0092:H09

SEQ ID NO:332 is the determined cDNA sequence for clone R0092:H10

SEQ ID NO:333 is the determined cDNA sequence for clone R0092:H11

SEQ ID NO:334 is the determined cDNA sequence for a clone from a primary normal colon library

SEQ ID NO:335 is the determined cDNA sequence for clone 89A9_C1410P

SEQ ID NO:336 is the determined cDNA sequence for clone 89C4_C1411P

SEQ ID NO:337 is the determined cDNA sequence for clone 89E2_C1412P

SEQ ID NO:338 is the determined cDNA sequence for clone 89G10_C1413P

SEQ ID NO:339 is the determined cDNA sequence for clone 89G2_C1407P

SEQ ID NO:340 is the determined cDNA sequence for clone 90C11_C1414P

SEQ ID NO:341 is the determined cDNA sequence for clone 90F8_C1408P

SEQ ID NO:342 is the determined cDNA sequence for clone 90H10_C1415P

SEQ ID NO:343 is the determ ined cDNA sequence for clone 91D6_C1416P

SEQ ID NO:344 is the determined cDNA sequence for clone 92B4_C1409P

SEQ ID NO:345 is the determined cDNA sequence for clone 92H6_C1417P

SEQ ID NO:346 is the determined cDNA sequence for clone 93F10_C1418P

SEQ ID NO:347 is the determined cDNA sequence for clone 94E8_C1419P

SEQ ID NO:348 is the determined cDNA sequence for clone 95D1_c592S

SEQ ID NO:349 is the determined cDNA sequence for clone 98F12_C1421P

SEQ ID NO:350 is the determined cDNA sequence for clone 98H6

SEQ ID NO:351 is the determined cDNA sequence for clone 99E5_C1401P

SEQ ID NO:352 is the determined cDNA sequence for clone 100G8_C1422P

SEQ ID NO:353 is the determined cDNA sequence for clone 101G6_C1402P

SEQ ID NO:354 is the determined cDNA sequence for clone 103F6

SEQ ID NO:355 is the determined cDNA sequence for clone 104C9_C1404P

SEQ ID NO:356 is the determined cDNA sequence for clone 109C2_C1405P

SEQ ID NO:357 is the determined cDNA sequence for clone 109E8_C1406P

SEQ ID NO:358 is the determined cDNA sequence for clone 95A4

SEQ ID NO:359 is the determined cDNA sequence for clone 93F12

SEQ ID NO:360 is the determined cDNA sequence for clone 93H11

SEQ ID NO:361 is the determined cDNA sequence for clone 110D9

SEQ ID NO:362 is the determined cDNA sequence for clone 102E7

SEQ ID NO:363 is the determined cDNA sequence for clone '59698.1

SEQ ID NO:364 is the determined cDNA sequence for clone '59699.3

SEQ ID NO:365 is the determined cDNA sequence for clone '59717.2

SEQ ID NO:366 is the determined cDNA sequence for clone '59717.4

SEQ ID NO:367 is the determined cDNA sequence for clone '59719.2

SEQ ID NO:368 is the determined cDNA sequence for clone '59719.4

SEQ ID NO:369 is the determined cDNA sequence for clone '59720.1

SEQ ID NO:370 is the determined cDNA sequence for clone '59721.1

SEQ ID NO:371 is the determined cDNA sequence for clone '60768.1

SEQ ID NO:372 is the determined cDNA sequence for clone '60769.1

SEQ ID NO:373 is the determined cDNA sequence for clone '60770.1

SEQ ID NO:374 is the determined cDNA sequence for clone '60773.1

SEQ ID NO:375 is the determined cDNA sequence for clone '60776.1

SEQ ID NO:376 is the determined cDNA sequence for clone '60777.1

SEQ ID NO:377 is the determined cDNA sequence for clone '60778.1

### DETAILED DESCRIPTION OF THE INVENTION

The present invention is directed generally to compositions and their use in the therapy and diagnosis of cancer, particularly colon cancer. As described further below, illustrative compositions of the present invention include, but are not restricted to, polypeptides, particularly immunogenic polypeptides, polynucleotides encoding such polypeptides, antibodies and other binding agents, antigen presenting cells (APCs) and immune system cells (e.g., T cells).

The practice of the present invention will employ, unless indicated specifically to the contrary, conventional methods of virology, immunology, microbiology, molecular biology and recombinant DNA techniques within the skill of the art, many of which are described below for the purpose of illustration. Such techniques are explained fully in the literature. See, e.g., Sambrook, et al. Molecular Cloning: A Laboratory Manual (2nd Edition, 1989); Maniatis et al. Molecular Cloning: A Laboratory Manual (1982); DNA Cloning: A Practical Approach, vol. I & II (D. Glover, ed.); Oligonucleotide Synthesis (N. Gait, ed., 1984); Nucleic Acid Hybridization (B. Hames & S. Higgins, eds., 1985); Transcription and Translation (B. Hames & S. Higgins, eds., 1984); Animal Cell Culture (R. Freshney, ed., 1986); Perbal, A Practical Guide to Molecular Cloning (1984).

All publications, patents and patent applications cited herein, whether supra or infra, are hereby incorporated by reference in their entirety.

As used in this specification and the appended claims, the singular forms “a,” “an” and “the” include plural references unless the content clearly dictates otherwise.

Polypeptide Compositions

As used herein, the term “polypeptide” is used in its conventional meaning, i.e., as a sequence of amino acids. The polypeptides are not limited to a specific length of the product; thus, peptides, oligopeptides, and proteins are included within the definition of polypeptide, and such terms may be used interchangeably herein unless specifically indicated otherwise. This term also does not refer to or exclude post-expression modifications of the polypeptide, for example, glycosylations, acetylations, phosphorylations and the like, as well as other modifications known in the art, both naturally occurring and non-naturally occurring. A polypeptide may be an entire protein, or a subsequence thereof. Particular polypeptides of interest in the context of this invention are amino acid subsequences comprising epitopes, i.e., antigenic determinants substantially responsible for the immunogenic properties of a polypeptide and being capable of evoking an immune response.

Particularly illustrative polypeptides of the present invention comprise those encoded by a polynucleotide sequence set forth in any one of SEQ ID NO:1-377, or a sequence that hybridizes under moderately stringent conditions, or, alternatively, under highly stringent conditions, to a polynucleotide sequence set forth in any one of SEQ ID NO:1-377.

The polypeptides of the present invention are sometimes herein referred to as colon tumor proteins or colon tumor polypeptides, as an indication that their identification has been based at least in part upon their increased levels of expression in colon tumor samples. Thus, a “colon tumor polypeptide” or “colon tumor protein,” refers generally to a polypeptide sequence of the present invention, or a polynucleotide sequence encoding such a polypeptide, that is expressed in a substantial proportion of colon tumor samples, for example preferably greater than about 20%, more preferably greater than about 30%, and most preferably greater than about 50% or more of colon tumor samples tested, at a level that is at least two fold, and preferably at least five fold, greater than the level of expression in normal tissues, as determined using a representative assay provided herein. A colon tumor polypeptide sequence of the invention, based upon its increased level of expression in tumor cells, has particular utility both as a diagnostic marker as well as a therapeutic target, as further described below.

In certain preferred embodiments, the polypeptides of the invention are immunogenic, i.e., they react detectably within an immunoassay (such as an ELISA or T-cell stimulation assay) with antisera and/or T-cells from a patient with colon cancer. Screening for immunogenic activity can be performed using techniques well known to the skilled artisan. For example, such screens can be performed using methods such as those described in Harlow and Lane, Antibodies: A Laboratory Manual, Cold Spring Harbor Laboratory, 1988. In one illustrative example, a polypeptide may be immobilized on a solid support and contacted with patient sera to allow binding of antibodies within the sera to the immobilized polypeptide. Unbound sera may then be removed and bound antibodies detected using, for example, ¹²⁵I-labeled Protein A.

As would be recognized by the skilled artisan, immunogenic portions of the polypeptides disclosed herein are also encompassed by the present invention. An “immunogenic portion,” as used herein, is a fragment of an immunogenic polypeptide of the invention that itself is immunologically reactive (i.e., specifically binds) with the B-cells and/or T-cell surface antigen receptors that recognize the polypeptide. Immunogenic portions may generally be identified using well known techniques, such as those summarized in Paul, Fundamental Immunology, 3rd ed., 243-247 (Raven Press, 1993) and references cited therein. Such techniques include screening polypeptides for the ability to react with antigen-specific antibodies, antisera and/or T-cell lines or clones. As used herein, antisera and antibodies are “antigen-specific” if they specifically bind to an antigen (i.e., they react with the protein in an ELISA or other immunoassay, and do not react detectably with unrelated proteins). Such antisera and antibodies may be prepared as described herein, and using well-known techniques.

In one preferred embodiment, an immunogenic portion of a polypeptide of the present invention is a portion that reacts with antisera and/or T-cells at a level that is not substantially less than the reactivity of the full-length polypeptide (e.g., in an ELISA and/or T-cell reactivity assay). Preferably, the level of immunogenic activity of the immunogenic portion is at least about 50%, preferably at least about 70% and most preferably greater than about 90% of the immunogenicity for the full-length polypeptide. In some instances, preferred immunogenic portions will be identified that have a level of immunogenic activity greater than that of the corresponding full-length polypeptide, e.g., having greater than about 100% or 150% or more immunogenic activity.

In certain other embodiments, illustrative immunogenic portions may include peptides in which an N-terminal leader sequence and/or transmembrane domain have been deleted. Other illustrative immunogenic portions will contain a small N- and/or C-terminal deletion (e.g., 1-30 amino acids, preferably 5-15 amino acids), relative to the mature protein.

In another embodiment, a polypeptide composition of the invention may also comprise one or more polypeptides that are immunologically reactive with T cells and/or antibodies generated against a polypeptide of the invention, particularly a polypeptide having an amino acid sequence disclosed herein, or to an immunogenic fragment or variant thereof.

In another embodiment of the invention, polypeptides are provided that comprise one or more polypeptides that are capable of eliciting T cells and/or antibodies that are immunologically reactive with one or more polypeptides described herein, or one or more polypeptides encoded by contiguous nucleic acid sequences contained in the polynucleotide sequences disclosed herein, or immunogenic fragments or variants thereof, or to one or more nucleic acid sequences which hybridize to one or more of these sequences under conditions of moderate to high stringency.

The present invention, in another aspect, provides polypeptide fragments comprising at least about 5, 10, 15, 20, 25, 50, or 100 contiguous amino acids, or more, including all intermediate lengths, of a polypeptide compositions set forth herein, such as those encoded by a polynucleotide sequence set forth in a sequence of SEQ ID NO:1-377.

In another aspect, the present invention provides variants of the polypeptide compositions described herein. Polypeptide variants generally encompassed by the present invention will typically exhibit at least about 70%, 75%, 80%, 85%, 90%, 91%, 92%, 93%, 94%, 95%, 96%, 97%, 98%, or 99% or more identity (determined as described below), along its length, to a polypeptide sequences set forth herein.

In one preferred embodiment, the polypeptide fragments and variants provided by the present invention are immunologically reactive with an antibody and/or T-cell that reacts with a full-length polypeptide specifically set forth herein.

In another preferred embodiment, the polypeptide fragments and variants provided by the present invention exhibit a level of immunogenic activity of at least about 50%, preferably at least about 70%, and most preferably at least about 90% or more of that exhibited by a full-length polypeptide sequence specifically set forth herein.

A polypeptide “variant,” as the term is used herein, is a polypeptide that typically differs from a polypeptide specifically disclosed herein in one or more substitutions, deletions, additions and/or insertions. Such variants may be naturally occurring or may be synthetically generated, for example, by modifying one or more of the above polypeptide sequences of the invention and evaluating their immunogenic activity as described herein and/or using any of a number of techniques well known in the art.

For example, certain illustrative variants of the polypeptides of the invention include those in which one or more portions, such as an N-terminal leader sequence or transmembrane domain, have been removed. Other illustrative variants include variants in which a small portion (e.g., 1-30 amino acids, preferably 5-15 amino acids) has been removed from the N- and/or C-terminal of the mature protein.

In many instances, a variant will contain conservative substitutions. A “conservative substitution” is one in which an amino acid is substituted for another amino acid that has similar properties, such that one skilled in the art of peptide chemistry would expect the secondary structure and hydropathic nature of the polypeptide to be substantially unchanged. As described above, modifications may be made in the structure of the polynucleotides and polypeptides of the present invention and still obtain a functional molecule that encodes a variant or derivative polypeptide with desirable characteristics, e.g., with immunogenic characteristics. When it is desired to alter the amino acid sequence of a polypeptide to create an equivalent, or even an improved, immunogenic variant or portion of a polypeptide of the invention, one skilled in the art will typically change one or more of the codons of the encoding DNA sequence according to Table 1.

For example, certain amino acids may be substituted for other amino acids in a protein structure without appreciable loss of interactive binding capacity with structures such as, for example, antigen-binding regions of antibodies or binding sites on substrate molecules. Since it is the interactive capacity and nature of a protein that defines that protein's biological functional activity, certain amino acid sequence substitutions can be made in a protein sequence, and, of course, its underlying DNA coding sequence, and nevertheless obtain a protein with like properties. It is thus contemplated that various changes may be made in the peptide sequences of the disclosed compositions, or corresponding DNA sequences which encode said peptides without appreciable loss of their biological utility or activity.

TABLE 1

| Amino Acids | | | Codons | | | | | |
|---|---|---|---|---|---|---|---|---|
| Alanine | Ala | A | GCA | GCC | GCG | GCU | | |
| Cysteine | Cys | C | UGC | UGU | | | | |
| Aspartic acid | Asp | D | GAC | GAU | | | | |
| Glutamic acid | Glu | E | GAA | GAG | | | | |
| Phenylalanine | Phe | F | UUC | UUU | | | | |
| Glycine | Gly | G | GGA | GGC | GGG | GGU | | |
| Histidine | His | H | CAC | CAU | | | | |
| Isoleucine | Ile | I | AUA | AUC | AUU | | | |
| Lysine | Lys | K | AAA | AAG | | | | |
| Leucine | Leu | L | UUA | UUG | CUA | CUC | CUG | CUU |
| Methionine | Met | M | AUG | | | | | |
| Asparagine | Asn | N | AAC | AAU | | | | |
| Proline | Pro | P | CCA | CCC | CCG | CCU | | |
| Glutamine | Gln | Q | CAA | CAG | | | | |
| Arginine | Arg | R | AGA | AGG | CGA | CGC | CGG | CGU |
| Serine | Ser | S | AGC | AGU | UCA | UCC | UCG | UCU |
| Threonine | Thr | T | ACA | ACC | ACG | ACU | | |
| Valine | Val | V | GUA | GUC | GUG | GUU | | |
| Tryptophan | Trp | W | UGG | | | | | |
| Tyrosine | Tyr | Y | UAC | UAU | | | | |

In making such changes, the hydropathic index of amino acids may be considered. The importance of the hydropathic amino acid index in conferring interactive biologic function on a protein is generally understood in the art (Kyte and Doolittle, 1982, incorporated herein by reference). It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like. Each amino acid has been assigned a hydropathic index on the basis of its hydrophobicity and charge characteristics (Kyte and Doolittle, 1982). These values are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

It is known in the art that certain amino acids may be substituted by other amino acids having a similar hydropathic index or score and still result in a protein with similar biological activity, i.e. still obtain a biological functionally equivalent protein. In making such changes, the substitution of amino acids whose hydropathic indices are within ±2 is preferred, those within ±1 are particularly preferred, and those within ±0.5 are even more particularly preferred. It is also understood in the art that the substitution of like amino acids can be made effectively on the basis of hydrophilicity. U.S. Pat. No. 4,554,101 (specifically incorporated herein by reference in its entirety), states that the greatest local average hydrophilicity of a protein, as governed by the hydrophilicity of its adjacent amino acids, correlates with a biological property of the protein.

As detailed in U.S. Pat. No. 4,554,101, the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). It is understood that an amino acid can be substituted for another having a similar hydrophilicity value and still obtain a biologically equivalent, and in particular, an immunologically equivalent protein. In such changes, the substitution of amino acids whose hydrophilicity values are within ±2 is preferred, those within ±1 are particularly preferred, and those within ±0.5 are even more particularly preferred.

As outlined above, amino acid substitutions are generally therefore based on the relative similarity of the amino acid side-chain substituents, for example, their hydrophobicity, hydrophilicity, charge, size, and the like. Exemplary substitutions that take various of the foregoing characteristics into consideration are well known to those of skill in the art and include: arginine and lysine; glutamate and aspartate; serine and threonine; glutamine and asparagine; and valine, leucine and isoleucine.

In addition, any polynucleotide may be further modified to increase stability in vivo. Possible modifications include, but are not limited to, the addition of flanking sequences at the 5′ and/or 3′ ends; the use of phosphorothioate or 2′ O-methyl rather than phosphodiesterase linkages in the backbone; and/or the inclusion of nontraditional bases such as inosine, queosine and wybutosine, as well as acetyl-, methyl-, thio- and other modified forms of adenine, cytidine, guanine, thymine and uridine.

Amino acid substitutions may further be made on the basis of similarity in polarity, charge, solubility, hydrophobicity, hydrophilicity and/or the amphipathic nature of the residues. For example, negatively charged amino acids include aspartic acid and glutamic acid; positively charged amino acids include lysine and arginine; and amino acids with uncharged polar head groups having similar hydrophilicity values include leucine, isoleucine and valine; glycine and alanine; asparagine and glutamine; and serine, threonine, phenylalanine and tyrosine. Other groups of amino acids that may represent conservative changes include: (1) ala, pro, gly, glu, asp, gin, asn, ser, thr; (2) cys, ser, tyr, thr; (3) val, ile, leu, met, ala, phe; (4) lys, arg, his; and (5) phe, tyr, trp, his. A variant may also, or alternatively, contain nonconservative changes. In a preferred embodiment, variant polypeptides differ from a native sequence by substitution, deletion or addition of five amino acids or fewer. Variants may also (or alternatively) be modified by, for example, the deletion or addition of amino acids that have minimal influence on the immunogenicity, secondary structure and hydropathic nature of the polypeptide.

As noted above, polypeptides may comprise a signal (or leader) sequence at the N-terminal end of the protein, which co-translationally or post-translationally directs transfer of the protein. The polypeptide may also be conjugated to a linker or other sequence for ease of synthesis, purification or identification of the polypeptide (e.g., poly-His), or to enhance binding of the polypeptide to a solid support. For example, a polypeptide may be conjugated to an immunoglobulin Fc region.

When comparing polypeptide sequences, two sequences are said to be “identical” if the sequence of amino acids in the two sequences is the same when aligned for maximum correspondence, as described below. Comparisons between two sequences are typically performed by comparing the sequences over a comparison window to identify and compare local regions of sequence similarity. A “comparison window” as used herein, refers to a segment of at least about 20 contiguous positions, usually 30 to about 75, 40 to about 50, in which a sequence may be compared to a reference sequence of the same number of contiguous positions after the two sequences are optimally aligned.

Optimal alignment of sequences for comparison may be conducted using the Megalign program in the Lasergene suite of bioinformatics software (DNASTAR, Inc., Madison, Wis.), using default parameters. This program embodies several alignment schemes described in the following references: Dayhoff, M. O. (1978) A model of evolutionary change in proteins—Matrices for detecting distant relationships. In Dayhoff, M. O. (ed.) Atlas of Protein Sequence and Structure, National Biomedical Research Foundation, Washington, D.C. Vol. 5, Suppl. 3, pp. 345-358; Hein J. (1990) Unified Approach to Alignment and Phylogenes pp. 626-645 Methods in Enzymology vol. 183, Academic Press, Inc., San Diego, Calif.; Higgins, D. G. and Sharp, P. M. (1989) CABIOS 5:151-153; Myers, E. W. and Muller W. (1988) CABIOS 4:11-17; Robinson, E. D. (1971) Comb. Theor 11:105; Saitou, N. Nei, M. (1987) Mol. Biol. Evol. 4:406-425; Sneath, P. H. A. and Sokal, R. R. (1973) Numerical Taxonomy—the Principles and Practice of Numerical Taxonomy, Freeman Press, San Francisco, Calif.; Wilbur, W. J. and Lipman, D. J. (1983) Proc. Natl. Acad., Sci. USA 80:726-730.

Alternatively, optimal alignment of sequences for comparison may be conducted by the local identity algorithm of Smith and Waterman (1981) Add. APL. Math 2:482, by the identity alignment algorithm of Needleman and Wunsch (1970) J. Mol. Biol. 48:443, by the search for similarity methods of Pearson and Lipman (1988) Proc. Natl. Acad. Sci. USA 85: 2444, by computerized implementations of these algorithms (GAP, BESTFIT, BLAST, FASTA, and TFASTA in the Wisconsin Genetics Software Package, Genetics Computer Group (GCG), 575 Science Dr., Madison, Wis.), or by inspection.

One preferred example of algorithms that are suitable for determining percent sequence identity and sequence similarity are the BLAST and BLAST 2.0 algorithms, which are described in Altschul et al. (1977) Nucl. Acids Res. 25:3389-3402 and Altschul et al. (1990) J. Mol. Biol. 215:403-410, respectively. BLAST and BLAST 2.0 can be used, for example with the parameters described herein, to determine percent sequence identity for the polynucleotides and polypeptides of the invention. Software for performing BLAST analyses is publicly available through the National Center for Biotechnology Information. For amino acid sequences, a scoring matrix can be used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment.

In one preferred approach, the “percentage of sequence identity” is determined by comparing two optimally aligned sequences over a window of comparison of at least 20 positions, wherein the portion of the polypeptide sequence in the comparison window may comprise additions or deletions (i.e., gaps) of 20 percent or less, usually 5 to 15 percent, or 10 to 12 percent, as compared to the reference sequences (which does not comprise additions or deletions) for optimal alignment of the two sequences. The percentage is calculated by determining the number of positions at which the identical amino acid residue occurs in both sequences to yield the number of matched positions, dividing the number of matched positions by the total number of positions in the reference sequence (i.e., the window size) and multiplying the results by 100 to yield the percentage of sequence identity.

Within other illustrative embodiments, a polypeptide may be a xenogeneic polypeptide that comprises an polypeptide having substantial sequence identity, as described above, to the human polypeptide (also termed autologous antigen) which served as a reference polypeptide, but which xenogeneic polypeptide is derived from a different, non-human species. One skilled in the art will recognize that “self” antigens are often poor stimulators of CD8⁺ and CD⁴+ T-lymphocyte responses, and therefore efficient immunotherapeutic strategies directed against tumor polypeptides require the development of methods to overcome immune tolerance to particular self tumor polypeptides. For example, humans immunized with prostase protein from a xenogeneic (non human) origin are capable of mounting an immune response against the counterpart human protein, e.g. the human prostase tumor protein present on human tumor cells. Accordingly, the present invention provides methods for purifying the xenogeneic form of the tumor proteins set forth herein, such as the polypeptides encoded by polynucleotide sequences set forth in SEQ ID NO:1-377.

Therefore, one aspect of the present invention provides xenogeneic variants of the polypeptide compositions described herein. Such xenogeneic variants generally encompassed by the present invention will typically exhibit at least about 70%, 75%, 80%, 85%, 90%, 91%, 92%, 93%, 94%, 95%, 96%, 97%, 98%, or 99% or more identity along their lengths, to a polypeptide sequences set forth herein.

More particularly, the invention is directed to mouse, rat, monkey, porcine and other non-human polypeptides which can be used as xenogeneic forms of human polypeptides set forth herein, to induce immune responses directed against tumor polypeptides of the invention.

Within other illustrative embodiments, a polypeptide may be a fusion polypeptide that comprises multiple polypeptides as described herein, or that comprises at least one polypeptide as described herein and an unrelated sequence, such as a known tumor protein. A fusion partner may, for example, assist in providing T helper epitopes (an immunological fusion partner), preferably T helper epitopes recognized by humans, or may assist in expressing the protein (an expression enhancer) at higher yields than the native recombinant protein. Certain preferred fusion partners are both immunological and expression enhancing fusion partners. Other fusion partners may be selected so as to increase the solubility of the polypeptide or to enable the polypeptide to be targeted to desired intracellular compartments. Still further fusion partners include affinity tags, which facilitate purification of the polypeptide.

Fusion polypeptides may generally be prepared using standard techniques, including chemical conjugation. Preferably, a fusion polypeptide is expressed as a recombinant polypeptide, allowing the production of increased levels, relative to a non-fused polypeptide, in an expression system. Briefly, DNA sequences encoding the polypeptide components may be assembled separately, and ligated into an appropriate expression vector. The 3′ end of the DNA sequence encoding one polypeptide component is ligated, with or without a peptide linker, to the 5′ end of a DNA sequence encoding the second polypeptide component so that the reading frames of the sequences are in phase. This permits translation into a single fusion polypeptide that retains the biological activity of both component polypeptides.

A peptide linker sequence may be employed to separate the first and second polypeptide components by a distance sufficient to ensure that each polypeptide folds into its secondary and tertiary structures. Such a peptide linker sequence is incorporated into the fusion polypeptide using standard techniques well known in the art. Suitable peptide linker sequences may be chosen based on the following factors: (1) their ability to adopt a flexible extended conformation; (2) their inability to adopt a secondary structure that could interact with functional epitopes on the first and second polypeptides; and (3) the lack of hydrophobic or charged residues that might react with the polypeptide functional epitopes. Preferred peptide linker sequences contain Gly, Asn and Ser residues. Other near neutral amino acids, such as Thr and Ala may also be used in the linker sequence. Amino acid sequences which may be usefully employed as linkers include those disclosed in Maratea et al., Gene 40:39-46, 1985; Murphy et al., Proc. Natl. Acad. Sci. USA 83:8258-8262, 1986; U.S. Pat. No. 4,935,233 and U.S. Pat. No. 4,751,180. The linker sequence may generally be from 1 to about 50 amino acids in length. Linker sequences are not required when the first and second polypeptides have non-essential N-terminal amino acid regions that can be used to separate the functional domains and prevent steric interference.

The ligated DNA sequences are operably linked to suitable transcriptional or translational regulatory elements. The regulatory elements responsible for expression of DNA are located only 5′ to the DNA sequence encoding the first polypeptides. Similarly, stop codons required to end translation and transcription termination signals are only present 3′ to the DNA sequence encoding the second polypeptide.

The fusion polypeptide can comprise a polypeptide as described herein together with an unrelated immunogenic protein, such as an immunogenic protein capable of eliciting a recall response. Examples of such proteins include tetanus, tuberculosis and hepatitis proteins (see, for example, Stoute et al. New Engl. J. Med., 336:86-91, 1997).

In one preferred embodiment, the immunological fusion partner is derived from a Mycobacterium sp., such as a Mycobacterium tuberculosis-derived Ral2 fragment. Ral2 compositions and methods for their use in enhancing the expression and/or immunogenicity of heterologous polynucleotide/polypeptide sequences is described in U.S. patent application No. 60/158,585, the disclosure of which is incorporated herein by reference in its entirety. Briefly, Ral2 refers to a polynucleotide region that is a subsequence of a Mycobacterium tuberculosis MTB32A nucleic acid. MTB32A is a serine protease of 32 KD molecular weight encoded by a gene in virulent and avirulent strains of M. tuberculosis. The nucleotide sequence and amino acid sequence of MTB32A have been described (for example, U.S. patent application No. 60/158,585; see also, Skeiky et al., Infection and Immun. (1999) 67:3998-4007, incorporated herein by reference). C-terminal fragments of the MTB32A coding sequence express at high levels and remain as a soluble polypeptides throughout the purification process. Moreover, Ra12 may enhance the immunogenicity of heterologous immunogenic polypeptides with which it is fused. One preferred Ra12 fusion polypeptide comprises a 14 KD C-terminal fragment corresponding to amino acid residues 192 to 323 of MTB32A. Other preferred Ra12 polynucleotides generally comprise at least about 15 consecutive nucleotides, at least about 30 nucleotides, at least about 60 nucleotides, at least about 100 nucleotides, at least about 200 nucleotides, or at least about 300 nucleotides that encode a portion of a Ra12 polypeptide. Ra12 polynucleotides may comprise a native sequence (i.e., an endogenous sequence that encodes a Ra12 polypeptide or a portion thereof) or may comprise a variant of such a sequence. Ra12 polynucleotide variants may contain one or more substitutions, additions, deletions and/or insertions such that the biological activity of the encoded fusion polypeptide is not substantially diminished, relative to a fusion polypeptide comprising a native Ra12 polypeptide. Variants preferably exhibit at least about 70% identity, more preferably at least about 80% identity and most preferably at least about 90% identity to a polynucleotide sequence that encodes a native Ra12 polypeptide or a portion thereof.

Within other preferred embodiments, an immunological fusion partner is derived from protein D, a surface protein of the gram-negative bacterium Haemophilus influenza B (WO 91/18926). Preferably, a protein D derivative comprises approximately the first third of the protein (e.g., the first N-terminal 100-110 amino acids), and a protein D derivative may be lipidated. Within certain preferred embodiments, the first 109 residues of a Lipoprotein D fusion partner is included on the N-terminus to provide the polypeptide with additional exogenous T-cell epitopes and to increase the expression level in E. coli (thus functioning as an expression enhancer). The lipid tail ensures optimal presentation of the antigen to antigen presenting cells. Other fusion partners include the non-structural protein from influenzae virus, NS1 (hemaglutinin). Typically, the N-terminal 81 amino acids are used, although different fragments that include T-helper epitopes may be used.

In another embodiment, the immunological fusion partner is the protein known as LYTA, or a portion thereof (preferably a C-terminal portion). LYTA is derived from Streptococcus pneumoniae, which synthesizes an N-acetyl-L-alanine amidase known as amidase LYTA (encoded by the LytA gene; Gene 43:265-292, 1986). LYTA is an autolysin that specifically degrades certain bonds in the peptidoglycan backbone. The C-terminal domain of the LYTA protein is responsible for the affinity to the choline or to some choline analogues such as DEAE. This property has been exploited for the development of E. coli C-LYTA expressing plasmids useful for expression of fusion proteins. Purification of hybrid proteins containing the C-LYTA fragment at the amino terminus has been described (see Biotechnology 10:795-798, 1992). Within a preferred embodiment, a repeat portion of LYTA may be incorporated into a fusion polypeptide. A repeat portion is found in the C-terminal region starting at residue 178. A particularly preferred repeat portion incorporates residues 188-305.

Yet another illustrative embodiment involves fusion polypeptides, and the polynucleotides encoding them, wherein the fusion partner comprises a targeting signal capable of directing a polypeptide to the endosomal/lysosomal compartment, as described in U.S. Pat. No. 5,633,234. An immunogenic polypeptide of the invention, when fused with this targeting signal, will associate more efficiently with MHC class II molecules and thereby provide enhanced in vivo stimulation of CD4⁺ T-cells specific for the polypeptide.

Polypeptides of the invention are prepared using any of a variety of well known synthetic and/or recombinant techniques, the latter of which are further described below. Polypeptides, portions and other variants generally less than about 150 amino acids can be generated by synthetic means, using techniques well known to those of ordinary skill in the art. In one illustrative example, such polypeptides are synthesized using any of the commercially available solid-phase techniques, such as the Merrifield solid-phase synthesis method, where amino acids are sequentially added to a growing amino acid chain. See Merrifield, J. Am. Chem. Soc. 85:2149-2146, 1963. Equipment for automated synthesis of polypeptides is commercially available from suppliers such as Perkin Elmer/Applied BioSystems Division (Foster City, Calif.), and may be operated according to the manufacturer's instructions.

In general, polypeptide compositions (including fusion polypeptides) of the invention are isolated. An “isolated” polypeptide is one that is removed from its original environment. For example, a naturally-occurring protein or polypeptide is isolated if it is separated from some or all of the coexisting materials in the natural system. Preferably, such polypeptides are also purified, e.g., are at least about 90% pure, more preferably at least about 95% pure and most preferably at least about 99% pure.

Polynucleotide Compositions

The present invention, in other aspects, provides polynucleotide compositions. The terms “DNA” and “polynucleotide” are used essentially interchangeably herein to refer to a DNA molecule that has been isolated free of total genomic DNA of a particular species. “Isolated,” as used herein, means that a polynucleotide is substantially away from other coding sequences, and that the DNA molecule does not contain large portions of unrelated coding DNA, such as large chromosomal fragments or other functional genes or polypeptide coding regions. Of course, this refers to the DNA molecule as originally isolated, and does not exclude genes or coding regions later added to the segment by the hand of man.

As will be understood by those skilled in the art, the polynucleotide compositions of this invention can include genomic sequences, extra-genomic and plasmid-encoded sequences and smaller engineered gene segments that express, or may be adapted to express, proteins, polypeptides, peptides and the like. Such segments may be naturally isolated, or modified synthetically by the hand of man.

As will be also recognized by the skilled artisan, polynucleotides of the invention may be single-stranded (coding or antisense) or double-stranded, and may be DNA (genomic, cDNA or synthetic) or RNA molecules. RNA molecules may include HnRNA molecules, which contain introns and correspond to a DNA molecule in a one-to-one manner, and mRNA molecules, which do not contain introns. Additional coding or non-coding sequences may, but need not, be present within a polynucleotide of the present invention, and a polynucleotide may, but need not, be linked to other molecules and/or support materials.

Polynucleotides may comprise a native sequence (i.e., an endogenous sequence that encodes a polypeptide/protein of the invention or a portion thereof) or may comprise a sequence that encodes a variant or derivative, preferably and immunogenic variant or derivative, of such a sequence.

Therefore, according to another aspect of the present invention, polynucleotide compositions are provided that comprise some or all of a polynucleotide sequence set forth in any one of SEQ ID NO:1-377, complements of a polynucleotide sequence set forth in any one of SEQ ID NO:1-377, and degenerate variants of a polynucleotide sequence set forth in any one of SEQ ID NO:1-377. In certain preferred embodiments, the polynucleotide sequences set forth herein encode immunogenic polypeptides, as described above.

In other related embodiments, the present invention provides polynucleotide variants having substantial identity to the sequences disclosed herein in SEQ ID NO:1-377, for example those comprising at least 70% sequence identity, preferably at least 75%, 80%, 85%, 90%, 95%, 96%, 97%, 98%, or 99% or higher, sequence identity compared to a polynucleotide sequence of this invention using the methods described herein, (e.g., BLAST analysis using standard parameters, as described below). One skilled in this art will recognize that these values can be appropriately adjusted to determine corresponding identity of proteins encoded by two nucleotide sequences by taking into account codon degeneracy, amino acid similarity, reading frame positioning and the like.

Typically, polynucleotide variants will contain one or more substitutions, additions, deletions and/or insertions, preferably such that the immunogenicity of the polypeptide encoded by the variant polynucleotide is not substantially diminished relative to a polypeptide encoded by a polynucleotide sequence specifically set forth herein). The term “variants” should also be understood to encompasses homologous genes of xenogeneic origin.

In additional embodiments, the present invention provides polynucleotide fragments comprising or consisting of various lengths of contiguous stretches of sequence identical to or complementary to one or more of the sequences disclosed herein. For example, polynucleotides are provided by this invention that comprise or consist of at least about 10, 15, 20, 30, 40, 50, 75, 100, 150, 200, 300, 400, 500 or 1000 or more contiguous nucleotides of one or more of the sequences disclosed herein as well as all intermediate lengths there between. It will be readily understood that “intermediate lengths”, in this context, means any length between the quoted values, such as 16, 17, 18, 19, etc.; 21, 22, 23, etc.; 30, 31, 32, etc.; 50, 51, 52, 53, etc.; 100, 101, 102, 103, etc.; 150, 151, 152, 153, etc.; including all integers through 200-500; 500-1,000, and the like. A polynucleotide sequence as described here may be extended at one or both ends by additional nucleotides not found in the native sequence. This additional sequence may consist of 1, 2, 3, 4, 5, 6, 7, 8, 9, 10, 11, 12, 13, 14, 15, 16, 17, 18, 19, or 20 nucleotides at either end of the disclosed sequence or at both ends of the disclosed sequence.

In another embodiment of the invention, polynucleotide compositions are provided that are capable of hybridizing under moderate to high stringency conditions to a polynucleotide sequence provided herein, or a fragment thereof, or a complementary sequence thereof. Hybridization techniques are well known in the art of molecular biology. For purposes of illustration, suitable moderately stringent conditions for testing the hybridization of a polynucleotide of this invention with other polynucleotides include prewashing in a solution of 5× SSC, 0.5% SDS, 1.0 mM EDTA (pH 8.0); hybridizing at 50° C.-60° C., 5× SSC, overnight; followed by washing twice at 65° C. for 20 minutes with each of 2×, 0.5× and 0.2× SSC containing 0.1% SDS. One skilled in the art will understand that the stringency of hybridization can be readily manipulated, such as by altering the salt content of the hybridization solution and/or the temperature at which the hybridization is performed. For example, in another embodiment, suitable highly stringent hybridization conditions include those described above, with the exception that the temperature of hybridization is increased, e.g., to 60-65° C. or 65-70° C.

In certain preferred embodiments, the polynucleotides described above, e.g., polynucleotide variants, fragments and hybridizing sequences, encode polypeptides that are immunologically cross-reactive with a polypeptide sequence specifically set forth herein. In other preferred embodiments, such polynucleotides encode polypeptides that have a level of immunogenic activity of at least about 50%, preferably at least about 70%, and more preferably at least about 90% of that for a polypeptide sequence specifically set forth herein.

The polynucleotides of the present invention, or fragments thereof, regardless of the length of the coding sequence itself, may be combined with other DNA sequences, such as promoters, polyadenylation signals, additional restriction enzyme sites, multiple cloning sites, other coding segments, and the like, such that their overall length may vary considerably. It is therefore contemplated that a nucleic acid fragment of almost any length may be employed, with the total length preferably being limited by the ease of preparation and use in the intended recombinant DNA protocol. For example, illustrative polynucleotide segments with total lengths of about 10,000, about 5000, about 3000, about 2,000, about 1,000, about 500, about 200, about 100, about 50 base pairs in length, and the like, (including all intermediate lengths) are contemplated to be useful in many implementations of this invention.

When comparing polynucleotide sequences, two sequences are said to be “identical” if the sequence of nucleotides in the two sequences is the same when aligned for maximum correspondence, as described below. Comparisons between two sequences are typically performed by comparing the sequences over a comparison window to identify and compare local regions of sequence similarity. A “comparison window” as used herein, refers to a segment of at least about 20 contiguous positions, usually 30 to about 75, 40 to about 50, in which a sequence may be compared to a reference sequence of the same number of contiguous positions after the two sequences are optimally aligned.

Optimal alignment of sequences for comparison may be conducted using the Megalign program in the Lasergene suite of bioinformatics software (DNASTAR, Inc., Madison, Wis.), using default parameters. This program embodies several alignment schemes described in the following references: Dayhoff, M. O. (1978) A model of evolutionary change in proteins—Matrices for detecting distant relationships. In Dayhoff, M. O. (ed.) Atlas of Protein Sequence and Structure, National Biomedical Research Foundation, Washington, D.C. Vol. 5, Suppl. 3, pp. 345-358; Hein J. (1990) Unified Approach to Alignment and Phylogenes pp. 626-645 Methods in Enzymology vol. 183, Academic Press, Inc., San Diego, Calif.; Higgins, D. G. and Sharp, P.M. (1989) CABIOS 5:151-153; Myers, E. W. and Muller W. (1988) CABIOS 4:11-17; Robinson, E. D. (1971) Comb. Theor 11:105; Santou, N. Nes, M. (1987) Mol. Biol. Evol. 4:406-425; Sneath, P. H. A. and Sokal, R. R. (1973) Numerical Taxonomy—the Principles and Practice of Numerical Taxonomy, Freeman Press, San Francisco, Calif.; Wilbur, W. J. and Lipman, D. J. (1983) Proc. Natl. Acad., Sci. USA 80:726-730.

Alternatively, optimal alignment of sequences for comparison may be conducted by the local identity algorithm of Smith and Waterman (1981) Add. APL. Math 2:482, by the identity alignment algorithm of Needleman and Wunsch (1970) J. Mol. Biol. 48:443, by the search for similarity methods of Pearson and Lipman (1988) Proc. Natl. Acad. Sci. USA 85: 2444, by computerized implementations of these algorithms (GAP, BESTFIT, BLAST, FASTA, and TFASTA in the Wisconsin Genetics Software Package, Genetics Computer Group (GCG), 575 Science Dr., Madison, Wis.), or by inspection.

One preferred example of algorithms that are suitable for determining percent sequence identity and sequence similarity are the BLAST and BLAST 2.0 algorithms, which are described in Altschul et al. (1977) Nucl. Acids Res. 25:3389-3402 and Altschul et al. (1990) J. Mol. Biol. 215:403-410, respectively. BLAST and BLAST 2.0 can be used, for example with the parameters described herein, to determine percent sequence identity for the polynucleotides of the invention. Software for performing BLAST analyses is publicly available through the National Center for Biotechnology Information. In one illustrative example, cumulative scores can be calculated using, for nucleotide sequences, the parameters M (reward score for a pair of matching residues; always >0) and N (penalty score for mismatching residues; always <0). Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment. The BLASTN program (for nucleotide sequences) uses as defaults a wordlength (W) of 11, and expectation (E) of 10, and the BLOSUM62 scoring matrix (see Henikoff and Henikoff (1989) Proc. Natl. Acad. Sci. USA 89:10915) alignments, (B) of 50, expectation (E) of 10, M=5, N=−4 and a comparison of both strands.

Preferably, the “percentage of sequence identity” is determined by comparing two optimally aligned sequences over a window of comparison of at least 20 positions, wherein the portion of the polynucleotide sequence in the comparison window may comprise additions or deletions (i.e., gaps) of 20 percent or less, usually 5 to 15 percent, or 10 to 12 percent, as compared to the reference sequences (which does not comprise additions or deletions) for optimal alignment of the two sequences. The percentage is calculated by determining the number of positions at which the identical nucleic acid bases occurs in both sequences to yield the number of matched positions, dividing the number of matched positions by the total number of positions in the reference sequence (i.e., the window size) and multiplying the results by 100 to yield the percentage of sequence identity.

It will be appreciated by those of ordinary skill in the art that, as a result of the degeneracy of the genetic code, there are many nucleotide sequences that encode a polypeptide as described herein. Some of these polynucleotides bear minimal homology to the nucleotide sequence of any native gene. Nonetheless, polynucleotides that vary due to differences in codon usage are specifically contemplated by the present invention. Further, alleles of the genes comprising the polynucleotide sequences provided herein are within the scope of the present invention. Alleles are endogenous genes that are altered as a result of one or more mutations, such as deletions, additions and/or substitutions of nucleotides. The resulting mRNA and protein may, but need not, have an altered structure or function. Alleles may be identified using standard techniques (such as hybridization, amplification and/or database sequence comparison).

Therefore, in another embodiment of the invention, a mutagenesis approach, such as site-specific mutagenesis, is employed for the preparation of immunogenic variants and/or derivatives of the polypeptides described herein. By this approach, specific modifications in a polypeptide sequence can be made through mutagenesis of the underlying polynucleotides that encode them. These techniques provides a straightforward approach to prepare and test sequence variants, for example, incorporating one or more of the foregoing considerations, by introducing one or more nucleotide sequence changes into the polynucleotide.

Site-specific mutagenesis allows the production of mutants through the use of specific oligonucleotide sequences which encode the DNA sequence of the desired mutation, as well as a sufficient number of adjacent nucleotides, to provide a primer sequence of sufficient size and sequence complexity to form a stable duplex on both sides of the deletion junction being traversed. Mutations may be employed in a selected polynucleotide sequence to improve, alter, decrease, modify, or otherwise change the properties of the polynucleotide itself, and/or alter the properties, activity, composition, stability, or primary sequence of the encoded polypeptide.

In certain embodiments of the present invention, the inventors contemplate the mutagenesis of the disclosed polynucleotide sequences to alter one or more properties of the encoded polypeptide, such as the immunogenicity of a polypeptide vaccine. The techniques of site-specific mutagenesis are well known in the art, and are widely used to create variants of both polypeptides and polynucleotides. For example, site-specific mutagenesis is often used to alter a specific portion of a DNA molecule. In such embodiments, a primer comprising typically about 14 to about 25 nucleotides or so in length is employed, with about 5 to about 10 residues on both sides of the junction of the sequence being altered.

As will be appreciated by those of skill in the art, site-specific mutagenesis techniques have often employed a phage vector that exists in both a single stranded and double stranded form. Typical vectors useful in site-directed mutagenesis include vectors such as the M13 phage. These phage are readily commercially-available and their use is generally well-known to those skilled in the art. Double-stranded plasmids are also routinely employed in site directed mutagenesis that eliminates the step of transferring the gene of interest from a plasmid to a phage.

In general, site-directed mutagenesis in accordance herewith is performed by first obtaining a single-stranded vector or melting apart of two strands of a double-stranded vector that includes within its sequence a DNA sequence that encodes the desired peptide. An oligonucleotide primer bearing the desired mutated sequence is prepared, generally synthetically. This primer is then annealed with the single-stranded vector, and subjected to DNA polymerizing enzymes such as E. coli polymerase I Klenow fragment, in order to complete the synthesis of the mutation-bearing strand. Thus, a heteroduplex is formed wherein one strand encodes the original non-mutated sequence and the second strand bears the desired mutation. This heteroduplex vector is then used to transform appropriate cells, such as E. coli cells, and clones are selected which include recombinant vectors bearing the mutated sequence arrangement.

The preparation of sequence variants of the selected peptide-encoding DNA segments using site-directed mutagenesis provides a means of producing potentially useful species and is not meant to be limiting as there are other ways in which sequence variants of peptides and the DNA sequences encoding them may be obtained. For example, recombinant vectors encoding the desired peptide sequence may be treated with mutagenic agents, such as hydroxylamine, to obtain sequence variants. Specific details regarding these methods and protocols are found in the teachings of Maloy et al., 1994; Segal, 1976; Prokop and Bajpai, 1991; Kuby, 1994; and Maniatis et al., 1982, each incorporated herein by reference, for that purpose.

As used herein, the term “oligonucleotide directed mutagenesis procedure” refers to template-dependent processes and vector-mediated propagation which result in an increase in the concentration of a specific nucleic acid molecule relative to its initial concentration, or in an increase in the concentration of a detectable signal, such as amplification. As used herein, the term “oligonucleotide directed mutagenesis procedure” is intended to refer to a process that involves the template-dependent extension of a primer molecule. The term template dependent process refers to nucleic acid synthesis of an RNA or a DNA molecule wherein the sequence of the newly synthesized strand of nucleic acid is dictated by the well-known rules of complementary base pairing (see, for example, Watson, 1987). Typically, vector mediated methodologies involve the introduction of the nucleic acid fragment into a DNA or RNA vector, the clonal amplification of the vector, and the recovery of the amplified nucleic acid fragment. Examples of such methodologies are provided by U.S. Pat. No. 4,237,224, specifically incorporated herein by reference in its entirety.

In another approach for the production of polypeptide variants of the present invention, recursive sequence recombination, as described in U.S. Pat. No. 5,837,458, may be employed. In this approach, iterative cycles of recombination and screening or selection are performed to “evolve” individual polynucleotide variants of the invention having, for example, enhanced immunogenic activity.

In other embodiments of the present invention, the polynucleotide sequences provided herein can be advantageously used as probes or primers for nucleic acid hybridization. As such, it is contemplated that nucleic acid segments that comprise or consist of a sequence region of at least about a 15 nucleotide long contiguous sequence that has the same sequence as, or is complementary to, a 15 nucleotide long contiguous sequence disclosed herein will find particular utility. Longer contiguous identical or complementary sequences, e.g., those of about 20, 30, 40, 50, 100, 200, 500, 1000 (including all intermediate lengths) and even up to full length sequences will also be of use in certain embodiments.

The ability of such nucleic acid probes to specifically hybridize to a sequence of interest will enable them to be of use in detecting the presence of complementary sequences in a given sample. However, other uses are also envisioned, such as the use of the sequence information for the preparation of mutant species primers, or primers for use in preparing other genetic constructions.

Polynucleotide molecules having sequence regions consisting of contiguous nucleotide stretches of 10-14, 15-20, 30, 50, or even of 100-200 nucleotides or so (including intermediate lengths as well), identical or complementary to a polynucleotide sequence disclosed herein, are particularly contemplated as hybridization probes for use in, e.g., Southern and Northern blotting. This would allow a gene product, or fragment thereof, to be analyzed, both in diverse cell types and also in various bacterial cells. The total size of fragment, as well as the size of the complementary stretch(es), will ultimately depend on the intended use or application of the particular nucleic acid segment. Smaller fragments will generally find use in hybridization embodiments, wherein the length of the contiguous complementary region may be varied, such as between about 15 and about 100 nucleotides, but larger contiguous complementarity stretches may be used, according to the length complementary sequences one wishes to detect.

The use of a hybridization probe of about 15-25 nucleotides in length allows the formation of a duplex molecule that is both stable and selective. Molecules having contiguous complementary sequences over stretches greater than 15 bases in length are generally preferred, though, in order to increase stability and selectivity of the hybrid, and thereby improve the quality and degree of specific hybrid molecules obtained. One will generally prefer to design nucleic acid molecules having gene-complementary stretches of 15 to 25 contiguous nucleotides, or even longer where desired.

Hybridization probes may be selected from any portion of any of the sequences disclosed herein. All that is required is to review the sequences set forth herein, or to any continuous portion of the sequences, from about 15-25 nucleotides in length up to and including the full length sequence, that one wishes to utilize as a probe or primer. The choice of probe and primer sequences may be governed by various factors. For example, one may wish to employ primers from towards the termini of the total sequence.

Small polynucleotide segments or fragments may be readily prepared by, for example, directly synthesizing the fragment by chemical means, as is commonly practiced using an automated oligonucleotide synthesizer. Also, fragments may be obtained by application of nucleic acid reproduction technology, such as the PCRTM technology of U.S. Pat. No. 4,683,202 (incorporated herein by reference), by introducing selected sequences into recombinant vectors for recombinant production, and by other recombinant DNA techniques generally known to those of skill in the art of molecular biology.

The nucleotide sequences of the invention may be used for their ability to selectively form duplex molecules with complementary stretches of the entire gene or gene fragments of interest. Depending on the application envisioned, one will typically desire to employ varying conditions of hybridization to achieve varying degrees of selectivity of probe towards target sequence. For applications requiring high selectivity, one will typically desire to employ relatively stringent conditions to form the hybrids, e.g., one will select relatively low salt and/or high temperature conditions, such as provided by a salt concentration of from about 0.02 M to about 0.15 M salt at temperatures of from about 50° C. to about 70° C. Such selective conditions tolerate little, if any, mismatch between the probe and the template or target strand, and would be particularly suitable for isolating related sequences.

Of course, for some applications, for example, where one desires to prepare mutants employing a mutant primer strand hybridized to an underlying template, less stringent (reduced stringency) hybridization conditions will typically be needed in order to allow formation of the heteroduplex. In these circumstances, one may desire to employ salt conditions such as those of from about 0.15 M to about 0.9 M salt, at temperatures ranging from about 20° C. to about 55° C. Cross-hybridizing species can thereby be readily identified as positively hybridizing signals with respect to control hybridizations. In any case, it is generally appreciated that conditions can be rendered more stringent by the addition of increasing amounts of formamide, which serves to destabilize the hybrid duplex in the same manner as increased temperature. Thus, hybridization conditions can be readily manipulated, and thus will generally be a method of choice depending on the desired results.

According to another embodiment of the present invention, polynucleotide compositions comprising antisense oligonucleotides are provided. Antisense oligonucleotides have been demonstrated to be effective and targeted inhibitors of protein synthesis, and, consequently, provide a therapeutic approach by which a disease can be treated by inhibiting the synthesis of proteins that contribute to the disease. The efficacy of antisense oligonucleotides for inhibiting protein synthesis is well established. For example, the synthesis of polygalactauronase and the muscarine type 2 acetylcholine receptor are inhibited by antisense oligonucleotides directed to their respective mRNA sequences (U.S. Pat. No. 5,739,119 and U.S. Pat. No. 5,759,829). Further, examples of antisense inhibition have been demonstrated with the nuclear protein cyclin, the multiple drug resistance gene (MDG1), ICAM-1, E-selectin, STK-1, striatal GABAA receptor and human EGF (Jaskulski et al., Science. Jun. 10, 1988;240(4858):1544-6; Vasanthakumar and Ahmed, Cancer Commun. 1989;1(4):225-32; Peris et al., Brain Res Mol Brain Res. Jun. 15, 1998;57(2):310-20; U. S. Pat. No. 5,801,154; U.S. Pat. No. 5,789,573; U.S. Pat. No. 5,718,709 and U.S. Pat. No. 5,610,288). Antisense constructs have also been described that inhibit and can be used to treat a variety of abnormal cellular proliferations, e.g cancer (U.S. Pat. No. 5,747,470; U.S. Pat. No. 5,591,317 and U.S. Pat. No. 5,783,683).

Therefore, in certain embodiments, the present invention provides oligonucleotide sequences that comprise all, or a portion of, any sequence that is capable of specifically binding to polynucleotide sequence described herein, or a complement thereof. In one embodiment, the antisense oligonucleotides comprise DNA or derivatives thereof. In another embodiment, the oligonucleotides comprise RNA or derivatives thereof. In a third embodiment, the oligonucleotides are modified DNAs comprising a phosphorothioated modified backbone. In a fourth embodiment, the oligonucleotide sequences comprise peptide nucleic acids or derivatives thereof. In each case, preferred compositions comprise a sequence region that is complementary, and more preferably substantially-complementary, and even more preferably, completely complementary to one or more portions of polynucleotides disclosed herein. Selection of antisense compositions specific for a given gene sequence is based upon analysis of the chosen target sequence and determination of secondary structure, Tₘ, binding energy, and relative stability. Antisense compositions may be selected based upon their relative inability to form dimers, hairpins, or other secondary structures that would reduce or prohibit specific binding to the target mRNA in a host cell. Highly preferred target regions of the mRNA, are those which are at or near the AUG translation initiation codon, and those sequences which are substantially complementary to 5′ regions of the mRNA. These secondary structure analyses and target site selection considerations can be performed, for example, using v.4 of the OLIGO primer analysis software and/or the BLASTN 2.0.5 algorithm software (Altschul et al., Nucleic Acids Res. 1997, 25(17):3389-402).

The use of an antisense delivery method employing a short peptide vector, termed MPG (27 residues), is also contemplated. The MPG peptide contains a hydrophobic domain derived from the fusion sequence of HIV gp41 and a hydrophilic domain from the nuclear localization sequence of SV40 T-antigen (Morris et al., Nucleic Acids Res. Jul. 15, 1997;25(14):2730-6). It has been demonstrated that several molecules of the MPG peptide coat the antisense oligonucleotides and can be delivered into cultured mammalian cells in less than 1 hour with relatively high efficiency (90%). Further, the interaction with MPG strongly increases both the stability of the oligonucleotide to nuclease and the ability to cross the plasma membrane.

According to another embodiment of the invention, the polynucleotide compositions described herein are used in the design and preparation of ribozyme molecules for inhibiting expression of the tumor polypeptides and proteins of the present invention in tumor cells. Ribozymes are RNA-protein complexes that cleave nucleic acids in a site-specific fashion. Ribozymes have specific catalytic domains that possess endonuclease activity (Kim and Cech, Proc Natl Acad Sci U S A. 1987 Dec;84(24):8788-92; Forster and Symons, Cell. Apr. 24, 1987;49(2):211-20). For example, a large number of ribozymes accelerate phosphoester transfer reactions with a high degree of specificity, often cleaving only one of several phosphoesters in an oligonucleotide substrate (Cech et al., Cell. 1981 December;27(3 Pt 2):487-96; Michel and Westhof, J Mol Biol. Dec. 5, 1990;216(3):585-610; Reinhold-Hurek and Shub, Nature. May 14, 1992;357(6374):173-6). This specificity has been attributed to the requirement that the substrate bind via specific base-pairing interactions to the internal guide sequence (“IGS”) of the ribozyme prior to chemical reaction.

Six basic varieties of naturally occurring enzymatic RNAs are known presently. Each can catalyze the hydrolysis of RNA phosphodiester bonds in trans (and thus can cleave other RNA molecules) under physiological conditions. In general, enzymatic nucleic acids act by first binding to a target RNA. Such binding occurs through the target binding portion of a enzymatic nucleic acid which is held in close proximity to an enzymatic portion of the molecule that acts to cleave the target RNA. Thus, the enzymatic nucleic acid first recognizes and then binds a target RNA through complementary base-pairing, and once bound to the correct site, acts enzymatically to cut the target RNA. Strategic cleavage of such a target RNA will destroy its ability to direct synthesis of an encoded protein. After an enzymatic nucleic acid has bound and cleaved its RNA target, it is released from that RNA to search for another target and can repeatedly bind and cleave new targets.

The enzymatic nature of a ribozyme is advantageous over many technologies, such as antisense technology (where a nucleic acid molecule simply binds to a nucleic acid target to block its translation) since the concentration of ribozyme necessary to affect a therapeutic treatment is lower than that of an antisense oligonucleotide. This advantage reflects the ability of the ribozyme to act enzymatically. Thus, a single ribozyme molecule is able to cleave many molecules of target RNA. In addition, the ribozyme is a highly specific inhibitor, with the specificity of inhibition depending not only on the base pairing mechanism of binding to the target RNA, but also on the mechanism of target RNA cleavage. Single mismatches, or base-substitutions, near the site of cleavage can completely eliminate catalytic activity of a ribozyme. Similar mismatches in antisense molecules do not prevent their action (Woolf et al., Proc Natl Acad Sci U S A. Aug. 15, 1992;89(16):7305-9). Thus, the specificity of action of a ribozyme is greater than that of an antisense oligonucleotide binding the same RNA site.

The enzymatic nucleic acid molecule may be formed in a hammerhead, hairpin, a hepatitis 6 virus, group I intron or RNaseP RNA (in association with an RNA guide sequence) or Neurospora VS RNA motif. Examples of hammerhead motifs are described by Rossi et al. Nucleic Acids Res. 1992 Sep 11;20(17):4559-65. Examples of hairpin motifs are described by Hampel et al. (Eur. Pat. Appl. Publ. No. EP 0360257), Hampel and Tritz, Biochemistry Jun. 13,1989;28(12):4929-33; Hampel et al., Nucleic Acids Res. Jan. 25, 1990;18(2):299-304 and U.S. Pat. No. 5,631,359. An example of the hepatitis δ virus motif is described by Perrotta and Been, Biochemistry. Dec. 1, 1992;31(47):11843-52; an example of the RNaseP motif is described by Guerrier-Takada et al., Cell. December 1983;35(3 Pt 2):849-57; Neurospora VS RNA ribozyme motif is described by Collins (Saville and Collins, Cell. May 18, 1990;61(4):685-96; Saville and Collins, Proc Natl Acad Sci U S A. Oct 1, 1991;88(19):8826-30; Collins and Olive, Biochemistry. Mar 23, 1993;32(11):2795-9); and an example of the Group I intron is described in (U.S. Pat. No. 4,987,071). All that is important in an enzymatic nucleic acid molecule of this invention is that it has a specific substrate binding site which is complementary to one or more of the target gene RNA regions, and that it have nucleotide sequences within or surrounding that substrate binding site which impart an RNA cleaving activity to the molecule. Thus the ribozyme constructs need not be limited to specific motifs mentioned herein.

Ribozymes may be designed as described in Int. Pat. Appl. Publ. No. WO 93/23569 and Int. Pat. Appl. Publ. No. WO 94/02595, each specifically incorporated herein by reference) and synthesized to be tested in vitro and in vivo, as described. Such ribozymes can also be optimized for delivery. While specific examples are provided, those in the art will recognize that equivalent RNA targets in other species can be utilized when necessary.

Ribozyme activity can be optimized by altering the length of the ribozyme binding arms, or chemically synthesizing ribozymes with modifications that prevent their degradation by serum ribonucleases (see e.g., Int. Pat. Appl. Publ. No. WO 92/07065; Int. Pat. Appl. Publ. No. WO 93/15187; Int. Pat. Appl. Publ. No. WO 91/03162; Eur. Pat. Appl. Publ. No. 92110298.4; U.S. Pat. No. 5,334,711; and Int. Pat. Appl. Publ. No. WO 94/13688, which describe various chemical modifications that can be made to the sugar moieties of enzymatic RNA molecules), modifications which enhance their efficacy in cells, and removal of stem II bases to shorten RNA synthesis times and reduce chemical requirements.

Sullivan et al. (Int. Pat. Appl. Publ. No. WO 94/02595) describes the general methods for delivery of enzymatic RNA molecules. Ribozymes may be administered to cells by a variety of methods known to those familiar to the art, including, but not restricted to, encapsulation in liposomes, by iontophoresis, or by incorporation into other vehicles, such as hydrogels, cyclodextrins, biodegradable nanocapsules, and bioadhesive microspheres. For some indications, ribozymes may be directly delivered ex vivo to cells or tissues with or without the aforementioned vehicles. Alternatively, the RNA/vehicle combination may be locally delivered by direct inhalation, by direct injection or by use of a catheter, infusion pump or stent. Other routes of delivery include, but are not limited to, intravascular, intramuscular, subcutaneous or joint injection, aerosol inhalation, oral (tablet or pill form), topical, systemic, ocular, intraperitoneal and/or intrathecal delivery. More detailed descriptions of ribozyme delivery and administration are provided in Int. Pat. Appl. Publ. No. WO 94/02595 and Int. Pat. Appl. Publ. No. WO 93/23569, each specifically incorporated herein by reference.

Another means of accumulating high concentrations of a ribozyme(s) within cells is to incorporate the ribozyme-encoding sequences into a DNA expression vector. Transcription of the ribozyme sequences are driven from a promoter for eukaryotic RNA polymerase I (pol I), RNA polymerase II (pol II), or RNA polymerase III (pol III). Transcripts from pol II or pol III promoters will be expressed at high levels in all cells; the levels of a given pol II promoter in a given cell type will depend on the nature of the gene regulatory sequences (enhancers, silencers, etc.) present nearby. Prokaryotic RNA polymerase promoters may also be used, providing that the prokaryotic RNA polymerase enzyme is expressed in the appropriate cells Ribozymes expressed from such promoters have been shown to function in mammalian cells. Such transcription units can be incorporated into a variety of vectors for introduction into mammalian cells, including but not restricted to, plasmid DNA vectors, viral DNA vectors (such as adenovirus or adeno-associated vectors), or viral RNA vectors (such as retroviral, semliki forest virus, sindbis virus vectors).

In another embodiment of the invention, peptide nucleic acids (PNAs) compositions are provided. PNA is a DNA mimic in which the nucleobases are attached to a pseudopeptide backbone (Good and Nielsen, Antisense Nucleic Acid Drug Dev. 1997 7(4) 431-37). PNA is able to be utilized in a number methods that traditionally have used RNA or DNA. Often PNA sequences perform better in techniques than the corresponding RNA or DNA sequences and have utilities that are not inherent to RNA or DNA. A review of PNA including methods of making, characteristics of, and methods of using, is provided by Corey (Trends Biotechnol June 1997;15(6):224-9). As such, in certain embodiments, one may prepare PNA sequences that are complementary to one or more portions of the ACE mRNA sequence, and such PNA compositions may be used to regulate, alter, decrease, or reduce the translation of ACE-specific mRNA, and thereby alter the level of ACE activity in a host cell to which such PNA compositions have been administered.

PNAs have 2-aminoethyl-glycine linkages replacing the normal phosphodiester backbone of DNA (Nielsen et al., Science Dec. 6, 1991;254(5037):1497-500; Hanvey et al., Science. Nov. 27, 1992;258(5087):1481-5; Hyrup and Nielsen, Bioorg Med Chem. January 1996; 4(1):5-23). This chemistry has three important consequences: firstly, in contrast to DNA or phosphorothioate oligonucleotides, PNAs are neutral molecules; secondly, PNAs are achiral, which avoids the need to develop a stereoselective synthesis; and thirdly, PNA synthesis uses standard Boc or Fmoc protocols for solid-phase peptide synthesis, although other methods, including a modified Merrifield method, have been used.

PNA monomers or ready-made oligomers are commercially available from PerSeptive Biosystems (Framingham, Mass.). PNA syntheses by either Boc or Fmoc protocols are straightforward using manual or automated protocols (Norton et al., Bioorg Med Chem. April 1995;3(4):437-45). The manual protocol lends itself to the production of chemically modified PNAs or the simultaneous synthesis of families of closely related PNAs.

As with peptide synthesis, the success of a particular PNA synthesis will depend on the properties of the chosen sequence. For example, while in theory PNAs can incorporate any combination of nucleotide bases, the presence of adjacent purines can lead to deletions of one or more residues in the product. In expectation of this difficulty, it is suggested that, in producing PNAs with adjacent purines, one should repeat the coupling of residues likely to be added inefficiently. This should be followed by the purification of PNAs by reverse-phase high-pressure liquid chromatography, providing yields and purity of product similar to those observed during the synthesis of peptides.

Modifications of PNAs for a given application may be accomplished by coupling amino acids during solid-phase synthesis or by attaching compounds that contain a carboxylic acid group to the exposed N-terminal amine. Alternatively, PNAs can be modified after synthesis by coupling to an introduced lysine or cysteine. The ease with which PNAs can be modified facilitates optimization for better solubility or for specific functional requirements. Once synthesized, the identity of PNAs and their derivatives can be confirmed by mass spectrometry. Several studies have made and utilized modifications of PNAs (for example, Norton et al., Bioorg Med Chem. April 1995;3(4):437-45; Petersen et al., J Pept Sci. May-June 1995;1(3):175-83; Orum et al., Biotechniques. September 1995;19(3):472-80; Footer et al., Biochemistry. Aug. 20, 1996; 35(33):10673-9; Griffith et al., Nucleic Acids Res. Aug. 11, 1995;23(15):3003-8; Pardridge et al., Proc Natl Acad Sci U S A. Jun. 6, 1995;92(12):5592-6; Boffa et al., Proc Natl Acad Sci U S A. Mar. 14, 1995;92(6):1901-5; Gambacorti-Passerini et al., Blood. Aug. 15, 1996;88(4):1411-7; Armitage et al., Proc Natl Acad Sci U S A. Nov 11, 1997;94(23):12320-5; Seeger et al., Biotechniques. Septmeber 1997;23(3):512-7). U.S. Pat. No. 5,700,922 discusses PNA-DNA-PNA chimeric molecules and their uses in diagnostics, modulating protein in organisms, and treatment of conditions susceptible to therapeutics.

Methods of characterizing the antisense binding properties of PNAs are discussed in Rose (Anal Chem. Dec. 15, 1993;65(24):3545-9) and Jensen et al. (Biochemistry. Apr. 22, 1997;36(16):5072-7). Rose uses capillary gel electrophoresis to determine binding of PNAs to their complementary oligonucleotide, measuring the relative binding kinetics and stoichiometry. Similar types of measurements were made by Jensen et al. using BIAcore™ technology.

Other applications of PNAs that have been described and will be apparent to the skilled artisan include use in DNA strand invasion, antisense inhibition, mutational analysis, enhancers of transcription, nucleic acid purification, isolation of transcriptionally active genes, blocking of transcription factor binding, genome cleavage, biosensors, in situ hybridization, and the like.

Polynucleotide Identification, Characterization and Expression

Polynucleotides compositions of the present invention may be identified, prepared and/or manipulated using any of a variety of well established techniques (see generally, Sambrook et al., Molecular Cloning: A Laboratory Manual, Cold Spring Harbor Laboratories, Cold Spring Harbor, N.Y., 1989, and other like references). For example, a polynucleotide may be identified, as described in more detail below, by screening a microarray of cDNAs for tumor-associated expression (i.e., expression that is at least two fold greater in a tumor than in normal tissue, as determined using a representative assay provided herein). Such screens may be performed, for example, using the microarray technology of Affymetrix, Inc. (Santa Clara, Calif.) according to the manufacturer's instructions (and essentially as described by Schena et al., Proc. Natl. Acad. Sci. USA 93:10614-10619, 1996 and Heller et al., Proc. Natl. Acad. Sci. USA 94:2150-2155, 1997). Alternatively, polynucleotides may be amplified from cDNA prepared from cells expressing the proteins described herein, such as tumor cells.

Many template dependent processes are available to amplify a target sequences of interest present in a sample. One of the best known amplification methods is the polymerase chain reaction (PCRTM) which is described in detail in U.S. Pat. Nos. 4,683,195, 4,683,202 and 4,800,159, each of which is incorporated herein by reference in its entirety. Briefly, in PCRTM, two primer sequences are prepared which are complementary to regions on opposite complementary strands of the target sequence. An excess of deoxynucleoside triphosphates is added to a reaction mixture along with a DNA polymerase (e.g., Taq polymerase). If the target sequence is present in a sample, the primers will bind to the target and the polymerase will cause the primers to be extended along the target sequence by adding on nucleotides. By raising and lowering the temperature of the reaction mixture, the extended primers will dissociate from the target to form reaction products, excess primers will bind to the target and to the reaction product and the process is repeated. Preferably reverse transcription and PCR™ amplification procedure may be performed in order to quantify the amount of mRNA amplified. Polymerase chain reaction methodologies are well known in the art.

Any of a number of other template dependent processes, many of which are variations of the PCR™ amplification technique, are readily known and available in the art. Illustratively, some such methods include the ligase chain reaction (referred to as LCR), described, for example, in Eur. Pat. Appl. Publ. No. 320,308 and U.S. Pat. No. 4,883,750; Qbeta Replicase, described in PCT Intl. Pat. Appl. Publ. No. PCT/US87/00880; Strand Displacement Amplification (SDA) and Repair Chain Reaction (RCR). Still other amplification methods are described in Great Britain Pat. Appl. No. 2 202 328, and in PCT Intl. Pat. Appl. Publ. No. PCT/US89/01025. Other nucleic acid amplification procedures include transcription-based amplification systems (TAS) (PCT Intl. Pat. Appl. Publ. No. WO 88/10315), including nucleic acid sequence based amplification (NASBA) and 3SR. Eur. Pat. Appl. Publ. No. 329,822 describes a nucleic acid amplification process involving cyclically synthesizing single-stranded RNA (“ssRNA”), ssDNA, and double-stranded DNA (dsDNA). PCT Intl. Pat. Appl. Publ. No. WO 89/06700 describes a nucleic acid sequence amplification scheme based on the hybridization of a promoter/primer sequence to a target single-stranded DNA (“ssDNA”) followed by transcription of many RNA copies of the sequence. Other amplification methods such as “RACE” (Frohman, 1990), and “one-sided PCR” (Ohara, 1989) are also well-known to those of skill in the art.

An amplified portion of a polynucleotide of the present invention may be used to isolate a full length gene from a suitable library (e.g., a tumor cDNA library) using well known techniques. Within such techniques, a library (cDNA or genomic) is screened using one or more polynucleotide probes or primers suitable for amplification. Preferably, a library is size-selected to include larger molecules. Random primed libraries may also be preferred for identifying 5′ and upstream regions of genes. Genomic libraries are preferred for obtaining introns and extending 5′ sequences.

For hybridization techniques, a partial sequence may be labeled (e.g., by nick-translation or end-labeling with 32p) using well known techniques. A bacterial or bacteriophage library is then generally screened by hybridizing filters containing denatured bacterial colonies (or lawns containing phage plaques) with the labeled probe (see Sambrook et al., Molecular Cloning: A Laboratory Manual, Cold Spring Harbor Laboratories, Cold Spring Harbor, N.Y., 1989). Hybridizing colonies or plaques are selected and expanded, and the DNA is isolated for further analysis. cDNA clones may be analyzed to determine the amount of additional sequence by, for example, PCR using a primer from the partial sequence and a primer from the vector. Restriction maps and partial sequences may be generated to identify one or more overlapping clones. The complete sequence may then be determined using standard techniques, which may involve generating a series of deletion clones. The resulting overlapping sequences can then assembled into a single contiguous sequence. A full length cDNA molecule can be generated by ligating suitable fragments, using well known techniques.

Alternatively, amplification techniques, such as those described above, can be useful for obtaining a full length coding sequence from a partial cDNA sequence. One such amplification technique is inverse PCR (see Triglia et al., Nucl. Acids Res. 16:8186, 1988), which uses restriction enzymes to generate a fragment in the known region of the gene. The fragment is then circularized by intramolecular ligation and used as a template for PCR with divergent primers derived from the known region. Within an alternative approach, sequences adjacent to a partial sequence may be retrieved by amplification with a primer to a linker sequence and a primer specific to a known region. The amplified sequences are typically subjected to a second round of amplification with the same linker primer and a second primer specific to the known region. A variation on this procedure, which employs two primers that initiate extension in opposite directions from the known sequence, is described in WO 96/38591. Another such technique is known as “rapid amplification of cDNA ends” or RACE. This technique involves the use of an internal primer and an external primer, which hybridizes to a polyA region or vector sequence, to identify sequences that are 5′ and 3′ of a known sequence. Additional techniques include capture PCR (Lagerstrom et al., PCR Methods Applic. 1:111-19, 1991) and walking PCR (Parker et al., Nucl. Acids. Res. 19:3055-60, 1991). Other methods employing amplification may also be employed to obtain a full length cDNA sequence.

In certain instances, it is possible to obtain a full length cDNA sequence by analysis of sequences provided in an expressed sequence tag (EST) database, such as that available from GenBank. Searches for overlapping ESTs may generally be performed using well known programs (e.g., NCBI BLAST searches), and such ESTs may be used to generate a contiguous full length sequence. Full length DNA sequences may also be obtained by analysis of genomic fragments.

In other embodiments of the invention, polynucleotide sequences or fragments thereof which encode polypeptides of the invention, or fusion proteins or functional equivalents thereof, may be used in recombinant DNA molecules to direct expression of a polypeptide in appropriate host cells. Due to the inherent degeneracy of the genetic code, other DNA sequences that encode substantially the same or a functionally equivalent amino acid sequence may be produced and these sequences may be used to clone and express a given polypeptide.

As will be understood by those of skill in the art, it may be advantageous in some instances to produce polypeptide-encoding nucleotide sequences possessing non-naturally occurring codons. For example, codons preferred by a particular prokaryotic or eukaryotic host can be selected to increase the rate of protein expression or to produce a recombinant RNA transcript having desirable properties, such as a half-life which is longer than that of a transcript generated from the naturally occurring sequence.

Moreover, the polynucleotide sequences of the present invention can be engineered using methods generally known in the art in order to alter polypeptide encoding sequences for a variety of reasons, including but not limited to, alterations which modify the cloning, processing, and/or expression of the gene product. For example, DNA shuffling by random fragmentation and PCR reassembly of gene fragments and synthetic oligonucleotides may be used to engineer the nucleotide sequences. In addition, site-directed mutagenesis may be used to insert new restriction sites, alter glycosylation patterns, change codon preference, produce splice variants, or introduce mutations, and so forth.

In another embodiment of the invention, natural, modified, or recombinant nucleic acid sequences may be ligated to a heterologous sequence to encode a fusion protein. For example, to screen peptide libraries for inhibitors of polypeptide activity, it may be useful to encode a chimeric protein that can be recognized by a commercially available antibody. A fusion protein may also be engineered to contain a cleavage site located between the polypeptide-encoding sequence and the heterologous protein sequence, so that the polypeptide may be cleaved and purified away from the heterologous moiety.

Sequences encoding a desired polypeptide may be synthesized, in whole or in part, using chemical methods well known in the art (see Caruthers, M. H. et al. (1980) Nucl. Acids Res. Symp. Ser. 215-223, Horn, T. et al. (1980) Nucl. Acids Res. Symp. Ser. 225-232). Alternatively, the protein itself may be produced using chemical methods to synthesize the amino acid sequence of a polypeptide, or a portion thereof. For example, peptide synthesis can be performed using various solid-phase techniques (Roberge, J. Y. et al. (1995) Science 269:202-204) and automated synthesis may be achieved, for example, using the ABI 431A Peptide Synthesizer (Perkin Elmer, Palo Alto, Calif.).

A newly synthesized peptide may be substantially purified by preparative high performance liquid chromatography (e.g., Creighton, T. (1983) Proteins, Structures and Molecular Principles, WH Freeman and Co., New York, N.Y.) or other comparable techniques available in the art. The composition of the synthetic peptides may be confirmed by amino acid analysis or sequencing (e.g., the Edman degradation procedure). Additionally, the amino acid sequence of a polypeptide, or any part thereof, may be altered during direct synthesis and/or combined using chemical methods with sequences from other proteins, or any part thereof, to produce a variant polypeptide.

In order to express a desired polypeptide, the nucleotide sequences encoding the polypeptide, or functional equivalents, may be inserted into appropriate expression vector, i.e., a vector which contains the necessary elements for the transcription and translation of the inserted coding sequence. Methods which are well known to those skilled in the art may be used to construct expression vectors containing sequences encoding a polypeptide of interest and appropriate transcriptional and translational control elements. These methods include in vitro recombinant DNA techniques, synthetic techniques, and in vivo genetic recombination. Such techniques are described, for example, in Sambrook, J. et al. (1989) Molecular Cloning, A Laboratory Manual, Cold Spring Harbor Press, Plainview, N.Y., and Ausubel, F. M. et al. (1989) Current Protocols in Molecular Biology, John Wiley & Sons, New York. N.Y.

A variety of expression vector/host systems may be utilized to contain and express polynucleotide sequences. These include, but are not limited to, microorganisms such as bacteria transformed with recombinant bacteriophage, plasmid, or cosmid DNA expression vectors; yeast transformed with yeast expression vectors; insect cell systems infected with virus expression vectors (e.g., baculovirus); plant cell systems transformed with virus expression vectors (e.g., cauliflower mosaic virus, CaMV; tobacco mosaic virus, TMV) or with bacterial expression vectors (e.g., Ti or pBR322 plasmids); or animal cell systems.

The “control elements” or “regulatory sequences” present in an expression vector are those non-translated regions of the vector—enhancers, promoters, 5′ and 3′ untranslated regions—which interact with host cellular proteins to carry out transcription and translation. Such elements may vary in their strength and specificity. Depending on the vector system and host utilized, any number of suitable transcription and translation elements, including constitutive and inducible promoters, may be used. For example, when cloning in bacterial systems, inducible promoters such as the hybrid lacZ promoter of the pBLUESCRIPT phagemid (Stratagene, La Jolla, Calif.) or pSPORT1 plasmid (Gibco BRL, Gaithersburg, Md.) and the like may be used. In mammalian cell systems, promoters from mammalian genes or from mammalian viruses are generally preferred. If it is necessary to generate a cell line that contains multiple copies of the sequence encoding a polypeptide, vectors based on SV40 or EBV may be advantageously used with an appropriate selectable marker.

In bacterial systems, any of a number of expression vectors may be selected depending upon the use intended for the expressed polypeptide. For example, when large quantities are needed, for example for the induction of antibodies, vectors which direct high level expression of fusion proteins that are readily purified may be used. Such vectors include, but are not limited to, the multifunctional E. coli cloning and expression vectors such as pBLUESCRIPT (Stratagene), in which the sequence encoding the polypeptide of interest may be ligated into the vector in frame with sequences for the amino-terminal Met and the subsequent 7 residues of .beta.-galactosidase so that a hybrid protein is produced; pIN vectors (Van Heeke, G. and S. M. Schuster (1989) J. Biol. Chem. 264:5503-5509); and the like. pGEX Vectors (Promega, Madison, Wis.) may also be used to express foreign polypeptides as fusion proteins with glutathione S-transferase (GST). In general, such fusion proteins are soluble and can easily be purified from lysed cells by adsorption to glutathione-agarose beads followed by elution in the presence of free glutathione. Proteins made in such systems may be designed to include heparin, thrombin, or factor XA protease cleavage sites so that the cloned polypeptide of interest can be released from the GST moiety at will.

In the yeast, Saccharomyces cerevisiae, a number of vectors containing constitutive or inducible promoters such as alpha factor, alcohol oxidase, and PGH may be used. For reviews, see Ausubel et al. (supra) and Grant et al. (1987) Methods Enzymol. 153:516-544.

In cases where plant expression vectors are used, the expression of sequences encoding polypeptides may be driven by any of a number of promoters. For example, viral promoters such as the 35S and 19S promoters of CaMV may be used alone or in combination with the omega leader sequence from TMV (Takamatsu, N. (1987) EMBO J. 6:307-311. Alternatively, plant promoters such as the small subunit of RUBISCO or heat shock promoters may be used (Coruzzi, G. et al. (1984) EMBO J 3:1671-1680; Broglie, R. et al. (1984) Science 224:838-843; and Winter, J. et al. (1991) Results Probl. Cell Differ. 17:85-105). These constructs can be introduced into plant cells by direct DNA transformation or pathogen-mediated transfection. Such techniques are described in a number of generally available reviews (see, for example, Hobbs, S. or Murry, L. E. in McGraw Hill Yearbook of Science and Technology (1992) McGraw Hill, New York, N.Y.; pp. 191-196).

An insect system may also be used to express a polypeptide of interest. For example, in one such system, Autographa califomica nuclear polyhedrosis virus (AcNPV) is used as a vector to express foreign genes in Spodoptera frugiperda cells or in Trichoplusia larvae. The sequences encoding the polypeptide may be cloned into a non-essential region of the virus, such as the polyhedrin gene, and placed under control of the polyhedrin promoter. Successful insertion of the polypeptide-encoding sequence will render the polyhedrin gene inactive and produce recombinant virus lacking coat protein. The recombinant viruses may then be used to infect, for example, S. frugiperda cells or Trichoplusia larvae in which the polypeptide of interest may be expressed (Engelhard, E. K. et al. (1994) Proc. Natl. Acad. Sci. 91 :3224-3227).

In mammalian host cells, a number of viral-based expression systems are generally available. For example, in cases where an adenovirus is used as an expression vector, sequences encoding a polypeptide of interest may be ligated into an adenovirus transcription/translation complex consisting of the late promoter and tripartite leader sequence. Insertion in a non-essential E1 or E3 region of the viral genome may be used to obtain a viable virus which is capable of expressing the polypeptide in infected host cells (Logan, J. and Shenk, T. (1984) Proc. Natl. Acad. Sci. 81:3655-3659). In addition, transcription enhancers, such as the Rous sarcoma virus (RSV) enhancer, may be used to increase expression in mammalian host cells.

Specific initiation signals may also be used to achieve more efficient translation of sequences encoding a polypeptide of interest. Such signals include the ATG initiation codon and adjacent sequences. In cases where sequences encoding the polypeptide, its initiation codon, and upstream sequences are inserted into the appropriate expression vector, no additional transcriptional or translational control signals may be needed. However, in cases where only coding sequence, or a portion thereof, is inserted, exogenous translational control signals including the ATG initiation codon should be provided. Furthermore, the initiation codon should be in the correct reading frame to ensure translation of the entire insert. Exogenous translational elements and initiation codons may be of various origins, both natural and synthetic. The efficiency of expression may be enhanced by the inclusion of enhancers which are appropriate for the particular cell system which is used, such as those described in the literature (Scharf, D. et al. (1994) Results Probl Cell Differ. 20:125-162).

In addition, a host cell strain may be chosen for its ability to modulate the expression of the inserted sequences or to process the expressed protein in the desired fashion. Such modifications of the polypeptide include, but are not limited to, acetylation, carboxylation. glycosylation, phosphorylation, lipidation, and acylation. Post-translational processing which cleaves a “prepro” form of the protein may also be used to facilitate correct insertion, folding and/or function. Different host cells such as CHO, COS, HeLa, MDCK, HEK293, and W138, which have specific cellular machinery and characteristic mechanisms for such post-translational activities, may be chosen to ensure the correct modification and processing of the foreign protein.

For long-term, high-yield production of recombinant proteins, stable expression is generally preferred. For example, cell lines which stably express a polynucleotide of interest may be transformed using expression vectors which may contain viral origins of replication and/or endogenous expression elements and a selectable marker gene on the same or on a separate vector. Following the introduction of the vector, cells may be allowed to grow for 1-2 days in an enriched media before they are switched to selective media. The purpose of the selectable marker is to confer resistance to selection, and its presence allows growth and recovery of cells which successfully express the introduced sequences. Resistant clones of stably transformed cells may be proliferated using tissue culture techniques appropriate to the cell type.

Any number of selection systems may be used to recover transformed cell lines. These include, but are not limited to, the herpes simplex virus thymidine kinase (Wigler, M. et al. (1977) Cell 11:223-32) and adenine phosphoribosyltransferase (Lowy, I. et al. (1990) Cell 22:817-23) genes which can be employed in tk.sup.- or aprt.sup.- cells, respectively. Also, antimetabolite, antibiotic or herbicide resistance can be used as the basis for selection; for example, dhfr which confers resistance to methotrexate (Wigler, M. et al. (1980) Proc. Natl. Acad. Sci. 77:3567-70); npt, which confers resistance to the aminoglycosides, neomycin and G-418 (Colbere-Garapin, F. et al (1981) J. Mol. Biol. 150:1-14); and als or pat, which confer resistance to chlorsulfuron and phosphinotricin acetyltransferase, respectively (Murry, supra). Additional selectable genes have been described, for example, trpb, which allows cells to utilize indole in place of tryptophan, or hisD, which allows cells to utilize histinol in place of histidine (Hartman, S. C. and R. C. Mulligan (1988) Proc. Natl. Acad. Sci. 85:8047-51). The use of visible markers has gained popularity with such markers as anthocyanins, beta-glucuronidase and its substrate GUS, and luciferase and its substrate luciferin, being widely used not only to identify transformants, but also to quantify the amount of transient or stable protein expression attributable to a specific vector system (Rhodes, C. A. et al. (1995) Methods Mol. Biol. 55:121-131).

Although the presence/absence of marker gene expression suggests that the gene of interest is also present, its presence and expression may need to be confirmed. For example, if the sequence encoding a polypeptide is inserted within a marker gene sequence, recombinant cells containing sequences can be identified by the absence of marker gene function. Alternatively, a marker gene can be placed in tandem with a polypeptide-encoding sequence under the control of a single promoter. Expression of the marker gene in response to induction or selection usually indicates expression of the tandem gene as well.

Alternatively, host cells that contain and express a desired polynucleotide sequence may be identified by a variety of procedures known to those of skill in the art. These procedures include, but are not limited to, DNA-DNA or DNA-RNA hybridizations and protein bioassay or immunoassay techniques which include, for example, membrane, solution, or chip based technologies for the detection and/or quantification of nucleic acid or protein.

A variety of protocols for detecting and measuring the expression of polynucleotide-encoded products, using either polyclonal or monoclonal antibodies specific for the product are known in the art. Examples include enzyme-linked immunosorbent assay (ELISA), radioimmunoassay (RIA), and fluorescence activated cell sorting (FACS). A two-site, monoclonal-based immunoassay utilizing monoclonal antibodies reactive to two non-interfering epitopes on a given polypeptide may be preferred for some applications, but a competitive binding assay may also be employed. These and other assays are described, among other places, in Hampton, R. et al. (1990; Serological Methods, a Laboratory Manual, APS Press, St Paul. Minn.) and Maddox, D. E. et al. (1983; J. Exp. Med. 158:1211-1216).

A wide variety of labels and conjugation techniques are known by those skilled in the art and may be used in various nucleic acid and amino acid assays. Means for producing labeled hybridization or PCR probes for detecting sequences related to polynucleotides include oligolabeling, nick translation, end-labeling or PCR amplification using a labeled nucleotide. Alternatively, the sequences, or any portions thereof may be cloned into a vector for the production of an mRNA probe. Such vectors are known in the art, are commercially available, and may be used to synthesize RNA probes in vitro by addition of an appropriate RNA polymerase such as T7, T3, or SP6 and labeled nucleotides. These procedures may be conducted using a variety of commercially available kits. Suitable reporter molecules or labels, which may be used include radionuclides, enzymes, fluorescent, chemiluminescent, or chromogenic agents as well as substrates, cofactors, inhibitors, magnetic particles, and the like.

Host cells transformed with a polynucleotide sequence of interest may be cultured under conditions suitable for the expression and recovery of the protein from cell culture. The protein produced by a recombinant cell may be secreted or contained intracellularly depending on the sequence and/or the vector used. As will be understood by those of skill in the art, expression vectors containing polynucleotides of the invention may be designed to contain signal sequences which direct secretion of the encoded polypeptide through a prokaryotic or eukaryotic cell membrane. Other recombinant constructions may be used to join sequences encoding a polypeptide of interest to nucleotide sequence encoding a polypeptide domain which will facilitate purification of soluble proteins. Such purification facilitating domains include, but are not limited to, metal chelating peptides such as histidine-tryptophan modules that allow purification on immobilized metals, protein A domains that allow purification on immobilized immunoglobulin, and the domain utilized in the FLAGS extension/affinity purification system (Immunex Corp., Seattle, Wash.). The inclusion of cleavable linker sequences such as those specific for Factor XA or enterokinase (Invitrogen. San Diego, Calif.) between the purification domain and the encoded polypeptide may be used to facilitate purification. One such expression vector provides for expression of a fusion protein containing a polypeptide of interest and a nucleic acid encoding 6 histidine residues preceding a thioredoxin or an enterokinase cleavage site. The histidine residues facilitate purification on IMIAC (immobilized metal ion affinity chromatography) as described in Porath, J. et al. (1992, Prot. Exp. Purif. 3:263-281) while the enterokinase cleavage site provides a means for purifying the desired polypeptide from the fusion protein. A discussion of vectors which contain fusion proteins is provided in Kroll, D. J. et al. (1993; DNA Cell Biol. 12:441-453).

In addition to recombinant production methods, polypeptides of the invention, and fragments thereof, may be produced by direct peptide synthesis using solid-phase techniques (Merrifield J. (1963) J. Am. Chem. Soc. 85:2149-2154). Protein synthesis may be performed using manual techniques or by automation. Automated synthesis may be achieved, for example, using Applied Biosystems 431A Peptide Synthesizer (Perkin Elmer). Alternatively, various fragments may be chemically synthesized separately and combined using chemical methods to produce the full length molecule.

Antibody Compositions, Fragments Thereof and Other Binding Agents

According to another aspect, the present invention further provides binding agents, such as antibodies and antigen-binding fragments thereof, that exhibit 210121.532 immunological binding to a tumor polypeptide disclosed herein, or to a portion, variant or derivative thereof. An antibody, or antigen-binding fragment thereof, is said to “specifically bind,” “immunogically bind,” and/or is “immunologically reactive” to a polypeptide of the invention if it reacts at a detectable level (within, for example, an ELISA assay) with the polypeptide, and does not react detectably with unrelated polypeptides under similar conditions.

Immunological binding, as used in this context, generally refers to the non-covalent interactions of the type which occur between an immunoglobulin molecule and an antigen for which the immunoglobulin is specific. The strength, or affinity of immunological binding interactions can be expressed in terms of the dissociation constant (K_{d}) of the interaction, wherein a smaller K_{d} represents a greater affinity. Immunological binding properties of selected polypeptides can be quantified using methods well known in the art. One such method entails measuring the rates of antigen-binding site/antigen complex formation and dissociation, wherein those rates depend on the concentrations of the complex partners, the affinity of the interaction, and on geometric parameters that equally influence the rate in both directions. Thus, both the “on rate constant” (Kₒₙ) and the “off rate constant” (K_{off}) can be determined by calculation of the concentrations and the actual rates of association and dissociation. The ratio of K_{off}/Kₒₙ enables cancellation of all parameters not related to affinity, and is thus equal to the dissociation constant K_{d}. See, generally, Davies et al. (1990) Annual Rev. Biochem. 59:439-473.

An “antigen-binding site,” or “binding portion” of an antibody refers to the part of the immunoglobulin molecule that participates in antigen binding. The antigen binding site is formed by amino acid residues of the N-terminal variable (“V”) regions of the heavy (“H”) and light (“L”) chains. Three highly divergent stretches within the V regions of the heavy and light chains are referred to as “hypervariable regions” which are interposed between more conserved flanking stretches known as “framework regions,” or “FRs”. Thus the term “FR” refers to amino acid sequences which are naturally found between and adjacent to hypervariable regions in immunoglobulins. In an antibody molecule, the three hypervariable regions of a light chain and the three hypervariable regions of a heavy chain are disposed relative to each other in three dimensional space to form an antigen-binding surface. The antigen-binding surface is complementary to the three-dimensional surface of a bound antigen, and the three hypervariable regions of each of the heavy and light chains are referred to as “complementarity-determining regions,” or “CDRs.”

Binding agents may be further capable of differentiating between patients with and without a cancer, such as colon cancer, using the representative assays provided herein. For example, antibodies or other binding agents that bind to a tumor protein will preferably generate a signal indicating the presence of a cancer in at least about 20% of patients with the disease, more preferably at least about 30% of patients. Alternatively, or in addition, the antibody will generate a negative signal indicating the absence of the disease in at least about 90% of individuals without the cancer. To determine whether a binding agent satisfies this requirement, biological samples (e.g., blood, sera, sputum, urine and/or tumor biopsies) from patients with and without a cancer (as determined using standard clinical tests) may be assayed as described herein for the presence of polypeptides that bind to the binding agent. Preferably, a statistically significant number of samples with and without the disease will be assayed. Each binding agent should satisfy the above criteria; however, those of ordinary skill in the art will recognize that binding agents may be used in combination to improve sensitivity.

Any agent that satisfies the above requirements may be a binding agent. For example, a binding agent may be a ribosome, with or without a peptide component, an RNA molecule or a polypeptide. In a preferred embodiment, a binding agent is an antibody or an antigen-binding fragment thereof. Antibodies may be prepared by any of a variety of techniques known to those of ordinary skill in the art. See, e.g., Harlow and Lane, Antibodies: A Laboratory Manual, Cold Spring Harbor Laboratory, 1988. In general, antibodies can be produced by cell culture techniques, including the generation of monoclonal antibodies as described herein, or via transfection of antibody genes into suitable bacterial or mammalian cell hosts, in order to allow for the production of recombinant antibodies. In one technique, an immunogen comprising the polypeptide is initially injected into any of a wide variety of mammals (e.g., mice, rats, rabbits, sheep or goats). In this step, the polypeptides of this invention may serve as the immunogen without modification. Alternatively, particularly for relatively short polypeptides, a superior immune response may be elicited if the polypeptide is joined to a carrier protein, such as bovine serum albumin or keyhole limpet hemocyanin. The immunogen is injected into the animal host, preferably according to a predetermined schedule incorporating one or more booster immunizations, and the animals are bled periodically. Polyclonal antibodies specific for the polypeptide may then be purified from such antisera by, for example, affinity chromatography using the polypeptide coupled to a suitable solid support.

Monoclonal antibodies specific for an antigenic polypeptide of interest may be prepared, for example, using the technique of Kohler and Milstein, Eur. J Immunol 6:511-519, 1976, and improvements thereto. Briefly, these methods involve the preparation of immortal cell lines capable of producing antibodies having the desired specificity (i.e., reactivity with the polypeptide of interest). Such cell lines may be produced, for example, from spleen cells obtained from an animal immunized as described above. The spleen cells are then immortalized by, for example, fusion with a myeloma cell fusion partner, preferably one that is syngeneic with the immunized animal. A variety of fusion techniques may be employed. For example, the spleen cells and myeloma cells may be combined with a nonionic detergent for a few minutes and then plated at low density on a selective medium that supports the growth of hybrid cells, but not myeloma cells. A preferred selection technique uses HAT (hypoxanthine, aminopterin, thymidine) selection. After a sufficient time, usually about 1 to 2 weeks, colonies of hybrids are observed. Single colonies are selected and their culture supernatants tested for binding activity against the polypeptide. Hybridomas having high reactivity and specificity are preferred.

Monoclonal antibodies may be isolated from the supernatants of growing hybridoma colonies. In addition, various techniques may be employed to enhance the yield, such as injection of the hybridoma cell line into the peritoneal cavity of a suitable vertebrate host, such as a mouse. Monoclonal antibodies may then be harvested from the ascites fluid or the blood. Contaminants may be removed from the antibodies by conventional techniques, such as chromatography, gel filtration, precipitation, and extraction. The polypeptides of this invention may be used in the purification process in, for example, an affinity chromatography step.

A number of therapeutically useful molecules are known in the art which comprise antigen-binding sites that are capable of exhibiting immunological binding properties of an antibody molecule. The proteolytic enzyme papain preferentially cleaves IgG molecules to yield several fragments, two of which (the “F(ab)” fragments) each comprise a covalent heterodimer that includes an intact antigen-binding site. The enzyme pepsin is able to cleave IgG molecules to provide several fragments, including the “F(ab′)₂ ” fragment which comprises both antigen-binding sites. An “Fv” fragment can be produced by preferential proteolytic cleavage of an IgM, and on rare occasions IgG or IgA immunoglobulin molecule. Fv fragments are, however, more commonly derived using recombinant techniques known in the art. The Fv fragment includes a non-covalent V_{H}::V_{L} heterodimer including an antigen-binding site which retains much of the antigen recognition and binding capabilities of the native antibody molecule. Inbar et al. (1972) Proc. Nat. Acad. Sci. USA 69:2659-2662; Hochman et al. (1976) Biochem 15:2706-2710; and Ehrlich et al. (1980) Biochem 19:4091-4096.

A single chain Fv (“sFv”) polypeptide is a covalently linked V_{H}::V_{L} heterodimer which is expressed from a gene fusion including V_{H}- and V_{L}-encoding genes linked by a peptide-encoding linker. Huston et al. (1988) Proc. Nat. Acad. Sci. USA 85(16):5879-5883. A number of methods have been described to discern chemical structures for converting the naturally aggregated—but chemically separated—light and heavy polypeptide chains from an antibody V region into an sFv molecule which will fold into a three dimensional structure substantially similar to the structure of an antigen-binding site. See, e.g., U.S. Pat. Nos. 5,091,513 and 5,132,405, to Huston et al.; and U.S. Pat. No. 4,946,778, to Ladner et al.

Each of the above-described molecules includes a heavy chain and a light chain CDR set, respectively interposed between a heavy chain and a light chain FR set which provide support to the CDRS and define the spatial relationship of the CDRs relative to each other. As used herein, the term “CDR set” refers to the three hypervariable regions of a heavy or light chain V region. Proceeding from the N-terminus of a heavy or light chain, these regions are denoted as “CDR1,” “CDR2,” and “CDR3” respectively. An antigen-binding site, therefore, includes six CDRs, comprising the CDR set from each of a heavy and a light chain V region. A polypeptide comprising a single CDR, (e.g., a CDR1, CDR2 or CDR3) is referred to herein as a “molecular recognition unit.” Crystallographic analysis of a number of antigen-antibody complexes has demonstrated that the amino acid residues of CDRs form extensive contact with bound antigen, wherein the most extensive antigen contact is with the heavy chain CDR3. Thus, the molecular recognition units are primarily responsible for the specificity of an antigen-binding site.

As used herein, the term “FR set” refers to the four flanking amino acid sequences which frame the CDRs of a CDR set of a heavy or light chain V region. Some FR residues may contact bound antigen; however, FRs are primarily responsible for folding the V region into the antigen-binding site, particularly the FR residues directly adjacent to the CDRS. Within FRs, certain amino residues and certain structural features are very highly conserved. In this regard, all V region sequences contain an internal disulfide loop of around 90 amino acid residues. When the V regions fold into a binding-site, the CDRs are displayed as projecting loop motifs which form an antigen-binding surface. It is generally recognized that there are conserved structural regions of FRs which influence the folded shape of the CDR loops into certain “canonical” structures—regardless of the precise CDR amino acid sequence. Further, certain FR residues are known to participate in non-covalent interdomain contacts which stabilize the interaction of the antibody heavy and light chains.

A number of “humanized” antibody molecules comprising an antigen-binding site derived from a non-human immunoglobulin have been described, including chimeric antibodies having rodent V regions and their associated CDRs fused to human constant domains (Winter et al. (1991) Nature 349:293-299; Lobuglio et al. (1989) Proc. Nat. Acad. Sci. USA 86:4220-4224; Shaw et al. (1987) J Immunol. 138:4534-4538; and Brown et al. (1987) Cancer Res. 47:3577-3583), rodent CDRs grafted into a human supporting FR prior to fusion with an appropriate human antibody constant domain (Riechmann et al. (1988) Nature 332:323-327; Verhoeyen et al. (1988) Science 239:1534-1536; and Jones et al. (1986) Nature 321:522-525), and rodent CDRs supported by recombinantly veneered rodent FRs (European Patent Publication No. 519,596, published Dec. 23, 1992). These “humanized” molecules are designed to minimize unwanted immunological response toward rodent antihuman antibody molecules which limits the duration and effectiveness of therapeutic applications of those moieties in human recipients.

As used herein, the terms “veneered FRs” and “recombinantly veneered FRs” refer to the selective replacement of FR residues from, e.g., a rodent heavy or light chain V region, with human FR residues in order to provide a xenogeneic molecule comprising an antigen-binding site which retains substantially all of the native FR polypeptide folding structure. Veneering techniques are based on the understanding that the ligand binding characteristics of an antigen-binding site are determined primarily by the structure and relative disposition of the heavy and light chain CDR sets within the antigen-binding surface. Davies et al. (1990) Ann. Rev. Biochem. 59:439-473. Thus, antigen binding specificity can be preserved in a humanized antibody only wherein the CDR structures, their interaction with each other, and their interaction with the rest of the V region domains are carefully maintained. By using veneering techniques, exterior (e.g., solvent-accessible) FR residues which are readily encountered by the immune system are selectively replaced with human residues to provide a hybrid molecule that comprises either a weakly immunogenic, or substantially non-immunogenic veneered surface.

The process of veneering makes use of the available sequence data for human antibody variable domains compiled by Kabat et al., in Sequences of Proteins of Immunological Interest, 4th ed., (U.S. Dept. of Health and Human Services, U.S. Government Printing Office, 1987), updates to the Kabat database, and other accessible U.S. and foreign databases (both nucleic acid and protein). Solvent accessibilities of V region amino acids can be deduced from the known three-dimensional structure for human and murine antibody fragments. There are two general steps in veneering a murine antigen-binding site. Initially, the FRs of the variable domains of an antibody molecule of interest are compared with corresponding FR sequences of human variable domains obtained from the above-identified sources. The most homologous human V regions are then compared residue by residue to corresponding murine amino acids. The residues in the murine FR which differ from the human counterpart are replaced by the residues present in the human moiety using recombinant techniques well known in the art. Residue switching is only carried out with moieties which are at least partially exposed (solvent accessible), and care is exercised in the replacement of amino acid residues which may have a significant effect on the tertiary structure of V region domains, such as proline, glycine and charged amino acids.

In this manner, the resultant “veneered” murine antigen-binding sites are thus designed to retain the murine CDR residues, the residues substantially adjacent to the CDRs, the residues identified as buried or mostly buried (solvent inaccessible), the residues believed to participate in non-covalent (e.g., electrostatic and hydrophobic) contacts between heavy and light chain domains, and the residues from conserved structural regions of the FRs which are believed to influence the “canonical” tertiary structures of the CDR loops. These design criteria are then used to prepare recombinant nucleotide sequences which combine the CDRs of both the heavy and light chain of a murine antigen-binding site into human-appearing FRs that can be used to transfect mammalian cells for the expression of recombinant human antibodies which exhibit the antigen specificity of the murine antibody molecule.

In another embodiment of the invention, monoclonal antibodies of the present invention may be coupled to one or more therapeutic agents. Suitable agents in this regard include radionuclides, differentiation inducers, drugs, toxins, and derivatives thereof. Preferred radionuclides include ⁹⁰Y, ¹²³I, ¹²⁵I, ¹³¹I, ¹⁸⁶Re, 188Re, ²¹¹At, and ²¹²Bi. Preferred drugs include methotrexate, and pyrimidine and purine analogs. Preferred differentiation inducers include phorbol esters and butyric acid. Preferred toxins include ricin, abrin, diptheria toxin, cholera toxin, gelonin, Pseudomonas exotoxin, Shigella toxin, and pokeweed antiviral protein.

A therapeutic agent may be coupled (e.g., covalently bonded) to a suitable monoclonal antibody either directly or indirectly (e.g., via a linker group). A direct reaction between an agent and an antibody is possible when each possesses a substituent capable of reacting with the other. For example, a nucleophilic group, such as an amino or sulfhydryl group, on one may be capable of reacting with a carbonyl-containing group, such as an anhydride or an acid halide, or with an alkyl group containing a good leaving group (e.g., a halide) on the other.

Alternatively, it may be desirable to couple a therapeutic agent and an antibody via a linker group. A linker group can finction as a spacer to distance an antibody from an agent in order to avoid interference with binding capabilities. A linker group can also serve to increase the chemical reactivity of a substituent on an agent or an antibody, and thus increase the coupling efficiency. An increase in chemical reactivity may also facilitate the use of agents, or functional groups on agents, which otherwise would not be possible.

It will be evident to those skilled in the art that a variety of bifunctional or polyfunctional reagents, both homo- and hetero-functional (such as those described in the catalog of the Pierce Chemical Co., Rockford, Ill.), may be employed as the linker group. Coupling may be effected, for example, through amino groups, carboxyl groups, sulfhydryl groups or oxidized carbohydrate residues. There are numerous references describing such methodology, e.g., U.S. Pat. No. 4,671,958, to Rodwell et al.

Where a therapeutic agent is more potent when free from the antibody portion of the immunoconjugates of the present invention, it may be desirable to use a linker group which is cleavable during or upon internalization into a cell. A number of different cleavable linker groups have been described. The mechanisms for the intracellular release of an agent from these linker groups include cleavage by reduction of a disulfide bond (e.g., U.S. Pat. No. 4,489,710, to Spitler), by irradiation of a photolabile bond (e.g., U.S. Pat. No. 4,625,014, to Senter et al.), by hydrolysis of derivatized amino acid side chains (e.g., U.S. Pat. No. 4,638,045, to Kohn et al.), by serum complement-mediated hydrolysis (e.g., U.S. Pat. No. 4,671,958, to Rodwell et al.), and acid-catalyzed hydrolysis (e.g., U.S. Pat. No. 4,569,789, to Blattler et al.).

It may be desirable to couple more than one agent to an antibody. In one embodiment, multiple molecules of an agent are coupled to one antibody molecule. In another embodiment, more than one type of agent may be coupled to one antibody. Regardless of the particular embodiment, immunoconjugates with more than one agent may be prepared in a variety of ways. For example, more than one agent may be coupled directly to an antibody molecule, or linkers that provide multiple sites for attachment can be used. Alternatively, a carrier can be used.

A carrier may bear the agents in a variety of ways, including covalent bonding either directly or via a linker group. Suitable carriers include proteins such as albumins (e.g., U.S. Pat. No. 4,507,234, to Kato et al.), peptides and polysaccharides such as aminodextran (e.g., U.S. Pat. No. 4,699,784, to Shih et al.). A carrier may also bear an agent by noncovalent bonding or by encapsulation, such as within a liposome vesicle (e.g., U.S. Pat. Nos. 4,429,008 and 4,873,088). Carriers specific for radionuclide agents include radiohalogenated small molecules and chelating compounds. For example, U.S. Pat. No. 4,735,792 discloses representative radiohalogenated small molecules and their synthesis. A radionuclide chelate may be formed from chelating compounds that include those containing nitrogen and sulfur atoms as the donor atoms for binding the metal, or metal oxide, radionuclide. For example, U.S. Pat. No. 4,673,562, to Davison et al. discloses representative chelating compounds and their synthesis.

T Cell Compositions

The present invention, in another aspect, provides T cells specific for a tumor polypeptide disclosed herein, or for a variant or derivative thereof. Such cells may generally be prepared in vitro or ex vivo, using standard procedures. For example, T cells may be isolated from bone marrow, peripheral blood, or a fraction of bone marrow or peripheral blood of a patient, using a commercially available cell separation system, such as the Isolex™ System, available from Nexell Therapeutics, Inc. (Irvine, CA; see also U.S. Pat. No. 5,240,856; U.S. Pat. No. 5,215,926; WO 89/06280; WO 91/16116 and WO 92/07243). Alternatively, T cells may be derived from related or unrelated humans, non-human mammals, cell lines or cultures.

T cells may be stimulated with a polypeptide, polynucleotide encoding a polypeptide and/or an antigen presenting cell (APC) that expresses such a polypeptide. Such stimulation is performed under conditions and for a time sufficient to permit the generation of T cells that are specific for the polypeptide of interest. Preferably, a tumor polypeptide or polynucleotide of the invention is present within a delivery vehicle, such as a microsphere, to facilitate the generation of specific T cells.

T cells are considered to be specific for a polypeptide of the present invention if the T cells specifically proliferate, secrete cytokines or kill target cells coated with the polypeptide or expressing a gene encoding the polypeptide. T cell specificity may be evaluated using any of a variety of standard techniques. For example, within a chromium release assay or proliferation assay, a stimulation index of more than two fold increase in lysis and/or proliferation, compared to negative controls, indicates T cell specificity. Such assays may be performed, for example, as described in Chen et al., Cancer Res. 54:1065-1070, 1994. Alternatively, detection of the proliferation of T cells may be accomplished by a variety of known techniques. For example, T cell proliferation can be detected by measuring an increased rate of DNA synthesis (e.g. by pulse-labeling cultures of T cells with tritiated thymidine and measuring the amount of tritiated thymidine incorporated into DNA). Contact with a tumor polypeptide (100 ng/ml-100 μg/ml, preferably 200 ng/ml-25 μg/ml) for 3-7 days will typically result in at least a two fold increase in proliferation of the T cells. Contact as described above for 2-3 hours should result in activation of the T cells, as measured using standard cytokine assays in which a two fold increase in the level of cytokine release (e.g., TNF or IFN-γ) is indicative of T cell activation (see Coligan et al., Current Protocols in Immunology, vol. 1, Wiley Interscience (Greene 1998)). T cells that have been activated in response to a tumor polypeptide, polynucleotide or polypeptide-expressing APC may be CD4⁺ and/or CD8⁺. Tumor polypeptide-specific T cells may be expanded using standard techniques. Within preferred embodiments, the T cells are derived from a patient, a related donor or an unrelated donor, and are administered to the patient following stimulation and expansion.

For therapeutic purposes, CD4⁺ or CD8⁺ T cells that proliferate in response to a tumor polypeptide, polynucleotide or APC can be expanded in number either in vitro or in vivo. Proliferation of such T cells in vitro may be accomplished in a variety of ways. For example, the T cells can be re-exposed to a tumor polypeptide, or a short peptide corresponding to an immunogenic portion of such a polypeptide, with or without the addition of T cell growth factors, such as interleukin-2, and/or stimulator cells that synthesize a tumor polypeptide. Alternatively, one or more T cells that proliferate in the presence of the tumor polypeptide can be expanded in number by cloning. Methods for cloning cells are well known in the art, and include limiting dilution.

T Cell Receptor Compositions

The T cell receptor (TCR) consists of 2 different, highly variable polypeptide chains, termed the T-cell receptor α and β chains, that are linked by a disulfide bond (Janeway, Travers, Walport. Immunobiology. Fourth Ed., 148-159. Elsevier Science Ltd/Garland Publishing. 1999). The α/β heterodimer complexes with the invariant CD3 chains at the cell membrane. This complex recognizes specific antigenic peptides bound to MHC molecules. The enormous diversity of TCR specificities is generated much like immunoglobulin diversity, through somatic gene rearrangement. The β chain genes contain over 50 variable (V), 2 diversity (D), over 10 joining (J) segments, and 2 constant region segments (C). The α chain genes contain over 70 V segments, and over 60 J segments but no D segments, as well as one C segment. During T cell development in the thymus, the D to J gene rearrangement of the β chain occurs, followed by the V gene segment rearrangement to the DJ. This functional VDJ_{β} exon is transcribed and spliced to join to a C_{β}. For the α chain, a V_{α} gene segment rearranges to a J_{α} gene segment to create the functional exon that is then transcribed and spliced to the C_{α}. Diversity is further increased during the recombination process by the random addition of P and N-nucleotides between the V, D, and J segments of the β chain and between the V and J segments in the α chain (Janeway, Travers, Walport. Immunobiology. Fourth Ed., 98 and 150. Elsevier Science Ltd/Garland Publishing. 1999).

The present invention, in another aspect, provides TCRs specific for a polypeptide disclosed herein, or for a variant or derivative thereof. In accordance with the present invention, polynucleotide and amino acid sequences are provided for the V-J or V-D-J junctional regions or parts thereof for the alpha and beta chains of the T-cell receptor which recognize tumor polypeptides described herein. In general, this aspect of the invention relates to T-cell receptors which recognize or bind tumor polypeptides presented in the context of MHC. In a preferred embodiment the tumor antigens recognized by the T-cell receptors comprise a polypeptide of the present invention. For example, cDNA encoding a TCR specific for a colon tumor peptide can be isolated from T cells specific for a tumor polypeptide using standard molecular biological and recombinant DNA techniques.

This invention further includes the T-cell receptors or analogs thereof having substantially the same function or activity as the T-cell receptors of this invention which recognize or bind tumor polypeptides. Such receptors include, but are not limited to, a fragment of the receptor, or a substitution, addition or deletion mutant of a T-cell receptor provided herein. This invention also encompasses polypeptides or peptides that are substantially homologous to the T-cell receptors provided herein or that retain substantially the same activity. The term “analog” includes any protein or polypeptide having an amino acid residue sequence substantially identical to the T-cell receptors provided herein in which one or more residues, preferably no more than 5 residues, more preferably no more than 25 residues have been conservatively substituted with a functionally similar residue and which displays the functional aspects of the T-cell receptor as described herein.

The present invention further provides for suitable mammalian host cells, for example, non-specific T cells, that are transfected with a polynucleotide encoding TCRs specific for a polypeptide described herein, thereby rendering the host cell specific for the polypeptide. The α and β chains of the TCR may be contained on separate expression vectors or alternatively, on a single expression vector that also contains an internal ribosome entry site (IRES) for cap-independent translation of the gene downstream of the IRES. Said host cells expressing TCRs specific for the polypeptide may be used, for example, for adoptive immunotherapy of colon cancer as discussed further below.

In further aspects of the present invention, cloned TCRs specific for a polypeptide recited herein may be used in a kit for the diagnosis of colon cancer. For example, the nucleic acid sequence or portions thereof, of tumor-specific TCRs can be used as probes or primers for the detection of expression of the rearranged genes encoding the specific TCR in a biological sample. Therefore, the present invention further provides for an assay for detecting messenger RNA or DNA encoding the TCR specific for a polypeptide.

Pharmaceutical Compositions

In additional embodiments, the present invention concerns formulation of one or more of the polynucleotide, polypeptide, T-cell, TCR, and/or antibody compositions disclosed herein in pharmaceutically-acceptable carriers for administration to a cell or an animal, either alone, or in combination with one or more other modalities of therapy.

It will be understood that, if desired, a composition as disclosed herein may be administered in combination with other agents as well, such as, e.g., other proteins or polypeptides or various pharmaceutically-active agents. In fact, there is virtually no limit to other components that may also be included, given that the additional agents do not cause a significant adverse effect upon contact with the target cells or host tissues. The compositions may thus be delivered along with various other agents as required in the particular instance. Such compositions may be purified from host cells or other biological sources, or alternatively may be chemically synthesized as described herein. Likewise, such compositions may further comprise substituted or derivatized RNA or DNA compositions.

Therefore, in another aspect of the present invention, pharmaceutical compositions are provided comprising one or more of the polynucleotide, polypeptide, antibody, TCR, and/or T-cell compositions described herein in combination with a physiologically acceptable carrier. In certain preferred embodiments, the pharmaceutical compositions of the invention comprise immunogenic polynucleotide and/or polypeptide compositions of the invention for use in prophylactic and theraputic vaccine applications. Vaccine preparation is generally described in, for example, M. F. Powell and M. J. Newman, eds., “Vaccine Design (the subunit and adjuvant approach),” Plenum Press (NY, 1995). Generally, such compositions will comprise one or more polynucleotide and/or polypeptide compositions of the present invention in combination with one or more immunostimulants.

It will be apparent that any of the pharmaceutical compositions described herein can contain pharmaceutically acceptable salts of the polynucleotides and polypeptides of the invention. Such salts can be prepared, for example, from pharmaceutically acceptable non-toxic bases, including organic bases (e.g., salts of primary, secondary and tertiary amines and basic amino acids) and inorganic bases (e.g., sodium, potassium, lithium, ammonium, calcium and magnesium salts).

In another embodiment, illustrative immunogenic compositions, e.g., vaccine compositions, of the present invention comprise DNA encoding one or more of the polypeptides as described above, such that the polypeptide is generated in situ. As noted above, the polynucleotide may be administered within any of a variety of delivery systems known to those of ordinary skill in the art. Indeed, numerous gene delivery techniques are well known in the art, such as those described by Rolland, Crit. Rev. Therap. Drug Carrier Systems 15:143-198, 1998, and references cited therein. Appropriate polynucleotide expression systems will, of course, contain the necessary regulatory DNA regulatory sequences for expression in a patient (such as a suitable promoter and terminating signal). Alternatively, bacterial delivery systems may involve the administration of a bacterium (such as Bacillus-Calmette-Guerrin) that expresses an immunogenic portion of the polypeptide on its cell surface or secretes such an epitope.

Therefore, in certain embodiments, polynucleotides encoding immunogenic polypeptides described herein are introduced into suitable mammalian host cells for expression using any of a number of known viral-based systems. In one illustrative embodiment, retroviruses provide a convenient and effective platform for gene delivery systems. A selected nucleotide sequence encoding a polypeptide of the present invention can be inserted into a vector and packaged in retroviral particles using techniques known in the art. The recombinant virus can then be isolated and delivered to a subject. A number of illustrative retroviral systems have been described (e.g., U.S. Pat. No. 5,219,740; Miller and Rosman (1989) BioTechniques 7:980-990; Miller, A. D. (1990) Human Gene Therapy 1:5-14; Scarpa et al. (1991) Virology 180:849-852; Bums et al. (1993) Proc. Natl. Acad. Sci. USA 90:8033-8037; and Boris-Lawrie and Temin (1993) Cur. Opin. Genet. Develop. 3:102-109.

In addition, a number of illustrative adenovirus-based systems have also been described. Unlike retroviruses which integrate into the host genome, adenoviruses persist extrachromosomally thus minimizing the risks associated with insertional mutagenesis (Haj-Ahmad and Graham (1986) J. Virol. 57:267-274; Bett et al. (1993) J. Virol. 67:5911-5921; Mittereder et al. (1994) Human Gene Therapy 5:717-729; Seth et al. (1994) J. Virol. 68:933-940; Barr et al. (1994) Gene Therapy 1:51-58; Berkner, K. L. (1988) BioTechniques 6:616-629; and Rich et al. (1993) Human Gene Therapy 4:461-476).

Various adeno-associated virus (AAV) vector systems have also been developed for polynucleotide delivery. AAV vectors can be readily constructed using techniques well known in the art. See, e.g., U.S. Pat. Nos. 5,173,414 and 5,139,941; International Publication Nos. WO 92/01070 and WO 93/03769; Lebkowski et al. (1988) Molec. Cell. Biol. 8:3988-3996; Vincent et al. (1990) Vaccines 90 (Cold Spring Harbor Laboratory Press); Carter, B. J. (1992) Current Opinion in Biotechnology 3:533-539; Muzyczka, N. (1992) Current Topics in Microbiol. and Immunol. 158:97-129; Kotin, R. M. (1994) Human Gene Therapy 5:793-801; Shelling and Smith (1994) Gene Therapy 1:165-169; and Zhou et al. (1994) J. Exp. Med. 179:1867-1875.

Additional viral vectors useful for delivering the polynucleotides encoding polypeptides of the present invention by gene transfer include those derived from the pox family of viruses, such as vaccinia virus and avian poxvirus. By way of example, vaccinia virus recombinants expressing the novel molecules can be constructed as follows. The DNA encoding a polypeptide is first inserted into an appropriate vector so that it is adjacent to a vaccinia promoter and flanking vaccinia DNA sequences, such as the sequence encoding thymidine kinase (TK). This vector is then used to transfect cells which are simultaneously infected with vaccinia. Homologous recombination serves to insert the vaccinia promoter plus the gene encoding the polypeptide of interest into the viral genome. The resulting TK.sup.(−) recombinant can be selected by culturing the cells in the presence of 5-bromodeoxyuridine and picking viral plaques resistant thereto.

A vaccinia-based infection/transfection system can be conveniently used to provide for inducible, transient expression or coexpression of one or more polypeptides described herein in host cells of an organism. In this particular system, cells are first infected in vitro with a vaccinia virus recombinant that encodes the bacteriophage T7 RNA polymerase. This polymerase displays exquisite specificity in that it only transcribes templates bearing T7 promoters. Following infection, cells are transfected with the polynucleotide or polynucleotides of interest, driven by a T7 promoter. The polymerase expressed in the cytoplasm from the vaccinia virus recombinant transcribes the transfected DNA into RNA which is then translated into polypeptide by the host translational machinery. The method provides for high level, transient, cytoplasmic production of large quantities of RNA and its translation products. See, e.g., Elroy-Stein and Moss, Proc. Natl. Acad. Sci. USA (1990) 87:6743-6747; Fuerst et al. Proc. Natl. Acad. Sci. USA (1986) 83:8122-8126.

Alternatively, avipoxviruses, such as the fowlpox and canarypox viruses, can also be used to deliver the coding sequences of interest. Recombinant avipox viruses, expressing immunogens from manunalian pathogens, are known to confer protective immunity when administered to non-avian species. The use of an Avipox vector is particularly desirable in human and other mammalian species since members of the Avipox genus can only productively replicate in susceptible avian species and therefore are not infective in mammalian cells. Methods for producing recombinant Avipoxviruses are known in the art and employ genetic recombination, as described above with respect to the production of vaccinia viruses. See, e.g., WO 91/12882; WO 89/03429; and WO 92/03545.

Any of a number of alphavirus vectors can also be used for delivery of polynucleotide compositions of the present invention, such as those vectors described in U.S. Pat. Nos. 5,843,723; 6,015,686; 6,008,035 and 6,015,694. Certain vectors based on Venezuelan Equine Encephalitis (VEE) can also be used, illustrative examples of which can be found in U.S. Pat. Nos. 5,505,947 and 5,643,576.

Moreover, molecular conjugate vectors, such as the adenovirus chimeric vectors described in Michael et al. J. Biol. Chem. (1993) 268:6866-6869 and Wagner et al. Proc. Natl. Acad. Sci. USA (1992) 89:6099-6103, can also be used for gene delivery under the invention.

Additional illustrative information on these and other known viral-based delivery systems can be found, for example, in Fisher-Hoch et al., Proc. Natl. Acad. Sci. USA 86:317-321, 1989; Flexner et al., Ann. N.Y. Acad. Sci. 569:86-103, 1989; Flexner et al., Vaccine 8:17-21, 1990; U.S. Pat. Nos. 4,603,112, 4,769,330, and 5,017,487; WO 89/01973; U.S. Pat. No. 4,777,127; GB 2,200,651; EP 0,345,242; WO 91/02805; Berkner, Biotechniques 6:616-627, 1988; Rosenfeld et al., Science 252:431-434, 1991; Kolls et al., Proc. Natl. Acad. Sci. USA 91:215-219, 1994; Kass-Eisler et al., Proc. Natl. Acad. Sci. USA 90:11498-11502, 1993; Guzman et al., Circulation 88:2838-2848, 1993; and Guzman et al., Cir. Res. 73:1202-1207, 1993.

In certain embodiments, a polynucleotide may be integrated into the genome of a target cell. This integration may be in the specific location and orientation via homologous recombination (gene replacement) or it may be integrated in a random, non-specific location (gene augmentation). In yet further embodiments, the polynucleotide may be stably maintained in the cell as a separate, episomal segment of DNA. Such polynucleotide segments or “episomes” encode sequences sufficient to permit maintenance and replication independent of or in synchronization with the host cell cycle. The manner in which the expression construct is delivered to a cell and where in the cell the polynucleotide remains is dependent on the type of expression construct employed.

In another embodiment of the invention, a polynucleotide is administered/delivered as “naked” DNA, for example as described in Ulmer et al., Science 259:1745-1749, 1993 and reviewed by Cohen, Science 259:1691-1692, 1993. The uptake of naked DNA may be increased by coating the DNA onto biodegradable beads, which are efficiently transported into the cells.

In still another embodiment, a composition of the present invention can be delivered via a particle bombardment approach, many of which have been described. In one illustrative example, gas-driven particle acceleration can be achieved with devices such as those manufactured by Powderject Pharmaceuticals PLC (Oxford, UK) and Powderject Vaccines Inc. (Madison, Wis.), some examples of which are described in U.S. Pat. Nos. 5,846,796; 6,010,478; 5,865,796; 5,584,807; and EP Patent No. 0500 799. This approach offers a needle-free delivery approach wherein a dry powder formulation of microscopic particles, such as polynucleotide or polypeptide particles, are accelerated to high speed within a helium gas jet generated by a hand held device, propelling the particles into a target tissue of interest.

In a related embodiment, other devices and methods that may be useful for gas-driven needle-less injection of compositions of the present invention include those provided by Bioject, Inc. (Portland, OR), some examples of which are described in U.S. Pat. Nos. 4,790,824; 5,064,413; 5,312,335; 5,383,851; 5,399,163; 5,520,639 and 5,993,412.

According to another embodiment, the pharmaceutical compositions described herein will comprise one or more immunostimulants in addition to the immunogenic polynucleotide, polypeptide, antibody, T-cell, TCR, and/or APC compositions of this invention. An immunostimulant refers to essentially any substance that enhances or potentiates an immune response (antibody and/or cell-mediated) to an exogenous antigen. One preferred type of immunostimulant comprises an adjuvant. Many adjuvants contain a substance designed to protect the antigen from rapid catabolism, such as aluminum hydroxide or mineral oil, and a stimulator of immune responses, such as lipid A, Bortadella pertussis or Mycobacterium tuberculosis derived proteins. Certain adjuvants are commercially available as, for example, Freund's Incomplete Adjuvant and Complete Adjuvant (Difco Laboratories, Detroit, Mich.); Merck Adjuvant 65 (Merck and Company, Inc., Rahway, N.J.); AS-2 (SmithKline Beecham, Philadelphia, Pa.); aluminum salts such as aluminum hydroxide gel (alum) or aluminum phosphate; salts of calcium, iron or zinc; an insoluble suspension of acylated tyrosine; acylated sugars; cationically or anionically derivatized polysaccharides; polyphosphazenes; biodegradable microspheres; monophosphoryl lipid A and quil A. Cytokines, such as GM-CSF, interleukin-2, -7, -12, and other like growth factors, may also be used as adjuvants.

Within certain embodiments of the invention, the adjuvant composition is preferably one that induces an immune response predominantly of the Thl type. High levels of Thl-type cytokines (e.g., IFN-γ, TNFα, IL-2 and IL-12) tend to favor the induction of cell mediated immune responses to an administered antigen. In contrast, high levels of Th2-type cytokines (e.g., IL-4, IL-5, IL-6 and IL-10) tend to favor the induction of humoral immune responses. Following application of a vaccine as provided herein, a patient will support an immune response that includes Th1- and Th2-type responses. Within a preferred embodiment, in which a response is predominantly Th1-type, the level of Th1-type cytokines will increase to a greater extent than the level of Th2-type cytokines. The levels of these cytokines may be readily assessed using standard assays. For a review of the families of cytokines, see Mosmann and Coffinan, Ann. Rev. Immunol. 7:145-173, 1989.

Certain preferred adjuvants for eliciting a predominantly Th1-type response include, for example, a combination of monophosphoryl lipid A, preferably 3-de-O-acylated monophosphoryl lipid A, together with an aluminum salt. MPL® adjuvants are available from Corixa Corporation (Seattle, WA; see, for example, U.S. Pat. Nos. 4,436,727; 4,877,611; 4,866,034 and 4,912,094). CpG-containing oligonucleotides (in which the CpG dinucleotide is unmethylated) also induce a predominantly Th1 response. Such oligonucleotides are well known and are described, for example, in WO 96/02555, WO 99/33488 and U.S. Pat. Nos. 6,008,200 and 5,856,462. Immunostimulatory DNA sequences are also described, for example, by Sato et al., Science 273:352, 1996. Another preferred adjuvant comprises a saponin, such as Quil A, or derivatives thereof, including QS21 and QS7 (Aquila Biopharmaceuticals Inc., Framingham, Mass.); Escin; Digitonin; or Gypsophila or Chenopodium quinoa saponins . Other preferred formulations include more than one saponin in the adjuvant combinations of the present invention, for example combinations of at least two of the following group comprising QS21, QS7, Quil A, β-escin, or digitonin.

Alternatively the saponin formulations may be combined with vaccine vehicles composed of chitosan or other polycationic polymers, polylactide and polylactide-co-glycolide particles, poly-N-acetyl glucosamine-based polymer matrix, particles composed of polysaccharides or chemically modified polysaccharides, liposomes and lipid-based particles, particles composed of glycerol monoesters, etc. The saponins may also be formulated in the presence of cholesterol to form particulate structures such as liposomes or ISCOMs. Furthermore, the saponins may be formulated together with a polyoxyethylene ether or ester, in either a non-particulate solution or suspension, or in a particulate structure such as a paucilamelar liposome or ISCOM. The saponins may also be formulated with excipients such as Carbopol® to increase viscosity, or may be formulated in a dry powder form with a powder excipient such as lactose.

In one preferred embodiment, the adjuvant system includes the combination of a monophosphoryl lipid A and a saponin derivative, such as the combination of QS21 and 3D-MPL® adjuvant, as described in WO 94/00153, or a less reactogenic composition where the QS21 is quenched with cholesterol, as described in WO 96/33739. Other preferred formulations comprise an oil-in-water emulsion and tocopherol. Another particularly preferred adjuvant formulation employing QS21, 3D-MPL® adjuvant and tocopherol in an oil-in-water emulsion is described in WO 95/17210.

Another enhanced adjuvant system involves the combination of a CpG-containing oligonucleotide and a saponin derivative particularly the combination of CpG and QS21 is disclosed in WO 00/09159. Preferably the formulation additionally comprises an oil in water emulsion and tocopherol.

Additional illustrative adjuvants for use in the pharmaceutical compositions of the invention include Montanide ISA 720 (Seppic, France), SAF (Chiron, Calif., United States), ISCOMS (CSL), MF-59 (Chiron), the SBAS series of adjuvants (e.g., SBAS-2 or SBAS-4, available from SmithKline Beecham, Rixensart, Belgium), Detox (Enhanzyn®) (Corixa, Hamilton, Mont.), RC-529 (Corixa, Hamilton, Mont.) and other aminoalkyl glucosaminide 4-phosphates (AGPs), such as those described in pending U.S. patent application Ser. Nos. 08/853,826 and 09/074,720, the disclosures of which are incorporated herein by reference in their entireties, and polyoxyethylene ether adjuvants such as those described in WO 99/52549A1.

Other preferred adjuvants include adjuvant molecules of the general formula

HO(CH₂CH₂O)ₙ—A—R, (I)

wherein, n is 1-50, A is a bond or —C(O)—, R is C₁₋₅₀ alkyl or Phenyl C₁₋₅₀ alkyl.

One embodiment of the present invention consists of a vaccine formulation comprising a polyoxyethylene ether of general formula (I), wherein n is between 1 and 50, preferably 4-24, most preferably 9; the R component is C₁₋₅₀, preferably C₄-C₂₀ alkyl and most preferably C₁₂ alkyl, and A is a bond. The concentration of the polyoxyethylene ethers should be in the range 0.1-20%, preferably from 0.1-10%, and most preferably in the range 0.1-1%. Preferred polyoxyethylene ethers are selected from the following group: polyoxyethylene-9-lauryl ether, polyoxyethylene-9-steoryl ether, polyoxyethylene-8-steoryl ether, polyoxyethylene-4-lauryl ether, polyoxyethylene-35-lauryl ether, and polyoxyethylene-23-lauryl ether. Polyoxyethylene ethers such as polyoxyethylene lauryl ether are described in the Merck index (12^{th} edition: entry 7717). These adjuvant molecules are described in WO 99/52549.

The polyoxyethylene ether according to the general formula (I) above may, if desired, be combined with another adjuvant. For example, a preferred adjuvant combination is preferably with CpG as described in the pending UK patent application GB 9820956.2.

According to another embodiment of this invention, an immunogenic composition described herein is delivered to a host via antigen presenting cells (APCs), such as dendritic cells, macrophages, B cells, monocytes and other cells that may be engineered to be efficient APCs. Such cells may, but need not, be genetically modified to increase the capacity for presenting the antigen, to improve activation and/or maintenance of the T cell response, to have anti-tumor effects per se and/or to be immunologically compatible with the receiver (i.e., matched HLA haplotype). APCs may generally be isolated from any of a variety of biological fluids and organs, including tumor and peritumoral tissues, and may be autologous, allogeneic, syngeneic or xenogeneic cells.

Certain preferred embodiments of the present invention use dendritic cells or progenitors thereof as antigen-presenting cells. Dendritic cells are highly potent APCs (Banchereau and Steinman, Nature 392:245-251, 1998) and have been shown to be effective as a physiological adjuvant for eliciting prophylactic or therapeutic antitumor immunity (see Timmerman and Levy, Ann. Rev. Med. 50:507-529, 1999). In general, dendritic cells may be identified based on their typical shape (stellate in situ, with marked cytoplasmic processes (dendrites) visible in vitro), their ability to take up, process and present antigens with high efficiency and their ability to activate naive T cell responses.

Dendritic cells may, of course, be engineered to express specific cell-surface receptors or ligands that are not commonly found on dendritic cells in vivo or ex vivo, and such modified dendritic cells are contemplated by the present invention. As an alternative to dendritic cells, secreted vesicles antigen-loaded dendritic cells (called exosomes) may be used within a vaccine (see Zitvogel et al., Nature Med. 4:594-600, 1998).

Dendritic cells and progenitors may be obtained from peripheral blood, bone marrow, tumor-infiltrating cells, peritumoral tissues-infiltrating cells, lymph nodes, spleen, skin, umbilical cord blood or any other suitable tissue or fluid. For example, dendritic cells may be differentiated ex vivo by adding a combination of cytokines such as GM-CSF, IL-4, IL-13 and/or TNFα to cultures of monocytes harvested from peripheral blood. Alternatively, CD34 positive cells harvested from peripheral blood, umbilical cord blood or bone marrow may be differentiated into dendritic cells by adding to the culture medium combinations of GM-CSF, IL-3, TNFα, CD40 ligand, LPS, flt3 ligand and/or other compound(s) that induce differentiation, maturation and proliferation of dendritic cells.

Dendritic cells are conveniently categorized as “immature” and “mature” cells, which allows a simple way to discriminate between two well characterized phenotypes. However, this nomenclature should not be construed to exclude all possible intermediate stages of differentiation. Immature dendritic cells are characterized as APC with a high capacity for antigen uptake and processing, which correlates with the high expression of Fcy receptor and mannose receptor. The mature phenotype is typically characterized by a lower expression of these markers, but a high expression of cell surface molecules responsible for T cell activation such as class I and class II MHC, adhesion molecules (e.g., CD54 and CD11) and costimulatory molecules (e.g., CD40, CD80, CD86 and 4-1BB).

APCs may generally be transfected with a polynucleotide of the invention (or portion or other variant thereof) such that the encoded polypeptide, or an immunogenic portion thereof, is expressed on the cell surface. Such transfection may take place ex vivo, and a pharmaceutical composition comprising such transfected cells may then be used for therapeutic purposes, as described herein. Alternatively, a gene delivery vehicle that targets a dendritic or other antigen presenting cell may be administered to a patient, resulting in transfection that occurs in vivo. In vivo and ex vivo transfection of dendritic cells, for example, may generally be performed using any methods known in the art, such as those described in WO 97/24447, or the gene gun approach described by Mahvi et al., Immunology and cell Biology 75:456-460, 1997. Antigen loading of dendritic cells may be achieved by incubating dendritic cells or progenitor cells with the tumor polypeptide, DNA (naked or within a plasmid vector) or RNA; or with antigen-expressing recombinant bacterium or viruses (e.g., vaccinia, fowlpox, adenovirus or lentivirus vectors). Prior to loading, the polypeptide may be covalently conjugated to an immunological partner that provides T cell help (e.g., a carrier molecule). Alternatively, a dendritic cell may be pulsed with a non-conjugated immunological partner, separately or in the presence of the polypeptide.

While any suitable carrier known to those of ordinary skill in the art may be employed in the pharmaceutical compositions of this invention, the type of carrier will typically vary depending on the mode of administration. Compositions of the present invention may be formulated for any appropriate manner of administration, including for example, topical, oral, nasal, mucosal, intravenous, intracranial, intraperitoneal, subcutaneous and intramuscular administration.

Carriers for use within such pharmaceutical compositions are biocompatible, and may also be biodegradable. In certain embodiments, the formulation preferably provides a relatively constant level of active component release. In other embodiments, however, a more rapid rate of release immediately upon administration may be desired. The formulation of such compositions is well within the level of ordinary skill in the art using known techniques. Illustrative carriers useful in this regard include microparticles of poly(lactide-co-glycolide), polyacrylate, latex, starch, cellulose, dextran and the like. Other illustrative delayed-release carriers include supramolecular biovectors, which comprise a non-liquid hydrophilic core (e.g., a cross-linked polysaccharide or oligosaccharide) and, optionally, an external layer comprising an amphiphilic compound, such as a phospholipid (see e.g., U.S. Pat. No. 5,151,254 and PCT applications WO 94/20078, WO/94/23701 and WO 96/06638). The amount of active compound contained within a sustained release formulation depends upon the site of implantation, the rate and expected duration of release and the nature of the condition to be treated or prevented.

In another illustrative embodiment, biodegradable microspheres (e.g., polylactate polyglycolate) are employed as carriers for the compositions of this invention. Suitable biodegradable microspheres are disclosed, for example, in U.S. Pat. Nos. 4,897,268; 5,075,109; 5,928,647; 5,811,128; 5,820,883; 5,853,763; 5,814,344, 5,407,609 and 5,942,252. Modified hepatitis B core protein carrier systems. Such as described in WO/99 40934, and references cited therein, will also be useful for many applications. Another illustrative carrier/delivery system employs a carrier comprising particulate-protein complexes, such as those described in U.S. Pat. No. 5,928,647, which are capable of inducing a class I-restricted cytotoxic T lymphocyte responses in a host.

In another illustrative embodiment, calcium phosphate core particles are employed as carriers, vaccine adjuvants, or as controlled release matrices for the compositions of this invention. Exemplary calcium phosphate particles are disclosed, for example, in published patent application No. WO/0046147.

The pharmaceutical compositions of the invention will often further comprise one or more buffers (e.g., neutral buffered saline or phosphate buffered saline), carbohydrates (e.g., glucose, mannose, sucrose or dextrans), mannitol, proteins, polypeptides or amino acids such as glycine, antioxidants, bacteriostats, chelating agents such as EDTA or glutathione, adjuvants (e.g., aluminum hydroxide), solutes that render the formulation isotonic, hypotonic or weakly hypertonic with the blood of a recipient, suspending agents, thickening agents and/or preservatives. Alternatively, compositions of the present invention may be formulated as a lyophilizate.

The pharmaceutical compositions described herein may be presented in unit-dose or multi-dose containers, such as sealed ampoules or vials. Such containers are typically sealed in such a way to preserve the sterility and stability of the formulation until use. In general, formulations may be stored as suspensions, solutions or emulsions in oily or aqueous vehicles. Alternatively, a pharmaceutical composition may be stored in a freeze-dried condition requiring only the addition of a sterile liquid carrier immediately prior to use.

The development of suitable dosing and treatment regimens for using the particular compositions described herein in a variety of treatment regimens, including e.g., oral, parenteral, intravenous, intranasal, and intramuscular administration and formulation, is well known in the art, some of which are briefly discussed below for general purposes of illustration.

In certain applications, the pharmaceutical compositions disclosed herein may be delivered via oral administration to an animal. As such, these compositions may be formulated with an inert diluent or with an assimilable edible carrier, or they may be enclosed in hard- or soft-shell gelatin capsule, or they may be compressed into tablets, or they may be incorporated directly with the food of the diet.

The active compounds may even be incorporated with excipients and used in the form of ingestible tablets, buccal tables, troches, capsules, elixirs, suspensions, syrups, wafers, and the like (see, for example, Mathiowitz et al., Nature Mar. 27, 1997;386(6623):410-4; Hwang et al., Crit Rev Ther Drug Carrier Syst 1998;15(3):243-84; U.S. Pat. No. 5,641,515; U.S. Pat. No. 5,580,579 and U.S. Pat. No. 5,792,451). Tablets, troches, pills, capsules and the like may also contain any of a variety of additional components, for example, a binder, such as gum tragacanth, acacia, cornstarch, or gelatin; excipients, such as dicalcium phosphate; a disintegrating agent, such as corn starch, potato starch, alginic acid and the like; a lubricant, such as magnesium stearate; and a sweetening agent, such as sucrose, lactose or saccharin may be added or a flavoring agent, such as peppermint, oil of wintergreen, or cherry flavoring. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar, or both. Of course, any material used in preparing any dosage unit form should be pharmaceutically pure and substantially non-toxic in the amounts employed. In addition, the active compounds may be incorporated into sustained-release preparation and formulations.

Typically, these formulations will contain at least about 0.1% of the active compound or more, although the percentage of the active ingredient(s) may, of course, be varied and may conveniently be between about 1 or 2% and about 60% or 70% or more of the weight or volume of the total formulation. Naturally, the amount of active compound(s) in each therapeutically useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

For oral administration the compositions of the present invention may alternatively be incorporated with one or more excipients in the form of a mouthwash, dentifrice, buccal tablet, oral spray, or sublingual orally-administered formulation. Alternatively, the active ingredient may be incorporated into an oral solution such as one containing sodium borate, glycerin and potassium bicarbonate, or dispersed in a dentifrice, or added in a therapeutically-effective amount to a composition that may include water, binders, abrasives, flavoring agents, foaming agents, and humectants. Alternatively the compositions may be fashioned into a tablet or solution form that may be placed under the tongue or otherwise dissolved in the mouth.

In certain circumstances it will be desirable to deliver the pharmaceutical compositions disclosed herein parenterally, intravenously, intramuscularly, or even intraperitoneally. Such approaches are well known to the skilled artisan, some of which are further described, for example, in U.S. Pat. No. 5,543,158; U.S. Pat. No. 5,641,515 and U.S. Pat. No. 5,399,363. In certain embodiments, solutions of the active compounds as free base or pharmacologically acceptable salts may be prepared in water suitably mixed with a surfactant, such as hydroxypropylcellulose. Dispersions may also be prepared in glycerol, liquid polyethylene glycols, and mixtures thereof and in oils. Under ordinary conditions of storage and use, these preparations generally will contain a preservative to prevent the growth of microorganisms.

Illustrative pharmaceutical forms suitable for injectable use include sterile aqueous solutions or dispersions and sterile powders for the extemporaneous preparation of sterile injectable solutions or dispersions (for example, see U.S. Pat. 5,466,468). In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and/or by the use of surfactants. The prevention of the action of microorganisms can be facilitated by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

In one embodiment, for parenteral administration in an aqueous solution, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, a sterile aqueous medium that can be employed will be known to those of skill in the art in light of the present disclosure. For example, one dosage may be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. Moreover, for human administration, preparations will of course preferably meet sterility, pyrogenicity, and the general safety and purity standards as required by FDA Office of Biologics standards.

In another embodiment of the invention, the compositions disclosed herein may be formulated in a neutral or salt form. Illustrative pharmaceutically-acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. Upon formulation, solutions will be administered in a manner compatible with the dosage formulation and in such amount as is therapeutically effective.

The carriers can further comprise any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions. The phrase “pharmaceutically-acceptable” refers to molecular entities and compositions that do not produce an allergic or similar untoward reaction when administered to a human.

In certain embodiments, the pharmaceutical compositions may be delivered by intranasal sprays, inhalation, and/or other aerosol delivery vehicles. Methods for delivering genes, nucleic acids, and peptide compositions directly to the lungs via nasal aerosol sprays has been described, e.g., in U.S. Pat. No. 5,756,353 and U.S. Pat. No. 5,804,212. Likewise, the delivery of drugs using intranasal microparticle resins (Takenaga et al., J Controlled Release Mar. 2, 1998;52(1-2):81-7) and lysophosphatidyl-glycerol compounds (U.S. Pat. No. 5,725,871) are also well-known in the pharmaceutical arts. Likewise, illustrative transmucosal drug delivery in the form of a polytetrafluoroetheylene support matrix is described in U.S. Pat. No. 5,780,045.

In certain embodiments, liposomes, nanocapsules, microparticles, lipid particles, vesicles, and the like, are used for the introduction of the compositions of the present invention into suitable host cells/organisms. In particular, the compositions of the present invention may be formulated for delivery either encapsulated in a lipid particle, a liposome, a vesicle, a nanosphere, or a nanoparticle or the like. Alternatively, compositions of the present invention can be bound, either covalently or non-covalently, to the surface of such carrier vehicles.

The formation and use of liposome and liposome-like preparations as potential drug carriers is generally known to those of skill in the art (see for example, Lasic, Trends Biotechnol July 1998;16(7):307-21; Takakura, Nippon Rinsho March 1998;56(3):691-5; Chandran et al., Indian J Exp Biol. August 1997;35(8):801-9; Margalit, Crit Rev Ther Drug Carrier Syst. 1995;12(2-3):233-61; U.S. Pat. No. 5,567,434; U.S. Pat. No. 5,552,157; U.S. Pat. No. 5,565,213; U.S. Pat. No. 5,738,868 and U.S. Pat. No. 5,795,587, each specifically incorporated herein by reference in its entirety).

Liposomes have been used successfully with a number of cell types that are normally difficult to transfect by other procedures, including T cell suspensions, primary hepatocyte cultures and PC 12 cells (Renneisen et al., J Biol Chem. Sep. 25, 1990;265(27):16337-42; Muller et al., DNA Cell Biol. April 1990;9(3):221-9). In addition, liposomes are free of the DNA length constraints that are typical of viral-based delivery systems. Liposomes have been used effectively to introduce genes, various drugs, radiotherapeutic agents, enzymes, viruses, transcription factors, allosteric effectors and the like, into a variety of cultured cell lines and animals. Furthermore, he use of liposomes does not appear to be associated with autoimmune responses or unacceptable toxicity after systemic delivery.

In certain embodiments, liposomes are formed from phospholipids that are dispersed in an aqueous medium and spontaneously form multilamellar concentric bilayer vesicles (also termed multilamellar vesicles (MLVs).

Alternatively, in other embodiments, the invention provides for pharmaceutically-acceptable nanocapsule formulations of the compositions of the present invention. Nanocapsules can generally entrap compounds in a stable and reproducible way (see, for example, Quintanar-Guerrero et al., Drug Dev Ind Pharm. December 1998;24(12):1113-28). To avoid side effects due to intracellular polymeric overloading, such ultrafine particles (sized around 0.1 μm) may be designed using polymers able to be degraded in vivo. Such particles can be made as described, for example, by Couvreur et al., Crit Rev Ther Drug Carrier Syst. 1988;5(1):1-20; zur Muhlen et al., Eur J Pharm Biopharm. March 1998;45(2):149-55; Zambaux et al. J Controlled Release. Jan. 2, 1998;50(1-3):31-40; and U.S. Pat. No. 5,145,684.

Cancer Therapeutic Methods

Immunologic approaches to cancer therapy are based on the recognition that cancer cells can often evade the body's defenses against aberrant or foreign cells and molecules, and that these defenses might be therapeutically stimulated to regain the lost ground, e.g. pgs. 623-648 in Klein, Immunology (Wiley-Interscience, New York, 1982). Numerous recent observations that various immune effectors can directly or indirectly inhibit growth of tumors has led to renewed interest in this approach to cancer therapy, e.g. Jager, et al., Oncology 2001;60(1):1-7; Renner, et al., Ann Hematol 2000 Dec;79(12):651-9.

Four-basic cell types whose function has been associated with antitumor cell immunity and the elimination of tumor cells from the body are: i) B-lymphocytes which secrete immunoglobulins into the blood plasma for identifying and labeling the nonself invader cells; ii) monocytes which secrete the complement proteins that are responsible for lysing and processing the immunoglobulin-coated target invader cells; iii) natural killer lymphocytes having two mechanisms for the destruction of tumor cells, antibody-dependent cellular cytotoxicity and natural killing; and iv) T-lymphocytes possessing antigen-specific receptors and having the capacity to recognize a tumor cell carrying complementary marker molecules (Schreiber, H., 1989, in Fundamental Immunology (ed). W. E. Paul, pp. 923-955).

Cancer immunotherapy generally focuses on inducing humoral immune responses, cellular immune responses, or both. Moreover, it is well established that induction of CD4⁺ T helper cells is necessary in order to secondarily induce either antibodies or cytotoxic CD8⁺ T cells. Polypeptide antigens that are selective or ideally specific for cancer cells, particularly colon cancer cells, offer a powerful approach for inducing immune responses against colon cancer, and are an important aspect of the present invention.

Therefore, in further aspects of the present invention, the pharmaceutical compositions described herein may be used to stimulate an immune response against cancer, particularly for the immunotherapy of colon cancer. Within such methods, the pharmaceutical compositions described herein are administered to a patient, typically a warm-blooded animal, preferably a human. A patient may or may not be afflicted with cancer. Pharmaceutical compositions and vaccines may be administered either prior to or following surgical removal of primary tumors and/or treatment such as administration of radiotherapy or conventional chemotherapeutic drugs. As discussed above, administration of the pharmaceutical compositions may be by any suitable method, including administration by intravenous, intraperitoneal, intramuscular, subcutaneous, intranasal, intradermal, anal, vaginal, topical and oral routes.

Within certain embodiments, immunotherapy may be active immunotherapy, in which treatment relies on the in vivo stimulation of the endogenous host immune system to react against tumors with the administration of immune response-modifying agents (such as polypeptides and polynucleotides as provided herein).

Within other embodiments, immunotherapy may be passive immunotherapy, in which treatment involves the delivery of agents with established tumor-immune reactivity (such as effector cells or antibodies) that can directly or indirectly mediate antitumor effects and does not necessarily depend on an intact host immune system. Examples of effector cells include T cells as discussed above, T lymphocytes (such as CD8⁺ cytotoxic T lymphocytes and CD4⁺ T-helper tumor-infiltrating lymphocytes), killer cells (such as Natural Killer cells and lymphokine-activated killer cells), B cells and antigen-presenting cells (such as dendritic cells and macrophages) expressing a polypeptide provided herein. T cell receptors and antibody receptors specific for the polypeptides recited herein may be cloned, expressed and transferred into other vectors or effector cells for adoptive immunotherapy. The polypeptides provided herein may also be used to generate antibodies or anti-idiotypic antibodies (as described above and in U.S. Pat. No. 4,918,164) for passive immunotherapy.

Monoclonal antibodies may be labeled with any of a variety of labels for desired selective usages in detection, diagnostic assays or therapeutic applications (as described in U.S. Pat. Nos. 6,090,365; 6,015,542; 5,843,398; 5,595,721; and 4,708,930, hereby incorporated by reference in their entirety as if each was incorporated individually). In each case, the binding of the labelled monoclonal antibody to the determinant site of the antigen will signal detection or delivery of a particular therapeutic agent to the antigenic determinant on the non-normal cell. A further object of this invention is to provide the specific monoclonal antibody suitably labelled for achieving such desired selective usages thereof.

Effector cells may generally be obtained in sufficient quantities for adoptive immunotherapy by growth in vitro, as described herein. Culture conditions for expanding single antigen-specific effector cells to several billion in number with retention of antigen recognition in vivo are well known in the art. Such in vitro culture conditions typically use intermittent stimulation with antigen, often in the presence of cytokines (such as IL-2) and non-dividing feeder cells. As noted above, immunoreactive polypeptides as provided herein may be used to rapidly expand antigen-specific T cell cultures in order to generate a sufficient number of cells for immunotherapy. In particular, antigen-presenting cells, such as dendritic, macrophage, monocyte, fibroblast and/or B cells, may be pulsed with immunoreactive polypeptides or transfected with one or more polynucleotides using standard techniques well known in the art. For example, antigen-presenting cells can be transfected with a polynucleotide having a promoter appropriate for increasing expression in a recombinant virus or other expression system. Cultured effector cells for use in therapy must be able to grow and distribute widely, and to survive long term in vivo. Studies have shown that cultured effector cells can be induced to grow in vivo and to survive long term in substantial numbers by repeated stimulation with antigen supplemented with IL-2 (see, for example, Cheever et al., Immunological Reviews 157:177, 1997).

Alternatively, a vector expressing a polypeptide recited herein may be introduced into antigen presenting cells taken from a patient and clonally propagated ex vivo for transplant back into the same patient. Transfected cells may be reintroduced into the patient using any means known in the art, preferably in sterile form by intravenous, intracavitary, intraperitoneal or intratumor administration.

Routes and frequency of administration of the therapeutic compositions described herein, as well as dosage, will vary from individual to individual, and may be readily established using standard techniques. In general, the pharmaceutical compositions and vaccines may be administered by injection (e.g., intracutaneous, intramuscular, intravenous or subcutaneous), intranasally (e.g., by aspiration) or orally. Preferably, between 1 and 10 doses may be administered over a 52 week period. Preferably, 6 doses are administered, at intervals of 1 month, and booster vaccinations may be given periodically thereafter. Alternate protocols may be appropriate for individual patients. A suitable dose is an amount of a compound that, when administered as described above, is capable of promoting an anti-tumor immune response, and is at least 10-50% above the basal (i.e., untreated) level. Such response can be monitored by measuring the anti-tumor antibodies in a patient or by vaccine-dependent generation of cytolytic effector cells capable of killing the patient's tumor cells in vitro. Such vaccines should also be capable of causing an immune response that leads to an improved clinical outcome (e.g., more frequent remissions, complete or partial or longer disease-free survival) in vaccinated patients as compared to non-vaccinated patients. In general, for pharmaceutical compositions and vaccines comprising one or more polypeptides, the amount of each polypeptide present in a dose ranges from about 25 μg to 5 mg per kg of host. Suitable dose sizes will vary with the size of the patient, but will typically range from about 0.1 mL to about 5 mL.

In general, an appropriate dosage and treatment regimen provides the active compound(s) in an amount sufficient to provide therapeutic and/or prophylactic benefit. Such a response can be monitored by establishing an improved clinical outcome (e.g., more frequent remissions, complete or partial, or longer disease-free survival) in treated patients as compared to non-treated patients. Increases in preexisting immune responses to a tumor protein generally correlate with an improved clinical outcome. Such immune responses may generally be evaluated using standard proliferation, cytotoxicity or cytokine assays, which may be performed using samples obtained from a patient before and after treatment.

Cancer Detection and Diagnostic Compositions, Methods and Kits

In general, a cancer may be detected in a patient based on the presence of one or more colon tumor proteins and/or polynucleotides encoding such proteins in a biological sample (for example, blood, sera, sputum urine and/or tumor biopsies) obtained from the patient. In other words, such proteins may be used as markers to indicate the presence or absence of a cancer such as colon cancer. In addition, such proteins may be useful for the detection of other cancers. The binding agents provided herein generally permit detection of the level of antigen that binds to the agent in the biological sample.

Polynucleotide primers and probes may be used to detect the level of mRNA encoding a tumor protein, which is also indicative of the presence or absence of a cancer. In general, a tumor sequence should be present at a level that is at least two-fold, preferably three-fold, and more preferably five-fold or higher in tumor tissue than in normal tissue of the same type from which the tumor arose. Expression levels of a particular tumor sequence in tissue types different from that in which the tumor arose are irrelevant in certain diagnostic embodiments since the presence of tumor cells can be confirmed by observation of predetermined differential expression levels, e.g., 2-fold, 5-fold, etc, in tumor tissue to expression levels in normal tissue of the same type.

Other differential expression patterns can be utilized advantageously for diagnostic purposes. For example, in one aspect of the invention, overexpression of a tumor sequence in tumor tissue and normal tissue of the same type, but not in other normal tissue types, e.g. PBMCs, can be exploited diagnostically. In this case, the presence of metastatic tumor cells, for example in a sample taken from the circulation or some other tissue site different from that in which the tumor arose, can be identified and/or confirmed by detecting expression of the tumor sequence in the sample, for example using RT-PCR analysis. In many instances, it will be desired to enrich for tumor cells in the sample of interest, e.g., PBMCs, using cell capture or other like techniques.

There are a variety of assay formats known to those of ordinary skill in the art for using a binding agent to detect polypeptide markers in a sample. See, e.g., Harlow and Lane, Antibodies: A Laboratory Manual, Cold Spring Harbor Laboratory, 1988. In general, the presence or absence of a cancer in a patient may be determined by (a) contacting a biological sample obtained from a patient with a binding agent; (b) detecting in the sample a level of polypeptide that binds to the binding agent; and (c) comparing the level of polypeptide with a predetermined cut-off value.

In a preferred embodiment, the assay involves the use of binding agent immobilized on a solid support to bind to and remove the polypeptide from the remainder of the sample. The bound polypeptide may then be detected using a detection reagent that contains a reporter group and specifically binds to the binding agent/polypeptide complex. Such detection reagents may comprise, for example, a binding agent that specifically binds to the polypeptide or an antibody or other agent that specifically binds to the binding agent, such as an anti-immunoglobulin, protein G, protein A or a lectin. Alternatively, a competitive assay may be utilized, in which a polypeptide is labeled with a reporter group and allowed to bind to the immobilized binding agent after incubation of the binding agent with the sample. The extent to which components of the sample inhibit the binding of the labeled polypeptide to the binding agent is indicative of the reactivity of the sample with the immobilized binding agent. Suitable polypeptides for use within such assays include full length colon tumor proteins and polypeptide portions thereof to which the binding agent binds, as described above.

The solid support may be any material known to those of ordinary skill in the art to which the tumor protein may be attached. For example, the solid support may be a test well in a microtiter plate or a nitrocellulose or other suitable membrane. Alternatively, the support may be a bead or disc, such as glass, fiberglass, latex or a plastic material such as polystyrene or polyvinylchloride. The support may also be a magnetic particle or a fiber optic sensor, such as those disclosed, for example, in U.S. Pat. No. 5,359,681. The binding agent may be immobilized on the solid support using a variety of techniques known to those of skill in the art, which are amply described in the patent and scientific literature. In the context of the present invention, the term “immobilization” refers to both noncovalent association, such as adsorption, and covalent attachment (which may be a direct linkage between the agent and functional groups on the support or may be a linkage by way of a cross-linking agent). Immobilization by adsorption to a well in a microtiter plate or to a membrane is preferred. In such cases, adsorption may be achieved by contacting the binding agent, in a suitable buffer, with the solid support for a suitable amount of time. The contact time varies with temperature, but is typically between about 1 hour and about 1 day. In general, contacting a well of a plastic microtiter plate (such as polystyrene or polyvinylchloride) with an amount of binding agent ranging from about 10 ng to about 10 μg, and preferably about 100 ng to about 1 μg, is sufficient to immobilize an adequate amount of binding agent.

Covalent attachment of binding agent to a solid support may generally be achieved by first reacting the support with a bifunctional reagent that will react with both the support and a finctional group, such as a hydroxyl or amino group, on the binding agent. For example, the binding agent may be covalently attached to supports having an appropriate polymer coating using benzoquinone or by condensation of an aldehyde group on the support with an amine and an active hydrogen on the binding partner (see, e.g., Pierce Immunotechnology Catalog and Handbook, 1991, at A12-A13).

In certain embodiments, the assay is a two-antibody sandwich assay. This assay may be performed by first contacting an antibody that has been immobilized on a solid support, commonly the well of a microtiter plate, with the sample, such that polypeptides within the sample are allowed to bind to the immobilized antibody. Unbound sample is then removed from the immobilized polypeptide-antibody complexes and a detection reagent (preferably a second antibody capable of binding to a different site on the polypeptide) containing a reporter group is added. The amount of detection reagent that remains bound to the solid support is then determined using a method appropriate for the specific reporter group.

More specifically, once the antibody is immobilized on the support as described above, the remaining protein binding sites on the support are typically blocked. Any suitable blocking agent known to those of ordinary skill in the art, such as bovine serum albumin or Tween 20™ (Sigma Chemical Co., St. Louis, Mo.). The immobilized antibody is then incubated with the sample, and polypeptide is allowed to bind to the antibody. The sample may be diluted with a suitable diluent, such as phosphate-buffered saline (PBS) prior to incubation. In general, an appropriate contact time (i.e., incubation time) is a period of time that is sufficient to detect the presence of polypeptide within a sample obtained from an individual with colon cancer at least about 95% of that achieved at equilibrium between bound and unbound polypeptide. Those of ordinary skill in the art will recognize that the time necessary to achieve equilibrium may be readily determined by assaying the level of binding that occurs over a period of time. At room temperature, an incubation time of about 30 minutes is generally sufficient.

Unbound sample may then be removed by washing the solid support with an appropriate buffer, such as PBS containing 0.1% Tween 20™. The second antibody, which contains a reporter group, may then be added to the solid support. Preferred reporter groups include those groups recited above.

The detection reagent is then incubated with the immobilized antibody-polypeptide complex for an amount of time sufficient to detect the bound polypeptide. An appropriate amount of time may generally be determined by assaying the level of binding that occurs over a period of time. Unbound detection reagent is then removed and bound detection reagent is detected using the reporter group. The method employed for detecting the reporter group depends upon the nature of the reporter group. For radioactive groups, scintillation counting or autoradiographic methods are generally appropriate. Spectroscopic methods may be used to detect dyes, luminescent groups and fluorescent groups. Biotin may be detected using avidin, coupled to a different reporter group (commonly a radioactive or fluorescent group or an enzyme). Enzyme reporter groups may generally be detected by the addition of substrate (generally for a specific period of time), followed by spectroscopic or other analysis of the reaction products.

To determine the presence or absence of a cancer, such as colon cancer, the signal detected from the reporter group that remains bound to the solid support is generally compared to a signal that corresponds to a predetermined cut-off value. In one preferred embodiment, the cut-off value for the detection of a cancer is the average mean signal obtained when the immobilized antibody is incubated with samples from patients without the cancer. In general, a sample generating a signal that is three standard deviations above the predetermined cut-off value is considered positive for the cancer. In an alternate preferred embodiment, the cut-off value is determined using a Receiver Operator Curve, according to the method of Sackett et al., Clinical Epidemiology: A Basic Science for Clinical Medicine, Little Brown and Co., 1985, p. 106-7. Briefly, in this embodiment, the cut-off value may be determined from a plot of pairs of true positive rates (i.e., sensitivity) and false positive rates (100%-specificity) that correspond to each possible cut-off value for the diagnostic test result. The cut-off value on the plot that is the closest to the upper left-hand corner (i.e., the value that encloses the largest area) is the most accurate cut-off value, and a sample generating a signal that is higher than the cut-off value determined by this method may be considered positive. Alternatively, the cut-off value may be shifted to the left along the plot, to minimize the false positive rate, or to the right, to minimize the false negative rate. In general, a sample generating a signal that is higher than the cut-off value determined by this method is considered positive for a cancer.

In a related embodiment, the assay is performed in a flow-through or strip test format, wherein the binding agent is immobilized on a membrane, such as nitrocellulose. In the flow-through test, polypeptides within the sample bind to the immobilized binding agent as the sample passes through the membrane. A second, labeled binding agent then binds to the binding agent-polypeptide complex as a solution containing the second binding agent flows through the membrane. The detection of bound second binding agent may then be performed as described above. In the strip test format, one end of the membrane to which binding agent is bound is immersed in a solution containing the sample. The sample migrates along the membrane through a region containing second binding agent and to the area of immobilized binding agent. Concentration of second binding agent at the area of immobilized antibody indicates the presence of a cancer. Typically, the concentration of second binding agent at that site generates a pattern, such as a line, that can be read visually. The absence of such a pattern indicates a negative result. In general, the amount of binding agent immobilized on the membrane is selected to generate a visually discernible pattern when the biological sample contains a level of polypeptide that would be sufficient to generate a positive signal in the two-antibody sandwich assay, in the format discussed above. Preferred binding agents for use in such assays are antibodies and antigen-binding fragments thereof. Preferably, the amount of antibody immobilized on the membrane ranges from about 25 ng to about 1 μg, and more preferably from about 50 ng to about 500 ng. Such tests can typically be performed with a very small amount of biological sample.

Of course, numerous other assay protocols exist that are suitable for use with the tumor proteins or binding agents of the present invention. The above descriptions are intended to be exemplary only. For example, it will be apparent to those of ordinary skill in the art that the above protocols may be readily modified to use tumor polypeptides to detect antibodies that bind to such polypeptides in a biological sample. The detection of such tumor protein specific antibodies may correlate with the presence of a cancer.

A cancer may also, or alternatively, be detected based on the presence of T cells that specifically react with a tumor protein in a biological sample. Within certain methods, a biological sample comprising CD4⁺ and/or CD8⁺ T cells isolated from a patient is incubated with a tumor polypeptide, a polynucleotide encoding such a polypeptide and/or an APC that expresses at least an immunogenic portion of such a polypeptide, and the presence or absence of specific activation of the T cells is detected. Suitable biological samples include, but are not limited to, isolated T cells. For example, T cells may be isolated from a patient by routine techniques (such as by Ficoll/Hypaque density gradient centrifugation of peripheral blood lymphocytes). T cells may be incubated in vitro for 2-9 days (typically 4 days) at 37° C. with polypeptide (e.g., 5-25 μg/ml). It may be desirable to incubate another aliquot of a T cell sample in the absence of tumor polypeptide to serve as a control. For CD4⁺ T cells, activation is preferably detected by evaluating proliferation of the T cells. For CD8⁺ T cells, activation is preferably detected by evaluating cytol ytic activity. A level of proliferation that is at least two fold greater and/or a level of cytolytic activity that is at least 20% greater than in disease-free patients indicates the presence of a cancer in the patient.

As noted above, a cancer may also, or alternatively, be detected based on the level of mRNA encoding a tumor protein in a biological sample. For example, at least two oligonucleotide primers may be employed in a polymerase chain reaction (PCR) based assay to amplify a portion of a tumor cDNA derived from a biological sample, wherein at least one of the oligonucleotide primers is specific for (i.e., hybridizes to) a polynucleotide encoding the tumor protein. The amplified cDNA is then separated and detected using techniques well known in the art, such as gel electrophoresis.

Similarly, oligonucleotide probes that specifically hybridize to a polynucleotide encoding a tumor protein may be used in a hybridization assay to detect the presence of polynucleotide encoding the tumor protein in a biological sample.

To permit hybridization under assay conditions, oligonucleotide primers and probes should comprise an oligonucleotide sequence that has at least about 60%, preferably at least about 75% and more preferably at least about 90%, identity to a portion of a polynucleotide encoding a tumor protein of the invention that is at least 10 nucleotides, and preferably at least 20 nucleotides, in length. Preferably, oligonucleotide primers and/or probes hybridize to a polynucleotide encoding a polypeptide described herein under moderately stringent conditions, as defined above. Oligonucleotide primers and/or probes which may be usefully employed in the diagnostic methods described herein preferably are at least 10-40 nucleotides in length. In a preferred embodiment, the oligonucleotide primers comprise at least 10 contiguous nucleotides, more preferably at least 15 contiguous nucleotides, of a DNA molecule having a sequence as disclosed herein. Techniques for both PCR based assays and hybridization assays are well known in the art (see, for example, Mullis et al., Cold Spring Harbor Symp. Quant. Biol., 51:263, 1987; Erlich ed., PCR Technology, Stockton Press, NY, 1989).

One preferred assay employs RT-PCR, in which PCR is applied in conjunction with reverse transcription. Typically, RNA is extracted from a biological sample, such as biopsy tissue, and is reverse transcribed to produce cDNA molecules. PCR amplification using at least one specific primer generates a cDNA molecule, which may be separated and visualized using, for example, gel electrophoresis. Amplification may be performed on biological samples taken from a test patient and from an individual who is not afflicted with a cancer. The amplification reaction may be performed on several dilutions of cDNA spanning two orders of magnitude. A two-fold or greater increase in expression in several dilutions of the test patient sample as compared to the same dilutions of the non-cancerous sample is typically considered positive.

In another aspect of the present invention, cell capture technologies may be used in conjunction, with, for example, real-time PCR to provide a more sensitive tool for detection of metastatic cells expressing colon tumor antigens. Detection of colon cancer cells in biological samples, e.g., bone marrow samples, peripheral blood, and small needle aspiration samples is desirable for diagnosis and prognosis in colon cancer patients.

Immunomagnetic beads coated with specific monoclonal antibodies to surface cell markers, or tetrameric antibody complexes, may be used to first enrich or positively select cancer cells in a sample. Various commercially available kits may be used, including Dynabeads® Epithelial Enrich (Dynal Biotech, Oslo, Norway), StemSep™ (StemCell Technologies, Inc., Vancouver, BC), and RosetteSep (StemCell Technologies). A skilled artisan will recognize that other methodologies and kits may also be used to enrich or positively select desired cell populations. Dynabeads® Epithelial Enrich contains magnetic beads coated with mAbs specific for two glycoprotein membrane antigens expressed on normal and neoplastic epithelial tissues. The coated beads may be added to a sample and the sample then applied to a magnet, thereby capturing the cells bound to the beads. The unwanted cells are washed away and the magnetically isolated cells eluted from the beads and used in further analyses.

RosetteSep can be used to enrich cells directly from a blood sample and consists of a cocktail of tetrameric antibodies that targets a variety of unwanted cells and crosslinks them to glycophorin A on red blood cells (RBC) present in the sample, forming rosettes. When centrifuged over Ficoll, targeted cells pellet along with the free RBC. The combination of antibodies in the depletion cocktail determines which cells will be removed and consequently which cells will be recovered. Antibodies that are available include, but are not limited to: CD2, CD3, CD4, CD5, CD8, CD10, CD11b, CD14, CD15, CD16, CD19, CD20, CD24, CD25, CD29, CD33, CD34, CD36, CD38, CD41, CD45, CD45RA, CD45RO, CD56, CD66B, CD66e, HLA-DR, IgE, and TCRαβ.

Additionally, it is contemplated in the present invention that mAbs specific for colon tumor antigens can be generated and used in a similar manner. For example, mAbs that bind to tumor-specific cell surface antigens may be conjugated to magnetic beads, or formulated in a tetrameric antibody complex, and used to enrich or positively select metastatic colon tumor cells from a sample. Once a sample is enriched or positively selected, cells may be lysed and RNA isolated. RNA may then be subjected to RT-PCR analysis using colon tumor-specific primers in a real-time PCR assay as described herein. One skilled in the art will recognize that enriched or selected populations of cells may be analyzed by other methods (e.g. in situ hybridization or flow cytometry).

In another embodiment, the compositions described herein may be used as markers for the progression of cancer. In this embodiment, assays as described above for the diagnosis of a cancer may be performed over time, and the change in the level of reactive polypeptide(s) or polynucleotide(s) evaluated. For example, the assays may be performed every 24-72 hours for a period of 6 months to 1 year, and thereafter performed as needed. In general, a cancer is progressing in those patients in whom the level of polypeptide or polynucleotide detected increases over time. In contrast, the cancer is not progressing when the level of reactive polypeptide or polynucleotide either remains constant or decreases with time.

Certain in vivo diagnostic assays may be performed directly on a tumor. One such assay involves contacting tumor cells with a binding agent. The bound binding agent may then be detected directly or indirectly via a reporter group. Such binding agents may also be used in histological applications. Alternatively, polynucleotide probes may be used within such applications.

As noted above, to improve sensitivity, multiple tumor protein markers may be assayed within a given sample. It will be apparent that binding agents specific for different proteins provided herein may be combined within a single assay. Further, multiple primers or probes may be used concurrently. The selection of tumor protein markers may be based on routine experiments to determine combinations that results in optimal sensitivity. In addition, or alternatively, assays for tumor proteins provided herein may be combined with assays for other known tumor antigens.

The present invention further provides kits for use within any of the above diagnostic methods. Such kits typically comprise two or more components necessary for performing a diagnostic assay. Components may be compounds, reagents, containers and/or equipment. For example, one container within a kit may contain a monoclonal antibody or fragment thereof that specifically binds to a tumor protein. Such antibodies or fragments may be provided attached to a support material, as described above. One or more additional containers may enclose elements, such as reagents or buffers, to be used in the assay. Such kits may also, or alternatively, contain a detection reagent as described above that contains a reporter group suitable for direct or indirect detection of antibody binding.

Alternatively, a kit may be designed to detect the level of mRNA encoding a tumor protein in a biological sample. Such kits generally comprise at least one oligonucleotide probe or primer, as described above, that hybridizes to a polynucleotide encoding a tumor protein. Such an oligonucleotide may be used, for example, within a PCR or hybridization assay. Additional components that may be present within such kits include a second oligonucleotide and/or a diagnostic reagent or container to facilitate the detection of a polynucleotide encoding a tumor protein.

The following Examples are offered by way of illustration and not by way of limitation.

### EXAMPLES

### Example 1

### Generation of Colon Adenocarcinoma-specific Subtracted cDNA Libraries

Colon tumor subtracted cDNA libraries were constructed. Briefly, a pool of tester mRNA was collected from three colon adenocarcinoma samples showing moderate histological differentiation and no evidence of metastasis. Eight normal tissues, including brain, pancreas, bone marrow, liver, heart, lung, stomach and small intestine were represented in the driver mRNA pool. cDNA synthesis, hybridization and PCR amplification were performed according to the methods of Clontech (Palo Alto, Calif.), with minor modifications. In a first subtraction, the restriction enzymes PvuII, Dral, MscI and Stul were used to digest cDNAs. The tester to driver ratio was 1:40. In a second subtraction, Dral, MscI and Stul were used for cDNA digestion. A tester to driver ratio of 1:76 was employed. Following the PCR amplification steps, the cDNAs were cloned into the pCR2.1 plasmid vector. The libraries resulting from the first and second subtractions, named CPS 1 and CPS2, respectively, were used to obtain clones for microarray analysis and sequencing. Inserts were PCR amplified and purified. Each clone was sequenced from one direction with either M13 Forward primer or M13 Reverse primer.

In another subtraction, a cDNA library was constructed in the PCR2.1 vector (Invitrogen, Carlsbad, Calif.) by subtracting a pool of three colon tumors with a pool of normal colon, spleen, brain, liver, kidney, lung, stomach and small intestine using PCR subtraction methodologies (Clontech, Palo Alto, CA). The subtraction was performed using a PCR-based protocol, which was modified to generate larger fragments. Within this protocol, tester and driver double stranded cDNA were separately digested with five restriction enzymes that recognize six-nucleotide restriction sites (MluI, MscI, PvuII, SalI and StuI). This digestion resulted in an average cDNA size of 600 bp, rather than the average size of 300 bp that results from digestion with RsaI according to the Clontech protocol. This modification did not affect the subtraction efficiency. Two tester populations were then created with different adapters, and the driver library remained without adapters.

The tester and driver libraries were then hybridized using excess driver cDNA. In the first hybridization step, driver was separately hybridized with each of the two tester cDNA populations. This resulted in populations of (a) unhybridized tester cDNAs, (b) tester cDNAs hybridized to other tester cDNAs, (c) tester cDNAs hybridized to driver cDNAs, and (d) unhybridized driver cDNAs. The two separate hybridization reactions were then combined, and rehybridized in the presence of additional denatured driver cDNA. Following this second hybridization, in addition to populations (a) through (d), a fifth population (e) was generated in which tester cDNA with one adapter hybridized to tester cDNA with the second adapter. Accordingly, the second hybridization step resulted in enriclunent of differentially expressed sequences which could be used as templates for PCR amplification with adaptor-specific primers.

The ends were then filled in, and PCR amplification was performed using adaptor-specific primers. Only population (e), which contained tester cDNA that did not hybridize to driver cDNA, was amplified exponentially. A second PCR amplification step was then performed, to reduce background and further enrich differentially expressed sequences. This PCR-based subtraction technique normalizes differentially expressed cDNAs so that rare transcripts that are over-expressed in colon tumor tissue may be recoverable. Such transcripts would be difficult to recover by traditional subtraction methods.

The determined cDNA sequences for 333 clones from the colon tumor subtracted libraries are provided in SEQ ID NO:1-333.

### Example 2

### Analysis of Subtracted cDNA Sequences by Microarray Analysis

In additional studies, subtracted cDNA sequences were analyzed by microarray analysis to evaluate their expression in tumor and normal tissues. Using this approach, cDNA sequences are PCR amplified and their mRNA expression profiles in tumor and normal tissues are examined using cDNA microarray technology essentially as described (Shena et al., 1995). In brief, the clones are arrayed onto glass slides as multiple replicas, with each location corresponding to a unique cDNA clone (as many as 5500 clones can be arrayed on a single slide, or chip). Each chip is hybridized with a pair of cDNA probes that are fluorescence-labeled with Cy3 and Cy5, respectively. Typically, 1 μg of polyA⁺ RNA is used to generate each cDNA probe. After hybridization, the chips are scanned and the fluorescence intensity recorded for both Cy3 and Cy5 channels. There are multiple built-in quality control steps. First, the probe quality is monitored using a panel of ubiquitously expressed genes. Secondly, the control plate also can include yeast DNA fragments of which complementary RNA may be spiked into the probe synthesis for measuring the quality of the probe and the sensitivity of the analysis. Currently, the technology offers a sensitivity of 1 in 100,000 copies of mRNA. Finally, the reproducibility of this technology can be ensured by including duplicated control cDNA elements at different locations.

Analysis of colon tumor subtracted clones by microarray analysis on Colon Chip 3 identified the sequences set forth in SEQ ID NO:335-377 as being at least two-fold overexpressed in colon tumors versus normal tissues.

### Example 3

### Identification of Normal Colon cDNAs

Clones were derived from a characterization of a primary normal colon library. Two normal colon tissue samples were represented in the mRNA pool. These clones were sequenced and data base searches performed. SEQ ID NO:334 disclosed herein showed no homology to known sequences.

### Example 4

### Analysis of C592S cDNA Expression Using Real-time PCR

The colon tumor antigen, C592S (SEQ ID NO:348), was isolated from the subtraction library described in Example 1 and was found by microarray analysis to be overexpressed in colon tumors as compared to normal colon tissue. This sequence shows no significant similarity to known sequences in Genbank. The expression pattern of this gene was further analyzed by real-time PCR, as described below, and was found to be overexpressed in colon tumor while it was expressed at lower levels in normal colon. No expression was observed in a panel of other normal tissues. This data indicates that C592S may be valuable as a tumor immunotherapeutic or diagnostic tool.

The first-strand cDNA to be used in the quantitative real-time PCR was synthesized from 20 μg of total RNA that had been treated with DNase I (Amplification Grade, Gibco BRL Life Technology, Gaithersburg, Md.), using Superscript Reverse Transcriptase (RT) (Gibco BRL Life Technology, Gaithersburg, Md.). Real-time PCR was performed with a GeneAmp™ 5700 sequence detection system (PE Biosystems, Foster City, Calif.). The 5700 system uses SYBR™ green, a fluorescent dye that only intercalates into double stranded DNA, and a set of gene-specific forward and reverse primers. The increase in fluorescence is monitored during the whole amplification process. The optimal concentration of primers was determined using a checkerboard approach and a pool of cDNAs from breast tumors was used in this process. The PCR reaction was performed in 25 μl volumes that include 2.5 μl of SYBR green buffer, 2 μl of cDNA template and 2.5 μl each of the forward and reverse primers for the gene of interest. The cDNAs used for RT reactions were diluted 1:10 for each gene of interest and 1:100 for the β-actin control. In order to quantitate the amount of specific cDNA (and hence initial mRNA) in the sample, a standard curve is generated for each run using the plasmid DNA containing the gene of interest. Standard curves were generated using the Ct values determined in the real-time PCR which were related to the initial cDNA concentration used in the assay. Standard dilution ranging from 20-2×10⁶ copies of the gene of interest was used for this purpose. In addition, a standard curve was generated for β-actin ranging from 200 fg-2000 fg. This enabled standardization of the initial RNA content of a tissue sample to the amount of β-actin for comparison purposes. The mean copy number for each group of tissues tested was normalized to a constant amount of β-actin, allowing the evaluation of the over-expression levels seen with each of the genes.

### EXAMPLE 5

### Peptide Priming of T-helper Lines

Generation of CD4⁺ T helper lines and identification of peptide epitopes derived from tumor-specific antigens that are capable of being recognized by CD4⁺ T cells in the context of HLA class II molecules, is carried out as follows:

Fifteen-mer peptides overlapping by 10 amino acids, derived from a tumor-specific antigen, are generated using standard procedures. Dendritic cells (DC) are derived from PBMC of a normal donor using GM-CSF and IL-4 by standard protocols. CD4⁺ T cells are generated from the same donor as the DC using MACS beads (Miltenyi Biotec, Auburn, Calif.) and negative selection. DC are pulsed overnight with pools of the 15-mer peptides, with each peptide at a final concentration of 0.25 μg/ml. Pulsed DC are washed and plated at 1×10⁴ cells/well of 96-well V-bottom plates and purified CD4⁺ T cells are added at 1×10⁵/well. Cultures are supplemented with 60 ng/ml IL-6 and 10 ng/ml IL-12 and incubated at 37° C. Cultures are restimulated as above on a weekly basis using DC generated and pulsed as above as antigen presenting cells, supplemented with 5 ng/ml IL-7 and 10 U/ml IL-2. Following 4 in vitro stimulation cycles, resulting CD4⁺ T cell lines (each line corresponding to one well) are tested for specific proliferation and cytokine production in response to the stimulating pools of peptide with an irrelevant pool of peptides used as a control.

### Example 6

### Generation of Tumor-specific CTL Lines Using In Vitro Whole-gene Priming

Using in vitro whole-gene priming with tumor antigen-vaccinia infected DC (see, for example, Yee et al, The Journal of Immunology, 157(9):4079-86, 1996), human CTL lines are derived that specifically recognize autologous fibroblasts transduced with a specific tumor antigen, as determined by interferon-y ELISPOT analysis. Specifically, dendritic cells (DC) are differentiated from monocyte cultures derived from PBMC of normal human donors by growing for five days in RPMI medium containing 10% human serum, 50 ng/ml human GM-CSF and 30 ng/ml human IL-4. Following culture, DC are infected overnight with tumor antigen-recombinant vaccinia virus at a multiplicity of infection (M.O.I) of five, and matured overnight by the addition of 3 μg/ml CD40 ligand. Virus is then inactivated by UV irradiation. CD8⁺ T cells are isolated using a magnetic bead system, and priming cultures are initiated using standard culture techniques. Cultures are restimulated every 7-10 days using autologous primary fibroblasts retrovirally transduced with previously identified tumor antigens. Following four stimulation cycles, CD8⁺ T cell lines are identified that specifically produce interferon-γ when stimulated with tumor antigen-transduced autologous fibroblasts. Using a panel of HLA-mismatched B-LCL lines transduced with a vector expressing a tumor antigen, and measuring interferon-γ production by the CTL lines in an ELISPOT assay, the HLA restriction of the CTL lines is determined.

### Example 7

### Generation and Characterization of Anti-tumor Antigen Monoclonal Antibodies

Mouse monoclonal antibodies are raised against E. coli derived tumor antigen proteins as follows: Mice are immunized with Complete Freund's Adjuvant (CFA) containing 50 μg recombinant tumor protein, followed by a subsequent intraperitoneal boost with Incomplete Freund's Adjuvant (IFA) containing 10 μg recombinant protein. Three days prior to removal of the spleens, the mice are immunized intravenously with approximately 50 μg of soluble recombinant protein. The spleen of a mouse with a positive titer to the tumor antigen is removed, and a single-cell suspension made and used for fusion to SP2/O myeloma cells to generate B cell hybridomas. The supernatants from the hybrid clones are tested by ELISA for specificity to recombinant tumor protein, and epitope mapped using peptides that spanned the entire tumor protein sequence. The mAbs are also tested by flow cytometry for their ability to detect tumor protein on the surface of cells stably transfected with the cDNA encoding the tumor protein.

What is claimed:

1. An isolated polynucleotide comprising a sequence selected from the group consisting of:
(a) sequences provided in SEQ ID NO:1-377;
(b) complements of the sequences provided in SEQ ID NO:1-377;
(c) sequences consisting of at least 20 contiguous residues of a sequence provided in SEQ ID NO:1-377;
(d) sequences that hybridize to a sequence provided in SEQ ID NO:1-377, under highly stringent conditions;
(e) sequences having at least 75% identity to a sequence of SEQ ID NO:1-377;
(f) sequences having at least 90% identity to a sequence of SEQ ID NO:1-377; and
(g) degenerate variants of a sequence provided in SEQ ID NO:1-377.

2. An isolated polypeptide comprising an amino acid sequence selected from the group consisting of:
(a) sequences encoded by a polynucleotide of claim 1; and
(b) sequences having at least 70% identity to a sequence encoded by a polynucleotide of claim 1; and
(c) sequences having at least 90% identity to a sequence encoded by a polynucleotide of claim 1.

3. An expression vector comprising a polynucleotide of claim 1 operably linked to an expression control sequence.

4. A host cell transformed or transfected with an expression vector according to claim 3.

5. An isolated antibody, or antigen-binding fragment thereof, that specifically binds to a polypeptide of claim 2.

6. A method for detecting the presence of a cancer in a patient, comprising the steps of:
(a) obtaining a biological sample from the patient;
(b) contacting the biological sample with a binding agent that binds to a polypeptide of claim 2;
(c) detecting in the sample an amount of polypeptide that binds to the binding agent; and
(d) comparing the amount of polypeptide to a predetermined cut-off value and therefrom determining the presence of a cancer in the patient.

7. A fusion protein comprising at least one polypeptide according to claim 2.

8. An oligonucleotide that hybridizes to a sequence recited in SEQ ID NO:1-377 under highly stringent conditions.

9. A method for stimulating and/or expanding T cells specific for a tumor protein, comprising contacting T cells with at least one component selected from the group consisting of:
(a) polypeptides according to claim 2;
(b) polynucleotides according to claim 1; and
(c) antigen-presenting cells that express a polynucleotide according to claim 1,
under conditions and for a time sufficient to permit the stimulation and/or expansion of T cells.

10. An isolated T cell population, comprising T cells prepared according to the method of claim 9.

11. A composition comprising a first component selected from the group consisting of physiologically acceptable carriers and immunostimulants, and a second component selected from the group consisting of:
(a) polypeptides according to claim 2;
(b) polynucleotides according to claim 1;
(c) antibodies according to claim 5;
(d) fusion proteins according to claim 7;
(e) T cell populations according to claim 10; and
(f) antigen presenting cells that express a polypeptide according to claim 2.

12. A method for stimulating an immune response in a patient, comprising administering to the patient a composition of claim 11.

13. A method for the treatment of a colon cancer in a patient, comprising administering to the patient a composition of claim 11.

14. A method for determining the presen ce of a cancer in a patient, comprising the steps of:
(a) obtaining a biological sample from the patient;
(b) contacting the biological sample with an oligonucleotide according to claim 8;
(c) detecting in the sample an amount of a polynucleotide that hybridizes to the oligonucleotide; and
(d) compare the amount of polynucleotide that hybridizes to the oligonucleotide to a predetermined cut-off value, and therefrom determining the presence of the cancer in the patient.

15. A diagnostic kit comprising at least one oligonucleotide according to claim 8.

16. A diagnostic kit comprising at least one antibody according to claim 5 and a detection reagent, wherein the detection reagent comprises a reporter group.

17. A method for the treatment of colon cancer in a patient, comprising the steps of:
(a) incubating CD4+ and/or CD8+ T cells isolated from a patient with at least one component selected from the group consisting of: (i) polypeptides according to claim 2; (ii) polynucleotides according to claim 1; and (iii) antigen presenting cells that express a polypeptide of claim 2, such that T cell proliferate;
(b) administering to the patient an effective amount of the proliferated T cells, and thereby inhibiting the development of a cancer in the patient.
